(19) United States
(12) Patent Application Publication
(10) Pub. No.: US 2026/0108606 A1
(43) Pub. Date: Apr. 23, 2026

(54) TREATMENT OF AUTOIMMUNE DISEASES HAVING A PATHOGENIC T CELL STATE

(71) Applicant: THE BRIGHAM AND WOMEN'S HOSPITAL, INC., Boston, MA (US)

(72) Inventors: Ana Carrizosa ANDERSON, Boston, MA (US); Davide MANGANI, Boston, MA (US)

(73) Assignee: THE BRIGHAM AND WOMEN'S HOSPITAL, INC., Boston, MA (US)

(21) Appl. No.: 19/367,293

(22) Filed: Oct. 23, 2025

Related U.S. Application Data
(63) Continuation of application No. PCT/US2024/026323, filed on Apr. 25, 2024.
(60) Provisional application No. 63/461,842, filed on Apr. 25, 2023.

(51) Int. Cl. A61K 40/36; A61K 40/11; A61P 37/06; C12N 15/85

(52) U.S. Cl. CPC A61K 40/36; A61K 40/11; A61P 37/06; C12N 15/85; C12N 2800/107

(57) ABSTRACT

The subject matter disclosed herein is generally directed to methods of treating autoimmune and inflammatory diseases characterized by a pathogenic immune cell state. Disclosed are novel gene dependencies in the pathogenic immune cell state. Also, disclosed are methods for modifying immune cells characterized by the pathogenic immune cell state.

### CROSS-REFERENCE TO RELATED APPLICATION

The present application is a continuation of International Application No. PCT/US2024/026323, filed on Apr. 25, 2024, and claims priority under 35 U.S.C. § 119 (e) to U.S. provisional patent application No. 63/461,842, entitled “TREATMENT OF AUTOIMMUNE DISEASES HAVING A PATHOGENIC T CELL STATE,” filed Apr. 25, 2023. The entire contents of the aforementioned patent applications are incorporated herein by reference.

### STATEMENT AS TO FEDERALLY SPONSORED RESEARCH

This invention was made with government support under Grant No. AI176341 awarded by the National Institutes of Health. The government has certain rights in the invention.

### TECHNICAL FIELD

The subject matter disclosed herein is generally directed to methods of treating autoimmune diseases having a pathogenic T cell state by targeting novel vulnerabilities in T cells expressing a pathogenic T cell signature or transcription factors implicit in various autoimmune diseases.

### SEQUENCE LISTING

The instant application contains a Sequence Listing which has been filed electronically in XML format and is hereby incorporated by reference in its entirety. Said XML file, created on Dec. 24, 2025, is named 114203-2741_SL.xml and is 35,814 Bytes in size.

### BACKGROUND

CD4+ T helper 17 (Th17) cells encompass a spectrum of states, including homeostatic cells that maintain physiological functions and pathogenic cells that drive autoimmune tissue inflammation. However, the regulatory landscape driving different Th17 cell states is poorly understood. As such, there exists a need for improved understanding of the Th17 regulatory landscape and interventions for improved autoimmune disease and inflammation management.

Citation or identification of any document in this application is not an admission that such a document is available as prior art to the present disclosure.

### SUMMARY

In one aspect, the disclosure provides for a method of treating an autoimmune disease in a subject, comprising contacting a Th17 cell or a plurality of Th17 cells with an exogenous T cell modulating agent in an amount sufficient to: decrease expression of at least one pro-inflammatory Th17 transcription factor, increase expression of at least one anti-inflammatory Th17 transcription factor, or a combination thereof, in order to treat the autoimmune disease.

In some embodiments, the autoimmune disease is selected from the group consisting of: acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopenia purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anemia; polyarteritis nodosa; polymyositis; primary biliary cirrhosis; primary myxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; Wegener's granulomatosis asthma, allergy, allergic rhinitis, allergic airway inflammation, atopic dermatitis (AD), chronic obstructive pulmonary disease (COPD), inflammatory bowel disease (IBD), Irritable bowel syndrome (IBS), arthritis, psoriasis, eosinophilic esophagitis, eosinophilic pneumonia, eosinophilic psoriasis, hypereosinophilic syndrome, graft-versus-host disease, uveitis, cardiovascular disease, pain, lupus, vasculitis, chronic idiopathic urticaria, and Eosinophilic Granulomatosis with Polyangiitis (Churg-Strauss Syndrome). In some embodiments, wherein the autoimmune disease is multiple sclerosis (MS), rheumatoid arthritis (RA), psoriasis, type-1 diabetes (TID), or inflammatory bowel disease (IBD).

In some embodiments, the at least one pro-inflammatory Th17 transcription factor or the at least one anti-inflammatory Th17 transcription factor is selected from the group consisting of: HEB (HeLa E-box binding protein), FOXO1 (Forkhead box 01), GABPA (GA-binding protein transcription factor alpha subunit), FLI1 (Friend leukemia integration 1 transcription factor), ELK4 (ETS domain-containing protein Elk-4), EGR1 (Early growth response 1), TCF1 (Transcription factor 1), ETS1 (ETS proto-oncogene 1, transcription factor), Jun (Jun proto-oncogene, AP-1 transcription factor subunit), Fosl2 (FOS like 2, AP-1 transcription factor subunit), Junb (Jun B proto-oncogene), Fos (Fos proto-oncogene, AP-1 transcription factor subunit), Jund (Jun D proto-oncogene), Tfe3 (Transcription factor binding to IGHM enhancer 3), Usf1 (Upstream transcription factor 1), Usf2 (Upstream transcription factor 2), Clock (Circadian locomotor output cycles kaput), Arntl (Aryl hydrocarbon receptor nuclear translocator-like protein 1), Arnt (Aryl hydrocarbon receptor nuclear translocator), Bhlhe40 (Basic helix-loop-helix family member e40), Tfeb (Transcription factor EB), Mxi1 (MAX interactor 1), Max (MYC associated factor X), Myc (MYC proto-oncogene), Nfyb (Nuclear transcription factor Y subunit beta), Nfya (Nuclear transcription factor Y subunit alpha), Nfyc (Nuclear transcription factor Y subunit gamma), Ddit3 (DNA damage inducible transcript 3), Atf4 (Activating transcription factor 4), Cebpg (CCAAT/enhancer binding protein gamma), Zbtb7a (Zinc finger and BTB domain containing 7A), Mbd2 (Methyl-CpG binding domain protein 2), Epas1 (Endothelial PAS domain protein 1), Hif1a (Hypoxia inducible factor 1 subunit alpha), Mecp2 (Methyl-CpG binding protein 2), Xbp1 (X-box binding protein 1), Cux1 (Cut-like homeobox 1), Zbtb33 (Zinc finger and BTB domain containing 33), E2f2 (E2F transcription factor 2), E2f5 (E2F transcription factor 5), Hinfp (Histone H4 transcription factor), Mtf1 (Metal regulatory transcription factor 1), Nrf1 (Nuclear respiratory factor 1), Pax5 (Paired box 5), Yy1 (Yin Yang 1), Pou1f1 (POU class 1 homeobox 1), Nfe2l1 (Nuclear factor, erythroid 2 like 1), Tead2 (TEA domain transcription factor 2), Arid5b (AT-rich interaction domain 5B), Sox5 (SRY-box transcription factor 5), Foxp2 (Forkhead box P2), Foxk1 (Forkhead box K1), Foxo3 (Forkhead box O3), Nr1d1 (Nuclear receptor subfamily 1 group D member 1), Nr1d2 (Nuclear receptor subfamily 1 group D member 2), Rora (RAR-related orphan receptor A), Rorc (RAR-related orphan receptor C), Stat5a (Signal transducer and activator of transcription 5A), Stat5b (Signal transducer and activator of transcription 5B), Stat3 (Signal transducer and activator of transcription 3), Stat4 (Signal transducer and activator of transcription 4), Rel (REL proto-oncogene, NF-kB subunit), Relb (REL proto-oncogene B), Rela (RELA proto-oncogene, NF-kB subunit), Nfkb1 (Nuclear factor kappa B subunit 1), Nfkb2 (Nuclear factor kappa B subunit 2), Maff (MAF bZIP transcription factor F), Mafk (MAF bZIP transcription factor K), Maf (MAF bZIP transcription factor), Mafg (MAF bZIP transcription factor G), Bach2 (BTB domain and CNC homolog 2), Nfe212 (Nuclear factor, erythroid 2 like 2), Bach1 (BTB domain and CNC homolog 1), Batf (Basic leucine zipper ATF-like transcription factor), Foxj3 (Forkhead box J3), Foxj2 (Forkhead box J2), Foxo4 (Forkhead box 04), Prdm1 (PR domain zinc finger protein 1), Irf7 (Interferon regulatory factor 7), Irf9 (Interferon regulatory factor 9), Lyl1 (Lymphoblastic leukemia derived sequence 1), Tcf12 (Transcription factor 12), Tcf3 (Transcription factor 3), Smad3 (SMAD family member 3), Smad2 (SMAD family member 2), Smad4 (SMAD family member 4), Mef2c (Myocyte enhancer factor 2C), Mef2a (Myocyte enhancer factor 2A), Mef2d (Myocyte enhancer factor 2D), Rfx3 (Regulatory factor X3), Rfx1 (Regulatory factor X1), Rfx2 (Regulatory factor X2), Rara (Retinoic acid receptor alpha), Rxra (Retinoid X receptor alpha), Pparg (Peroxisome proliferator-activated receptor gamma), Nr1h3 (Nuclear receptor subfamily 1 group H member 3), Nr2c2 (Nuclear receptor subfamily 2 group C member 2), Ar (Androgen receptor), Nr3c1 (Nuclear receptor subfamily 3 group C member 1), Pbx1 (Pre-B-cell leukemia homeobox 1), Pknox1 (PBX/knotted 1 homeobox 1), Esrra (Estrogen related receptor alpha), Esrrg (Estrogen related receptor gamma), Esr1 (Estrogen receptor 1), Rarg (Retinoic acid receptor gamma), Stat6 (Signal transducer and activator of transcription 6), Nfatc1 (Nuclear factor of activated T cells 1), Nfatc2 (Nuclear factor of activated T cells 2), Nfatc3 (Nuclear factor of activated T cells 3), Ebf1 (Early B-cell factor 1), Prdm16 (PR domain containing 16), Atf2 (Activating transcription factor 2), Crem (CAMP responsive element modulator), Atf1 (Activating transcription factor 1), Creb1 (CAMP responsive element binding protein 1), Nr2c1 (Nuclear receptor subfamily 2 group C member 1), Rxrb (Retinoid X receptor beta), Mecom (MDS1 and EVI1 complex locus), Gata1 (GATA binding protein 1), Gata3 (GATA binding protein 3), Prdm5 (PR domain containing 5), Tcf4 (Transcription factor 4), Zeb1 (Zinc finger E-box binding homeobox 1), Hsf1 (Heat shock transcription factor 1), Hsf2 (Heat shock transcription factor 2), Ikzf1 (IKAROS family zinc finger 1), Ctcf (CCCTC-binding factor), Zfx (Zinc finger protein X-linked), Bcl6 (B-cell lymphoma 6 protein), Nfia (Nuclear factor I A), Nfic (Nuclear factor I C), Smarca5 (SWI/SNF related, matrix associated, actin dependent regulator of chromatin, subfamily a, member 5), Thap11 (THAP domain containing 11), Dbp (D site of albumin promoter binding protein), Nfil3 (Nuclear factor interleukin 3 regulated), Thra (Thyroid hormone receptor alpha), Tbp (TATA-box binding protein), Pou2f1 (POU class 2 homeobox 1), Pou2f2 (POU class 2 homeobox 2), Zkscan1 (Zinc finger with KRAB and SCAN domains 1), Gfi1 (Growth factor independent 1 transcription repressor), Pbx2 (Pre-B-cell leukemia homeobox 2), Rest (REI-silencing transcription factor), Srf (Serum response factor), Nr4a1 (Nuclear receptor subfamily 4 group A member 1), Prdm9 (PR domain containing 9), Rbpj (Recombination signal binding protein for immunoglobulin kappa J region), Tbx21 (T-box transcription factor 21), Srebf1 (Sterol regulatory element binding transcription factor 1), Srebf2 (Sterol regulatory element binding transcription factor 2), Taf1 (TATA-box binding protein associated factor 1), Tbx20 (T-box transcription factor 20), Meis2 (Meis homeobox 2), Tgif1 (TGFB induced factor homeobox 1), Maz (MYC associated zinc finger protein), Klf3 (Kruppel-like factor 3), Klf6 (Kruppel-like factor 6), Sp1 (Sp1 transcription factor), Zbtb17 (Zinc finger and BTB domain containing 17), Sp4 (Sp4 transcription factor), Sp2 (Sp2 transcription factor), Sp3 (Sp3 transcription factor), Egr1 (Early growth response 1), Egr2 (Early growth response 2), E2f6 (E2F transcription factor 6), E2f1 (E2F transcription factor 1), E2f4 (E2F transcription factor 4), Spi1 (Spi-1 proto-oncogene), Spib (Spi-B transcription factor), Ets1 (ETS proto-oncogene 1), Elf1 (E74-like factor 1), Elk1 (ETS domain-containing protein Elk-1), Gabpa (GA-binding protein transcription factor alpha subunit), Elk4 (ETS domain-containing protein Elk-4), Fli1 (Friend leukemia integration 1 transcription factor), Ets2 (ETS proto-oncogene 2), Elf2 (E74-like factor 2), Etv6 (ETS variant transcription factor 6), Cbfb (Core-binding factor beta subunit), Runx3 (Runt-related transcription factor 3), Runx1 (Runt-related transcription factor 1), Runx2 (Runt-related transcription factor 2), Lef1 (Lymphoid enhancer-binding factor 1), Tcf7 (Transcription factor 7), Irf4 (Interferon regulatory factor 4), Irf8 (Interferon regulatory factor 8), Irf1 (Interferon regulatory factor 1), Irf2 (Interferon regulatory factor 2), Irf3 (Interferon regulatory factor 3), Stat1 (Signal transducer and activator of transcription 1), Stat2 (Signal transducer and activator of transcription 2).

In some embodiments, the at least one pro-inflammatory Th17 transcription factor is selected from the group consisting of RUNX1, SP1, STAT3, IRF4, RORG, RORA, STAT1, BATF, JUNB, and FOSL2. In some embodiments, the at least one pro-inflammatory Th17 transcription factor is selected from the group consisting of TCF1, Lef1, Bach2, and Rora.

In some embodiments, a transcription level or a protein expression level of the at least one pro-inflammatory Th17 transcription factor is decreased. In some embodiments, the at least one anti-inflammatory Th17 transcription factor is selected from the group consisting of HEB, FOXO1, GABPA, FLI1, ELK4, EGR1, TCF1, and ETS1. In some embodiments, a transcription level or a protein expression level of the at least one anti-inflammatory Th17 transcription factor is increased. In some embodiments, the at least one anti-inflammatory Th17 transcription factor is TCF1.

In some embodiments, expression of Purinergic receptor P2X 7 (P2rx7), Purinergic receptor P2X 4 (P2rx4), Interleukin 17A (Il17a), Interleukin 21 receptor (Il21r), Colony stimulating factor 2 (Csf2), Interleukin 1 receptor type 1 (Il1r1), Fas ligand (Fasl), Stearoyl-CoA desaturase 1 (Scd1), Hydroxyacyl-CoA dehydrogenase (Hadh), Solute carrier family 2 member 1 (Slc2a1), AKT serine/threonine kinase 2 (Akt2), Interleukin 21 (Il21), Granzyme B (Gzmb), Lymphocyte-specific protein tyrosine kinase (Lck), SRC proto-oncogene, non-receptor tyrosine kinase (Src), RAR-related orphan receptor A (Rora), RAR-related orphan receptor C (Rorc), Basic helix-loop-helix family member e40 (Bhlhe40), Peroxisome proliferator activated receptor gamma (Pparg), ATP binding cassette subfamily A member 1 (Abca1), Hexokinase 2 (Hk2), Glycogen phosphorylase, liver form (Pygl), ADAM metallopeptidase domain 19 (Adam 19), Integrin subunit beta 1 (Itgfb1), CD47 molecule (Cd47), C-C motif chemokine receptor 6 (Ccr6), C—X—C motif chemokine receptor 6 (Cxc4r6), TNF superfamily member 14 (Tnfsf14), Colony stimulating factor 1 (Csf1), BCL2 apoptosis regulator (Bcl2), Transmembrane protein 59 (Tmem59), Solute carrier family 2 member 3 (Slc2a3), G protein-coupled receptor 65 (Gpr65), Integrin subunit beta 7 (Itgb7), Integrin subunit beta 3 (Itgb3), or any combination thereof is decreased. In some embodiments, expression of Abhydrolase Domain Containing Acylglycerol Lipase 2 (Abhd2), Oxysterol Binding Protein Like 9 (Osbp19), Low Density Lipoprotein Receptor Adaptor Protein 1 (Ldlrap1), Propionyl-CoA Carboxylase Alpha Subunit (Pcca), Cyclin D Binding Myb Like Transcription Factor 1 (Dmtf1), B Cell Translocation Gene 1 (Btg1), Structural Maintenance of Chromosomes 6 (Smc6), Fanconi Anemia Complementation Group I (Fanci), Ubiquitin Specific Peptidase 28 (Usp28), BCL6 Corepressor (Bcor), SP100 Nuclear Antigen (Sp100), Forkhead Box O1 (Foxo1), DNA Methyltransferase 3 Alpha (Dnmt3a), Protein Phosphatase, Mg2+/Mn2+Dependent 1G (Ppm1g), Transforming Growth Factor Beta Receptor II (Tgfbr2), SMAD Family Member 5 (Smad5), Interleukin 10 Receptor Subunit Beta (Il10rb), Interleukin 10 (Il10), Dual Specificity Phosphatase 2 (Dusp2), Dual Specificity Phosphatase 4 (Dusp4), Transforming Growth Factor Beta Receptor III (Tgfbr3), and Left-Right Determination Factor 1 (Lefty1), or any combination thereof is increased.

In some embodiments, the exogenous T cell modulating agent increases TCF1 expression. In some embodiments, the exogenous T cell modulating agent inhibits TCF1 expression from decreasing. In some embodiments, the exogenous T cell modulating agent that decreases expression of the at least one pro-inflammatory Th17 transcription factor is selected from the group consisting of: a. one or more vectors for knocking out one or more genes from Table 1A and/or Table 1B; b. a RNAi agent for decreasing expression of one or more genes from Table 1A and/or Table 1B; c. a genetic modification system linked to or otherwise capable of complexing with a transcription repressor that blocks transcription of one or more genes from Table 1A and/or Table 1B; d. a genetic modification system capable of making one or more epigenetic edits to the cell genome such that expression of one or more genes from Table 1A and/or Table 1B is silenced; e. a genetic modification system configured to modify one or more enhancers controlling expression of one or more genes in Table 1A and/or Table 1B such that binding of transcription factors or other regulatory proteins needed to initiate transcription is reduced; f. a genetic modification system configured to remove or modify one or more promoters controlling expression of one or more genes in Table 1A and/or Table 1B, or configured to replace one or more promoters with a weakened promoter, such that binding of transcription factors and/or RNA polymerase binding is blocked or weakened; g. a genetic modification system configured to introduce a silencer element, or to modify an existing silencer element, in a non-coding region controlling expression of one or more genes from Table 1A and/or Table 1B, such that transcriptional repressors that block or decrease expression are recruited to the non-coding region; h. a genetic modification system configured to disrupt or replace an existing insulator region controlling expression of one or more genes from Table 1A and/or Table 1B such that insulator function on silencer elements or repressive chromatin structures is reduced; I a genetic modification system configured to introduce a frame-shift mutation in a coding region of one or more genes from Table 1A and/or Table 1B such that a pre-mature stop codon is introduced; j. a genetic modification system configured to introduce insertions, deletions, or substitutions in coding regions of one or more genes from Table 1A and/or Table 1B that encode one or more functional domains of the expressed gene product such that a non-functional gene or gene products with reduced function or activity are produced; k. a genetic modification system configured to introduce one or more modification at one or more exon-intron boundaries or splice sites leading to aberrant splicing or production of non-functional proteins or triggering of non-sense mediated RNA decay; 1. a genetic modification system configured to introduce one or more modifications to a regulatory element within the coding regions of one or more genes from Table 1A and/or Table 1B such the expression is decreased; m. a genetic modification system configured to modify sequences encoding one or more post-translational modification (PTM) sites such that one or more activating PTM is removed, or one or more inhibitory PTM that decreases protein function or stability or increases protein degradation is increased; n. a genetic modification system configured to introduce or remove one or more chromatin loops controlling expression of one or more genes in Table 1A and/or Table 1B such that gene expression is reduced; and o. combinations thereof.

In some embodiments, the genetic modification system linked to or otherwise capable of complexing with a transcription repressor that blocks transcription is catalytically inactive.

In some embodiments, the genetic modification system is a CRISPR-Cas system, a base editing system, a prime editing system, a CAST system, a non-LTR retrotransposon, or a Cas-directed recombinase. In some embodiments, wherein the genetic modification system is catalytically inactive Cas linked to or otherwise associated with an epigenetic modifier.

In some embodiments, the exogenous T cell modulating agent which increases expression of at least one anti-inflammatory Th17 transcription factor is selected from the group consisting of: a. one or more vectors for introducing one or more additional copies of one or more genes from Table 2A and/or Table 2B; b. a genetic modification system configured to modify one or more enhancer regions controlling expression of one or more genes in Table 2A and/or Table 2B such that binding of transcription factors or other regulatory proteins is increased; c. a genetic modification system configured to modify one or more promoters controlling expression of one or more genes of Table 2A and/or Table 2B such that expression is increased; d. a genetic modification system configured to modify one or more silencer regions controlling expression of one or more genes in Table 2A and/or Table 2B, such that binding of transcriptional repressor is blocked or reduced and gene expression is increased; e. a genetic modification system configured to modify or remove one or more regulatory elements in a coding sequence of one or more genes in Table 2A and/or Table 2B; f. a genetic modification system configured to introduce or strengthen one or more insulator regions controlling expression of one or more genes in Table 2A and/or Table 2B; g. a genetic modification system configured to introduce one or more modifications in a coding region that increases gene product stability, folding, or resistance to degradation or enhances the function or activity of the gene product; h. a genetic modification system configured to remove one or more sequences encoding an inhibitory PTM, remove one or more ubiquitination sites, and/or add one or more activating PTM sites; i. a genetic modification system configured to introduce or remove one or more chromatin loops controlling expression of one or more genes in Table 2A and/or Table 2B such that gene expression is reduced; and j. combinations thereof.

In some embodiments, the genetic modification system is a CRISPR-Cas system, a base editing system, a prime editing system, a CAST system, a non-LTR retrotransposon, or a Cas-directed recombinase.

In some embodiments, the disclosure provides for a method of discovering an agent capable of altering an immune response in an immune cell comprising the steps of: a. providing an agent or plurality of agents; b. providing a population of Th17 cells that express P2rx7, P2rx4, Il17a, Il21r, Csf2, Il1r1, Fasl, Scd1, Hadh, Slc2al, Akt2, 1121, Gzmb, Lck, Src, Rora, Rorc, Bhlhe40, Pparg, Abca1, Hk2, Pygl, Adam19, Itgfb1, Cd47, Ccr6, Cxc4r6, Tnfsf14, Csf1, Bcl2, Tmem59, Slc2a3, Gpr65, Itgb7, Itgb3, Abhd2, Osbpl9, Ldlrap1, Pcca, Dmtf1, Btg1, Smc6, Fanci, Usp28, Bcor, Sp100, Foxo1, Dnmt3a, Ppm1g, Tgfbr2, Smad5, Il10rb, 1110, Dusp2, Dusp4, Tgfbr3, and/or Lefty 1; c. contacting the agent or plurality of agents with the population of Th17 cells; d. detecting a level of expression of P2rx7, P2rx4, Il17a, Il21r, Csf2, Il1r1, Fasl, Scd1, Hadh, Slc2al, Akt2, 1121, Gzmb, Lck, Src, Rora, Rorc, Bhlhe40, Pparg, Abca1, Hk2, Pygl, Adam19, Itgfb1, Cd47, Ccr6, Cxc4r6, Tnfsf14, Csf1, Bcl2, Tmem59, Slc2a3, Gpr65, Itgb7, Itgb3, Abhd2, Osbp19, Ldlrap1, Pcca, Dmtf1, Btg1, Smc6, Fanci, Usp28, Bcor, Sp100, Foxo1, Dnmt3a, Ppm1g, Tgfbr2, Smad5, Il10rb, 1110, Dusp2, Dusp4, Tgfbr3, or Lefty 1; e. comparing the detected level to a control population of Th17 cells not contacted with the agent or plurality of agents; f. evaluating the difference between the detected level and a control level to determine if the agent or plurality of agents alters an immune response in the population of Th17 cells.

In some embodiments, the immune cell is a CD4+ T cell, optionally wherein the CD4+ T cell is a Th17 cell.

In some embodiments, the agent or plurality of agents increases expression of Abhd2, Osbp19, Ldlrap1, Pcca, Dmtf1, Btg1, Smc6, Fanci, Usp28, Bcor, Sp100, Foxo1, Dnmt3a, Ppm1g, Tgfbr2, Smad5, Il10rb, 1110, Dusp2, Dusp4, Tgfbr3, Lefty 1, or any combination thereof.

In some embodiments, the agent or plurality of agents decreases expression of P2rx7, P2rx4, Il17a, Il21r, Csf2, Il1r1, Fasl, Scd1, Hadh, Slc2al, Akt2, Il21, Gzmb, Lck, Src, Rora, Rorc, Bhlhe40, Pparg, Abca1, Hk2, Pygl, Adam19, Itgfb1, Cd47, Ccr6, Cxc4r6, Tnfsf14, Csf1, Bcl2, Tmem59, Slc2a3, Gpr65, Itgb7, Itgb3, or any combination thereof.

In some embodiments, the agent or plurality of agents is: a. one or more vectors for knocking out one or more genes from Table 1A and/or Table 1B; b. a genetic modification system configured to modify one or more enhancers controlling expression of one or more genes in Table 1A and/or Table 1B such that binding of transcription factors or other regulatory proteins needed to initiate transcription is reduced; c. a genetic modification system configured to remove or modify one or more promoters controlling expression of one or more genes in Table 1A and/or Table 1B, or configured to replace one or more promoter with a weakened promoter, such that binding of transcription factors and/or RNA polymerase binding is blocked or weakened; d. a genetic modification system configured to introduce a silencer element, or to modify an existing silencer element, in a non-coding region controlling expression of one or more genes from Table 1A and/or Table 1B, such that transcriptional repressors that block or decrease expression are recruited to the non-coding region; e. a genetic modification system configured to disrupt or replace an existing insulator region controlling expression of one or more genes from Table 1A and/or Table 1B such that insulator function on silencer elements or repressive chromatin structures is reduced; f. a genetic modification system configured to introduce a frame-shift mutation in a coding region of one or more genes from Table 1A and/or Table 1B such that a pre-mature stop codon is introduced; g. a genetic modification system configured to introduce insertions, deletions, or substitutions in coding regions of one or more genes from Table 1A and/or Table 1B that encode one or more functional domains of the expressed gene product such that a non-functional gene or gene products with reduced function or activity are produced; h. a genetic modification system configured to introduce one or more modification at one or more exon-intron boundaries or splice sites leading to aberrant splicing or production of non-functional proteins or triggering of non-sense mediated RNA decay; i. a genetic modification system configured to introduce one or more modifications to a regulatory element within the coding regions of one or more genes from Table 1A and/or Table 1B such the expression is decreased; j. a genetic modification system configured to modify sequences encoding one or more post-translational modification (PTM) sites such that one or more activating PTM is removed, or one or more inhibitory PTM that decreases protein function or stability or increases protein degradation is increased; or k. a genetic modification system configured to introduce or remove one or more chromatin loops controlling expression of one or more genes in Table 1A and/or Table 1B such that gene expression is reduced.

In some embodiments, the genetic modification system is a CRISPR-Cas system, a base editing system, a prime editing system, a CAST system, or a non-LTR retrotransposon.

In some embodiments, the agent or plurality of agents is: a. one or more vectors for introducing one or more additional copies of one or more genes from Table 2A and/or Table 2B; b. a genetic modification system configured to modify one or more enhancer regions controlling expression of one or more genes in Table 2A and/or Table 2B such that binding of transcription factors or other regulatory proteins is increased; c. a genetic modification system configured to modify one or more promoters controlling expression of one or more genes of Table 2A and/or Table 2B such that expression is increased; d. a genetic modification system configured to modify one or more silencer regions controlling expression of one or more genes in Table 2A and/or Table 2B, such that binding of transcriptional repressor is blocked or reduced and gene expression is increased; e. a genetic modification system configured to modify or remove one or more regulatory elements in a coding sequence of one or more genes in Table 2A and/or Table 2B; f. a genetic modification system configured to introduce or strengthen one or more insulator regions controlling expression of one or more genes in Table 2A and/or Table 2B; g. a genetic modification system configured to introduce one or more modifications in a coding region that increases gene product stability, folding, or resistance to degradation or enhances the function or activity of the gene product; h. a genetic modification system configured to remove one or more sequences encoding an inhibitory PTM, remove one or more ubiquitination sites, and/or add one or more activating PTM sites; i. a genetic modification system configured to introduce or remove one or more chromatin loops controlling expression of one or more genes in Table 2A and/or Table 2B such that gene expression is reduced; or j. combinations thereof.

In some embodiments, the genetic modification system is a CRISPR-Cas system, a base editing system, a prime editing system, a CAST system, a non-LTR retrotransposon, or a Cas-directed recombinase.

In another aspect, the disclosure provides for a method of treating an autoimmune disease in a subject, the method comprising administering to a subject in need thereof an effective amount of an agent or plurality of agents discovered by the methods disclosed herein.

In some embodiments, the disclosure provides for an engineered immune cell modified to: decrease expression of one or more genes selected from Table 1A (Pygl, Rora, Fbxo27, Adam8, Hk2, Dennd5a, Abhd15, Cpt1a, P2ry1, Fbxl21, Ndrg1, Abca1, Khnyn, Nedd4, Bhlhe40, Chsy1, Pim1, Rorc, Myo1f, Rarg, Cchcr1, Lsp1, Mgll, Ppp1r9b, Per1, Edaradd, Nr1d1, Ltb4r1, Tmem231, Srebf2, Gpr132, Tmem64, Dipk2a, Hip1r, Litaf, Nod1, Etv6, Fam 160a2, Ptprj, Cpd, Resf1, Bcl2, Tmem59, Zfp120, Hid1, Runx2, Glu1, Furin, Inpp5d, Crybg3, Snx20, Dok2, Atp2b1, Il17re, Tiam1, Asx12, Fam20a, Ncoa7, Zyx, Tktl1, Ccr2, Cipc, Ly6a, Clic4, Ssbp2, Lamc1, St3gal6, Slco3a1, Cd7, Ccr8, Firre, Bicral, Ppp1r12a, Myadm, Nhsl2, Dse, Kcnq5, Thsd7b, Tnfsf14, Cyth4, Mdfic, Fgl2, Trim 16, Fam 107b, Traf1, Cxcr6, Slc25a24, Tspan2, Atxn1, Csf1, Ccr6, Il18r1, Irak2, Itgb3, Socs2, Tsc22d3, Itgb7, Usp20, Rai1, Klrb1f, Mgat5, Il2rb, Mturn, Gfpt2, Mapkapk3, Gm32856, Podnl1, Galnt2, Itga2, Fcho2, Hpgds, Gm2a, Ccr4, Il2ra, Rftn1, Pik3ap1, Serpinb1a, Adam 19, AI506816, Sla, Cd47, Itgb1, Slc2a3, B3galt5, Gna15, Ly6e, Tmprss13, Lmnb1, Gpr65, Ifngr1, St6galnac3, Selplg, Sorl1, Capn3, Maf, Bik, Kcnk5, Mmp25, Rampl, Rbl2, Slamf1, Smad3, Cry1, Lmo4, Pparg, Rcbtb2, Lilr4b, Lilrb4a, Apbb1, Acsbg1, Glipr2, Il18rap, Plekhf1, Atp8b4, Rbpj, Cish, Tlr1, Aopep, Krt83, Il10ra, I123r, Fam124b, Il7r, Klrkl, Hip1, Smpdl3a, Map6, Il17ra, Itgae, Cd82, Gabarap12, Coro2a, Galm, Zfyvel, Cd101, Tmem176a, Tmem176b, Actn2, Rab11fip4, Pla2g6, Snx32, Adamts6, Hlf, Ogfod2, Dvl2, Repin1, Dbp, Gm6277, Tmed1, Macrod2, Mnt, Hic1, Pdk2, Ankrd9, Mir5130, Dusp6, Cstad, Fkbpl, Arrdc4, Ruscl, Gm7932, Per3, Arhgap33, Mir17, Gon7, Gm2788, St3gal3, Espn, Phldb3, Snail, Serpinel, BC053393, Mif4gd, Cox7a1, 1700067K01Rik, 4930430F21Rik, Mier1, Mir6951, Nanos3, Rtn3, Wdfy2, Mir6974, Fstl3, Fosl2, Frs3, Gamt, Mir23a, Arhgap23, Klf12, Cd9, Ptprv, Mcmbp, Dlecl, Nlgn2, Kbtbd6, Pagr1a, Ptpre, Gpr157, Slc25a22, Mafk, 4833412C05Rik, Cdknla, Tmem159, Tpcn1, Llgl1, Nfic, Cxxc5, Thoc2, Tle6, Camkk1, Mir132, Klf4, Ino80, 0610040F04Rik, Pogk, Tspoap1, Adgra2, A330009N23Rik, Zc3h18, Arpclb, Fam13b, Nfil3, Pkd112, Usp44, Lipe, Mir574, Cuedc2, Rpl18, Fam219b, Odad1, Tmem221, Gm14486, Sigirr, 1700060C20Rik, Sh2b3, Stx11, Chst11, Ctnnb1, Dok1, Fbx12, Stxla, Unc5b, Adssl1, Micall2, Rasgeflb, Nbeal2, Pletlos, 6430571L13Rik, Mir8105, Stk32c, Tmem17, Dapk1, Fam 122a, Tex44, Fosb, Eif3d, F2r, Kif17, Rab26, Nkapd1, Elapor1, Gnaz, Acads, Crnde, Tctn1, B9d2, Dnajb2, A730081D07Rik, Mir193a, B3gat3, Gulp1, Ubxn11, Jaml, Rabgap11, Snrpd3, Fkbp5, Tnfrsf18, Hc, St3gal5, Asb6, Ftx, Mapkapk2, Napb, Vasp, Tef, Dock5, Gabbr1, Pde8a, Lmf1, Ust, 114i1, Nfe2, Aplml, Ppplr3f, Gm29776, Rhbdl3, B230217012Rik, Ergic1, Heg1, Plcb4, Vax2, Prodh, Cd247, Kdr, Ptpn18, Nxnl1, Kcnk7, Mir7216, Csnkld, N4bp1, Dnal4, Grk2, Morrbid, Prrt1, Arl15, Slc9al, Mir6380, Sdr39u1, Fgfrl, Crygn, Bptf, Tsc22dl, Fam136a, 4932435022Rik, Wdfy4, Tmem5los1, Cercam, Cnot2, Sergef, Pgap3, Arid3a, Impdhl, Tbcld2, Wdr73, Gm15417, Sh3bgrl2, 1700096J18Rik, Chmp6, Unc13d, Aldoa, 0610009L18Rik, Sgsm2, Hook2, 4921507G05Rik, Pou2f2, Gba, Slc16a3, Nuak2, Ptp4a2, Lrp8, Stam, Eefld, Banf1, Tnfrsf21, Selenoo, Spg7, Alkbh5, Sp8, Slc16a6, H4c6, H4c8, Parp1, Zfp511, Phactr4, Gnaq, Mga, Zc3h12c, Nsd1, Fosl1, Dennd2c, Tmem 121b, Galnt3, Mknk1, Gnl1, Thbd, Gnall, Batf, Ifitm 10, Pura, Acot11, Tmf1, Slc9a4, Cfap61, Mei4, Crispld2, Yifla, Shpk, Impa2, Stard8, Tpx2, Gm17767, Lif, Rmdn3, Wrnipl, Plekhm1, Rhbdf2, Lrp5, E4f1, Ildr1, 1700126H18Rik, Celal, Ahil, Rmil, AV039307, Acadsb, Lsm4, Ap3m2, Lrrc25, Colgalt2, Hnrnpl, Irf2bp2, Plec, Qars, Tead3, Gas211, Vmpl, 6430710C18Rik, 1700020M21Rik, Gm7904, Prr29, Slc25a19, Tnk2, Anp32a, Xcll, Hmgb1, Map2k3, Snx25, Tmem30b, Zfp654, Cd48, Gsap, Mett124, Rnf224, Zmiz1, 1110028F18Rik, Gpd11, Ahr, Mdfic2, Con5, Ccr1, Lrrn2, Gbp6, Gm19510, Capzb, Gm20110, Smarcel, Trem2, 3830408C21Rik, Art2a, Fars2, Shisal2b, 4930563M21Rik, Bbs4, Jcad, Smug1, Ldlrad1, Vps54, Gm11482, Btn19, Rgs2, Lonrf3, Rufyl, Micall, Wdr44, A530013C23Rik, Itpkb, Slc25al, AU019990, Lrrc8d, Phc3, Slc37al, Ms4a20, Tiparp, Hectd1, Phosphol, Grik1, Enpp6, Gm19345, Tex33, Wincr1, Snap23, Ccdc146, Gm46545, Samd11, Cast, Nod2, 5830428M24Rik, Zfp608, Rgsl1, 9530068E07Rik, Olfr1372-ps1, Fmnl1, Kifla, Magi3, Slc35f5, Em14, Med4, 4921513103Rik, Lamtor4, Gzmb, Medag, Cep170b, Nptn, Agbl1, Gm11413, Cubn, Mir7688, Igfbp7, Dot11, Gm12532, Lrrc8c, 4930526H09Rik, Rnf141, 4930515G16Rik, Hacd4, Trpm2, Itprip12, Poli, Muc3, Rnpep, Platr14, Crybb1, Fh12, Crybg1, Nup214, 4930522017Rik, Lrp1, Rbm47, Cdk13, Srp68, Gdpd5, 1121, Klh125, Tm2d3, Nek6, Plet1, Cep43, Lyl1, Spicel, Pmaipl, Acvr2a, Tnfrsf9, Mccc2, Dnah 12, Rasl12, Ankrd44, Gpr107, Gm15441, Rassfl, Synj2, Sdccag8, Tlr9, Cdh26, Sbk1, Sdc1, C5arl, Galnt12, Tango6, Gm13031, Spata3, Csk, Pik3cg, Abr, E530011L22Rik, P2ryl4, Ctss, Muc13, Dtnb, Pex7, Tdrd5, Fes, Igsf5, Mthfs, Mthfsl, Lax1, Hmga2, Itprid2, 4930556J02Rik, Smg6, Kcnmb1, Nfkbill, Itsn1, Pde6h, Pik3ca, Slc22a8, Arap1, Ccdc88b, Cmip, Plekhg3, Art4, Dhrs9, Tbkbp1, Gm 10640, Rab 19, Lipm, Abca2, Adap1, Nmnat2, Rab22a, P2rx3, Txndc5, Cep192, Uba7, Asgr2, Vashl, Armc2, Ccr9, Cybrd1, Sp3os, 2810459M11Rik, Ahdc1, Mir7094-2, Colq, Mir29b-1, Pde6d, Psd2, Sdhaf1, Mir684-2, Fasl, Il1r1, Mfn2, Slc2a5, Btbd9, Tg, Acadl, Denndlb, Pogz, Adgrvl, Efr3a, Epx, Fos, Haao, S100a10, Nucks1, Cdyl2, Marchf3, Oser1, Ctcf, Vmn2r85, Alpk2, Rnh1, Txnrdl, Ankrd31, Rad18, Mtx3, Crtc3, Mir7094-1, P2rx7, Pdella, D230030E09Rik, Mir365-2, Ccr3, Hsf5, Zfp395, Ablim3, Gucy2c, Mucl, Kit, Il21r, Kcna3, Krt27, Cass4, Mrtfb, Zbtb16, Mkrn2os, P2rx4, Plin3, Snrk, Mex3c, Ppplr12b, 6820426E19Rik, Brip1, A230108P19Rik, Pttglip, Adgb, Cd86, Zfp664, Pelo, Mtmr7, Retnlb, B4galnt2, Creb5, AU040320, Comt, Csf2, Gm12185, F13a1, Hs2st1, Susd3, Ncor1, Il12a, Mir1928, 5430437J10Rik, Serpine2, Syt12, Gpatch2, Hspb8, Midl, Tex2, 4833427F10Rik, Eps8, Tut4, Mir7683, Rgs13, Serp2, Snora16a, Fam 169b, Cic, Jazf1, Ddx19a, 1700064M15Rik, Aak1, Hccs, Rpap2, Tcf711, Bmerb1, Rnf2, 2900040C04Rik, ccdc 198, Nosip, Filip1, Tomm5, Gm34816, Tap2, D5Ertd579e, A930011G23Rik, Anln, Dcunld2, Glrp1, Glis2, Eml1, Nsmaf, Dsc3, Itpr1, Mexis, Syne2, Il17a, Tyrobp, Cox6al, Gimap3, Havcr2, Pkib, Sv2c, Zswim6, Ikzf2, Srgap3, Grk3, Rrp12, Blk, Galnt14, Shc4, Timp2, Gm3716, Oacyl, Ralgps1, Stk10, Cmc2, Vps13d, Efcab3, Gm12709, Hmox2, Tbllx, Eif2ak1, Rcsdl, Spag9, Moxd2, Agpat4, Togaram1, Dthd1, Bbs9, Slcllal, Ypel2, 1700001K23Rik, Akrlel, Ap4b1, Mocs1, Mtrr, Mbp, Ttc21b, Cxcl9, Mir8120, Tcllb1, Slcla5, Tmem38b, Mllt10, Rlf, Dok6, Olfr525, Henmt1, Fancc, Gm27162, Bpifc, Nfatc2, Snx1, Gm38414, Polrlhas, Mir6387, Acap2, Tmtc4, B4galnt4, Tmem 154, C430042M11Rik, Casc3, Ptpn11, Gm13582, Pou5f1-rs4, Krt28, Zfp362, Fam172a, Pla2g2c, Nfkb2, Axdnd1, Prss30, Mylk2, Cacnals, Mir6906, Vdacl, Icosl, Brcal, 4930431P03Rik, Ak5, Ern1, Myod1, C330024C12Rik, Car5b, Gm11696, Ubac2, Bcl2115, Abcb9, Nfe212, Mboat1, Tmem260, Klrblc, Nrsn2, Fbxw11, Gas7, Bnip2, Gm1720, Gcg, Elf1, Runx1, 4930519G04Rik, Irgc1, Mir1892, BC049352, Ip6k1, 1700016L21Rik, 4930434J06Rik, Pon2, Gm527, Sik1, Pla2g12b, Slc15a2, Gm13710, Rims2, Mov10, Atad1, Ticam1, Hmcn2, Sumo3, Fyco1, Ifitm3, Paox, Snx31, Kcnc1, Gm17745, Uggt1, Plcxd2, Tnks2, Ccl3, Ostf1, Stum, Ncapg, Shmt2, Sgf29, Synpo, Rad51b, Plcb3, Zfp746, Gvin-ps3, Gvin-ps5, Sub1, 4932438H23Rik, Serinc5, Mir1953, Itpr2, Shcbp11, H2-Q5, H2-Q6, Mroh8, Nop58, Pstpip1, Tspyl2, Wdr95, Susd1, Synm, Atp8al, Zzef1, Myo16, Tex43, 4933405E24Rik, Lrrc49, Gm11529, Dnajc27, Nup2101, Ache, Btbd10, Cacng2, Pebpl, Ptprs, Klhdc9, Mppel, Camk2b, E330011O21Rik, Hdac4, Scgblal, Nefl, Nos3, Ppp3ca, Sh2d4a, 3300005D01Rik, Dock8, Sgip1, Spryd7, Tpst2, Ehd1, Prss12, Rhobtb1, Smap2, Creb1, Dock10, AA536875, Metrnl, Reps1, Tm6sf1, Muc4, Eef2kmt, Srl, 4930598F16Rik, Slc6a19os, Icos, Med10, Ifi204, Igtp, Slc38al, 4930465M20Rik, 1700061F12Rik, Clcn6, Emb, Tnfrsf17, Cdc51, Lrrc42, 4921529L05Rik, Ldlrad4, Milrl, Adamts14, Stau2, Trp53bp2, Chmp4b, Lcmt2, Ppplcc, Cmklrl, Rab20, Copg1, Ltb4r2, A430057M04Rik, Tmem171, Pvt1, Sh2d2a, Ncapd3, St6galnac5, Ndfip1, Rps18-ps4, Aktip, Col6a4, Abcc4, Cyp2el, Cxcr5, Ifnar2, Tab2, Noxal, Metap2, Mtmrl, Nadk, Qprt, Cask, Jdp2, C030005K06Rik, Ctso, Olfr530, 1700007L15Rik, Ndufb 10, St8sia4, Etv3, Kcnd3, Rfx2, Cep19, Gm30726, Dus11, Rcan1, Srprb, Calhm6, Fas, Mta3, 4930578N18Rik, Lss, Aktls1, Hnf1b, Ralgapa2, Tbx21, 5830454E08Rik, Arhgap19, Dct, Nup107, Gabrr3, G6pc3, Ppmlk, Cwh43, Hmg20a, 4930428021Rik, Mlap, Msh4, Dnah11, Tsg101, Prok1, Spopl, Abtb2, Gpr89, Mrpl14, Stmnd1, Jpt1, Cpa3, Batf3, Slmap, Coq9, Dgcr2, Wipfl, Zfp874a, Arpc2, Cx3crl, Adam17, Stxbp4, Exoc6b, Gadd45b, Bcl6, 1700012B07Rik, Cpb1, Hif1an, Pabpc2, Cdh3, Prkacb, Gent2, Olfr531, 4930505K 14Rik, Pigv, Cnr2, Ttl111, Tulp1, Adam 15, Bcl6b, Cbr1, Alg14, Me1, H2bc18, Cyp7a1, Gm30173, 119, Nmrk1, Rpl11, Gmeb1, Zfp36, Ppm1e, Plxnc1, Gyg, 4930559C10Rik, Fam91a1, Dox12, Fth1, Pten, Nrg2, Swap70, Gzmk, St6galnac2, Zfp57, Adcy4, Rps6 kb2, E230029C05Rik, Mib1, Smpd1, Sgms2, Arrb1, Nr3cl, Hdac11, Kif5c, Mfsd13a, Btrc, Tnfrsflb, Pacs2, Calcocol, Pelil, Mrps27, Plekha6, Rtkn2, Hacd2, Btg3, Pip5klb, Ptges, Trib1, Vamp1, C030018K13Rik, Raetlc, Lrrk1, Adrb1, Stk24, Itga3, Gnal, Zfp948, Adat1, Eef2k, Selenom, Scrg1, Mapkbp1, Ston2, Niban2, Dtx1, Hk1, Dhx34, Krt80, Saysd1, 1700012B09Rik, Gm8439, Myoc, Golga4, Krt222, Tnpo2, Otogl, Grb7, Bbs7, Eif3a, Ubl3, Cmtm7, Srgn, Cpn2, Inpp5k, Trpv2, Samsn1, Ciart, 1700025F24Rik, Tspyl3, Clip1, Pafah1b2, Brwd1, Coa8, Ankrd6, Cabin1, Mir133b, Agpat5, 1600020E01Rik, 4930513D17Rik, Ptpra, Mbd5, Olfm3, Vps28, 5430421F17Rik, Tmprss6, Gm10804, Cdc73, 1810013L24Rik, F630206G17Rik, Zfp397, Cox412, Rgs10, Olfr524, Prpf4b, Slc2al, Egfr, Gpr12, Aifm1, Fam76b, Fgd6, Fhodl, Akt2, Tmbim6, Itpr3, Tepl, Otulin, Swt1, Ap3dl, Ccdc6, Sema6d, Rab39, Slc36a3, Trmt61a, Uaca, Tnfsf10, Fam 193a, AA986860, Phb, 1700030L20Rik, Mitf, Plekho2, Foxn2, Dennd4a, Fam117a, Ptpn2, Clnk, Trappc31, Capza2, Zhx1, Xpc, H2-D1, 115, Tfdp2, Ub15, Cand1, Eloa, Ikzf3, Pkdcc, Tbcld31, Crybg2, Cd84, Soat1, Trim8, Rnf19b, Ap2al, Nkg7, Entpd7, Serpina3f, Kcnmb4, Xcr1, Slc25a43, Tbcld9b, 4930572013Rik, Gm38425, Ptgfrn, Scart2, 4833419F23Rik, A430090L17Rik, H4cl, Plekhh3, Atf3, Lefty2, Sogal, Fads6, Ptk2b, Cluh, Ppplr16b, Fam180a, I117f, Abcc3, Hadh, Slc27a6, Hemk1, Tnfrsfla, Cerk, Rbpms2, A330023F24Rik, Gm17746, Gxylt1, Ercc8, Ppplr14c, Gstcd, Nebl, Cdon, Ctif, Dpy 1913, Frmd4a, Pdcdllg2, Slc22a16, Dusp22, Stard7, Xrral, Scd1, Fam50b, 2700049A03Rik, Mfsd5, Ak2, Disp1, Slc38a10, Megf10, Ttc39b, Ccdc88c, Car11, Psmc6, Pcbp3, Gm20750, Plxnd1, Klrblb, Sp4, Ppp3cc, Zfp638, Gatad2a, Vmnlr3, Kat6b, Kif23, Erich6, Art2b, Os9, Adarb2, Kmo, Ndufa9, Serpina4-psl, Ccdc84, Tjp1, Degs2, Tasor, Serpinblb, Ttc1, Ric1, Nrnl, Tmem247, Gimap4, Ms4a4c, Myb12, Prdm2, Prep, Lonp2, Lyrm9, Adgrl2, Pax5, Smpdl3b, Pik3cd, Slc15al, Spock2, Mef2a, Sit1, Kcna2, Mir3970, Zfp704, Adam 12, Ski, Cpped1, Ttyh1, Ccdc112, Spata2, Anp32b, Itgax, Ube2f, 9130015A21Rik, Atf7ip, Scaper, Amph, Rbmx, Phldb2, Sufu, Kbtbd2, Tbck, Dnajc5b, Pde4b, 8030474K03Rik, 4921525009Rik, Mark2, Frrs1, Gng2, Hdc, Hgsnat, Zhx3, Thgll, Corolc, Syne3, Cfap126, Pdzd2, Gm33104, Med30, Zbtb1, Rasgeflc, Enpp7, Aven, Cul5, Pde4a, Stard3nl, Mir5118, Ncoa3, Gpr183, Nckap5, Nin, Pdhx, 1115, Vrk2, Clec12a, Fabp12, Dynll2, Plekha5, Olfr1423, Prr51, AI467606, Mirlet7i, Tgm2, Prss39, Trpm6, Col15al, Sema4d, A830029E22Rik, Ly86, Apol9b, Sectmla, Map3k20, Matn2, Sema4b, Zdhhc22, Gm38560, N4bp2, Gm29685, Gm5111, Zfp148, Ccdc63, Iffo2, Hfm1, Vwf, Trim27, Vps53, Torlaipl, Mogat2, Snx30, Scamp2, Marf1, Tusc3, Aptx, Rad50, Fchsd2, Nsmce2, Thada, C2cd3, Gpatch21, Ssbp3, Minpp1, Rab2a, Dio2, Map4, Rbl1, Aurkb, Sytl2, Nfkbiz, Fbxo40, Glrx, Pdcl3, Ppp2r5a, Fbxw7, Ddrl, Glb1, 2310069B03Rik, Mapk14, Fut7, H2-T3, Iqsec1, Gm15569, Tmem256, Raplb, Rtn4rl1, Dlgap4, Ccll, Yars, Hnrnpull, Klf9, 5730460C07Rik, Fryl, Mettl7al, Syngr4, Cox10, Irf2bpl, Airn, Prex1, Macroh2al, Plekha3, Unc13a, Ankrd50, Dglucy, Ear7, Plekhol, Ly96, Mtap, Rai14, Golm1, Ltf, Kif3a, Nbn, Slc17a4, Cldn14, Gm15506, Psmd4, Arhgap31, Ctps2, Parl, Cd302, Tbrg4, Tmc6, Hdnr, Immt, Ptcd3, Fnbp1, Vamp4, Vmn2r100, Snx27, Gfi1, Ncbp3, Fabp5, Pabpc1, Galnt1, Hif1a, Tsen2, Sh3bp2, Apbblip, Xxylt1, Ddx3x, Rtf2, Vps26b, Asic2, IlIrl1, Slc22a15, Eps813, Rbm24, 4930539M17Rik, Sv2b, Tle3, Cab391, Atl1, 4933406K04Rik, Acpp, lyd, Samd4b, Cacnb1, Ncald, 4930471C04Rik, Crot, Ankrd16, Utp6, Aurkaip1, Wdr66, Rad23b, Sdcbp2, Grap2, Tmem 135, Srbd1, Dars, Ly6d, Appl1, Cldn2, Denn2b, Fastkd3, Tedcl, Noamt1, Shisa5, Nol10, Akirin1, Plxdcl, Sik2, Cables1, Cfap44, 4930546K05Rik, Nrp2, Pigl, Lrrc71, Parp9, Fggy, Ccdc711, Gm12171, Cep63, Gm9949, Olfr186, Cyp4f39, Kctd13, Plod3, Zfp472, Slc24a2, Ap5b1, Rras2, Hectd2, Unc5c, Slc43a2, Dnajb6, Sbf2, Stk 17b, Arhgap25, Kcna6, 4930578M01Rik, Wars2, Gtf2ird2, Lef1, Gm4632, Larp1, Map3kl, Ndufa10, Rdx, Rttn, Afg312, Myo9b, Btbd19, Nfkbia, Ptpn1, A630019I02Rik, Rd3, Arid5a, B3gnt3, Cops2, Trim55, Arnt2, Gpn2, Dcaf12, Inf2, Suco, Oxtr, Serpina12, Gmpr, Supt20, Tanc2, Fgf6, Rara, 1700025M24Rik, Fam83a, Zfp438, Slc35b4, Rassf5, Septin9, Adgre5, Cwc25, Yy1, Poc1a, Gdi1, Lgals1, Pias4, Ppp2r3d, Ier2, Retregl, Dand5, 2810021J22Rik, Hoxc5, Tmie, Camk2nl, Mir3089, Card10, Med14, Mcrip1, Osbpl2, Rrnad1, Rassf8, Tecprl, Cox5a, Mfap4, Slc18a2, Tspan18, Mob3a, Tfip11, Wdr90, Bcl211, Dhrs11, Noc21, Col16al, Msc, 2310003N18Rik, Tapbp, Lrrc47, Chmp2a, Prrg2, Ttc39c, Itpkc, Ppp2ca, Nol41, Olfr279, Gm 13003, Map3k7cl, Plcl1, Csrnp1, Stogalnac6, Fstl4, Mast3, Oga, Azi2, Crem, Kdm4a, Stk16, Krt42, Zbtb34, Camk2g, Kdm4d, Lck, Src, Ssbp4, Cd79b, Plcd1, Nrros, Smbd1, Mir142, Dnajb 12, Trerf1, Clrn3, Hunk, Cdh23, Snrnp200, Wscdl, Cradd, Sema4a, 4930594021Rik, Nmur1, 4933406J09Rik, and/or Hilpda) and/or one or more transcription factors selected from Table 1B (RUNX1, SP1, STAT3, IRF4, RORG, RORA, STAT1, BATF, JUNB, FOSL2, Jun, Fosl2, Junb, Fos, Jund, Tfe3, Usf1, Usf2, Clock, Arntl, Arnt, Bhlhe40, Tfeb, Mxi1, Max, Myc, Nfyb, Nfya, Nfyc, Ddit3, Atf4, Cebpg, Zbtb7a, Mbd2, Epas1, Hif1a, Mecp2, Xbp1, Cux1, Zbtb33, E2f2, E2f5, Hinfp, Mtf1, Nrf1, Pax5, Yy1, Poulf1, Nfe2l1, Tead2, Arid5b, Sox5, Foxp2, Foxo1, Foxk1, Foxo3, Nrld1, Nrld2, Rora, Rorc, Stat5a, Stat5b, Stat3, Stat4, Rel, Relb, Rela, Nfkb1, Nfkb2, Maff, Mafk, Maf, Mafg, Bach2, Nfe212, Bach1, Batf, Foxj3, Foxj2, Foxo4, Prdm 1, Irf7, Irf9, Lyl1, Tcf12, Tcf3, Smad3, Smad2, Smad4, Mef2c, Mef2a, Mef2d, Rfx3, Rfx1, Rfx2, Rara, Rxra, Pparg, Nr1h3, Nr2c2, Ar, Nr3cl, Pbx1, Pknox1, Esrra, Esrrg, Esr1, Rarg, Stat6, Nfatc1, Nfatc2, Nfatc3, Ebf1, Prdm 16, Atf2, Crem, Atf1, Creb1, Nr2c1, Rxrb, Mecom, Gata1, Gata3, Prdm5, Tcf4, Zeb1, Hsf1, Hsf2, Ikzf1, Ctcf, Zfx, Bcl6, Nfia, Nfic, Smarca5, Thap11, Dbp, Nfil3, Thra, Tbp, Pou2f1, Pou2f2, Zkscan1, Gfi1, Pbx2, Rest, Srf, Nr4a1, Prdm9, Rbpj, Tbx21, Srebf1, Srebf2, Taf1, Tbx20, Meis2, Tgif1, Maz, Klf3, Klf6, Sp1, Zbtb17, Sp4, Sp2, Sp3, Egr1, Egr2, E2f6, E2f1, E2f4, Spi1, Spib, Ets1, Elf1, Elk1, Gabpa, Elk4, Fli1, Ets2, Elf2, Etv6, Cbfb, Runx3, Runx1, Runx2, Lef1, Tcf7, Irf4, Irf8, Irf1, Irf2, Irf3, Stat1, and/or Stat2); or increase expression of one or more genes selected from Table 2A (Tcf7, Viprl, Cd52, Kif1b, Acvrl1, Gsn, Bach2, Fntb, F2rl1, Dop1a, Ttc28, Rnf167, Patj, Slc44a2, Arsb, Ndrg3, Wdr46, Usp7, Lmna, Rpl12, Cd6, Ranbp10, Slc41a3, Tecr, Il16, Klhdc2, Klh13, Rgs3, Klf13, Tbcld1, Ibtk, Sh3kbp1, Il6st, Cct6a, Adh1, Cpm, Rab3ip, Gramd3, Arhgef11, Utrn, Ublcp1, Slc16a5, Spsb1, Ctnnbl1, Ccnh, Slc28a2, Esyt2, Ecm1, Foxo1, Relt, Dnmt3a, Tcf20, Izumo1r, Tmem1311, Tbcld30, Malat1, Lipa, Hpcal1, Rbm38, Oxct1, Lats2, Map4k4, Galnt6, Dapl1, Add3, Chd2, Txk, Rhoh, A630023P12Rik, Setx, Ggtal, Me2, Ccr7, Sidt1, Pdlim4, Slc25a12, Myl12a, Rev31, Hsd11, Inpp4b, Ppm1g, Lrrfip1, Adk, Ldhb, Slamf6, Rpal, Arl4c, Chd7, Rflnb, Gstp3, Jak1, Bcor, Tnrc6a, Smc6, Sypl, Prkcq, Pdk1, Ppp2rlb, Cers6, Tsn, Abcbla, Adpgk, Trappc12, Fyb, Inpp5f, Fanci, Myo10, Sp100, Slprl, Usp28, Ikbke, Ift80, St3gal1, Gm15708, Scp2, Actn1, Sipa111, Idh2, Shld1, Abhd2, Smarca2, Ldlrap1, Nr4a3, Eif3m, Pcca, Setd4, Osbp19, Prkab1, Fam111a, Naip2, Afp, Rnf145, Hbs11, Il1rl2, Rcan3, Dmtf1, Nsg2, Klf7, Mctp2, G3bp2, Dnajc10, Btg1, Psma6, Dennd2d, Itga7, Ccdc126, Mthfd11, Rapgef6, Tdrp, A430093F15Rik, Casp8, Lgmn, Inpp5b, Tars, D030024E09Rik, Vav2, Fli1, Myl12b, Rad51c, Tapt1, Gpr174, Zfp644, Galnt10, Prorp, Wasf2, Gch1, Stt3b, Gm6639, Abhd17b, Parp4, Rasal, Gm5617, Smarcd2, Stra6, Glis3, Fgd2, Tirap, Adgra3, Atf6, Marchf10, Nsmcel, 2200002D01Rik, Dad1, Nrip1, Rftn2, Iqgap2, Atrnl1, Lekr1, 1700012111Rik, Clqtnf2, Eefsec, Tcf712, Chrm3, Ccser2, Gpbp1, Polh, Aox2, P4ha2, Lncenc1, Tpbg, Glod4, Ldha, Ccdc186, Asap1, Ncl, Dph6, Spats21, Vmn2r99, Gltp, Sestd1, Hrh2, Sptbn1, Tespal, Cdh17, Cilkl, Gapvd1, Virma, Ldb1, Nrde2, Suclg2, Gm8369, Mmd, Pdpk1, Uhrf1bp11, Cp, Qpct, Akap 17b, Prtg, Cep350, Oit3, Arap2, Wipil, Fbx118, Slc6a6, Akap12, Gorasp2, 4930523C07Rik, Cnot10, Garn13, Flnb, Polg, Ankrd13a, Fnip1, Aim2, Hapln1, Klf2, Gm17546, Nid1, Nudcd2, Rnls, Bag3, Ccdc142, Tbcld5, Septin8, Trpml, Arhgap12, Pxn, Atplal, Mett127, Abcd3, Fafl, Phtfl, Abcb7, Rb1, Gfod2, Anapc 10, Vcl, Zfp60, Tmem245, 4933432G23Rik, Nlk, Spsb4, Ddx46, Qrich1, Brd1, Slc16a12, Sp5, Syt6, Cpeb2, Rsul, Dcunld5, Dnajc15, Gphb5, Ilrun, 1700100110Rik, Pardog, C530044C16Rik, Cdin1, Lgi1, Sell, Fgfbp1, Tet2, Eif4g3, Usp47, Mbnl2, Trip12, Cntln, Mterfla, Mterf1b, Kdm7a, Ms4a4b, Dclk2, Stag1, Tbk1, Lysmd3, Nav2, A330040F15Rik, Dhx40, Olal, Bco2, Hs3st3al, Numb, Lrrn3, Itga4, Sord, Phkb, Wdr92, Mir6349, Pdia3, Ms4a4a, Bazla, Ctnnd2, 4732490B19Rik, Mad211, Zfp536, Fam25c, Ifit1b12, Rab11fip5, Nfe213, 1110002J07Rik, Mmp27, 4930447K03Rik, Zc3hav1, Tbcd, Klf8, Lrp6, Spred2, Edem1, Znrf3, a, Sec24b, Dgki, Efcab11, Klrd1, Diaph2, Zranb3, Vmn2r91, Vmn2r92, Kif5b, Atp2b4, Epha2, Ltc4s, Lhcgr, Alas1, Tnr, Agps, Smcla, Abhd17c, Lnx2, B430306N03Rik, Atp2cl, Zfp53, Zcchc10, Atp6ap2, Nfyb, Pfkfb2, Dnajc1, Usp32, Ptprc, Trim5, Nlrp4g, Rybp, Clvs1, Fam20b, Gm1587, Igsf10, Camk4, Med121, Sugp2, St18, Zfp560, Mcu, Aadacl2fm2, Endod1, Rin3, Sgk3, Amn1, Scfd2, AI197445, Clmp, Lmntd1, Sgtb, 1700110C19Rik, 1700123J17Rik, Lrig1, Nsf, Pym1, Kif13b, Zcchc17, Csnklg3, Speccll, Bpgm, Inppl, Mett122, Atpllc, Jarid2, Upb1, A830052D11Rik, Gabbr2, Marchf1, Crebrf, Exoc4, Psd3, Ube2k, Ap1g1, Chst10, Map2, 4933440J02Rik, Pir, Ryr3, Pid1, Atp6vlg3, Clccl, Slc7al, Nxphl, Rnaseh2b, 1122ra2, Zbtb40, Cdyl, Tmem217, Aff1, Wdr59, Hira, Mrps5, Oasl1, Ube2s, Atp6vlgl, Herc1, Nbas, Synel, Cul3, Tent5a, Sec1411, Zmat4, Lpcat1, Gm5108, Sipall2, Amotl1, Pgpep11, Ptpn6, Zmynd8, Dapk2, Ifnlr1, Phldb1, Apls3, Clpb, 1700060C16Rik, Lratd1, Tlel, Exoc2, Wfikkn2, Arid3b, Mir7014, 4930568G15Rik, Dnm3, Epb4112, Gimap9, Fam 102a, Mfap3, Dnajc22, Usp18, Hsf2 bp, Prkd3, Gtpbp4, Gpatch8, Marcks, Prpf18, Park7, Trpc5, Dnah2, Ppt1, Golph3, Slx4ip, 4930432M17Rik, Pgm2, Spaglla, Ub17, Rdm1, Trib2, Nutm1, Smyd2, Cep112, Lpar4, Ccdc174, Tspan5, Usp6n1, Kmt5b, 4933427D14Rik, Usp38, Ces2d-ps, Mdgal, Ccdc150, Cux1, Ube2e3, Dner, Man1a, Rasa2, Yaf2, Bace2, 4930590L20Rik, Fabp7, Myh11, Tbcld19, Gm19466, Syt13, Mtmr10, Zswim1, Ccdc148, Gjb6, Acs14, Hdac9, Marveld2, Slc30a5, Camtal, Pum2, Mcph1, 1700019E08Rik, Tlcd2, Pilrb1, Pilrb2, Fundc1, Zfr2, Rnf11, Tnfsf4, 4930518J21Rik, 4933412E24Rik, Ralgps2, Pldi, Fbxw4, Mcl1, 6030440G07Rik, Dclk1, Arvcf, Sema7a, Mir331, Ywhaz, Dock9, Tmbim7, Arhgef15, Elov15, Tcf4, Lrch3, Cdhr3, Tnkslbp1, Macfl, Ago2, Mboat4, Tm4sf20, Mylk4, Zbtb17, Atp11b, Cga, 4930402K13Rik, Oxr1, Hnrnpa3, Plekhal, 1700031P21Rik, Zfp462, Lix1, Gna12, Fbxo45, Sbnol, Platr30, Ifng, Slc7a13, Atf2, Pskh1, Nrip3, Oxsm, Rbm33, Gdap111, Luc713, Megf11, Irx4, Tmem 18, Flywch1, Map4k3, Lyst, Pcgf5, Ccdc141, Fgd3, Scd2, Cont2, Bach1, Tmem26, Zfp407, Adh6b, Rere, Cacnalb, Hmgcr, Ctr9, Mir5113, Rab11b, Cdc40, Aplp2, Ociad2, Cyb5a, Zfp217, Mob3b, Cmah, Fto, Fam240a, Ankrd12, Utp25, Tnfsf11, Washc4, Gnai3, Map2k5, Add1, Cmssl, Irgm2, Emsy, Nipa2, Tmem94, Rap2b, Fndc3a, Gtf2b, Umad1, Phactr2, Phf3, Larp4b, Pds5a, Svep1, Npepps, Dcunld1, Mfsd4b5, Mir101b, Ppp3cb, Pdlim5, Raph1, Chst2, 1700021F02Rik, Ube2e2, Gm10512, 4930554106Rik, Bicdl1, Ercc6, Bcl3, Il1b, Cstb, Dop1b, Gmds, Selenbp1, Vwa8, Asb15, Brd4, Clec4g, Gm31641, Arid4b, Map4k5, Secllc, Btbd11, Trappc10, Nrd1, 4930415L06Rik, Sppl2a, Tanc1, Zbtb20, Prc1, Btbd7, Actn4, Fpr2, Babam2, Gpr18, Gpc1, Tnfsf18, Btnl2, Gtf2i, Gm15706, Ism1, Slc35b3, Enthd1, Gzma, Rab11fip2, Max, Rab33b, Ctnnd1, Arih2, Hopx, Atpsckmt, Kif3b, Nepro, Robol, Acs16, Erc1, Neb, Kif13a, Spef2, Gm8817, Zfp748, Cds2, Dtwd2, Fam221a, Tex50, Mettl17, Cld, Pdcd1, Maml1, Wdr19, Pip5kla, Ptger3, Bc19, Mir680-3, Sytl3, Dynlt1f, Gm29719, Olfr1510, Arnt, Tslp, Prkcz, Stmn1, Glt6d1, Camk1g, Slc2a10, 1810053B23Rik, Smurf1, Pacsin2, Atel, Pik3r5, Clybl, Septin6, Pip4k2a, Usp33, Acsll, Rnf32, Tmem141, Tnrc6c, Ppp2r3a, Troap, Adam22, Slc29al, Uvrag, Scml4, Slc43al, Pnoc, Setbp1, Rad54b, Taf4, Sumol, Mms221, Selenot, Hipk2, Lrpprc, Smox, Cd2bp2, 2610206C17Rik, Itk, Vps37b, Cd55, Olfr9, Ddx59, Stk4, Cldn4, Uck2, Rbfox3, Mtus2, Ephb4, Kntcl, Shq1, Slc16a10, 1700123012Rik, Hhat, Cyp26b1, Dync21i1, C230014012Rik, Zfp286, Luc 712, Lyzl4, Pkp4, Ralgapb, Gcnt3, Il6, Abca17, Ints7, Meak7, 1700021F07Rik, Sf3b1, Acin1, Usp15, Psen2, Ube2el, Steap4, Cnst, Pmel, Rnf157, Olfr266, Pip4k2b, Rasa3, Agfg1, Fam217a, Hivep2, Myo3b, Sfswap, Fyn, Ttbk2, Rps6ka5, Cd226, Gadl1, Tox2, 1810055G02Rik, BC004004, Nans, Myo5a, Cfap54, Hmbox1, Suclg1, Abcg2, Gne, Gm609, Ciao2a, Kif18b, Dnaaf5, Dcaf11, Secl, Akirin2, Grb2, Cers4, Gsk3b, Pea15a, Ube2n, Gcc2, Nemp2, Map2k4, Msra, Asah1, Hmgxb3, Marchf6, Reln, Dstn, Trappc9, D730003115Rik, Elfnl, Mdm4, Rap2a, 2610037D02Rik, Sorcs2, Aig1, Xrcc4, Tcf12, Scn3b, Sec23ip, Xpa, Tbcld22b, Tmc3, Tspan13, 4930544G11Rik, Atp13a3, Gys2, Prkch, Commd8, Cog2, Taf3, Dnase113, Sdk2, Susd6, Akap6, Gphn, Thumpd2, Ef11, Ireb2, Ltbr, Cldn10, Efna5, Esrp1, Ndufaf6, Sntb1, Hmgbl-rs17, Rcorl, Gan, Gbfl, Btg2, Gm12354, Gm10791, Pak1, Arhgap11a, Brf1, Ddc, Cox7a21, Empl, Gimap7, Mapkap1, Foxo4, Thyl, Ldb3, Stat5b, Enc1, Rpl34, Gm10790, Gclc, Gm14634, Pdss2, Dab2, Gm6498, Kcnj 16, Tgfbr2, Nyapl, Cemip2, Gm20609, Pros1, Clasp2, Tent5c, Sugt1, Atplb3, Efr3b, Map3k13, Ddx10, Gak, Osbpl8, Ralbp1, Trappc8, Atg7, Lbh, Fam 168a, 4930512J16Rik, Klral, Mogat1, Col12al, Fgd5, Ppp2r5e, Rpp21, Agtpbp1, Cdc42, Nnmt, Smad5, Gm10432, Gm35722, Ptprn2, Gm13848, Sfpq, App, Bcar3, Cep68, Gigyf2, Zfp831, Odc1, Mlxipl, Nck2, Pias2, Eif4e3, Nfkb1, 6530403H02Rik, Niban1, Mtx2, Tprg, Cnot61, Fbxo15, Hdac8, Wdr89, Yeats2, Lnpep, Ttf1, Mical2, St8sia6, 4930554C24Rik, L3mbtl3, Rbbp6, Gm4278, Tec, Nudt16, Tet1, Dach2, Glil, Zfp862-ps, Ly6c1, Gpat3, Nbeall, Snx29, Fus, Gramd4, Entpd4b, C7, Panx1, Csrnp3, Sec31a, Lncpint, Arhgap18, Galnt7, Lrrc4b, Satb1, Eri3, Tbcld14, Dyrk2, Rtn1, Mtif3, Ppplcb, Adarb1, Fam126a, Nab1, Gm26685, Pcgf3, Dnttip1, Psma8, 9430014N10Rik, Fbxo28, Nudt4, Ppa2, Svil, Gm20740, Hs6st1, Atp10d, Voppl, Anxa2, Alox12, Smagp, Arl5b, Rem2, Cngb1, Kalrn, Kcnmb4os1, Unc5cl, Mink1, Orai2, Cfap52, 1700030F04Rik, Kctd1, Plscr5, Fgfr2, Atp9b, Efhcl, 8430430B14Rik, Tesk2, Klhdc1, Specc1, Ets1, Osbpl6, Slc34al, Mrpl15, Zcchc9, Fry, Tom 112, Ube2d2a, Mgat4a, Arl4a, Lrrc28, C430002N11Rik, Sfrp2, Ncoa5, Ggact, Tmem9b, Fchol, Haus8, Fpr-rs4, A930006K02Rik, Chaserr, Stk381, Cep851, Oprml, Disc1, Ifit3, Hrh1, Rgs12, Aqp9, Phrf1, Tnfsf8, Eiprl, Kdm3b, Mlec, Chchd3, Gas2, Chm, Epas1, Farpl, Fbxo47, Vwce, 2410022M11Rik, Dhrs4, Apln, Fam114al, Gemin5, Triqk, Frmd7, Smyd3, Gm5083, Gm29686, Klrg1, Mzt1, Dusp11, Slc9a9, Plag11, Plppl, Ssh2, Fer116, Pkp2, Ube2z, Iqgap1, Rgs1, Gbp10, 1700104L18Rik, Igf2bp3, Vmnlr236, 4933407L21Rik, Wapl, Mirt1, Sesn3, Fbxo32, Spata48, Tomm20, Cyrib, Dym, Itpk1, Pias1, Ctnnall, Sun2, Selp, Gm17399, Sema4f, 5830416119Rik, Mak16, Kmt2a, BC039771, Coa7, 4921511C10Rik, 4930595D18Rik, Zfp335, Gem, Slc28al, Ccdc3, Asprv1, Tmem178b, Ophn1, Slclla2, Ext1, Zc3h12d, Dlgap5, Elavl1, Leprotl1, Limk2, Zyg11b, Jak2, 4930426L09Rik, Atp6vlc1, Cd200r2, Lrba, Phldal, Zfp446, Gtf2ird1, Nudcd3, 4930556N09Rik, Gm33619, Hephl1, Synpo2, Baz2b, Fbxo33, Kcnb1, Manla2, Aqr, Serpinb5, Proc, Cpeb1, Fubp1, Stk39, Aebp2, Rprd2, Rasgrp1, Rph3al, Rspryl, Eeal, Nfl, Gnas, Ube2h, Fam241a, Rhbdd1, Ptprn, 4921511117Rik, Tspan9, Slc4a4, Nars, Tnrc6b, Pcytla, Rbms3, 1700034P13Rik, Afdn, Gm13807, Gm20743, Gm5086, Edem3, Med131, D030045P18Rik, Tollip, Capn2, Gfra2, Elmol, Cep164, Phf2, Stim1, Immp21, Etv31, 2700069118Rik, Pdp1, Col26al, C730036E19Rik, Lmod3, Abcblb, ligp1, Igf2bp2, Zfand3, Tnik, Zfp277, Zc3h12a, Nhsl1, Txlnb, Gm10030, Mosmo, Slfn1, 4933406118Rik, Cd200r4, Uqcrfs1, Gdi2, Spag1, Osbplla, Fbxo25, Klra6, Msl2, Pcx, Birc6, Cenpu, Ugcg, Depdc5, Trp53inpl, Plekhg6, Gm12505, Prdm10, Mir5626, Diaph3, Platr10, Got1, Slc38a2, Mmaa, Ezh1, Vmn2r101, Olfr1414, Col6a6, Gm14317, Ankrd55, Nkap, Olfr312, Stapl, Nsd3, 4930512M02Rik, Slc4a10, Ubap21, Col28al, Ensa, Wdyhv1, 4933430H16Rik, Htt, Alox 12e, Dennd11, Gm2245, Adcy9, Ccdc170, Gm19585, Lrrk2, Farsb, Zfp281, Lypd6b, Slc26a8, 4933432103Rik, Wnk1, Map3k14, Sfil, Pde10a, Bcdin3d, Snap47, Gbp9, Pvr, 4933424G06Rik, St8sial, 9330179D12Rik, Epb4114a, Eif2ak3, Msl3, Borcs5, Sema3f, Palmd, 1700042G15Rik, Odf1, A330093E20Rik, Itgam, Sydel, Aplf, Ab12, 5031425E22Rik, Ep300, Tbl1xr1, Pik3r1, Tmsb10, Pitpnb, Gm4285, Hspbpl, Rrn3, Foxql, Skor1, Gypc, Srfbp1, 1700028K03Rik, Arl5c, 2210417A02Rik, Thtpa, Ccdc57, Dock4, Akt3, Ntrk3, B3gntl1, Galntl6, Gm36229, Trim33, Rreb1, Shroom1, Prss32, Sbk2, Prkca, Vezf1, Stra8, Eif2b2, Gm19705, Fndc7, Arl14, Gm19303, 112, Ctsf, Setd3, 1700010K23Rik, Pank4, Spi1, Cd200, Slc22a2, Eci1, Ppil4, D830013020Rik, Lmtk2, Plaur, Eif2a, Lgals9, Plcg1, Zdhhc2, Parp11, 4930546C10Rik, Tshr, Ppp6r2, Trp53rkb, Jakmip1, Wipf2, Dennd4c, Garem1, Foxpl, Atp6vlel, Myo5b, Capl, Hspb11, Kmt2c, Arpp21, Epha3, Vang12, Map3k4, Pex14, Thnsll, Arhgef10, Klh15, Dlg3, Mark3, Ncoa2, Mab2113, Taok3, Trpsl, Flt3, Myole, Ttc27, Xdh, Slc17a8, Drd3, Kcnip1, C730014E05Rik, Prdm1, Map3k5, Lama4, Fgr, Fndcl, Mfge8, Armc8, Rheb, Otud7a, Gm32141, Ofccl, Cyb5b, Atp10a, Hmces, Map3k7, Alox5ap, Lpin1, Ranbp9, Bcas1, Prss2, Dnajb4, Vmnlr1, Taf1b, 4930406D18Rik, Colca2, Boll, Shisa7, Tacc2, Cnn3, Lor, Olfr658, Zfand6, Camk2d, Farl, Gm1123, Bambi-psl, Plbd1, Tspan3, Fez2, Slc25a48, Bvht, 5530601H04Rik, Ppcdc, Zfp870, Btbd2, Stambpl1, Zer1, Ralgds, Wnk2, Nfia, Dlg1, Maml2, Myo6, 9130230L23Rik, Manlcl, Slc30a7, Ier51, AI504432, Camkld, Cfap74, Myh9, 1700066B17Rik, Eif2b3, Pmm2, Stim2, Arhgap39, Pkd1, Wfdc2, Alkbh8, Prml, Gm2447, Rassf2, Rbm17, Gtf3c3, Pou6f1, Cbx1, 1700049E22Rik, Tsen15, Plekhs1, 1700025G04Rik, Atg10, Chd1, Map10, Phf11a, Bys1, Crmp1, Mia2, 1810041H14Rik, Cntrl, Herc3, Lrrc3b, Rrp1, Wsb2, Mir146, Btc, Ccnel, Riox1, Kcnn3, Actr10, BC016579, Mgmel, C030034122Rik, Kcnq1, Anks1, Slx4, Ehd3, Gnaol, Cpne5, Cyb5r4, Zfp800, Fbx117, Irf4, Akap14, Pard3, Gria3, Trpm8, Helz, Nek10, Capzal, Katnall, Armcx6, Nfatc1, Smarcal, Gabrb1, Dnajb14, Gm15270, Lat, Plekha8, Ctla4, Irf5, Tigit, Vps13b, Mxi1, Prrcl, Ryr2, Stard9, Slc37a3, Ext13, 1700063D05Rik, Siah3, Cpsf3, Olfr1364, Iqch, Atoh7, Pum1, Dcaf5, Mllt3, Tex30, Ppip5k2, Dppa5a, Gpr19, Dcp2, Gsx1, Nglyl, 4933433H22Rik, Gja5, Tead1, Tox, Samd91, Strada, B930092H01Rik, Evl, Eci3, Fxr1, Spata5, Olfr1509, Arid4a, Dph5, Topbp1, Nyap2, Sinhcaf, Mafb, 1110032F04Rik, Dgkg, Slc47a2, Inpp4a, Ikbkg, Wwox, Atp6v0d2, Hipk1, Prrc2c, Uch15, Mir21c, Enppl, Polrla, Gm14718, Ppargcla, Usp12, Gm26839, Prim2, Cblb, Igsf23, Gm5907, Vav3, Otulinl, Tlk1, Mbnl1, Ica1, Fra10ac1, Lpp, 113, Rnf220, Tab3, Arhgap17, Fam210a, Trim36, Ppfibp2, Slc23a4, 2310022B05Rik, Kif26b, Kcnj2, E430016F16Rik, Ogt, Clec16a, Napepld, Mtmr3, Tnfrsf19, Acbd3, Egf, Ehmt1, Phf6, Gm30790, Znrf1, Xpo4, Gm8709, Ss18, 4930563J15Rik, Dnah17, Smapl, Gm34068, Try4, Ttc30a2, Pgapl, Sertad2, Slc28a2b, Ppp2r5c, Mir30c-2, Ube2cbp, Cfap100, Pde3b, Eps15, Trak2, Gm20139, Spink13, Zfp629, Ifngas1, Nxf1, 4931403E22Rik, Acaa2, Prickle1, Map3k9, Traf5, Smad7, Lmo2, Slc45a4, Coq2, Vapb, Lrig2, Ccdc152, Mdml, Usol, Fibcd1, Zdhhc20, Xylt1, B020018J22Rik, Cflar, CreId1, Btla, Cobll1, Ptchd3, St6gall, Naa20, Pde7a, Daam1, Ttl15, Cdc14a, Ubap2, Smim4, Dgkd, Gm33337, Kidins220, Bsn, Snap25, Speer4f1, Fam71b, Malt1, Cd83, Dbx1, Lasp1, Hecw2, Naa30, 2210408F21Rik, Itih5, Tagap, Zfp984, Tnni1, Ubr2, 1500009L16Rik, Asap2, Ubap1, Gorab, Zfp819, 8430426J06Rik, Trim56, Pycr2, Smim14, Tesc, Hspa13, Mbd2, Mideas, Crhbp, Ubqln1, Dis312, Nme7, Mindy4, Cst7, Kcmf1, Msrb3, Prf1, 1120ra, Mir6395, Snhg16, Zbtb38, Ect21, Mylip, Znrf2, Mgat1, Cfap97d2, Slain1, 1110020A21Rik, Asb13, Gm13986, BB123696, Il10rb, Arhgap6, Fam216b, Fkbpla, Lnpk, Rnf43, Rnf17, Gucd1, Rictor, Bcat1, 2610307P16Rik, Ttc17, Gxylt2, 6030443J06Rik, Akap13, C130026L21Rik, Mrpl3, Nwd1, Scarb2, Dusp16, Snx12, Sox5, 4933440M02Rik, Mx2, Lbp, Snx18, 3110056K07Rik, Dtnbp1, Ext2, Tnn, Krr1, Wdfyl, Edil3, Fbxl12os, Cldn5, Pde2a, Rab6b, Man2al, Pemt, 9630013K17Rik, Cfap20dc, Cog3, Hs3st5, Mrpl1, Rbm20, Arhgef101, 3425401B19Rik, Smg1, Impg1, Cyth3, Gm32200, Enpp3, Olfr464, Tpk1, Cbl, Cdk12, Gpr141, Tdrd3, C2cd5, Sh3rf1, Atrn, Gm30214, Gng13, Pptc7, Ercc61, Gm5122, Lrch1, Snd1, Fam222a, Zfp787, Olig3, Bcr, Fam163b, Abhd6, Ppmlb, Usp24, Insyn2b, Ywhah, AI597479, Cdkn2aipnl, Kl, Gm20758, Sec63, Dusp10, Arhgef12, Cfap45, Gm12381, Ccni, Lims1, Ubtd2, Tmem267, Zswim2, Dnajc7, B3gnt2, Gucylal, Tnip3, Gm36738, Mfhas1, Sugct, Fam43a, Myo9a, 9330104G04Rik, Rasgrp4, Tyrp1, Hamp, Hamp2, Cabcocol, Elovl6, Nrarp, Cggbp1, Glg1, Rfx4, Mir6356, Tmem 123, Micu2, Secisbp21, Xrn2, Mir21a, Otud1, Plcl2, Ppmla, Uxsl, Anxa3, Magi2, Chi15, Plin2, Prag1, Stpgl, Rfx8, Lsm14a, Cysltr2, Map3k8, Slc39a10, 4930459C07Rik, Ogdh, Rbm11, Itm2a, Cdr2, Gm805, Bckdhb, Lclat1, Hlcs, Ptpn22, 4930534H03Rik, Ambral, A630001012Rik, Kcnj15, Ly6c2, Zfp930, Gm29687, Mir692-1, Gfpt1, Pou2af1, Znhit6, I110, Ptch2, Bicra, Pou2f1, Bbx, Rnf38, Tbcld2b, 1700074H08Rik, Ubtd1, Smad4, Tet3, Cacna2dl, Cyfip2, Dusp4, Ptprk, Rps6ka3, Kdm8, Zscan2, Cacnb3, Fblnl, Rabgefl, Raly, Cascl, Gna13, Ikzf4, BC065403, Gabpb2, Tmem40, Bod11, Fam3c, Il12rb2, Rbms2, Klra3, Klra9, Noct, Slcla7, Guca2b, Tfam, Ly75, Ifi47, Lipk, Rapgef4, Gm6551, Ikbkb, Dusp2, Spin1, Selenof, Calhm5, Mtss1, Msi2, Snora26, Ascc3, Dpp4, Arc, 2410004B18Rik, Tmod3, Slcla4, Chl1, 4930529K09Rik, Gm11110, Setd2, Atxn7, Heatr1, Epb4114b, Hivep1, Itgb6, Sos1, Stat4, Cdkall, Rad52, Blm, Chp1, Lamp2, Mtpn, Fbx15, Pisd-ps2, Tank, Actg2, Tnks, Ocrl, Zwint, Gm7538, Manba, Rnf138, Adck1, Kansl11, Csnkle, Garrel, Slc2a2, Actr2, Cpa4, Seclla, Zfp592, Gm5544, Gm20544, Irs1, Msh3, Zfp821, Fcrl1, Nup210, Adamts20, Neto2, Cyp20al, Scgb3al, 1810006J02Rik, Antxr2, Wdr37, Casp4, 9530052C20Rik, Eepd1, Bspry, Pgr151, Uimcl, Zfp14, Cog5, Mmut, Rassf3, Melk, Zfp280d, 4930469K13Rik, Stat3, Gcm2, 1700017B05Rik, Pcnx, 2810408111Rik, Ucp1, Dtx4, Ifi208, Fcmr, Txndc11, Nup153, Zbtb43, Zbtb25, Gimap6, Tbca, Cpeb4, Cd2, Tns4, Kcnc2, Kif11, Abcg1, Cd28, Glcci1, She, Spata6, Sla2, Col18a1, Etnk1, B430212C06Rik, AU041133, Olfr401, Slit3, Oosp1, Rnf216, Cdc20b, Itm2b, Msgnl, Tbcld7, Brdt, Car12, Redrum, Ankrd46, Tafa4, Cntn5, Ppp2cb, Gm5127, Stx3, Cdk17, Nr2c2, Bri3, Sspn, Papolg, Vdac3, Dnm2, Ift81, Pam, Cyria, Rxfpl, Fam118a, Evi5, Ifit2, Stk26, Gm12108, Mrtfa, Capn8, Rexo2, Eeflaknmt, Gtdcl, Gm12216, AI839979, B230323A14Rik, Dydc2, Rsphl, Hexa, Mphosph6, 4732471J01Rik, Plekhf2, Sec31b, Csgalnact1, Gm15326, Gm19589, Nek7, BC051019, Ntn1, Tmigd3, Nfat5, Ago3, Gm8013, Cdh22, Mett18, Lrrcl, Jmy, Paxbp1, Dipklb, Sdf4, Ercc3, Erg, Lhfp, Kctd19, Skor2, Racl, Gvin2, Ccdc15, Ppplr13b, 4930583K01Rik, Mad111, Adgrg6, Gm16630, Platr4, Als2, Cit, Gm28043, Pik3r6, Rnf19a, Snrpn, Gprc5c, Ppard, 1110059E24Rik, Pde4dip, Phf21a, Atp6v0al, Ggcx, Pcedlb, Cenpcl, Fkbplb, Csad, Pagl, Cnksr3, Tmem243, Dennd3, Prkce, Lhxlos, Lrrc6, Sema6a, Lrp12, Kcp, Vwa3a, Dnajbl, Ecel, Rundcl, Sag, Rnase10, Lrmp, Slc24a3, Shisall, Cdh5, Gab1, Pja2, Cacnald, Lpar5, Il12b, Ldlrad3, Npl, Snx4, Slc23a2, Pan3, Scin, Tiall, Acoxl, Ikzf1, Atp8b1, Taar4, Ntpcr, 4930565D16Rik, Ift43, Prdm5, Prss43, Slc17a6, Unc80, Klf3, Usp9x, Ttc3, Ms4a4d, Reep3, Gm16793, Herc2, Plekhm3, Mir568, Atxn21, Tmem184b, Ninj2, Rplp1, Naip1, S100a6, Chn2, Cbr3, Nyx, Ttc7b, Trmt11, Numal, Snrpe, Ptbp3, Mnat1, Tnni3, Supt6, Epc1, H2al2a, Cdc42se2, Zfp41, Asb1, Rhobtb2, Spata13, Arhgef17, Scaf8, Spata32, Camkmt, Tbcld22a, Clasp1, Prokr1, Gnl31, Stard13, Kdelr2, Smad2, Zp3r, Dcdc2b, Wdr25, Asb7, Ell, Nedd41, Trim71, Hmcn1, Apaf1, Tbcel, Decr1, Gm9530, 1700029J03Rik, Hexb, Sec61b, Bcas3, Usp3, Lyzl1, 1700027J07Rik, Vrk1, Relch, Abi2, Dguok, Crbn, Mrgbp, Itga6, Serpini2, Rfc1, Manea, Gm5434, Kif16b, E130114P18Rik, Zfat, Rnaset2b, Sael, Myold, Supt71, Thrb, Rgs9, Kcnf1, Car2, Spidr, Mtal, Spin2c, Gbel, Sp2, 1700016K19Rik, Foxo3, Coro2b, Tk2, Arl6ip1, Apobec1, Uril, Map3k19, Adcy6, Fam81a, Zfp51, Gng12, 2510009E07Rik, Klra10, Pwwp3a, Deupl, Gm11981, Smul, Slc6a7, Sreklip1, Ddx31, Dux, Rab10, Dlg2, Srpk2, 4930425L21Rik, Wdr20, Cd200r3, Tigd2, Speer2, Pak2, Tmem132c, Kat8, Dusp27, Fndc5, Slc25a30, Fam234a, Gm27217, Por, Prkar2b, Dmbt1, Ccnq, Dleu2, Heyl, Agr3, Gpr15, B230303A05Rik, Nova2, Gm29461, Srgap1, Mir5127, Osmr, Hslbp3, Srp14, Ptk2, Derl1, Rptoros, Stx16, Srd5a3, Ube2w, Dnajc3, Wdtcl, Itga9, Mfsd2a, Tle4, Txn1, Gsdme, Prdm16, Arhgap10, Rab27a, B4galt5, Armcx1, Sdk1, Nedd9, Spata31dlb, Dipk1a, Fam120a, Nup98, Zbtb9, Cd2ap, Mpp6, Akna, Srpk1, Slc44a3, Ccser1, Adcy1, Arid1b, Iqsec2, 1810046K07Rik, Rin2, Isoc1, Gm35584, S100b, Micu1, Myo3a, Ical1, Klh12, Cops9, Tex9, Pfdn2, Tnnt1, Arid1a, Slc26a4, Gm20268, Acta2, Dennd1a, Wdr38, At12, Fam78a, D430041D05Rik, Slc7a8, Hnf1a, Tm2d1, Ccny, Sik3, Slc12a7, Zfp622, Apc, Gm4814, Arhgap15, Mr1, Atf7, Ube4a, Mfsd4a, 3830406C13Rik, Zzz3, Dpy 1911, Prg4, Dusp5, Mtmr6, Phf14, Rps6 kb1, C230024C17Rik, Lamb3, Actr3b, Arhgef18, Oscp1, Stk38, Nbea, Ywhaq, Akr7a5, Tmem11, Wscd2, Rock2, Sgpp1, Cbx5, Tmem114, Bmt2, C330011F03Rik, Slc52a3, Piwil2, Slc36a1, Adamts14, Lcp1, Sppl3, 2210010C04Rik, 1700020N01Rik, Plch2, 4930519D14Rik, Slc13a3, A730043L09Rik, Mam13, Pck1, Cgn, Rpap3, Cdc7, Sltm, Lcn9, Fau, Arhgef3, Sesn1, 1700027F09Rik, Lhfpl3, Pik3c2b, Sardh, Gm5069, Rapgef2, Pmepal, Sgk1, Tbcld16, Efnb2, Npsr1, Gpr180, Itsn2, 4933411E08Rik, Cdk19, Riok1, Slc25a40, Sh3pxd2a, Ube2r2, Slc19a3, Aldh5a1, Nt5c2, Gm4668, Plat, Gm5532, Ippk, Fut8, Acsbg3, Serbp1, Vps45, Basp1, Gm16675, Arfip1, Tram1, Nkx2-4, Gabrr1, Pnrc1, Tgfbr3, Tns1, Gm14424, Sys1, Rubie, Foxj2, Pld2, Creb311, Sft2d2, Sntb2, Shb, B4galnt3, Cd40, Dnai3, Whrn, 1700109G15Rik, Prdm9, Capsl, 1700021N21Rik, Bcl2114, 1600002D24Rik, 4930515B02Rik, Traf3, Ppmll, Ppp4rl, Zfand2a, Cracd, Trim25, 4930529N20Rik, Tmc8, A730085K08Rik, Akrld1, Slc35cl, Myo18a, Rell1, Iqub, Nox1, F2, Postn, Fsip1, Adrb2, Grik4, Mir7678, Paqr5, Mir9-3hg, Wnt5b, Cep128, Deptor, 4932438A13Rik, Galnt15, Sez61, Calm2, Pdgfb, Stx8, Cd36, Nup160, Hesx1, Fhdcl, Gsx2, Mmp15, Tafa3, Akain1, Cabp1, Mir7-2, Rapgef1, 2610035D17Rik, Twsg1, Ccnd2, Carm1, Slc39a11, Dpysl2, Cyld, Gm1647, Rab15, Pde8b, Tob1, Klf17, Prkcb, Ckap4, Mpp2, Gabrr2, Ptpn13, Gap43, Lrrc63, 119r, Tnfrsf8, 2010010A06Rik, Slain2, Npas2, Apod, Gcc1, D030025E07Rik, Ripor3, Ltbp2, Arhgap29, Ppplr21, G530011006Rik, Ints6, Mir3965, 2810001G20Rik, Cap2, Ulk4, Ripor2, Afap1, Thap3, Axin2, 4930556M19Rik, Ifne, Zfp677, Gm31108, Zfp983, 1700109K24Rik, Zfp365, Kcnh5, Psmal, Enpp2, Vsig8, Ap3 ml, Card11, 4930402H24Rik, Celf2, Ano10, Lama5, Runx3, Btbd6, Tmem201, Eps811, Gm13032, Trub1, Hykk, Mical3, Aff3, Zeb1, Prss16, Spring1, Trp53bp1, Foxn3, Adamts3, Lefty 1, Bmp2k, Camk2a, Snn, Zkscan16, Fam214b, Med13, Slc16al, Tmem131, Trak1, 9030616G12Rik, Eomes, Tmem 104, Slc36alos, Suds3, Atp6v0a4, Frs2, Gm14164, Gm16863, Gm5148, Gramdlc, Cnot6, Tshz1, Ostm1, Slc30a9, 4930486L24Rik, Ksrl, Kcnj12, E230016M11Rik, Prmt3, Arid5b, Dtl, Ppm1h, 4921517D22Rik, Polr3e, Vtila, Gpnmb, Prelid3b, Cd300a, Exog, D630024D03Rik, Hspb1, SIpr3, Shf, Timm8a2, Exoc6, Fam 186b, Prkarlb, Dusp7, Gpr179, Zmym6, Dip2b, Myrfl, Gm572, Smim41, Zfp839, 8430436N08Rik, Acox2, Ascc1, Phf20, Arid2, Mir467h, Clstn3, Slc5al, Fhit, Gm 14569, Wee2, Il15ra, Mex3b, Spata31dla, Slc6a9, Tmcc3, Xpnpep2, Gm1604b, Rps6ka2, Pat12, Hoxb 13, Slc22a1, D16Ertd472e, Myo5c, Ccn6, Mtarc1, Pla2g10, Mcoln3, Vps18, Gnb11, Arsi, Zfp658, Adamts10, Ppargclb, B930018H19Rik, Prkag2, 4933433G08Rik, Arsg, Grip2, Siahla, Lad1, Ythdc1, Irf2, Drosha, Upk1a, Thsd1, Trio, Nat9, Lin52, Rbpms, Gm5893, Sh2d1a, Shc3, Gnptab, Zfp236, Klh131, Smg9, Tlcd5, Arhgap5, Braf, Ccdc162, Pipox, Ppp6r1, A330076C08Rik, Clip2, Wnt2b, 1700028D13Rik, Pcnx2, Chrna9, Sdr9c7, Gm5535, Cacna2d4, Cytip, Wnt3, Map2k2, Ncf1, Traf3ip3, Neurl1b, 4921504E06Rik, 5430427M07Rik, Gm38404, Mett19, Mir6897, Thap12, 1700066B19Rik, Lsm3, Lrrc74a, Smim23, Calr4, Pde4d, Ptpn14, Pds5b, Trpv3, Dlk1, BB019430, Ilf3, Car8, Gm20098, Actn3, Gm29684, Rhob, Lrp4, Spry4, Prune2, Akr1b8, Fam168b, Casp3, Rfx3, Focad, Mpp7, Npy, Ggt7, Gm10857, Rab8b, Cyth1, Tshz2, 1700016G22Rik, Calcr1, Ptpru, Thap4, Slc20a2, Sall3, Agk, Arf1, Lcp2, Ubash3b, Dnah6, Ncor2, Trim24, Gm12610, Pfdn1, Akap81, Herpud1, Angpt4, Cpa6, Cenpf, Elfn2, Spacal, Bank1, Cited2, Gadd45g, 4632415L05Rik, Dlgap1, Hnrnpf, Pdgfc, Sil1, Samd8, Snrpf, Zfp330, Nkx1-1, C130080G10Rik, Gm33677, Hs6st2, Osbpl10, Morn2, Spol1, Gpsm2, Mgat4f, Htatip2, Pppor3, 0, 0, 0, 0, Tcf7, Vipr1, Cd52, Kiflb, Acvrll, Gsn, Bach2, Fntb, F2rl1, Dopla, Ttc28, Rnf167, Patj, Slc44a2, Arsb, Ndrg3, Wdr46, Usp7, Lmna, Rpl12, Cd6, Ranbp10, Slc41a3, Tecr, Il16, Klhdc2, Klh13, Rgs3, Klf13, Tbcld1, Ibtk, Sh3kbp1, Il6st, Cct6a, Adh1, Cpm, Rab3ip, Gramd3, Arhgef11, Utrn, Ublcp1, Slc16a5, Spsb1, Ctnnbl1, Ccnh, Slc28a2, Esyt2, Ecm1, Foxo1, Relt, Dnmt3a, Tcf20, Izumo1r, Tmem1311, Tbcld30, Malat1, Lipa, Hpcal1, Rbm38, Oxct1, Lats2, Map4k4, Galnt6, Dapl1, Add3, Chd2, Txk, Rhoh, A630023P12Rik, Setx, Ggtal, Me2, Ccr7, Sidt1, Pdlim4, Slc25a12, Myl12a, Rev31, Hsdl1, Inpp4b, Ppm1g, Lrrfip1, Adk, Ldhb, Slamf6, Rpal, Arl4c, Chd7, Rflnb, Gstp3, Jak1, Bcor, Tnrc6a, Smc6, Sypl, Prkcq, Pdk1, Ppp2rlb, Cers6, Tsn, Abcbla, Adpgk, Trappc 12, Fyb, Inpp5f, Fanci, Myo10, Sp100, Slprl, Usp28, Ikbke, Ift80, St3gall, Gm15708, Scp2, Actn1, Sipall1, Idh2, Shld1, Abhd2, Smarca2, Ldlrap1, Nr4a3, Eif3m, Pcca, Setd4, Osbpl9, Prkab1, Fam111a, Naip2, Afp, Rnf145, Hbs11, Il1rl2, Rcan3, Dmtfl, Nsg2, Klf7, Mctp2, G3bp2, Dnajc10, Btg1, Psma6, Dennd2d, Itga7, Ccdc126, Mthfd11, Rapgef6, Tdrp, A430093F15Rik, Casp8, Lgmn, Inpp5b, and/or Tars) and/or one or more transcription factors selected from Table 2B (HEB, FOXO1, GABPA, FLI1, ELK4, EGR1, TCF1, ETS1, Jun, Fosl2, Junb, Fos, Jund, Tfe3, Usf1, Usf2, Clock, Arntl, Arnt, Bhlhe40, Tfeb, Mxi1, Max, Myc, Nfyb, Nfya, Nfyc, Ddit3, Atf4, Cebpg, Zbtb7a, Mbd2, Epas1, Hif1a, Mecp2, Xbp1, Cux1, Zbtb33, E2f2, E2f5, Hinfp, Mtf1, Nrf1, Pax5, Yy1, Pou1f1, Nfe2l1, Tead2, Arid5b, Sox5, Foxp2, Foxo1, Foxk1, Foxo3, Nrld1, Nrld2, Rora, Rorc, Stat5a, Stat5b, Stat3, Stat4, Rel, Relb, Rela, Nfkb1, Nfkb2, Maff, Mafk, Maf, Mafg, Bach2, Nfe212, Bach1, Batf, Foxj3, Foxj2, Foxo4, Prdm1, Irf7, Irf9, Lyl1, Tcf12, Tcf3, Smad3, Smad2, Smad4, Mef2c, Mef2a, Mef2d, Rfx3, Rfx1, Rfx2, Rara, Rxra, Pparg, Nr1h3, Nr2c2, Ar, Nr3c1, Pbx1, Pknox1, Esrra, Esrrg, Esr1, Rarg, Stat6, Nfatc1, Nfatc2, Nfatc3, Ebf1, Prdm 16, Atf2, Crem, Atf1, Creb1, Nr2c1, Rxrb, Mecom, Gata1, Gata3, Prdm5, Tcf4, Zeb1, Hsf1, Hsf2, Ikzf1, Ctcf, Zfx, Bcl6, Nfia, Nfic, Smarca5, Thap11, Dbp, Nfil3, Thra, Tbp, Pou2f1, Pou2f2, Zkscan1, Gfi1, Pbx2, Rest, Srf, Nr4a1, Prdm9, Rbpj, Tbx21, Srebf1, Srebf2, Taf1, Tbx20, Meis2, Tgif1, Maz, Klf3, Klf6, Sp1, Zbtb17, Sp4, Sp2, Sp3, Egr1, Egr2, E2f6, E2f1, E2f4, Spi1, Spib, Ets1, Elf1, Elk1, Gabpa, Elk4, Fli1, Ets2, Elf2, Etv6, Cbfb, Runx3, Runx1, Runx2, Lef1, Tcf7, Irf4, Irf8, Irf1, Irf2, Irf3, Stat1, and/or Stat2); or a combination thereof. The engineered immune cell may be an iPSC, a hematopoietic stem cell, a T cell progenitor, a CD4+ T cell, or a Th17 cell.

The engineered immune cells cell may be modified to knock-out one or more genes from Table 1A and/or Table 1B; comprise one or more modifications to a non-coding region controlling expression of the one or more genes from Table 1A and/or Table 1B; comprise one or more modifications of a coding region of the one or more genes from Table 1A and/or Table 1B, such that expression of the one or more genes from Table 1A and/or Table 1B is decreased, or the function or activity of an expressed gene product is reduced; comprise one or more modifications of one or more chromosome loops such that expression of one or more genes from Table 1A and/or Table 1B is decreased compared to an unmodified cell; or a combination thereof.

The engineered immune cell may be modified to include one or more additional functional copies of one or more genes of Table 2A and/or Table 2B; to comprise one or more modifications in one or more non-coding regions that control expression of the one or more genes of Table 2A and/or Table 2B such that expression of the one or more genes is increased; to comprise one or more modifications of one or more coding regions of one or more genes in Table 2A and/or Table 2B such that expression of the one or more genes is increased, or the function or activity of an expressed gene product is increased; to replace one or more dysfunctional copies of one or more genes of Table 2A and/or Table 2B with one or more functional copies; to introduce or remove one or more chromosome loops such that expression of one or more genes from Table 2A and/or Table 2B is increased; or a combination thereof.

The engineered immune cell may be further modified to express a chimeric antigen receptor (CAR) or an exogenous T-cell receptor (TCR). The TCR or CAR is capable of binding an antigen to decrease expression of one or more genes from Table 1A and/or Table 1B, or increase expression of one or more genes selected from Table 2A and/or Table 2B. The TCR or CAR may be expanded in the cell. The engineer cells may be autologous or allogenic.

In one embodiment, a method of preparing an engineered immune cell for use in therapeutic compositions, comprises; administering one or more agents to the immune cell that can decrease the expression of one or more genes selected from Table 1A and/or Table 1B, or increase expression of one or more genes selected from Table 2A and/or Table 2B, or a combination thereof.

The agent to decrease expression of one or more genes from Table 1A and/or Table 1B may be: one or more vectors for knocking out one or more genes from Table 1A and/or Table 1B; a genetic modification system configured to modify one or more enhancers controlling expression of one or more genes in Tables 1A and/or 1B such that binding of transcription factors or other regulatory proteins needed to initiate transcription is reduced; a genetic modification system configured to remove or modify one or more promoters controlling expression of one or more genes in Tables 1A and/or 1B, or to replace one or more promoter with a weakened promoter, such that binding of transcription factors and/or RNA polymerase binding is blocked or weakened; a genetic modification system configured to introduce a silencer element, or to modify an existing silencer element, in a non-coding region controlling expression of one or more genes from Tables 1A or 1B, such that transcriptional repressors that block or decrease expression are recruited to the non-coding region; a genetic modification system configured to disrupt or replace an existing insulator region controlling expression of one or more genes from Table 1A and/or 1B such that insulator function on silencer elements or repressive chromatin structures is reduced; a genetic modification system configured to introduce a frame-shift mutation in a coding region of one or more genes from Table 1A and/or 1B such that a pre-mature stop codon is introduced; a genetic modification system configured to introduce insertions, deletions, or substitutions in coding regions of one or more genes from Tables 1A and/or 1B that encode one or more functional domains of the expressed gene product such that a non-functional or gene products with reduced function or activity are produced; a genetic modification system configured to introduce one or more modification at one or more exon-intron boundaries or splice sites leading to aberrant splicing or production of non-functional proteins or triggering of non-sense mediated RNA decay; a genetic modification system configured to introduce one or more modifications to a regulatory element within the coding regions of one or more genes from Tables 1A and/or 1B such the expression is decreased; a genetic modification system configured to modify sequences encoding one or more post-translational modification (PTM) sites such that one or more activating PTM is removed, or one or more an inhibitory PTM that decreases protein function or stability or increases protein degradation is increased; a genetic modification system configured to introduce or remove one or more chromatin loops controlling expression of one or more genes in Tables 1A or 1B such that gene expression is reduced, or a combination thereof.

The agent to increase expression of one or more genes from Table 2A and/or one Table 2B may be: one or more vectors for introducing one or more additional copies of one or more genes from Table 2A and/or Table 2B; a genetic modification system configured to modify one or more enhancer regions controlling expression of one or more genes in Tables 2A and/or 2B such that binding of transcription factors or other regulatory proteins is increased; a genetic modification system configured to modify one or more promoters controlling expression of one or more genes of Tables 2A and/or 2B such that expression is increased; a genetic modification system configured to modify one or more silencer regions controlling expression of one or more genes in Tables 2A and/or 2B, such that binding of transcriptional repressor is blocked or reduced and gene expression is increased; a genetic modification system configured to modify or remove one or more regulatory elements in a coding sequence of one or more genes in Tables 2A and/or 2B; a genetic modification system configured to introduce or strengthen one or more insulator regions controlling expression of one or more genes in Tables 2A and/or 2B; a genetic modification system configured to introduce one or more modifications in a coding region that increases gene product stability, folding, or resistance to degradation or enhances the function or activity of the gene product; a genetic modification system configured to remove one or more sequences encoding an inhibitory PTM, remove one or more ubiquitination sites, and/or add one or more activating PTM sites; a genetic modification system configured to introduce or remove one or more chromatin loops controlling expression of one or more genes in Tables 2A or 2B such that gene expression is reduced; or a combination thereof.

The genetic modification system may be a CRISPR-Cas system, a base editing system, a prime editing system, a CAST system, a non-LTR retrotransposon, or a Cas-directed recombinase.

The engineered cells may be formulated as a pharmaceutical composition.

A method of treating an inflammatory disorder comprising administering to a subject in need thereof the engineered cells disclosed herein. The inflammatory disease or disorder may be selected from a group consisting of acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopeni purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anemia; polyarteritis nodosa; polymyositis; primary biliary cirrhosis; primary myxedema; psoriasis; rheumatic fever; rheumatoid arthritis; Reiter's syndrome; scleroderma; Sjogren's syndrome; systemic lupus erythematosus; Takayasu's arteritis; temporal arteritis; vitiligo; warm autoimmune hemolytic anemia; Wegener's granulomatosis asthma, allergy, allergic rhinitis, allergic airway inflammation, atopic dermatitis (AD), chronic obstructive pulmonary disease (COPD), inflammatory bowel disease (IBD), Irritable bowel syndrome (IBS), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

In some embodiments, a method of treating an inflammatory disease and/or disorder in a subject in need thereof comprising, administering to the subject one or more agents that decreases expression of one or more genes selected from Table 1A and/or Table 1B; increases expression of one or more genes selected from Table 2A and/or Table 2B; or a combination thereof.

The one or more agents for decreasing expression of one or more genes selected from Table 2A and/or 2B may be: one or more vectors for knocking out one or more genes from Table 1A and/or Table 1B; a RNAi agent for decreasing expression of one or more genes from Table 1A and/or Table 1B; a genetic modification system linked to or otherwise capable of complexing with a transcription repressor that blocks transcription of one or more genes from Table 1A and/or Table 1B; a genetic modification system capable of making one or more epigenetic edits to the cell genome such that expression of one or more genes from Table A and/or Table 1B is silenced; a genetic modification system configured to modify one or more enhancers controlling expression of one or more genes in Tables 1A and/or 1B such that binding of transcription factors or other regulatory proteins needed to initiate transcription is reduced; a genetic modification system configured to remove or modify one or more promoters controlling expression of one or more genes in Tables 1A and/or 1B, or to replace one or more promoter with a weakened promoter, such that binding of transcription factors and/or RNA polymerase binding is blocked or weakened; a genetic modification system configured to introduce a silencer element, or to modify an existing silencer element, in a non-coding region controlling expression of one or more genes from Tables 1A or 1B, such that transcriptional repressors that block or decrease expression are recruited to the non-coding region; a genetic modification system configured to disrupt or replace an existing insulator region controlling expression of one or more genes from Table 1A and/or 1B such that insulator function on silencer elements or repressive chromatin structures is reduced; a genetic modification system configured to introduce a frame-shift mutation in a coding region of one or more genes from Table 1A and/or 1B such that a pre-mature stop codon is introduced; a genetic modification system configured to introduce insertions, deletions, or substitutions in coding regions of one or more genes from Tables 1A and/or 1B that encode one or more functional domains of the expressed gene product such that a non-functional or gene products with reduced function or activity are produced; a genetic modification system configured to introduce one or more modification at one or more exon-intron boundaries or splice sites leading to aberrant splicing or production of non-functional proteins or triggering of non-sense mediated RNA decay; a genetic modification system configured to introduce one or more modifications to a regulatory element within the coding regions of one or more genes from Tables 1A and/or 1B such the expression is decreased; a genetic modification system configured to modify sequences encoding one or more post-translational modification (PTM) sites such that one or more activating PTM is removed, or one or more an inhibitory PTM that decreases protein function or stability or increases protein degradation is increased; a genetic modification system configured to introduce or remove one or more chromatin loops controlling expression of one or more genes in Tables 1A or 1B such that gene expression is reduced; or a combination thereof. The genetic modification system for (d)-(n) is a CRISPR-Cas system, a base editing system, a prime editing system, a CAST system, a non-LTR retrotransposon, or a Cas-directed recombinase. The genetic modification for making one or more epigenetic edits may be Cas linked to or otherwise associated with an epigenetic modifier.

The one or more agents to increase expression of one or more genes from Table 2A and/or Table 2B may be: one or more vectors for introducing one or more genes from Table 2A and/or one or more transcription factors from Table 2B; a genetic modification system linked to or otherwise capable of complexing with a transcription initiator, constitutively or inducibly, that increases expression of one or more genes from Table 2A and/or one or more transcription factors from Table 2B; a genetic modification system capable of making one or more epigenetic edits to the cell genome such that expression of one or more genes from Table 2A and/or one or more transcription factors from Table 2B is increased compared to an unmodified cell; a genetic modification system configured to modify one or more enhancer regions controlling expression of one or more genes in Tables 2A and/or 2B such that binding of transcription factors or other regulatory proteins is increased; a genetic modification system configured to modify one or more promoters controlling expression of one or more genes of Tables 2A and/or 2B such that expression is increased; a genetic modification system configured to modify one or more silencer regions controlling expression of one or more genes in Tables 2A and/or 2B, such that binding of transcriptional repressor is blocked or reduced and gene expression is increased; a genetic modification system configured to modify or remove one or more regulatory elements in a coding sequence of one or more genes in Tables 2A and/or 2B; a genetic modification system configured to introduce or strengthen one or more insulator regions controlling expression of one or more genes in Tables 2A and/or 2B; a genetic modification system configured to introduce one or more modifications in a coding region that increases gene product stability, folding, or resistance to degradation or enhances the function or activity of the gene product; a genetic modification system configured to remove one or more sequences encoding an inhibitory PTM, remove one or more ubiquitination sites, and/or add one or more activating PTM sites; a genetic modification system configured to introduce or remove one or more chromatin loops controlling expression of one or more genes in Tables 2A or 2B such that gene expression is reduced, or a combination thereof. The genetic modification system may be a CRISPR-Cas system, a base editing system, a prime editing system, a CAST system, a non-LTR retrotransposon, or a Cas-directed recombinase.

In some embodiments, a method for identifying an agent capable of modifying or modulating an immune cells, in particular a Th17 cell or progenitor thereof, comprises applying a candidate agent to the isolated immune cell or immune cell population; and detecting modulation of one or more phenotypic aspects of the isolated immune cell or immune cell population by the candidate agent, thereby identifying the agent. The agent may be capable of modulating the proliferation, differentiation, maturation, migration, cytokine expression, cytotoxicity and/or viability of the isolated immune cell or immune cell population, optionally, wherein the agent is capable of inducing or repressing the proliferation, differentiation, maturation, migration, cytokine expression, cytotoxicity and/or viability of the isolated immune cell or immune cell population.

In some embodiments, the disclosure provides for a method of treating an autoimmune disorder in a subject in need thereof comprising administering an gent to the subject, wherein the agent is identified according to a method disclosed herein.

In some embodiments, a method for treating or preventing an inflammatory or immune disorder comprising: detecting one or more genes selected from Table 1A and/or Table 2A in a subject thereby creating a gene signature; and assigning a differential accessibility (DA) or differential expression (DE) score to the gene signature by comparing the detected one or more genes selected from Table 1A and/or Table 2A to a naïve cell; and clustering the one or more genes in the gene signature that are operatively connected to transcription factors selected from Table 1B and/or Table 2B; determining one or more chromatic accessibility scores and/or transcription factor motif scores and/or a enrichment score associated with the one or more genes linked to the transcription factors, thereby determining target regulatory elements; and administering a therapeutic if a chromatic accessibility score and/or transcription factor motif score is detected.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1A-1H-Differential regulation of TCF1 in Th17 subtypes is driven by IL-23. Naïve CD4⁺ T cells from IL-17a-GFP mice (FIG. 1A), Tcf7-GFP reporter (FIG. 1B), or WT mice (FIG. 1C) were differentiated into np (TGFβ1+IL-6) and pTh17 (IL-1β+IL-6+IL-23). (FIG. 1A) IL-17A-GFP cells were sorted and Tcf7-L and Axin2 mRNA expression quantified by qPCR from the indicated Th17 subtypes (n=6). (FIG. 1B) Median fluorescence intensity (MFI) of Tcf7 locus reporter activity in the indicated Th17 subtypes compared to naïve CD4 T cells (n=4). (FIG. 1C) Representative histograms of TCF1 expression and summary data of MFI in Th17 subtypes (n=4). (FIG. 1D) Mice were immunized for EAE with MOG₃₅₋₅₅ peptide emulsified in CFA and Mycobacterium tuberculosis H37Ra extract. Pertussis toxin (100 ng/mouse) was administered i.v. into the tail vein on day 0 and day 2 post immunization. Summary data of MFI and representative histogram of TCF1 expression in the indicated Th17 populations (n=5). (FIG. 1E) Representative flow cytometry data and summary plots of the frequency of TCF1^{hi}RORγt⁻ and TCF1^{low}RORγt⁺ populations in the indicated Th17 subtypes at day 5 (n=4). (FIG. 1F) Naïve CD4⁺ T cells were differentiated with IL-1β+IL-6 and graded doses of IL-23. Summary data of MFI and representative histogram of TCF1 expression are shown (n=6). (FIG. 1G) Naïve CD4+ T cells from IL23r-GFP mice were differentiated into pTh17. IL-23R-GFP+ and IL-23R-GFP− cells were sorted and mRNA expression of the indicated genes examined (n=6). (FIG. 1H) WT, p19−/−, and IL-23R−/− mice were immunized as in (FIG. 1D). Summary data of MFI and representative histogram of TCF1 expression in Th17 (CD4⁺TCRβ⁺RORγt⁺) harvested from the dLN of the indicated groups (WT and p19-KO n=5, IL-23R-KO n=4). (FIG. 1A-1G) ns, not significant; *p<0.05, **p<0.01, ***p<0.001, ****p<0.0001 Mann-Whitney test, two-tailed. (FIG. 1H) *p<0.05 Kruskal-Wallis One-Way ANOVA test. Mean±SEM are shown. Data are representative of at least 2 independent experiments.

FIG. 2A-2H—TCF1 deficiency increases Th17 pathogenicity. WT C57BL/6J mice were adoptively transferred with either 2×10⁶ 2D2 TCF1 WT (2d2⁺Il17a-gfp⁺Tef7^{fl/fl}) or 2D2 TCF1cKO (2d2⁺Il17a-gfp⁺dLck-Cre⁺Tcf7^{fl/fl}) pTh17 cells. (FIG. 2A) Mean EAE score is shown (n=5 per group), **p<0.01, linear mixed model. Data are representative of more than 3 independent experiments. (FIG. 2B) Summary data and representative histopathology images of spinal cord sections from recipients of 2D2 TCF1 WT (n=5) or TCF1cKO (n=4) cells. Left panels, meningeal and parenchymal mononuclear cell infiltrates in spinal cord, typical of EAE (arrows) (Scale Bars=250 μm). Middle panels, higher power of boxed area in left panel. Right panels, parenchymal mononuclear cell infiltrates in the cerebellar white matter (WM). ML, molecular layer (Scale Bars=50 μm). (FIG. 2C-2G) CNS-infiltrating immune cells were harvested from mice at peak of disease (EAE score 3-5) and analyzed by flow cytometry. (C) Summary data and representative flow cytometry plots of the frequency and absolute numbers of RORγt⁺ cells within the 2D2 cell population (n=8 per group). (FIGS. 2D-2G) Immune cells were re-stimulated ex vivo followed by intracellular staining. (FIG. 2D) Representative flow cytometry plots of IL-17A-producing cells and summary data of the frequency of 2D2 cells producing the indicated cytokines (n=8 per group). (FIG. 2E) Representative flow cytometry plots and summary data of the frequency and absolute number of 2D2 cells producing IL-17A and IFNγ. (FIG. 2F) Representative flow cytometry plots and summary data of the frequency and absolute number of 2D2 cells producing IL-17A and GM-CSF. (FIG. 2G) Representative flow cytometry plots and summary data of the frequency and absolute number of 2D2 cells producing GM-CSF and IFNγ. Ns, not significant; *p<0.05, **p<0.01, Mann-Whitney test, two-tailed. Mean±SEM are shown. Data are representative of two pooled experiments. (FIG. 2H) WT C57BL/6J mice were adoptively transferred with 2D2 TCF1cKO Th17_{INF} cells and treated either with mouse IgG1 isotype control or anti-IL17A (200 μg/mouse) on days 0, 3, 6 and 9. Mean EAE score is shown (n=10 per group) Data are representative of two pooled independent experiments.

FIG. 3A-3G—Sustained TCF1 expression dampens Th17 pathogenicity. WT C57BL/6J mice were adoptively transferred with either 3×10⁶ 2D2 TCF1 WT (2d2⁺) or 2D2 TCF1-Long (2d2⁺CD2-Cre⁺Tcf7-L) pTh17 cells. (FIG. 3A) Mean EAE score is shown (n=5 per group), **p<0.01, linear mixed model. Data are representative of 3 independent experiments. (FIG. 3B) Summary data and representative histopathology images of spinal cord sections from recipients of 2D2 TCF1 WT (n=5) or TCF1-Long (n=5) cells. Left panels, arrows indicate areas of immune cell infiltration. Right panels, magnification of areas indicated in the left panels (Scale Bars=250 μm). (FIG. 3C) Representative flow cytometry plots and summary data of the frequency and absolute numbers of RORγt⁺ cells within the 2D2 cell population (n=9 per group). (FIGS. 3D-3G) CNS-infiltrating immune cells were re-stimulated ex vivo followed by intracellular staining. (FIG. 3D) Representative flow cytometry plots and summary data of the frequency of 2D2 cells producing the indicated cytokines (WT n=9; TCF1-Long n=8). (FIG. 3E) Representative flow cytometry plots and summary data of the frequency and absolute number of 2D2 cells producing IL-17A and IFNγ. (FIG. 3F) Representative flow cytometry plots and summary data of the frequency and absolute number of 2D2 cells producing IL-17A and GM-CSF. (FIG. 3G) Representative flow cytometry plots and summary data of the frequency and absolute number of 2D2 cells producing GM-CSF and IFNγ. ns, not significant; *p<0.05, ****p<0.0001, Mann-Whitney test, two-tailed. Mean±SEM are shown. Data are representative of two pooled experiments. Data points with a value of “0” were not displayed in plots using logarithmic scale.

FIG. 4A-4H—TCF1 deficiency confers pathogenicity to npTh17 cells. WT C57BL/6J mice were adoptively transferred with either 4×10⁶ 2D2 TCF1 WT (2d2⁺Il17a-gfp⁺Tcf^{fl/fl}) or 2D2 TCF1cKO (2d2⁺Il17a-gfp⁺dLck-Cre⁺Tef^{fl/fl}) npTh17 cells. (FIG. 4A) Mean EAE score is shown (n=5 per group), **p<0.01, linear mixed model. Data are representative of more than 3 independent experiments. (FIG. 4B) Summary data and representative histopathology images of spinal cord sections from recipients of 2D2 TCF1 WT (n=6) or TCF1cKO (n=7) cells. Left panels: spinal cord images displaying areas of parenchymal immune infiltration. Arrows indicate fields magnified in middle panels. Right panels: leptomeninges and spinal nerve root areas displaying marked immune infiltration in recipients of 2D2 TCF1cKO compared to 2D2 TCF1 WT cells (right panels). Arrow indicates immune infiltrate in the spinal nerve root (Scale Bars=250 μm). (FIG. 4C) Summary data of the absolute number and frequency of 2D2 cells within the total CD45⁺ population infiltrating the CNS. (FIG. 4D) Representative flow cytometry plots and summary data of the frequency of RORγt⁺ cells within the 2D2 cell population (n=7 per group). (FIG. 4E-4H) CNS-infiltrating immune cells were re-stimulated ex vivo followed by intracellular staining (n=7 per group). (FIG. 4E) Summary data of 2D2 cells producing the indicated cytokines and representative flow cytometry plots of IL-17A. (FIG. 4F) Representative flow cytometry plots and summary data of the frequency and absolute number of 2D2 cells producing IL-17A and IFNγ. (FIG. 4G) Representative flow cytometry plots and summary data of the frequency and absolute number of 2D2 cells producing IL-17A and GM-CSF. (FIG. 4H) Representative flow cytometry plots and summary data of the frequency and absolute number of 2D2 cells producing GM-CSF and IFNγ·ns, not significant; *p<0.05, **p<0.01, ***p<0.001, Mann-Whitney test, two-tailed. Mean±SEM are shown. Data are from two pooled experiments.

FIG. 5A-5H—TCF1 deficiency obviates the need for IL-23 for Th17 pathogenicity. p19^{-/-} mice were adoptively transferred with either 2×10⁶ 2D2 TCF1 WT (2d2⁺Il17a-gfp⁺Tcf^{fl/fl}) or TCF1cKO (2d2⁺Il17a-gfp⁺dLck-Cre⁺Tcf^{fl/fl}) cells differentiated with IL-1β+IL6. (FIG. 5A) Mean EAE score is shown (WT n=13; TCF1cKO n=12), ***p<0.001, linear mixed model. Data are representative of 3 independent experiments. (FIG. 5B) Summary data of the absolute number of CD45 cells infiltrating the CNS of TCF1 WT (n=11) or TCF1cKO (n=12) recipient mice. (FIG. 5C) Summary data of the absolute number and frequency of 2D2 cells within the CD45 CNS-infiltrating cells (WT n=15; KO n=16). (FIG. 5D) Summary data and representative flow cytometry plot of the frequency of RORγt⁺ and T-bet cells within the 2D2 cell population infiltrating the CNS (WT n=11; KO n=12). (FIG. 5E-5H) CNS-infiltrating immune cells were re-stimulated ex vivo followed by intracellular staining (WT n=10; KO n=12). (FIG. 5E) Summary data of 2D2 cells producing the indicated cytokines. (FIG. 5F) Representative flow cytometry plots and summary data of the frequency and absolute number of 2D2 cells producing IL-17A and IFNγ. (FIG. 5G) Representative flow cytometry plots and summary data of the frequency and absolute number of 2D2 cells producing IL-17A and GM-CSF. (FIG. 5H) Representative flow cytometry plots and summary data of the frequency and absolute number of 2D2 cells producing GM-CSF and IFNγ. ns, not significant; *p<0.05, **p<0.01, ***p<0.001, ****p<0.0001 Mann-Whitney test, two-tailed. Mean±SEM are shown. Data are representative of at least two pooled experiments.

FIG. 6A-6I—TCF1cKO npTh17 cells resemble IL-23-induced pathogenic Th17 cells. WT C57BL/6J mice were adoptively transferred with either 4×10⁶ 2D2 TCF1 WT (2d2⁺Il17a-gfp⁺Tcf^{fl/fl}) or TCF1cKO (2d2 Il17a-gfp⁺dLck-Cre⁺Tcf^{fl/fl}) npTh17 cells. (FIG. 6A) Heatmap of differentially expressed (DE) genes between 2D2 TCF1 WT and TCF1cKO npTh17 cells isolated from the dLN of transferred mice. Tick marks indicate selected genes associated with Th17 biology. (FIG. 6B) Box plot showing the pathogenic Th17 signature score in 2D2 TCF1 WT and TCF1cKO npTh17 cells (p=0.00018). (FIG. 6C) UMAP of the single-cell RNA profiles of Th17 cells from the spleen of mice with active EAE. Projection of the CXCR6⁺ Th17 cell signature (left). Projection of the TCF1cKO Th17 cell signature (middle). Correlation of the TCF1cKO and CXCR6⁺ Th17 cell signatures (right). (FIG. 6D) Violin plots showing the TCF1cKO signature score in Th17 cells infiltrating the CNS or residing in the draining lymph nodes (dLN) of mice with active EAE. (FIG. 6E) Violin plots showing the TCF1cKO signature score in CD4 T cells from the cerebrospinal fluid (CSF) or peripheral blood (PBMCs) of patients with MS. (FIG. 6F) Heatmap of differentially accessible (DA) chromatin regions between 2D2 TCF1 WT and TCF1cKO npTh17 cells. Tick marks indicate selected loci of genes associated with Th17 biology. (FIG. 6G) DA chromatin regions (boxes) between 2D2 WT and TCF1cKO npTh17 in the Rorc and Il17a loci. (FIG. 6H) Box plot showing the signature score of genes associated with Th17 pathogenicity in the DA chromatin regions in 2D2 TCF1 WT and TCF1cKO npTh17 cells (p=0.00217). (FIG. 6I) Pie chart showing the overlap of DA regions between 2D2 TCF1cKO and CXCR6 Th17 cells.

FIG. 7A-7F-A TCF1-driven transcriptional network determines Th17 cell state. (FIG. 7A) Transcription factor to target gene (TF: target) regulatory network in 2D2 TCF1 WT versus TCF1cKO npTh17. TCF1 WT-(boxes at left, containing HEB through ETS1) or TCF1cKO-specific (boxes at right, containing RUNX1 through FOSL2) TFs are aligned in the center. Round nodes represent target gene modules that display: (i) increased chromatin accessibility and mRNA expression in TCF1 WT (solid circles at top left) or TCF1cKO (solid circles at lower right) designating active modules that are connected to TFs through solid edges, or (ii) increased chromatin accessibility without differential mRNA expression in TCF1 WT (light circles at top right) or TCF1cKO (light circles at lower left) designating poised modules that are connected to TFs through dashed edges. Edge width indicates the connectivity between the TF and the target module (the proportion of genes within the module that is regulated by the connecting TF). The network was pruned to only include edges with absolute or relative high connectivity (>0.3 or Chi-square test p<0.05, Methods, Example 1). Node size is proportional to node degree (the number of TFs regulating the module in the pruned network). Representative genes and processes are displayed for each gene module. (FIG. 7B) Heatmap showing the overlap between the 2D2 WT and TCF1cKO DE genes and RORγt-bound genes (Xiao et al., 2014). Selected genes important for Th17 cell biology are highlighted. (FIG. 7C) Box plot showing the score of the RORγt core gene signature in 2D2 WT and TCF1cKO npTh17 cells (p=0.00006). (FIG. 7D) Western blot analysis of protein lysates of WT and TCF1cKO Th17 cells. Input and samples immunoprecipitated with either rat IgG control or antibody against RORγt are shown. (FIG. 7E) Box plot showing the score of RORγt-TCF1 bound genes in DA chromatin regions of 2D2 TCF1 WT and TCF1cKO npTh17 cells (p<0.00001). (FIG. 7F) Western blot analysis of protein lysates of WT and TCF1cKO Th17 cells treated with DNAse I. Input and samples immunoprecipitated with an antibody against Rorγt are shown.

FIG. 8A-8F—Differential regulation of TCF1 in Th17 subtypes is driven by IL-23 (related to FIG. 1A-1H). (FIG. 8A) Schematic of Th17 differentiation in vitro. (FIG. 8B) Summary data of the ratio of TCF1 and RORγt MFI during pTh17 and npTh17 differentiation (left) and TCF1 and RORγt MFI expression at the indicated days (right). (FIG. 8C) MFI of Tcf7 locus reporter activity in Th17 cells differentiated with IL-1b+IL-6 with and without IL-23 (n=4). (FIG. 8D) Representative flow cytometry plots and summary data of the frequency of TCF1^{lo}RORγt^{hi} in pTh17 cells that either received IL-23 or not during the resting phase. (FIG. 8E) Summary data of TCF1 and RORγt MFI of re-stimulated pTh17 that either received IL-23 or not during the resting phase. (FIG. 8F) Summary of data for TCF1 MFI in a STAT4 knockout background. *p<0.05 Kruskal-Wallis One-Way ANOVA test (B). *p<0.05, **p<0.01, Mann-Whitney test, two-tailed (C-E). Mean±SEM are shown. Data are representative of at least 2 independent experiments.

FIG. 9A-9I—TCF1 deficiency increases Th17 pathogenicity (Related to FIG. 2A-2G). (FIG. 9A) Representative flow cytometry plots of TCF1 expression in peripheral naïve CD4+ T cells harvested from 6-8 week old TCF1 WT or TCF1cKO mice. (FIG. 9B) Representative flow cytometry plots of CD4 and CD8 expression in thymic T cells harvested from 6-8-week-old TCF1 WT or TCF1cKO mice. (FIG. 9C) Representative flow cytometry plots of IL-17A-GFP expression of WT or TCF1cKO naïve CD4+ T cells differentiated into the indicated Th17 subsets. (FIG. 9D) Summary data of cytokine secretion throughout pTh17 and npTh17 differentiation of WT or TCF1cKO CD4+ T cells. (FIG. 9E) Schematic of pTh17 in vitro differentiation for adoptive transfer experiments. (FIG. 9F-9I) CNS-infiltrating immune cells were harvested from mice at peak of disease (EAE score 3-5) and analyzed by flow cytometry. (FIG. 9F) Summary data of the absolute number of the total CD45+ population infiltrating the CNS (n=8 per group). (FIG. 9G) Summary data of the absolute number and frequency of 2D2 cells within the CD45+ CNS-infiltrating cells (n=8 per group). (FIG. 9H) Summary data and representative flow cytometry plots of T-bet expression in 2D2 WT or TCF1cKO cells infiltrating the CNS. FMO: fluorescence minus one control. (n=8 per group). (FIG. 9I) Summary data of cytokine production by host CD4+ T cells infiltrating the CNS (n=8 per group) *p<0.05, **p<0.01, ***p<0.001, ****p<0.0001, Two-way ANOVA followed by Sidak post-hoc test. Data are representative of three independent experiments (FIG. 9D). ns, not significant; Mann-Whitney test, two-tailed (FIG. 9F-9I). Mean±SEM are shown. Data are representative of two pooled experiments. (FIG. 9J) Summary data of the absolute number of 2D2 cells producing the indicated cytokines. (FIG. 9K) Summary data of the frequency of 2D2 WT or TCF1cKO producing the indicated cytokines ex vivo without further re-stimulation (n=5 per group). (FIG. 9L) Summary data of cytokine production by host CD⁴⁺ T cells infiltrating the CNS (n=8 per group). (FIG. 9M) Congenically marked 2D2 WT (CD45.2⁺) or TCF1cKO (CD45.1⁺CD45.2⁺) Th17_{INF} cells were co-transferred into WT recipient mice, harvested at peak of disease, and analyzed by flow cytometry. Summary data of the frequency of 2D2 WT or TCF1cKO producing the indicated cytokines ex vivo without further re-stimulation (n=3 per group).

FIG. 10A-10H—Sustained TCF1 expression dampens Th17 pathogenicity (related to FIG. 3A-3G). (FIG. 10A) Representative flow cytometry plots of IL-17A expression of WT or TCF1-Long naïve CD4+ T cells differentiated into the indicated Th17 subsets. (FIG. 10B) Summary data of cytokine secretion throughout pTh17 and npTh17 differentiation of WT or TCF1-Long CD4 T cells. (FIG. 10C) Schematic of pTh17 in vitro differentiation for adoptive transfer experiments. (FIG. 10D-10H) CNS-infiltrating immune cells were harvested from mice at peak of disease (EAE score 3-5) and analyzed by flow cytometry. (FIG. 10D) Representative histograms of TCF1 expression in 2D2 WT or TCF1-Long pTh17 cells. (FIG. 10E) Summary data of the absolute number of the total CD45+ population infiltrating the CNS (WT n=9; TCF1-Long n=8). (FIG. 10F) Summary data of the absolute number and frequency of 2D2 cells within the CD45+ CNS-infiltrating cells (WT n=9; TCF1-Long n=8). (FIG. 10G) Summary data of T-bet expression in 2D2 WT or TCF1-Long cells infiltrating the CNS (WT n=9; TCF1-Long n=8). (FIG. 10H) Summary data of cytokine production by host CD4+ T cells infiltrating the CNS (WT n=9; TCF1-Long n=8). *p<0.05, **p<0.01, Two-way ANOVA followed by Sidak post-hoc test. Data are representative of three independent experiments (B). *p<0.05, ****p<0.0001; Mann-Whitney test, two-tailed (E-F). Mean±SEM are shown. Data are representative of two pooled experiments.

FIG. 11A-11H—TCF1 deficiency confers pathogenicity to npTh17 cells (related to FIG. 4A-4H). (FIG. 11A) Schematic of npTh17 in vitro differentiation for adoptive transfer experiments. (FIG. 11B) Summary data of IL9 secretion by WT or TCF1cKO npTh17 cells. (FIG. 11C-11H) CNS-infiltrating immune cells (FIG. 11C-11F) or immune cells from draining lymph nodes (dLN) (FIG. 11G-11H) were harvested from mice at peak of disease (EAE score 1-3) and analyzed by flow cytometry. (FIG. 11C) Summary data of the absolute number of the total CD45+ population infiltrating the CNS (n=7 per group). (FIG. 11D) Summary data of the frequency of 2D2 cells expressing T-bet (n=7 per group). (FIG. 11E) Summary data of the frequency of 2D2 cells expressing Foxp3 (n=7 per group). (FIG. 11F) Summary data of cytokine production by host CD4+ T cells infiltrating the CNS (n=7 per group). (FIG. 11G) Summary data of the frequency of 2D2 cells within total CD45+ in the draining lymph nodes (dLN) (n=7 per group). (FIG. 11H) Summary data of the frequency of 2D2 WT or TCF1cKO cells expressing the indicated transcription factors (n=7 per group). ns, not significant, *p<0.05, **p<0.01, ***p<0.001; Mann-Whitney test, two-tailed (FIG. 11B-11H). Mean±SEM are shown. Data are representative of three independent experiments (B) or two pooled experiments (FIG. 11C-11H).

FIG. 12A-12K—TCF1 deficiency obviates the need for IL-23 for Th17 pathogenicity. (FIG. 12A) Schematic view of in vitro differentiation with IL-1b and IL-6 for adoptive transfer experiments. (FIG. 12B-12D) CNS-infiltrating immune cells (FIG. 12B-12C) or immune cells from draining lymph nodes (dLN) (FIG. 12D) were harvested from mice at peak of disease (EAE score 1-5) and analyzed by flow cytometry. (FIG. 12B) Summary data of the frequency of Foxp3+ cells within the 2D2 cell population infiltrating the CNS (WT n=11; KO n=12). (FIG. 12C) Summary data of cytokine production by host CD4+ T cells infiltrating the CNS (WT n=10; KO n=12). (FIG. 12D) Summary data of the frequency of SLAMF6+ and CXCR6+ cells within 2D2+ cells in the dLN of recipients of 2D2 TCF1 WT (n=8) and TCF1cKO (n=10) cells. (FIG. 12E-F) RAG1−/− mice were adoptively transferred with 106 naïve 2D2 TCF1 WT (2d2+Il17a-gfp+Tcf7fl/fl) or TCF1cKO (2d2+Il17a-gfp+dLck-Cre+Tcf7fl/fl) cells and followed for the development of neurological symptoms. (FIG. 12E) Mean EAE score is shown, p=0.0563, linear mixed model. (WT n=5, TCF1cKO n=5). (FIG. 12F) Summary data of the frequency of RORγt+ and T-bet+ 2D2 cells infiltrating the CNS of RAG1−/− mice (WT n=4; TCF1cKO n=5). (FIG. 12G) Glycolytic rate assay using Seahorse of WT (Tcf7fl/fl) or TCF1cKO (dLck-Cre+Tcf7fl/fl) CD4+ T cells at day 5 post-differentiation in the indicated conditions. (FIG. 12H) Induced glycolytic rate assay following acute injection of PMA and ionomycin by Seahorse of WT (Tcf7fl/fl) or TCF1cKO (dLck-Cre+Tcf7fl/fl) CD4+ T cells at day 5 post differentiation in the indicated conditions. (FIG. 12I-12J) CNS-infiltrating 2D2 cells were harvested and FACS-sorted from mice at peak of disease (EAE score 1-5) and analyzed by confocal microscopy. (FIG. 12I) Summary data of LipidTox MFI in 2D2 TCF1 WT and TCF1cKO harvested from the CNS. (FIG. 12J) Representative confocal images of LipidTox staining of 2D2 TCF1 WT and TCF1cKO harvested from the CNS of diseased mice (left). Summary data of staining analyses from randomly collected images (right). ns, not significant, *p<0.05, **p<0.01, ***p<0.001, ****p<0.0001; Mann-Whitney test, two-tailed (FIG. 12B-12D, 12F, 12I-12J). Mean±SEM are shown. Data are representative of at least two experiments. (FIG. 12E) p=0.0563, linear mixed model. (FIG. 12G-12H) **p<0.01, ***p<0.001; two-way ANOVA followed by Sidak post-hoc test. (FIG. 12K) (FIG. 12G) Representative flow cytometry data of RORγt expression in naïve 2D2 WT and TCF1cKO cells.

FIG. 13—TCF1cKO npTh17 cells resemble IL-23-induced pathogenic Th17 cells (related to FIG. 6A-61). FIG. 13 shows the proportion of open chromatin regions (OCR) containing TCF1 binding motifs in CXCR6+ and SLAMF6+Th17 cells.

FIG. 14A-B—A TCF1-driven transcriptional network determines Th17 cell state (related to FIG. 7A-7F). (FIG. 14A) Top-ranking transcription factors enriched in TCF1 WT- or TCF1cKO-specific differentially accessible chromatin regions according to HOMER motif enrichment analysis. p-values were capped at 10⁻⁵⁰ (Tcf7(HMG) p=10⁻⁴⁴⁰; RORg(NR) p=10⁻²⁵⁴; RORa(NR) p=10⁻²³⁷). (FIG. 14B) Western blot analysis of protein lysates of HEK293 transfected with TCF1-HA or Rorγt-Flag alone or in combination. Cell input and immunoprecipitation with HA-immunobeads in the indicated conditions are shown.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosure. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, optionally a mammal, including a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment but may. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the disclosure. For example, in the appended claims, any of the claimed embodiments can be used in any combination.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present disclosure provides for one or more therapeutic agents against combinations of targets identified. Targeting the identified combinations may provide for enhanced or otherwise previously unknown activity in the treatment of disease.

The term “in need of treatment” as used herein refers to a judgment made by a caregiver (e.g., physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compositions and therapeutic agents described herein.

The term “adjuvant therapy” as used herein refers to any treatment given after primary therapy to increase the chance of long-term disease-free survival. The term “neoadjuvant therapy” as used herein refers to any treatment given before primary therapy. The term “primary therapy” as used herein refers to the main treatment used to reduce or eliminate the disease, such as, cancer.

All publications, published patent documents, and patent applications cited herein are hereby incorporated by reference to the same extent as though each individual publication, published patent document, or patent application was specifically and individually indicated as being incorporated by reference.

### OVERVIEW

Immune cells like CD4⁺ T helper 17 (Th17) cells, encompass a spectrum of cell states including cells that maintain homeostatic tissue functions, such as mucosal barrier integrity, and pro-inflammatory cells that drive autoimmune tissue inflammation/damage. Th17 cells are critical for the maintenance of tissue homeostasis and for host defense against extracellular bacterial and fungal pathogens. Th17 cells are also key mediators of pathogenesis in several autoimmune diseases, including multiple sclerosis, rheumatoid arthritis, psoriasis, type-1 diabetes, and inflammatory bowel disease. The diverse roles of Th17 cells can be explained by the existence of several cell states in between two extremes marked by homeostatic or non-pathogenic and inflammatory or pathogenic.

As used herein, “homeostatic” and “non-pathogenic” Th17 cells (npTH17) refer to a Th17 cell state marked by production of IL-17A, IL-22, and IL-10, supportive of maintaining mucosal barrier function and typically rely mainly on oxidative phosphorylation for their energetic demands. (Lee et al., 2012, Omenetti et al. 2019). npTh17 cells are induced and maintained by the gut microbiota in vivo and by in vitro differentiation of naïve CD4+ T cells with TGFβ1+IL-6. In one embodiment, a npTh17 cell state is defined by increased expression of the genes in Tables 2A and/or 2B. As used herein “inflammatory” or “pathogenic” Th17 cells (pTh17) refer to Th17 cells that are induced by inflammatory states in vivo and by IL-1β+IL-6+IL-23 in vitro. pTh17 cells have the ability to fuel destructive autoimmunity and rely on both aerobic glycolysis and oxidative phosphorylation for their energetic demands (Bettelli et al, 2008; Lee et al, 2012; Omenetti et al. 2019; Qiu et al., 2020; Wagner et al., 2021; Xu et al. 2021). In one embodiment, pTh17 cells are defined by increased expression of the genes in Tables 1A and/or 1B.

The present disclosure has identified regulators that determine these cell states and provide means to control tissue inflammation without compromising the physiological functions of Th17 cells. These regulators and Th17 cell states have not been previously identified because the subsets could only be identified by tissue and cell specific analyses as described herein for the first time. Accordingly, in one aspect, embodiments disclosed herein provide for methods of treating autoimmune and inflammatory diseases. In one example embodiment, methods of treating autoimmune and inflammatory diseases comprise administering one or more agents that maintain a Th17 cell in a homeostatic state and/or shift a Th17 cell away from a pathogenic cell state. In another aspect, embodiments disclosed herein are directed to immune cells genetically modified ex vivo to increase or decrease expression of key immune cell regulators that maintain the immune cell in a homeostatic cell state and/or shift a Th17 cell away from pathogenic cell state and use of said cells in the treatment of inflammatory disorders.

### Methods of Treating Autoimmune or Inflammatory Disease/Disorders

In certain embodiments, administering one or more agents that modulate a Th17 cell systemically as discussed herein may be used to treat inflammatory diseases or disorders. In certain embodiments, administering an agent to induce modification or modulation of the underlying drivers of pathogenicity in the subject may be used to treat inflammatory diseases or disorders. As used herein “inflammatory disease” or “inflammatory disorder” refers to a disease or disorder caused by inflammation resulting from immune cells attacking the body's own cells or tissues. and may further include autoimmune diseases or disorders. As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

In example embodiments, a method of treating an inflammatory disease or disorder comprises administering to a subject in need thereof one or more agents that decrease expression of one or more genes from Table 1A and/or Table 1B or increase expression of one or more genes from Tables 2A and/or Table 2B (collectively, “target genes”), or a combination thereof.

Table 1A provides a list of genes whose RNA are more highly expressed in pTH17 cell states and whose expression are repressed by TCF1.

TABLE 1A
Genes Expressed in a Pathogenic Immune Cell State

| | | | | |
|---|---|---|---|---|
| Pygl | Rora | Fbxo27 | Adam8 | Hk2 |
| Dennd5a | Abhd15 | Cpt1a | P2ry1 | Fbxl21 |
| Ndrg1 | Abca1 | Khnyn | Nedd4 | Bhlhe40 |
| Chsy1 | Pim1 | Rorc | Myo1f | Rarg |
| Cchcr1 | Lsp1 | Mgll | Ppp1r9b | Per1 |
| Edaradd | Nr1d1 | Ltb4r1 | Tmem231 | Srebf2 |
| Gpr132 | Tmem64 | Dipk2a | Hip1r | Litaf |
| Nod1 | Etv6 | Fam160a2 | Ptprj | Cpd |
| Resf1 | Bcl2 | Tmem59 | Zfp120 | Hid1 |
| Runx2 | Glul | Furin | Inpp5d | Crybg3 |
| Snx20 | Dok2 | Atp2b1 | Il17re | Tiam1 |
| Asx12 | Fam20a | Ncoa7 | Zyx | Tktl1 |
| Ccr2 | Cipc | Ly6a | Clic4 | Ssbp2 |
| Lamc1 | St3gal6 | Slco3a1 | Cd7 | Ccr8 |
| Firre | Bicral | Ppp1r12a | Myadm | Nhsl2 |
| Dse | Kcnq5 | Thsd7b | Tnfsf14 | Cyth4 |
| Mdfic | Fgl2 | Trim16 | Fam107b | Traf1 |
| Cxcr6 | Slc25a24 | Tspan2 | Atxn1 | Csf1 |
| Ccr6 | Il18r1 | Irak2 | Itgb3 | Socs2 |
| Tsc22d3 | Itgb7 | Usp20 | Rai1 | Klrb1f |
| Mgat5 | I12rb | Mturn | Gfpt2 | Mapkapk3 |
| Gm32856 | Podnl1 | Galnt2 | Itga2 | Fcho2 |
| Hpgds | Gm2a | Ccr4 | Il2ra | Rftn1 |
| Pik3ap1 | Serpinb1a | Adam19 | AI506816 | Sla |
| Cd47 | Itgb1 | Slc2a3 | B3galt5 | Gna15 |
| Ly6e | Tmprss13 | Lmnb1 | Gpr65 | Ifngr1 |
| St6galnac3 | Selplg | Sor11 | Capn3 | Maf |
| Bik | Kcnk5 | Mmp25 | Ramp1 | Rbl2 |
| Slamf1 | Smad3 | Cry1 | Lmo4 | Pparg |
| Rcbtb2 | Lilr4b | Lilrb4a | Apbb1 | Acsbg1 |
| Glipr2 | Il18rap | Plekhf1 | Atp8b4 | Rbpj |
| Cish | Tlr1 | Aopep | Krt83 | Il10ra |
| Il23r | Fam124b | Il7r | Klrk1 | Hip1 |
| Smpdl3a | Map6 | Il17ra | Itgae | Cd82 |
| Gabarap12 | Coro2a | Galm | Zfyve1 | Cd101 |
| Tmem176a | Tmem176b | Actn2 | Rab11fip4 | Pla2g6 |
| Snx32 | Adamts6 | Hlf | Ogfod2 | Dvl2 |
| Repin1 | Dbp | Gm6277 | Tmed1 | Macrod2 |
| Mnt | Hic1 | Pdk2 | Ankrd9 | Mir5130 |
| Dusp6 | Cstad | Fkbpl | Arrdc4 | Rusc1 |
| Gm7932 | Per3 | Arhgap33 | Mir17 | Gon7 |
| Gm2788 | St3gal3 | Espn | Phldb3 | Snai1 |
| Serpine1 | BC053393 | Mif4gd | Cox7a1 | 1700067K01Rik |
| 4930430F21Rik | Mier1 | Mir6951 | Nanos3 | Rtn3 |
| Wdfy2 | Mir6974 | Fstl3 | Fosl2 | Frs3 |
| Gamt | Mir23a | Arhgap23 | Klf12 | Cd9 |
| Ptprv | Mcmbp | Dlec1 | Nlgn2 | Kbtbd6 |
| Pagr1a | Ptpre | Gpr157 | Slc25a22 | Mafk |
| 4833412C05Rik | Cdkn1a | Tmem159 | Tpcn1 | Llgl1 |
| Nfic | Cxxc5 | Thoc2 | Tle6 | Camkk1 |
| Mir132 | Klf4 | Ino80 | 0610040F04Rik | Pogk |
| Tspoap1 | Adgra2 | A330009N23Rik | Zc3h18 | Arpc1b |
| Fam13b | Nfil3 | Pkd1l2 | Usp44 | Lipe |
| Mir574 | Cuedc2 | Rpl18 | Fam219b | Odad1 |
| Tmem221 | Gm14486 | Sigirr | 1700060C20Rik | Sh2b3 |
| Stx11 | Chst11 | Ctnnb1 | Dok1 | Fbxl2 |
| Stx1a | Unc5b | Adssl1 | Micall2 | Rasgef1b |
| Nbeal2 | Plet1os | 6430571L13Rik | Mir8105 | Stk32c |
| Tmem17 | Dapk1 | Fam122a | Tex44 | Fosb |
| Eif3d | F2r | Kif17 | Rab26 | Nkapd1 |
| Elapor1 | Gnaz | Acads | Crnde | Tctn1 |
| B9d2 | Dnajb2 | A730081D07Rik | Mir193a | B3gat3 |
| Gulp1 | Ubxn11 | Jaml | Rabgap1l | Snrpd3 |
| Fkbp5 | Tnfrsf18 | Hc | St3gal5 | Asb6 |
| Ftx | Mapkapk2 | Napb | Vasp | Tef |
| Dock5 | Gabbr1 | Pde8a | Lmf1 | Ust |
| Il4i1 | Nfe2 | Ap1m1 | Ppp1r3f | Gm29776 |
| Rhbdl3 | B230217012Rik | Ergic1 | Heg1 | Plcb4 |
| Vax2 | Prodh | Cd247 | Kdr | Ptpn18 |
| Nxnl1 | Kcnk7 | Mir7216 | Csnk1d | N4bp1 |
| Dnal4 | Grk2 | Morrbid | Prrt1 | Arl15 |
| Slc9a1 | Mir6380 | Sdr39u1 | Fgfr1 | Crygn |
| Bptf | Tsc22d1 | Fam136a | 4932435O22Rik | Wdfy4 |
| Tmem51os1 | Cercam | Cnot2 | Sergef | Pgap3 |
| Arid3a | Impdh1 | Tbc1d2 | Wdr73 | Gm15417 |
| Sh3bgrl2 | 1700096J18Rik | Chmp6 | Unc13d | Aldoa |
| 0610009L18Rik | Sgsm2 | Hook2 | 4921507G05Rik | Pou2f2 |
| Gba | Slc16a3 | Nuak2 | Ptp4a2 | Lrp8 |
| Stam | Eef1d | Banf1 | Tnfrsf21 | Selenoo |
| Spg7 | Alkbh5 | Sp8 | Slc16a6 | H4c6 |
| H4c8 | Parp1 | Zfp511 | Phactr4 | Gnaq |
| Mga | Zc3h12c | Nsd1 | Fosl1 | Dennd2c |
| Tmem121b | Galnt3 | Mknk1 | Gnl1 | Thbd |
| Gna11 | Batf | Ifitm10 | Pura | Acot11 |
| Tmf1 | Slc9a4 | Cfap61 | Mei4 | Crispld2 |
| Yif1a | Shpk | Impa2 | Stard8 | Tpx2 |
| Gm17767 | Lif | Rmdn3 | Wrnip1 | Plekhm1 |
| Rhbdf2 | Lrp5 | E4f1 | Ildr1 | 1700126H18Rik |
| Cela1 | Ahi1 | Rmi1 | AV039307 | Acadsb |
| Lsm4 | Ap3m2 | Lrrc25 | Colgalt2 | Hnrnpl |
| Irf2bp2 | Plec | Qars | Tead3 | Gas2l1 |
| Vmp1 | 6430710C18Rik | 1700020M21Rik | Gm7904 | Prr29 |
| Slc25a19 | Tnk2 | Anp32a | Xcl1 | Hmgb1 |
| Map2k3 | Snx25 | Tmem30b | Zfp654 | Cd48 |
| Gsap | Mettl24 | Rnf224 | Zmiz1 | 1110028F18Rik |
| Gpdl1 | Ahr | Mdfic2 | Ccn5 | Ccr1 |
| Lrrn2 | Gbp6 | Gm19510 | Capzb | Gm20110 |
| Smarcel | Trem2 | 3830408C21Rik | Art2a | Fars2 |
| Shisal2b | 4930563M21Rik | Bbs4 | Jcad | Smug1 |
| Ldlrad1 | Vps54 | Gm11482 | Btnl9 | Rgs2 |
| Lonrf3 | Rufy1 | Mical1 | Wdr44 | A530013C23Rik |
| Itpkb | Slc25a1 | AU019990 | Lrrc8d | Phc3 |
| Slc37a1 | Ms4a20 | Tiparp | Hectd1 | Phospho1 |
| Grik1 | Enpp6 | Gm19345 | Tex33 | Wincr1 |
| Snap23 | Ccdc146 | Gm46545 | Samd11 | Cast |
| Nod2 | 5830428M24Rik | Zfp608 | Rgsl1 | 9530068E07Rik |
| Olfr1372-ps1 | Fmnl1 | Kif1a | Magi3 | Slc35f5 |
| Eml4 | Med4 | 4921513I03Rik | Lamtor4 | Gzmb |
| Medag | Cep170b | Nptn | Agbl1 | Gm11413 |
| Cubn | Mir7688 | Igfbp7 | Dot1l | Gm12532 |
| Lrrc8c | 4930526H09Rik | Rnf141 | 4930515G16Rik | Hacd4 |
| Trpm2 | Itprip12 | Poli | Muc3 | Rnpep |
| Platr14 | Crybb1 | Fhl2 | Crybg1 | Nup214 |
| 4930522017Rik | Lrp1 | Rbm47 | Cdkl3 | Srp68 |
| Gdpd5 | Il21 | Klhl25 | Tm2d3 | Nek6 |
| Plet1 | Cep43 | Lyl1 | Spice1 | Pmaip1 |
| Acvr2a | Tnfrsf9 | Mccc2 | Dnah12 | Rasl12 |
| Ankrd44 | Gpr107 | Gm15441 | Rassf1 | Synj2 |
| Sdccag8 | Tlr9 | Cdh26 | Sbk1 | Sdc1 |
| C5ar1 | Galnt12 | Tango6 | Gm13031 | Spata3 |
| Csk | Pik3cg | Abr | E530011L22Rik | P2ry14 |
| Ctss | Muc13 | Dtnb | Pex7 | Tdrd5 |
| Fes | Igsf5 | Mthfs | Mthfsl | Lax1 |
| Hmga2 | Itprid2 | 4930556J02Rik | Smg6 | Kcnmb1 |
| Nfkbil1 | Itsn1 | Pde6h | Pik3ca | Slc22a8 |
| Arap1 | Ccdc88b | Cmip | Plekhg3 | Art4 |
| Dhrs9 | Tbkbp1 | Gm10640 | Rab19 | Lipm |
| Abca2 | Adap1 | Nmnat2 | Rab22a | P2rx3 |
| Txndc5 | Cep192 | Uba7 | Asgr2 | Vash1 |
| Armc2 | Ccr9 | Cybrd1 | Sp3os | 2810459M11Rik |
| Ahdc1 | Mir7094-2 | Colq | Mir29b-1 | Pde6d |
| Psd2 | Sdhaf1 | Mir684-2 | Fasl | Il1r1 |
| Mfn2 | Slc2a5 | Btbd9 | Tg | Acadl |
| Dennd1b | Pogz | Adgrv1 | Efr3a | Epx |
| Fos | Haao | S100a10 | Nucks1 | Cdyl2 |
| Marchf3 | Oser1 | Ctcf | Vmn2r85 | Alpk2 |
| Rnh1 | Txnrd1 | Ankrd31 | Rad18 | Mtx3 |
| Crtc3 | Mir7094-1 | P2rx7 | Pdella | D230030E09Rik |
| Mir365-2 | Ccr3 | Hsf5 | Zfp395 | Ablim3 |
| Gucy2c | Muc1 | Kit | Il21r | Kcna3 |
| Krt27 | Cass4 | Mrtfb | Zbtb16 | Mkrn2os |
| P2rx4 | Plin3 | Snrk | Mex3c | Ppp1r12b |
| 6820426E19Rik | Brip1 | A230108P19Rik | Pttglip | Adgb |
| Cd86 | Zfp664 | Pelo | Mtmr7 | Retnlb |
| B4galnt2 | Creb5 | AU040320 | Comt | Csf2 |
| Gm12185 | F13a1 | Hs2st1 | Susd3 | Ncor1 |
| Il12a | Mir1928 | 5430437J10Rik | Serpine2 | Syt12 |
| Gpatch2 | Hspb8 | Mid1 | Tex2 | 4833427F10Rik |
| Eps8 | Tut4 | Mir7683 | Rgs13 | Serp2 |
| Snora16a | Fam169b | Cic | Jazf1 | Ddx19a |
| 1700064M15Rik | Aak1 | Hccs | Rpap2 | Tcf7l1 |
| Bmerb1 | Rnf2 | 2900040C04Rik | ccdc198 | Nosip |
| Filip1 | Tomm5 | Gm34816 | Tap2 | D5Ertd579e |
| A930011G23Rik | Anln | Dcun1d2 | Glrp1 | Glis2 |
| Eml1 | Nsmaf | Dsc3 | Itpr1 | Mexis |
| Syne2 | Il17a | Tyrobp | Cox6a1 | Gimap3 |
| Havcr2 | Pkib | Sv2c | Zswim6 | Ikzf2 |
| Srgap3 | Grk3 | Rrp12 | Blk | Galnt14 |
| Shc4 | Timp2 | Gm3716 | Oacyl | Ralgps1 |
| Stk10 | Cmc2 | Vps13d | Efcab3 | Gm12709 |
| Hmox2 | Tbl1x | Eif2ak1 | Rcsd1 | Spag9 |
| Moxd2 | Agpat4 | Togaram1 | Dthd1 | Bbs9 |
| Slc11a1 | Ypel2 | 1700001K23Rik | Akr1e1 | Ap4b1 |
| Mocs1 | Mtrr | Mbp | Ttc21b | Cxcl9 |
| Mir8120 | Tcl1b1 | Slc1a5 | Tmem38b | Mllt10 |
| Rlf | Dok6 | Olfr525 | Henmt1 | Fancc |
| Gm27162 | Bpifc | Nfatc2 | Snx1 | Gm38414 |
| Polr1has | Mir6387 | Acap2 | Tmtc4 | B4galnt4 |
| Tmem154 | C430042M11Rik | Casc3 | Ptpn11 | Gm13582 |
| Pou5fl-rs4 | Krt28 | Zfp362 | Fam172a | Pla2g2c |
| Nfkb2 | Axdnd1 | Prss30 | Mylk2 | Cacna1s |
| Mir6906 | Vdac1 | Icosl | Brca1 | 4930431P03Rik |
| Ak5 | Ern1 | Myod1 | C330024C12Rik | Car5b |
| Gm11696 | Ubac2 | Bcl2l15 | Abcb9 | Nfe2l2 |
| Mboat1 | Tmem260 | Klrb1c | Nrsn2 | Fbxw11 |
| Gas7 | Bnip2 | Gm1720 | Gcg | Elf1 |
| Runx1 | 4930519G04Rik | Irgc1 | Mir1892 | BC049352 |
| Ip6k1 | 1700016L21Rik | 4930434J06Rik | Pon2 | Gm527 |
| Sik1 | Pla2g12b | Slc15a2 | Gm13710 | Rims2 |
| Mov10 | Atad1 | Ticam1 | Hmcn2 | Sumo3 |
| Fyco1 | Ifitm3 | Paox | Snx31 | Kcnc1 |
| Gm17745 | Uggt1 | Plcxd2 | Tnks2 | Ccl3 |
| Ostf1 | Stum | Ncapg | Shmt2 | Sgf29 |
| Synpo | Rad51b | Plcb3 | Zfp746 | Gvin-ps3 |
| Gvin-ps5 | Sub1 | 4932438H23Rik | Serinc5 | Mir1953 |
| Itpr2 | Shcbp1l | H2-Q5 | H2-Q6 | Mroh8 |
| Nop58 | Pstpip1 | Tspyl2 | Wdr95 | Susd1 |
| Synm | Atp8a1 | Zzef1 | Myo16 | Tex43 |
| 4933405E24Rik | Lrrc49 | Gm11529 | Dnajc27 | Nup210l |
| Ache | Btbd10 | Cacng2 | Pebp1 | Ptprs |
| Klhdc9 | Mppe1 | Camk2b | E330011021Rik | Hdac4 |
| Scgb1a1 | Nefl | Nos3 | Ppp3ca | Sh2d4a |
| 3300005D01Rik | Dock8 | Sgip1 | Spryd7 | Tpst2 |
| Ehd1 | Prss12 | Rhobtb1 | Smap2 | Creb1 |
| Dock10 | AA536875 | Metrnl | Reps1 | Tm6sf1 |
| Muc4 | Eef2kmt | Srl | 4930598F16Rik | Slc6a19os |
| Icos | Med10 | Ifi204 | Igtp | Slc38a1 |
| 4930465M20Rik | 1700061F12Rik | Clcn6 | Emb | Tnfrsf17 |
| Cdc5l | Lrrc42 | 4921529L05Rik | Ldlrad4 | Milr1 |
| Adamtsl4 | Stau2 | Trp53bp2 | Chmp4b | Lcmt2 |
| Ppp1cc | Cmklr1 | Rab20 | Copg1 | Ltb4r2 |
| A430057M04Rik | Tmem171 | Pvt1 | Sh2d2a | Ncapd3 |
| St6galnac5 | Ndfip1 | Rps18-ps4 | Aktip | Col6a4 |
| Abcc4 | Cyp2e1 | Cxcr5 | Ifnar2 | Tab2 |
| Noxa1 | Metap2 | Mtmr1 | Nadk | Qprt |
| Cask | Jdp2 | C030005K06Rik | Ctso | Olfr530 |
| 1700007L15Rik | Ndufb10 | St8sia4 | Etv3 | Kcnd3 |
| Rfx2 | Cep19 | Gm30726 | Dus11 | Rcan1 |
| Srprb | Calhm6 | Fas | Mta3 | 4930578N18Rik |
| Lss | Akt1s1 | Hnf1b | Ralgapa2 | Tbx21 |
| 5830454E08Rik | Arhgap19 | Dct | Nup107 | Gabrr3 |
| G6pc3 | Ppm1k | Cwh43 | Hmg20a | 4930428O21Rik |
| M1ap | Msh4 | Dnah11 | Tsg101 | Prok1 |
| Spopl | Abtb2 | Gpr89 | Mrpl14 | Stmnd1 |
| Jpt1 | Cpa3 | Batf3 | Slmap | Coq9 |
| Dgcr2 | Wipf1 | Zfp874a | Arpc2 | Cx3cr1 |
| Adam17 | Stxbp4 | Exoc6b | Gadd45b | Bcl6 |
| 1700012B07Rik | Cpb1 | Hif1an | Pabpc2 | Cdh3 |
| Prkacb | Gcnt2 | Olfr531 | 4930505K14Rik | Pigv |
| Cnr2 | Ttll11 | Tulp1 | Adam15 | Bcl6b |
| Cbr1 | Alg14 | Me1 | H2bc18 | Cyp7a1 |
| Gm30173 | Il9 | Nmrk1 | Rpl11 | Gmeb 1 |
| Zfp36 | Ppm1e | Plxnc1 | Gyg | 4930559C10Rik |
| Fam91a1 | Doxl2 | Fth1 | Pten | Nrg2 |
| Swap70 | Gzmk | St6galnac2 | Zfp57 | Adcy4 |
| Rps6kb2 | E230029C05Rik | Mib1 | Smpd1 | Sgms2 |
| Arrb1 | Nr3cl | Hdac11 | Kif5c | Mfsd13a |
| Btrc | Tnfrsf1b | Pacs2 | Calcoco1 | Peli1 |
| Mrps27 | Plekha6 | Rtkn2 | Hacd2 | Btg3 |
| Pip5k1b | Ptges | Trib1 | Vamp1 | C030018K13Rik |
| Raet1c | Lrrk1 | Adrb1 | Stk24 | Itga3 |
| Gnal | Zfp948 | Adat1 | Eef2k | Selenom |
| Scrg1 | Mapkbp1 | Ston2 | Niban2 | Dtx1 |
| Hk1 | Dhx34 | Krt80 | Saysd1 | 1700012B09Rik |
| Gm8439 | Myoc | Golga4 | Krt222 | Tnpo2 |
| Otogl | Grb7 | Bbs7 | Eif3a | Ub13 |
| Cmtm7 | Srgn | Cpn2 | Inpp5k | Trpv2 |
| Samsn1 | Ciart | 1700025F24Rik | Tspyl3 | Clip1 |
| Pafah1b2 | Brwd1 | Coa8 | Ankrd6 | Cabin1 |
| Mir133b | Agpat5 | 1600020E01Rik | 4930513D17Rik | Ptpra |
| Mbd5 | Olfm3 | Vps28 | 5430421F17Rik | Tmprss6 |
| Gm10804 | Cdc73 | 1810013L24Rik | F630206G17Rik | Zfp397 |
| Cox4i2 | Rgs10 | Olfr524 | Prpf4b | Slc2a1 |
| Egfr | Gpr12 | Aifm1 | Fam76b | Fgd6 |
| Fhod1 | Akt2 | Tmbim6 | Itpr3 | Tep1 |
| Otulin | Swt1 | Ap3d1 | Ccdc6 | Sema6d |
| Rab39 | Slc36a3 | Trmt61a | Uaca | Tnfsf10 |
| Fam193a | AA986860 | Phb | 1700030L20Rik | Mitf |
| Plekho2 | Foxn2 | Dennd4a | Fam117a | Ptpn2 |
| Clnk | Trappc3l | Capza2 | Zhx1 | Xpc |
| H2-D1 | Il5 | Tfdp2 | Ubl5 | Cand1 |
| Eloa | Ikzf3 | Pkdcc | Tbc1d31 | Crybg2 |
| Cd84 | Soat1 | Trim8 | Rnf19b | Ap2a1 |
| Nkg7 | Entpd7 | Serpina3f | Kcnmb4 | Xcr1 |
| Slc25a43 | Tbc1d9b | 4930572O13Rik | Gm38425 | Ptgfrn |
| Scart2 | 4833419F23Rik | A430090L17Rik | H4c1 | Plekhh3 |
| Atf3 | Lefty2 | Soga1 | Fads6 | Ptk2b |
| Cluh | Ppp1r16b | Fam180a | Il17f | Abcc3 |
| Hadh | Slc27a6 | Hemk1 | Tnfrsf1a | Cerk |
| Rbpms2 | A330023F24Rik | Gm17746 | Gxylt1 | Ercc8 |
| Ppp1r14c | Gstcd | Nebl | Cdon | Ctif |
| Dpy19l3 | Frmd4a | Pdcd1lg2 | Slc22a16 | Dusp22 |
| Stard7 | Xrra1 | Scd1 | Fam50b | 2700049A03Rik |
| Mfsd5 | Ak2 | Disp1 | Slc38a10 | Megf10 |
| Ttc39b | Ccdc88c | Carl1 | Psmc6 | Pcbp3 |
| Gm20750 | Plxnd1 | Klrb1b | Sp4 | Ppp3cc |
| Zfp638 | Gatad2a | Vmn1r3 | Kat6b | Kif23 |
| Erich6 | Art2b | Os9 | Adarb2 | Kmo |
| Ndufa9 | Serpina4-ps1 | Ccdc84 | Tjp1 | Degs2 |
| Tasor | Serpinb1b | Ttc1 | Ric1 | Nrn1 |
| Tmem247 | Gimap4 | Ms4a4c | Mybl2 | Prdm2 |
| Prep | Lonp2 | Lyrm9 | Adgrl2 | Pax5 |
| Smpdl3b | Pik3cd | Slc15a1 | Spock2 | Mef2a |
| Sit1 | Kcna2 | Mir3970 | Zfp704 | Adam12 |
| Ski | Cpped1 | Ttyh1 | Ccdc112 | Spata2 |
| Anp32b | Itgax | Ube2f | 9130015A21Rik | Atf7ip |
| Scaper | Amph | Rbmx | Phldb2 | Sufu |
| Kbtbd2 | Tbck | Dnajc5b | Pde4b | 8030474K03Rik |
| 4921525O09Rik | Mark2 | Frrs1 | Gng2 | Hdc |
| Hgsnat | Zhx3 | Thg1l | Corolc | Syne3 |
| Cfap126 | Pdzd2 | Gm33104 | Med30 | Zbtb1 |
| Rasgef1c | Enpp7 | Aven | Cul5 | Pde4a |
| Stard3nl | Mir5118 | Ncoa3 | Gpr183 | Nckap5 |
| Nin | Pdhx | Il15 | Vrk2 | Clec12a |
| Fabp12 | Dynll2 | Plekha5 | Olfr1423 | Prr51 |
| AI467606 | Mirlet7i | Tgm2 | Prss39 | Trpm6 |
| Col15a1 | Sema4d | A830029E22Rik | Ly86 | Apol9b |
| Sectm1a | Map3k20 | Matn2 | Sema4b | Zdhhc22 |
| Gm38560 | N4bp2 | Gm29685 | Gm5111 | Zfp148 |
| Ccdc63 | Iffo2 | Hfm1 | Vwf | Trim27 |
| Vps53 | Torlaip1 | Mogat2 | Snx30 | Scamp2 |
| Marf1 | Tusc3 | Aptx | Rad50 | Fchsd2 |
| Nsmce2 | Thada | C2cd3 | Gpatch2l | Ssbp3 |
| Minpp1 | Rab2a | Dio2 | Map4 | Rbl1 |
| Aurkb | Sytl2 | Nfkbiz | Fbxo40 | Glrx |
| Pdcl3 | Ppp2r5a | Fbxw7 | Ddr1 | Glb1 |
| 2310069B03Rik | Mapk14 | Fut7 | H2-T3 | Iqsec1 |
| Gm15569 | Tmem256 | Rap1b | Rtn4rl1 | Dlgap4 |
| Ccl1 | Yars | Hnrnpul1 | Klf9 | 5730460C07Rik |
| Fryl | Mett17a1 | Syngr4 | Cox10 | Irf2bpl |
| Airn | Prex 1 | Macroh2a1 | Plekha3 | Unc13a |
| Ankrd50 | Dglucy | Ear7 | Plekho1 | Ly96 |
| Mtap | Rai14 | Golm1 | Ltf | Kif3a |
| Nbn | Slc17a4 | Cldn14 | Gm15506 | Psmd4 |
| Arhgap31 | Ctps2 | Parl | Cd302 | Tbrg4 |
| Tmc6 | Hdnr | Immt | Ptcd3 | Fnbp1 |
| Vamp4 | Vmn2r100 | Snx27 | Gfi1 | Ncbp3 |
| Fabp5 | Pabpc1 | Galnt1 | Hifla | Tsen2 |
| Sh3bp2 | Apbb1ip | Xxylt1 | Ddx3x | Rtf2 |
| Vps26b | Asic2 | Il1rl1 | Slc22a15 | Eps8l3 |
| Rbm24 | 4930539M17Rik | Sv2b | Tle3 | Cab39l |
| Atl1 | 4933406K04Rik | Acpp | Iyd | Samd4b |
| Cacnb1 | Ncald | 4930471C04Rik | Crot | Ankrd16 |
| Utp6 | Aurkaip1 | Wdr66 | Rad23b | Sdcbp2 |
| Grap2 | Tmem135 | Srbd1 | Dars | Ly6d |
| Appl1 | Cldn2 | Denn2b | Fastkd3 | Tedc1 |
| N6amt1 | Shisa5 | Nol10 | Akirin1 | Plxdc1 |
| Sik2 | Cables1 | Cfap44 | 4930546K05Rik | Nrp2 |
| Pigl | Lrrc71 | Parp9 | Fggy | Ccdc71l |
| Gm12171 | Cep63 | Gm9949 | Olfr186 | Cyp4f39 |
| Kctd13 | Plod3 | Zfp472 | Slc24a2 | Ap5b1 |
| Rras2 | Hectd2 | Unc5c | Slc43a2 | Dnajb6 |
| Sbf2 | Stk17b | Arhgap25 | Kcna6 | 4930578M01Rik |
| Wars2 | Gtf2ird2 | Lef1 | Gm4632 | Larp1 |
| Map3k1 | Ndufa10 | Rdx | Rttn | Afg3l2 |
| Myo9b | Btbd19 | Nfkbia | Ptpn1 | A630019I02Rik |
| Rd3 | Arid5a | B3gnt3 | Cops2 | Trim55 |
| Arnt2 | Gpn2 | Dcaf12 | Inf2 | Suco |
| Oxtr | Serpina12 | Gmpr | Supt20 | Tanc2 |
| Fgf6 | Rara | 1700025M24Rik | Fam83a | Zfp438 |
| Slc35b4 | Rassf5 | Septin9 | Adgre5 | Cwc25 |
| Yy1 | Poc1a | Gdi1 | Lgals1 | Pias4 |
| Ppp2r3d | Ier2 | Retreg1 | Dand5 | 2810021J22Rik |
| Hoxc5 | Tmie | Camk2n1 | Mir3089 | Card10 |
| Med14 | Mcrip1 | Osbpl2 | Rrnad1 | Rassf8 |
| Tecpr1 | Cox5a | Mfap4 | Slc18a2 | Tspan18 |
| Mob3a | Tfip11 | Wdr90 | Bcl2l1 | Dhrs11 |
| Noc2l | Col16a1 | Msc | 2310003N18Rik | Tapbp |
| Lrrc47 | Chmp2a | Prrg2 | Ttc39c | Itpkc |
| Ppp2ca | Nol4l | Olfr279 | Gm13003 | Map3k7cl |
| Plcl1 | Csrnp1 | St6galnac6 | Fst14 | Mast3 |
| Oga | Azi2 | Crem | Kdm4a | Stk16 |
| Krt42 | Zbtb34 | Camk2g | Kdm4d | Lck |
| Src | Ssbp4 | Cd79b | Plcd1 | Nrros |
| Smbd1 | Mir142 | Dnajb12 | Trerf1 | Clrn3 |
| Hunk | Cdh23 | Snrnp200 | Wscd1 | Cradd |
| Sema4a | 4930594021Rik | Nmur1 | 4933406J09Rik | Hilpda |

Table 1B provides a list of transcription factors whose binding motifs are specifically enriched in genes expressed in the pTh17 cell state and therefore are likely drivers of the pTh17 cell state.

TABLE 1B
Transcription Factors Expressed in a Pathogenic Immune Cell State

| | | | | |
|---|---|---|---|---|
| RUNX1 | SP1 | STAT3 | IRF4 | RORG |
| RORA | STAT1 | BATF | JUNB | FOSL2 |
| Jun | Fosl2 | Junb | Fos | Jund |
| Tfe3 | Usf1 | Usf2 | Clock | Arntl |
| Arnt | Bhlhe40 | Tfeb | Mxi1 | Max |
| Myc | Nfyb | Nfya | Nfyc | Ddit3 |
| Atf4 | Cebpg | Zbtb7a | Mbd2 | Epas1 |
| Hif1a | Mecp2 | Xbp1 | Cux1 | Zbtb33 |
| E2f2 | E2f5 | Hinfp | Mtf1 | Nrf1 |
| Pax5 | Yy1 | Pou1f1 | Nfe2l1 | Tead2 |
| Arid5b | Sox5 | Foxp2 | Foxo1 | Foxk1 |
| Foxo3 | Nr1d1 | Nr1d2 | Rora | Rorc |
| Stat5a | Stat5b | Stat3 | Stat4 | Rel |
| Relb | Rela | Nfkb1 | Nfkb2 | Maff |
| Mafk | Maf | Mafg | Bach2 | Nfe2l2 |
| Bach1 | Batf | Foxj3 | Foxj2 | Foxo4 |
| Prdm1 | Irf7 | Irf9 | Lyl1 | Tcf12 |
| Tcf3 | Smad3 | Smad2 | Smad4 | Mef2c |
| Mef2a | Mef2d | Rfx3 | Rfx1 | Rfx2 |
| Rara | Rxra | Pparg | Nr1h3 | Nr2c2 |
| Ar | Nr3c1 | Pbx1 | Pknox1 | Esrra |
| Esrrg | Esr1 | Rarg | Stat6 | Nfatc1 |
| Nfatc2 | Nfatc3 | Ebf1 | Prdm16 | Atf2 |
| Crem | Atf1 | Creb1 | Nr2c1 | Rxrb |
| Mecom | Gata1 | Gata3 | Prdm5 | Tcf4 |
| Zeb1 | Hsf1 | Hsf2 | Ikzf1 | Ctcf |
| Zfx | Bcl6 | Nfia | Nfic | Smarca5 |
| Thap11 | Dbp | Nfil3 | Thra | Tbp |
| Pou2f1 | Pou2f2 | Zkscan1 | Gfi1 | Pbx2 |
| Rest | Srf | Nr4a1 | Prdm9 | Rbpj |
| Tbx21 | Srebf1 | Srebf2 | Taf1 | Tbx20 |
| Meis2 | Tgif1 | Maz | Klf3 | Klf6 |
| Sp1 | Zbtb17 | Sp4 | Sp2 | Sp3 |
| Egr1 | Egr2 | E2f6 | E2f1 | E2f4 |
| Spi1 | Spib | Ets1 | Elf1 | Elk1 |
| Gabpa | Elk4 | Fli1 | Ets2 | Elf2 |
| Etv6 | Cbfb | Runx3 | Runx1 | Runx2 |
| Lef1 | Tcf7 | Irf4 | Irf8 | Irf1 |
| Irf2 | Irf3 | Stat1 | Stat2 | |

Table 2A provides a list of genes whose RNA are more highly expressed in the npTH17 cell state and whose expression is maintained by TCF1.

TABLE 2A
Genes Expressed in a Homeostatic Immune Cell State

| | | | | |
|---|---|---|---|---|
| Tcf7 | Vipr1 | Cd52 | Kif1b | Acvrl1 |
| Gsn | Bach2 | Fntb | F2rl1 | Dop1a |
| Ttc28 | Rnf167 | Patj | Slc44a2 | Arsb |
| Ndrg3 | Wdr46 | Usp7 | Lmna | Rpl12 |
| Cd6 | Ranbp10 | Slc41a3 | Tecr | Il16 |
| Klhdc2 | Klhl3 | Rgs3 | Klf13 | Tbc1d1 |
| Ibtk | Sh3kbp1 | Il6st | Cct6a | Adh1 |
| Cpm | Rab3ip | Gramd3 | Arhgef11 | Utrn |
| Ublcp1 | Slc16a5 | Spsb1 | Ctnnbl1 | Ccnh |
| Slc28a2 | Esyt2 | Ecm1 | Foxo1 | Relt |
| Dnmt3a | Tcf20 | Izumo1r | Tmem131l | Tbc1d30 |
| Malat1 | Lipa | Hpcal1 | Rbm38 | Oxct1 |
| Lats2 | Map4k4 | Galnt6 | Dapl1 | Add3 |
| Chd2 | Txk | Rhoh | A630023P12Rik | Setx |
| Ggta1 | Me2 | Ccr7 | Sidt1 | Pdlim4 |
| Slc25a12 | Myl12a | Rev3l | Hsdl1 | Inpp4b |
| Ppm1g | Lrrfip1 | Adk | Ldhb | Slamf6 |
| Rpa1 | Arl4c | Chd7 | Rflnb | Gstp3 |
| Jak1 | Bcor | Tnrc6a | Smc6 | Sypl |
| Prkcq | Pdk1 | Ppp2r1b | Cers6 | Tsn |
| Abcb1a | Adpgk | Trappc12 | Fyb | Inpp5f |
| Fanci | Myo10 | Sp100 | S1pr1 | Usp28 |
| Ikbke | Ift80 | St3gal1 | Gm15708 | Scp2 |
| Actn1 | Sipa1l1 | Idh2 | Shld1 | Abhd2 |
| Smarca2 | Ldlrap1 | Nr4a3 | Eif3m | Pcca |
| Setd4 | Osbpl9 | Prkab1 | Fam111a | Naip2 |
| Afp | Rnf145 | Hbs1l | Il1rl2 | Rcan3 |
| Dmtf1 | Nsg2 | Klf7 | Mctp2 | G3bp2 |
| Dnajc10 | Btg1 | Psma6 | Dennd2d | Itga7 |
| Ccdc126 | Mthfd1l | Rapgef6 | Tdrp | A430093F15Rik |
| Casp8 | Lgmn | Inpp5b | Tars | D030024E09Rik |
| Vav2 | Fli1 | Myl12b | Rad51c | Tapt1 |
| Gpr174 | Zfp644 | Galnt10 | Prorp | Wasf2 |
| Gch1 | Stt3b | Gm6639 | Abhd17b | Parp4 |
| Rasa1 | Gm5617 | Smarcd2 | Stra6 | Glis3 |
| Fgd2 | Tirap | Adgra3 | Atf6 | Marchf10 |
| Nsmce1 | 2200002D01Rik | Dad1 | Nrip1 | Rftn2 |
| Iqgap2 | Atrnl1 | Lekr1 | 1700012I11Rik | C1qtnf2 |
| Eefsec | Tcf7l2 | Chrm3 | Ccser2 | Gpbp1 |
| Polh | Aox2 | P4ha2 | Lncenc1 | Tpbg |
| Glod4 | Ldha | Ccdc186 | Asap1 | Ncl |
| Dph6 | Spats2l | Vmn2r99 | Gltp | Sestd1 |
| Hrh2 | Sptbn1 | Tespa1 | Cdh17 | Cilk1 |
| Gapvd1 | Virma | Ldb1 | Nrde2 | Suclg2 |
| Gm8369 | Mmd | Pdpk1 | Uhrf1bp1l | Cp |
| Qpct | Akap17b | Prtg | Cep350 | Oit3 |
| Arap2 | Wipi1 | Fbxl18 | Slc6a6 | Akap12 |
| Gorasp2 | 4930523C07Rik | Cnot10 | Garnl3 | Flnb |
| Polg | Ankrd13a | Fnip1 | Aim2 | Hapln1 |
| Klf2 | Gm17546 | Nid1 | Nudcd2 | Rnls |
| Bag3 | Ccdc142 | Tbc1d5 | Septin8 | Trpm1 |
| Arhgap12 | Pxn | Atp1a1 | Mettl27 | Abcd3 |
| Faf1 | Phtf1 | Abcb7 | Rb1 | Gfod2 |
| Anapc10 | Vcl | Zfp60 | Tmem245 | 4933432G23Rik |
| Nlk | Spsb4 | Ddx46 | Qrich1 | Brd1 |
| Slc16a12 | Sp5 | Syt6 | Cpeb2 | Rsu1 |
| Dcun1d5 | Dnajc15 | Gphb5 | Ilrun | 1700100I10Rik |
| Pard6g | C530044C16Rik | Cdin1 | Lgi1 | Sell |
| Fgfbp1 | Tet2 | Eif4g3 | Usp47 | Mbnl2 |
| Trip12 | Cntln | Mterf1a | Mterf1b | Kdm7a |
| Ms4a4b | Dclk2 | Stag1 | Tbk1 | Lysmd3 |
| Nav2 | A330040F15Rik | Dhx40 | Ola1 | Bco2 |
| Hs3st3a1 | Numb | Lrrn3 | Itga4 | Sord |
| Phkb | Wdr92 | Mir6349 | Pdia3 | Ms4a4a |
| Baz1a | Ctnnd2 | 4732490B19Rik | Mad2l1 | Zfp536 |
| Fam25c | Ifit1bl2 | Rab11fip5 | Nfe2l3 | 1110002J07Rik |
| Mmp27 | 4930447K03Rik | Zc3hav1 | Tbcd | Klf8 |
| Lrp6 | Spred2 | Edem1 | Znrf3 | a |
| Sec24b | Dgki | Efcab11 | Klrd1 | Diaph2 |
| Zranb3 | Vmn2r91 | Vmn2r92 | Kif5b | Atp2b4 |
| Epha2 | Ltc4s | Lhcgr | Alas1 | Tnr |
| Agps | Smc1a | Abhd17c | Lnx2 | B430306N03Rik |
| Atp2c1 | Zfp53 | Zcchc10 | Atp6ap2 | Nfyb |
| Pfkfb2 | Dnajc1 | Usp32 | Ptprc | Trim5 |
| Nlrp4g | Rybp | Clvs1 | Fam20b | Gm1587 |
| Igsf10 | Camk4 | Med12l | Sugp2 | St18 |
| Zfp560 | Mcu | Aadacl2fm2 | Endod1 | Rin3 |
| Sgk3 | Amn1 | Scfd2 | AI197445 | Clmp |
| Lmntd1 | Sgtb | 1700110C19Rik | 1700123J17Rik | Lrig1 |
| Nsf | Pym1 | Kif13b | Zcchc17 | Csnk1g3 |
| Specc1l | Bpgm | Inpp1 | Mettl22 | Atp11c |
| Jarid2 | Upb1 | A830052D11Rik | Gabbr2 | Marchf1 |
| Crebrf | Exoc4 | Psd3 | Ube2k | Ap1g1 |
| Chst10 | Map2 | 4933440J02Rik | Pir | Ryr3 |
| Pid1 | Atp6v1g3 | Clcc1 | Slc7a1 | Nxph1 |
| Rnaseh2b | Il22ra2 | Zbtb40 | Cdyl | Tmem217 |
| Aff1 | Wdr59 | Hira | Mrps5 | Oasl1 |
| Ube2s | Atp6v1g1 | Herc1 | Nbas | Syne1 |
| Cul3 | Tent5a | Sec14l1 | Zmat4 | Lpcat1 |
| Gm5108 | Sipa1l2 | Amotl1 | Pgpep1l | Ptpn6 |
| Zmynd8 | Dapk2 | Ifnlr1 | Phldb1 | Ap1s3 |
| Clpb | 1700060C16Rik | Lratd1 | Tle1 | Exoc2 |
| Wfikkn2 | Arid3b | Mir7014 | 4930568G15Rik | Dnm3 |
| Epb41l2 | Gimap9 | Fam102a | Mfap3 | Dnajc22 |
| Usp18 | Hsf2bp | Prkd3 | Gtpbp4 | Gpatch8 |
| Marcks | Prpf18 | Park7 | Trpc5 | Dnah2 |
| Ppt1 | Golph3 | Slx4ip | 4930432M17Rik | Pgm2 |
| Spag11a | Ubl7 | Rdm1 | Trib2 | Nutm1 |
| Smyd2 | Cep112 | Lpar4 | Ccdc174 | Tspan5 |
| Usp6nl | Kmt5b | 4933427D14Rik | Usp38 | Ces2d-ps |
| Mdga1 | Ccdc150 | Cux1 | Ube2e3 | Dner |
| Man1a | Rasa2 | Yaf2 | Bace2 | 4930590L20Rik |
| Fabp7 | Myh11 | Tbc1d19 | Gm19466 | Syt13 |
| Mtmr10 | Zswim1 | Ccdc148 | Gjb6 | Acsl4 |
| Hdac9 | Marveld2 | Slc30a5 | Camta1 | Pum2 |
| Mcph1 | 1700019E08Rik | Tlcd2 | Pilrb1 | Pilrb2 |
| Fundc1 | Zfr2 | Rnf11 | Tnfsf4 | 4930518J21Rik |
| 4933412E24Rik | Ralgps2 | Pldi | Fbxw4 | Mcl1 |
| 6030440G07Rik | Dclk1 | Arvcf | Sema7a | Mir331 |
| Ywhaz | Dock9 | Tmbim7 | Arhgef15 | Elovl5 |
| Tcf4 | Lrch3 | Cdhr3 | Tnks1bp1 | Macf1 |
| Ago2 | Mboat4 | Tm4sf20 | Mylk4 | Zbtb17 |
| Atp11b | Cga | 4930402K13Rik | Oxr1 | Hnrnpa3 |
| Plekha1 | 1700031P21Rik | Zfp462 | Lix1 | Gna12 |
| Fbxo45 | Sbno1 | Platr30 | Ifng | Slc7a13 |
| Atf2 | Pskh1 | Nrip3 | Oxsm | Rbm33 |
| Gdap1l1 | Luc7l3 | Megf11 | Irx4 | Tmem18 |
| Flywch1 | Map4k3 | Lyst | Pcgf5 | Ccdc141 |
| Fgd3 | Scd2 | Ccnt2 | Bach1 | Tmem26 |
| Zfp407 | Adh6b | Rere | Cacna1b | Hmgcr |
| Ctr9 | Mir5113 | Rab11b | Cdc40 | Aplp2 |
| Ociad2 | Cyb5a | Zfp217 | Mob3b | Cmah |
| Fto | Fam240a | Ankrd12 | Utp25 | Tnfsf11 |
| Washc4 | Gnai3 | Map2k5 | Add1 | Cmss1 |
| Irgm2 | Emsy | Nipa2 | Tmem94 | Rap2b |
| Fndc3a | Gtf2b | Umad1 | Phactr2 | Phf3 |
| Larp4b | Pds5a | Svep1 | Npepps | Dcun1d1 |
| Mfsd4b5 | Mir101b | Ppp3cb | Pdlim5 | Raph1 |
| Chst2 | 1700021F02Rik | Ube2e2 | Gm10512 | 4930554I06Rik |
| Bicdl1 | Ercc6 | Bcl3 | Il1b | Cstb |
| Dop1b | Gmds | Selenbp1 | Vwa8 | Asb15 |
| Brd4 | Clec4g | Gm31641 | Arid4b | Map4k5 |
| Sec11c | Btbd11 | Trappc10 | Nrd1 | 4930415L06Rik |
| Sppl2a | Tanc1 | Zbtb20 | Prc1 | Btbd7 |
| Actn4 | Fpr2 | Babam2 | Gpr18 | Gpc1 |
| Tnfsf18 | Btnl2 | Gtf2i | Gm15706 | Ism1 |
| Slc35b3 | Enthd1 | Gzma | Rab11fip2 | Max |
| Rab33b | Ctnnd1 | Arih2 | Hopx | Atpsckmt |
| Kif3b | Nepro | Robo1 | Acsl6 | Erc1 |
| Neb | Kif13a | Spef2 | Gm8817 | Zfp748 |
| Cds2 | Dtwd2 | Fam221a | Tex50 | Mettl17 |
| C1d | Pdcd1 | Maml1 | Wdr19 | Pip5k1a |
| Ptger3 | Bcl9 | Mir680-3 | Sytl3 | Dynlt1f |
| Gm29719 | Olfr1510 | Arnt | Tslp | Prkcz |
| Stmn1 | Glt6d1 | Camk1g | Slc2a10 | 1810053B23Rik |
| Smurf1 | Pacsin2 | Ate1 | Pik3r5 | Clybl |
| Septin6 | Pip4k2a | Usp33 | Acsl1 | Rnf32 |
| Tmem141 | Tnrc6c | Ppp2r3a | Troap | Adam22 |
| Slc29a1 | Uvrag | Scml4 | Slc43a1 | Pnoc |
| Setbp1 | Rad54b | Taf4 | Sumo1 | Mms22l |
| Selenot | Hipk2 | Lrpprc | Smox | Cd2bp2 |
| 2610206C17Rik | Itk | Vps37b | Cd55 | Olfr9 |
| Ddx59 | Stk4 | Cldn4 | Uck2 | Rbfox3 |
| Mtus2 | Ephb4 | Kntc1 | Shq1 | Slc16a10 |
| 1700123O12Rik | Hhat | Cyp26b1 | Dync2li1 | C230014O12Rik |
| Zfp286 | Luc7l2 | Lyzl4 | Pkp4 | Ralgapb |
| Gcnt3 | Il6 | Abca17 | Ints7 | Meak7 |
| 1700021F07Rik | Sf3b1 | Acin1 | Usp15 | Psen2 |
| Ube2e1 | Steap4 | Cnst | Pmel | Rnf157 |
| Olfr266 | Pip4k2b | Rasa3 | Agfg1 | Fam217a |
| Hivep2 | Myo3b | Sfswap | Fyn | Ttbk2 |
| Rps6ka5 | Cd226 | Gadl1 | Tox2 | 1810055G02Rik |
| BC004004 | Nans | Myo5a | Cfap54 | Hmbox1 |
| Suclg1 | Abcg2 | Gne | Gm609 | Ciao2a |
| Kif18b | Dnaaf5 | Dcaf11 | Sec1 | Akirin2 |
| Grb2 | Cers4 | Gsk3b | Pea15a | Ube2n |
| Gcc2 | Nemp2 | Map2k4 | Msra | Asah1 |
| Hmgxb3 | Marchf6 | Reln | Dstn | Trappc9 |
| D730003I15Rik | Elfn1 | Mdm4 | Rap2a | 2610037D02Rik |
| Sorcs2 | Aig1 | Xrcc4 | Tcf12 | Scn3b |
| Sec23ip | Xpa | Tbc1d22b | Tmc3 | Tspan13 |
| 4930544G11Rik | Atp13a3 | Gys2 | Prkch | Commd8 |
| Cog2 | Taf3 | Dnase1l3 | Sdk2 | Susd6 |
| Akap6 | Gphn | Thumpd2 | Efl1 | Ireb2 |
| Ltbr | Cldn10 | Efna5 | Esrp1 | Ndufaf6 |
| Sntb1 | Hmgb1-rs17 | Rcor1 | Gan | Gbf1 |
| Btg2 | Gm12354 | Gm10791 | Pak1 | Arhgap11a |
| Brf1 | Ddc | Cox7a2l | Emp1 | Gimap7 |
| Mapkap1 | Foxo4 | Thy1 | Ldb3 | Stat5b |
| Enc1 | Rpl34 | Gm10790 | Gclc | Gm14634 |
| Pdss2 | Dab2 | Gm6498 | Kcnj16 | Tgfbr2 |
| Nyap1 | Cemip2 | Gm20609 | Pros1 | Clasp2 |
| Tent5c | Sugt1 | Atp1b3 | Efr3b | Map3k13 |
| Ddx10 | Gak | Osbpl8 | Ralbp1 | Trappc8 |
| Atg7 | Lbh | Fam168a | 4930512J16Rik | Klra1 |
| Mogat1 | Col12a1 | Fgd5 | Ppp2r5e | Rpp21 |
| Agtpbp1 | Cdc42 | Nnmt | Smad5 | Gm10432 |
| Gm35722 | Ptprn2 | Gm13848 | Sfpq | App |
| Bcar3 | Cep68 | Gigyf2 | Zfp831 | Odc1 |
| Mlxipl | Nck2 | Pias2 | Eif4e3 | Nfkb1 |
| 6530403H02Rik | Niban1 | Mtx2 | Tprg | Cnot6l |
| Fbxo15 | Hdac8 | Wdr89 | Yeats2 | Lnpep |
| Ttf1 | Mical2 | St8sia6 | 4930554C24Rik | L3mbtl3 |
| Rbbp6 | Gm4278 | Tec | Nudt16 | Tet1 |
| Dach2 | Gli1 | Zfp862-ps | Ly6c1 | Gpat3 |
| Nbeal1 | Snx29 | Fus | Gramd4 | Entpd4b |
| C7 | Panx1 | Csrnp3 | Sec31a | Lncpint |
| Arhgap18 | Galnt7 | Lrrc4b | Satb1 | Eri3 |
| Tbc1d14 | Dyrk2 | Rtn1 | Mtif3 | Ppp1cb |
| Adarb1 | Fam126a | Nab1 | Gm26685 | Pcgf3 |
| Dnttip1 | Psma8 | 9430014N10Rik | Fbxo28 | Nudt4 |
| Ppa2 | Svil | Gm20740 | Hs6st1 | Atp10d |
| Vopp1 | Anxa2 | Alox12 | Smagp | Arl5b |
| Rem2 | Cngb1 | Kalrn | Kcnmb4os1 | Unc5cl |
| Mink1 | Orai2 | Cfap52 | 1700030F04Rik | Kctd1 |
| Plscr5 | Fgfr2 | Atp9b | Efhc1 | 8430430B14Rik |
| Tesk2 | Klhdc1 | Specc1 | Ets1 | Osbpl6 |
| Slc34a1 | Mrpl15 | Zcchc9 | Fry | Tom1l2 |
| Ube2d2a | Mgat4a | Arl4a | Lrrc28 | C430002N11Rik |
| Sfrp2 | Ncoa5 | Ggact | Tmem9b | Fcho1 |
| Haus8 | Fpr-rs4 | A930006K02Rik | Chaserr | Stk38l |
| Cep85l | Oprm1 | Disc1 | Ifit3 | Hrh1 |
| Rgs12 | Aqp9 | Phrf1 | Tnfsf8 | Eipr1 |
| Kdm3b | Mlec | Chchd3 | Gas2 | Chm |
| Epas1 | Farp1 | Fbxo47 | Vwce | 2410022M11Rik |
| Dhrs4 | Apln | Fam114a1 | Gemin5 | Triqk |
| Frmd7 | Smyd3 | Gm5083 | Gm29686 | Klrg1 |
| Mzt1 | Dusp11 | Slc9a9 | Plagl1 | Plpp1 |
| Ssh2 | Fer1l6 | Pkp2 | Ube2z | Iqgap1 |
| Rgs1 | Gbp10 | 1700104L18Rik | Igf2bp3 | Vmn1r236 |
| 4933407L21Rik | Wapl | Mirt1 | Sesn3 | Fbxo32 |
| Spata48 | Tomm20 | Cyrib | Dym | Itpk1 |
| Pias1 | Ctnnal1 | Sun2 | Selp | Gm17399 |
| Sema4f | 5830416I19Rik | Mak16 | Kmt2a | BC039771 |
| Coa7 | 4921511C10Rik | 4930595D18Rik | Zfp335 | Gem |
| Slc28a1 | Ccdc3 | Asprv1 | Tmem178b | Ophn1 |
| Slc11a2 | Ext1 | Zc3h12d | Dlgap5 | Elavl1 |
| Leprotl1 | Limk2 | Zyg11b | Jak2 | 4930426L09Rik |
| Atp6v1c1 | Cd200r2 | Lrba | Phlda1 | Zfp446 |
| Gtf2ird1 | Nudcd3 | 4930556N09Rik | Gm33619 | Hephl1 |
| Synpo2 | Baz2b | Fbxo33 | Kcnb1 | Man1a2 |
| Aqr | Serpinb5 | Proc | Cpeb1 | Fubp1 |
| Stk39 | Aebp2 | Rprd2 | Rasgrp1 | Rph3al |
| Rspry1 | Eea1 | Nf1 | Gnas | Ube2h |
| Fam241a | Rhbdd1 | Ptprn | 4921511I17Rik | Tspan9 |
| Slc4a4 | Nars | Tnrc6b | Pcyt1a | Rbms3 |
| 1700034P13Rik | Afdn | Gm13807 | Gm20743 | Gm5086 |
| Edem3 | Med13l | D030045P18Rik | Tollip | Capn2 |
| Gfra2 | Elmo1 | Cep164 | Phf2 | Stim1 |
| Immp2l | Etv3l | 2700069I18Rik | Pdp1 | Col26a1 |
| C730036E19Rik | Lmod3 | Abcb1b | Iigp1 | Igf2bp2 |
| Zfand3 | Tnik | Zfp277 | Zc3h12a | Nhsl1 |
| Txlnb | Gm10030 | Mosmo | Slfn1 | 4933406I18Rik |
| Cd200r4 | Uqcrfs1 | Gdi2 | Spag1 | Osbpl1a |
| Fbxo25 | Klra6 | Msl2 | Pcx | Birc6 |
| Cenpu | Ugcg | Depdc5 | Trp53inp1 | Plekhg6 |
| Gm12505 | Prdm10 | Mir5626 | Diaph3 | Platr10 |
| Got1 | Slc38a2 | Mmaa | Ezh1 | Vmn2r101 |
| Olfr1414 | Col6a6 | Gm14317 | Ankrd55 | Nkap |
| Olfr312 | Stap1 | Nsd3 | 4930512M02Rik | Slc4a10 |
| Ubap2l | Col28a1 | Ensa | Wdyhv1 | 4933430H16Rik |
| Htt | Alox12e | Dennd11 | Gm2245 | Adcy9 |
| Ccdc170 | Gm19585 | Lrrk2 | Farsb | Zfp281 |
| Lypd6b | Slc26a8 | 4933432I03Rik | Wnk1 | Map3k14 |
| Sfi1 | Pde10a | Bcdin3d | Snap47 | Gbp9 |
| Pvr | 4933424G06Rik | St8sia1 | 9330179D12Rik | Epb41l4a |
| Eif2ak3 | Msl3 | Borcs5 | Sema3f | Palmd |
| 1700042G15Rik | Odf1 | A330093E20Rik | Itgam | Syde1 |
| Aplf | Abl2 | 5031425E22Rik | Ep300 | Tbl1xr1 |
| Pik3r1 | Tmsb10 | Pitpnb | Gm4285 | Hspbp1 |
| Rrn3 | Foxq1 | Skor1 | Gypc | Srfbp1 |
| 1700028K03Rik | Arl5c | 2210417A02Rik | Thtpa | Ccdc57 |
| Dock4 | Akt3 | Ntrk3 | B3gntl1 | Galntl6 |
| Gm36229 | Trim33 | Rreb1 | Shroom1 | Prss32 |
| Sbk2 | Prkca | Vezf1 | Stra8 | Eif2b2 |
| Gm19705 | Fndc7 | Arl14 | Gm19303 | Il2 |
| Ctsf | Setd3 | 1700010K23Rik | Pank4 | Spi1 |
| Cd200 | Slc22a2 | Eci1 | Ppil4 | D830013O20Rik |
| Lmtk2 | Plaur | Eif2a | Lgals9 | Plcg1 |
| Zdhhc2 | Parp11 | 4930546C10Rik | Tshr | Ppp6r2 |
| Trp53rkb | Jakmip1 | Wipf2 | Dennd4c | Garem1 |
| Foxp1 | Atp6v1e1 | Myo5b | Cap1 | Hspb11 |
| Kmt2c | Arpp21 | Epha3 | Vangl2 | Map3k4 |
| Pex14 | Thnsl1 | Arhgef10 | Klhl5 | Dlg3 |
| Mark3 | Ncoa2 | Mab21l3 | Taok3 | Trps1 |
| Flt3 | Myo1e | Ttc27 | Xdh | Slc17a8 |
| Drd3 | Kcnip1 | C730014E05Rik | Prdm1 | Map3k5 |
| Lama4 | Fgr | Fndc1 | Mfge8 | Armc8 |
| Rheb | Otud7a | Gm32141 | Ofcc1 | Cyb5b |
| Atp10a | Hmces | Map3k7 | Alox5ap | Lpin1 |
| Ranbp9 | Bcas1 | Prss2 | Dnajb4 | Vmn1r1 |
| Taf1b | 4930406D18Rik | Colca2 | Boll | Shisa7 |
| Tacc2 | Cnn3 | Lor | Olfr658 | Zfand6 |
| Camk2d | Far1 | Gm1123 | Bambi-ps1 | Plbd1 |
| Tspan3 | Fez2 | Slc25a48 | Bvht | 5530601H04Rik |
| Ppcdc | Zfp870 | Btbd2 | Stambpl1 | Zer1 |
| Ralgds | Wnk2 | Nfia | Dlg1 | Maml2 |
| Myo6 | 9130230L23Rik | Man1c1 | Slc30a7 | Ier5l |
| AI504432 | Camk1d | Cfap74 | Myh9 | 1700066B17Rik |
| Eif2b3 | Pmm2 | Stim2 | Arhgap39 | Pkd1 |
| Wfdc2 | Alkbh8 | Prm1 | Gm2447 | Rassf2 |
| Rbm17 | Gtf3c3 | Pou6f1 | Cbx1 | 1700049E22Rik |
| Tsen15 | Plekhs1 | 1700025G04Rik | Atg10 | Chd1 |
| Map10 | Phf11a | Bysl | Crmp1 | Mia2 |
| 1810041H14Rik | Cntrl | Herc3 | Lrrc3b | Rrp1 |
| Wsb2 | Mir146 | Btc | Ccne1 | Riox1 |
| Kcnn3 | Actr10 | BC016579 | Mgme1 | C030034I22Rik |
| Kcnq1 | Anks1 | Slx4 | Ehd3 | Gnao1 |
| Cpne5 | Cyb5r4 | Zfp800 | Fbxl17 | Irf4 |
| Akap14 | Pard3 | Gria3 | Trpm8 | Helz |
| Nek10 | Capza1 | Katnal1 | Armcx6 | Nfatc1 |
| Smarca1 | Gabrb1 | Dnajb14 | Gm15270 | Lat |
| Plekha8 | Ctla4 | Irf5 | Tigit | Vps13b |
| Mxi1 | Prrc1 | Ryr2 | Stard9 | Slc37a3 |
| Extl3 | 1700063D05Rik | Siah3 | Cpsf3 | Olfr1364 |
| Iqch | Atoh7 | Pum1 | Dcaf5 | Mllt3 |
| Tex30 | Ppip5k2 | Dppa5a | Gpr19 | Dcp2 |
| Gsx1 | Ngly1 | 4933433H22Rik | Gja5 | Tead1 |
| Tox | Samd9l | Strada | B930092H01Rik | Evl |
| Eci3 | Fxr1 | Spata5 | Olfr1509 | Arid4a |
| Dph5 | Topbp1 | Nyap2 | Sinhcaf | Mafb |
| 1110032F04Rik | Dgkg | Slc47a2 | Inpp4a | Ikbkg |
| Wwox | Atp6v0d2 | Hipk1 | Prrc2c | Uchl5 |
| Mir21c | Enpp1 | Polr1a | Gm14718 | Ppargc1a |
| Usp12 | Gm26839 | Prim2 | Cblb | Igsf23 |
| Gm5907 | Vav3 | Otulinl | Tlk1 | Mbnl1 |
| Ica1 | Fra10ac1 | Lpp | Il3 | Rnf220 |
| Tab3 | Arhgap17 | Fam210a | Trim36 | Ppfibp2 |
| Slc23a4 | 2310022B05Rik | Kif26b | Kcnj2 | E430016F16Rik |
| Ogt | Clec16a | Napepld | Mtmr3 | Tnfrsf19 |
| Acbd3 | Egf | Ehmt1 | Phf6 | Gm30790 |
| Znrf1 | Xpo4 | Gm8709 | Ss18 | 4930563J15Rik |
| Dnah17 | Smap1 | Gm34068 | Try4 | Ttc30a2 |
| Pgap1 | Sertad2 | Slc28a2b | Ppp2r5c | Mir30c-2 |
| Ube2cbp | Cfap100 | Pde3b | Eps15 | Trak2 |
| Gm20139 | Spink13 | Zfp629 | Ifngas1 | Nxf1 |
| 4931403E22Rik | Acaa2 | Prickle1 | Map3k9 | Traf5 |
| Smad7 | Lmo2 | Slc45a4 | Coq2 | Vapb |
| Lrig2 | Ccdc152 | Mdm1 | Uso1 | Fibcd1 |
| Zdhhc20 | Xylt1 | B020018J22Rik | Cflar | Creld1 |
| Btla | Cobll1 | Ptchd3 | St6gal1 | Naa20 |
| Pde7a | Daam1 | Ttll5 | Cdc14a | Ubap2 |
| Smim4 | Dgkd | Gm33337 | Kidins220 | Bsn |
| Snap25 | Speer4f1 | Fam71b | Malt1 | Cd83 |
| Dbx1 | Lasp1 | Hecw2 | Naa30 | 2210408F21Rik |
| Itih5 | Tagap | Zfp984 | Tnni1 | Ubr2 |
| 1500009L16Rik | Asap2 | Ubap1 | Gorab | Zfp819 |
| 8430426J06Rik | Trim56 | Pycr2 | Smim14 | Tesc |
| Hspa13 | Mbd2 | Mideas | Crhbp | Ubqln1 |
| Dis3l2 | Nme7 | Mindy4 | Cst7 | Kcmf1 |
| Msrb3 | Prf1 | Il20ra | Mir6395 | Snhg16 |
| Zbtb38 | Ect2l | Mylip | Znrf2 | Mgat1 |
| Cfap97d2 | Slain1 | 1110020A21Rik | Asb13 | Gm13986 |
| BB123696 | Il10rb | Arhgap6 | Fam216b | Fkbp1a |
| Lnpk | Rnf43 | Rnf17 | Gucd1 | Rictor |
| Bcat1 | 2610307P16Rik | Ttc17 | Gxylt2 | 6030443J06Rik |
| Akap13 | C130026L21Rik | Mrpl3 | Nwd1 | Scarb2 |
| Dusp16 | Snx12 | Sox5 | 4933440M02Rik | Mx2 |
| Lbp | Snx18 | 3110056K07Rik | Dtnbp1 | Ext2 |
| Tnn | Krr1 | Wdfy1 | Edil3 | Fbxl12os |
| Cldn5 | Pde2a | Rab6b | Man2a1 | Pemt |
| 9630013K17Rik | Cfap20dc | Cog3 | Hs3st5 | Mrpl1 |
| Rbm20 | Arhgef10l | 3425401B19Rik | Smg1 | Impg1 |
| Cyth3 | Gm32200 | Enpp3 | Olfr464 | Tpk1 |
| Cbl | Cdk12 | Gpr141 | Tdrd3 | C2cd5 |
| Sh3rf1 | Atrn | Gm30214 | Gng13 | Pptc7 |
| Ercc6l | Gm5122 | Lrch1 | Snd1 | Fam222a |
| Zfp787 | Olig3 | Bcr | Fam163b | Abhd6 |
| Ppm1b | Usp24 | Insyn2b | Ywhah | AI597479 |
| Cdkn2aipnl | Kl | Gm20758 | Sec63 | Dusp10 |
| Arhgef12 | Cfap45 | Gm12381 | Ccni | Lims1 |
| Ubtd2 | Tmem267 | Zswim2 | Dnajc7 | B3gnt2 |
| Gucy1a1 | Tnip3 | Gm36738 | Mfhas1 | Sugct |
| Fam43a | Myo9a | 9330104G04Rik | Rasgrp4 | Tyrp1 |
| Hamp | Hamp2 | Cabcoco1 | Elovl6 | Nrarp |
| Cggbp1 | Glg1 | Rfx4 | Mir6356 | Tmem123 |
| Micu2 | Secisbp2l | Xrn2 | Mir21a | Otud1 |
| Plcl2 | Ppm1a | Uxs1 | Anxa3 | Magi2 |
| Chil5 | Plin2 | Prag1 | Stpg1 | Rfx8 |
| Lsm14a | Cysltr2 | Map3k8 | Slc39a10 | 4930459C07Rik |
| Ogdh | Rbm11 | Itm2a | Cdr2 | Gm805 |
| Bckdhb | Lclat1 | Hlcs | Ptpn22 | 4930534H03Rik |
| Ambra1 | A630001O12Rik | Kcnj15 | Ly6c2 | Zfp930 |
| Gm29687 | Mir692-1 | Gfpt1 | Pou2af1 | Znhit6 |
| Il10 | Ptch2 | Bicra | Pou2f1 | Bbx |
| Rnf38 | Tbc1d2b | 1700074H08Rik | Ubtd1 | Smad4 |
| Tet3 | Cacna2d1 | Cyfip2 | Dusp4 | Ptprk |
| Rps6ka3 | Kdm8 | Zscan2 | Cacnb3 | Fbln1 |
| Rabgef1 | Raly | Casc1 | Gna13 | Ikzf4 |
| BC065403 | Gabpb2 | Tmem40 | Bod1l | Fam3c |
| Il12rb2 | Rbms2 | Klra3 | Klra9 | Noct |
| Slc1a7 | Guca2b | Tfam | Ly75 | Ifi47 |
| Lipk | Rapgef4 | Gm6551 | Ikbkb | Dusp2 |
| Spin1 | Selenof | Calhm5 | Mtss1 | Msi2 |
| Snora26 | Ascc3 | Dpp4 | Arc | 2410004B18Rik |
| Tmod3 | Slc1a4 | Chl1 | 4930529K09Rik | Gm11110 |
| Setd2 | Atxn7 | Heatr1 | Epb41l4b | Hivep1 |
| Itgb6 | Sos1 | Stat4 | Cdkal1 | Rad52 |
| Blm | Chp1 | Lamp2 | Mtpn | Fbxl5 |
| Pisd-ps2 | Tank | Actg2 | Tnks | Ocrl |
| Zwint | Gm7538 | Manba | Rnf138 | Adck1 |
| Kansl1l | Csnk1e | Garre1 | Slc2a2 | Actr2 |
| Cpa4 | Sec11a | Zfp592 | Gm5544 | Gm20544 |
| Irs1 | Msh3 | Zfp821 | Fcrl1 | Nup210 |
| Adamts20 | Neto2 | Cyp20a1 | Scgb3a1 | 1810006J02Rik |
| Antxr2 | Wdr37 | Casp4 | 9530052C20Rik | Eepd1 |
| Bspry | Pgr15l | Uimc1 | Zfp14 | Cog5 |
| Mmut | Rassf3 | Melk | Zfp280d | 4930469K13Rik |
| Stat3 | Gcm2 | 1700017B05Rik | Pcnx | 2810408I11Rik |
| Ucp1 | Dtx4 | Ifi208 | Fcmr | Txndc11 |
| Nup153 | Zbtb43 | Zbtb25 | Gimap6 | Tbca |
| Cpeb4 | Cd2 | Tns4 | Kcnc2 | Kif11 |
| Abcg1 | Cd28 | Glcci1 | She | Spata6 |
| Sla2 | Col18a1 | Etnk1 | B430212C06Rik | AU041133 |
| Olfr401 | Slit3 | Oosp1 | Rnf216 | Cdc20b |
| Itm2b | Msgn1 | Tbc1d7 | Brdt | Car12 |
| Redrum | Ankrd46 | Tafa4 | Cntn5 | Ppp2cb |
| Gm5127 | Stx3 | Cdk17 | Nr2c2 | Bri3 |
| Sspn | Papolg | Vdac3 | Dnm2 | Ift81 |
| Pam | Cyria | Rxfp1 | Fam118a | Evi5 |
| Ifit2 | Stk26 | Gm12108 | Mrtfa | Capn8 |
| Rexo2 | Eef1aknmt | Gtdc1 | Gm12216 | AI839979 |
| B230323A14Rik | Dydc2 | Rsph1 | Hexa | Mphosph6 |
| 4732471J01Rik | Plekhf2 | Sec31b | Csgalnact1 | Gm15326 |
| Gm19589 | Nek7 | BC051019 | Ntn1 | Tmigd3 |
| Nfat5 | Ago3 | Gm8013 | Cdh22 | Mettl8 |
| Lrrc1 | Jmy | Paxbp1 | Dipk1b | Sdf4 |
| Ercc3 | Erg | Lhfp | Kctd19 | Skor2 |
| Rac1 | Gvin2 | Ccdc15 | Ppp1r13b | 4930583K01Rik |
| Mad1l1 | Adgrg6 | Gm16630 | Platr4 | Als2 |
| Cit | Gm28043 | Pik3r6 | Rnf19a | Snrpn |
| Gprc5c | Ppard | 1110059E24Rik | Pde4dip | Phf21a |
| Atp6v0a1 | Ggcx | Pced1b | Cenpc1 | Fkbp1b |
| Csad | Pag1 | Cnksr3 | Tmem243 | Dennd3 |
| Prkce | Lhx1os | Lrrc6 | Sema6a | Lrp12 |
| Kcp | Vwa3a | Dnajb1 | Ece1 | Rundc1 |
| Sag | Rnase10 | Lrmp | Slc24a3 | Shisal1 |
| Cdh5 | Gab1 | Pja2 | Cacna1d | Lpar5 |
| Il12b | Ldlrad3 | Npl | Snx4 | Slc23a2 |
| Pan3 | Scin | Tial1 | Acoxl | Ikzf1 |
| Atp8b1 | Taar4 | Ntpcr | 4930565D16Rik | Ift43 |
| Prdm5 | Prss43 | Slc17a6 | Unc80 | Klf3 |
| Usp9x | Ttc3 | Ms4a4d | Reep3 | Gm16793 |
| Herc2 | Plekhm3 | Mir568 | Atxn2l | Tmem184b |
| Ninj2 | Rplp1 | Naip1 | S100a6 | Chn2 |
| Cbr3 | Nyx | Ttc7b | Trmt1l | Numa1 |
| Snrpe | Ptbp3 | Mnat1 | Tnni3 | Supt6 |
| Epc1 | H2al2a | Cdc42se2 | Zfp41 | Asb1 |
| Rhobtb2 | Spata13 | Arhgef17 | Scaf8 | Spata32 |
| Camkmt | Tbc1d22a | Clasp1 | Prokr1 | Gnl3l |
| Stard13 | Kdelr2 | Smad2 | Zp3r | Dcdc2b |
| Wdr25 | Asb7 | Ell | Nedd4l | Trim71 |
| Hmcn1 | Apaf1 | Tbcel | Decr1 | Gm9530 |
| 1700029J03Rik | Hexb | Sec61b | Bcas3 | Usp3 |
| Lyzl1 | 1700027J07Rik | Vrk1 | Relch | Abi2 |
| Dguok | Crbn | Mrgbp | Itga6 | Serpini2 |
| Rfc1 | Manea | Gm5434 | Kif16b | E130114P18Rik |
| Zfat | Rnaset2b | Sae1 | Myo1d | Supt7l |
| Thrb | Rgs9 | Kcnf1 | Car2 | Spidr |
| Mta1 | Spin2c | Gbe1 | Sp2 | 1700016K19Rik |
| Foxo3 | Coro2b | Tk2 | Arl6ip1 | Apobec1 |
| Uri1 | Map3k19 | Adcy6 | Fam81a | Zfp51 |
| Gng12 | 2510009E07Rik | Klra10 | Pwwp3a | Deup1 |
| Gm11981 | Smu1 | Slc6a7 | Srek1ip1 | Ddx31 |
| Dux | Rab10 | Dlg2 | Srpk2 | 4930425L21Rik |
| Wdr20 | Cd200r3 | Tigd2 | Speer2 | Pak2 |
| Tmem132c | Kat8 | Dusp27 | Fndc5 | Slc25a30 |
| Fam234a | Gm27217 | Por | Prkar2b | Dmbt1 |
| Ccnq | Dleu2 | Hey1 | Agr3 | Gpr15 |
| B230303A05Rik | Nova2 | Gm29461 | Srgap1 | Mir5127 |
| Osmr | Hs1bp3 | Srp14 | Ptk2 | Derl1 |
| Rptoros | Stx16 | Srd5a3 | Ube2w | Dnajc3 |
| Wdtc1 | Itga9 | Mfsd2a | Tle4 | Txn1 |
| Gsdme | Prdm16 | Arhgap10 | Rab27a | B4galt5 |
| Armcx1 | Sdk1 | Nedd9 | Spata31d1b | Dipk1a |
| Fam120a | Nup98 | Zbtb9 | Cd2ap | Mpp6 |
| Akna | Srpk1 | Slc44a3 | Ccser1 | Adcy1 |
| Arid1b | Iqsec2 | 1810046K07Rik | Rin2 | Isoc1 |
| Gm35584 | S100b | Micu1 | Myo3a | Ica1l |
| Klhl2 | Cops9 | Tex9 | Pfdn2 | Tnnt1 |
| Arid1a | Slc26a4 | Gm20268 | Acta2 | Dennd1a |
| Wdr38 | Atl2 | Fam78a | D430041D05Rik | Slc7a8 |
| Hnf1a | Tm2d1 | Ccny | Sik3 | Slc12a7 |
| Zfp622 | Apc | Gm4814 | Arhgap15 | Mr1 |
| Atf7 | Ube4a | Mfsd4a | 3830406C13Rik | Zzz3 |
| Dpy19l1 | Prg4 | Dusp5 | Mtmr6 | Phf14 |
| Rps6kb1 | C230024C17Rik | Lamb3 | Actr3b | Arhgef18 |
| Oscp1 | Stk38 | Nbea | Ywhaq | Akr7a5 |
| Tmem11 | Wscd2 | Rock2 | Sgpp1 | Cbx5 |
| Tmem114 | Bmt2 | C330011F03Rik | Slc52a3 | Piwil2 |
| Slc36a1 | Adamts14 | Lcp1 | Sppl3 | 2210010C04Rik |
| 1700020N01Rik | Plch2 | 4930519D14Rik | Slc13a3 | A730043L09Rik |
| Maml3 | Pck1 | Cgn | Rpap3 | Cdc7 |
| Sltm | Lcn9 | Fau | Arhgef3 | Sesn1 |
| 1700027F09Rik | Lhfpl3 | Pik3c2b | Sardh | Gm5069 |
| Rapgef2 | Pmepa1 | Sgk1 | Tbc1d16 | Efnb2 |
| Npsr1 | Gpr180 | Itsn2 | 4933411E08Rik | Cdk19 |
| Riok1 | Slc25a40 | Sh3pxd2a | Ube2r2 | Slc19a3 |
| Aldh5a1 | Nt5c2 | Gm4668 | Plat | Gm5532 |
| Ippk | Fut8 | Acsbg3 | Serbp1 | Vps45 |
| Basp1 | Gm16675 | Arfip1 | Tram1 | Nkx2-4 |
| Gabrr1 | Pnrc1 | Tgfbr3 | Tns1 | Gm14424 |
| Sys1 | Rubie | Foxj2 | Pld2 | Creb3l1 |
| Sft2d2 | Sntb2 | Shb | B4galnt3 | Cd40 |
| Dnai3 | Whrn | 1700109G15Rik | Prdm9 | Capsl |
| 1700021N21Rik | Bcl2l14 | 1600002D24Rik | 4930515B02Rik | Traf3 |
| Ppm1l | Ppp4r1 | Zfand2a | Cracd | Trim25 |
| 4930529N20Rik | Tmc8 | A730085K08Rik | Akr1d1 | Slc35c1 |
| Myo18a | Rell1 | Iqub | Nox1 | F2 |
| Postn | Fsip1 | Adrb2 | Grik4 | Mir7678 |
| Paqr5 | Mir9-3hg | Wnt5b | Cep128 | Deptor |
| 4932438A13Rik | Galnt15 | Sez6l | Calm2 | Pdgfb |
| Stx8 | Cd36 | Nup160 | Hesx1 | Fhdc1 |
| Gsx2 | Mmp15 | Tafa3 | Akain1 | Cabp1 |
| Mir7-2 | Rapgef1 | 2610035D17Rik | Twsg1 | Ccnd2 |
| Carm1 | Slc39a11 | Dpysl2 | Cyld | Gm1647 |
| Rab15 | Pde8b | Tob1 | Klf17 | Prkcb |
| Ckap4 | Mpp2 | Gabrr2 | Ptpn13 | Gap43 |
| Lrrc63 | Il9r | Tnfrsf8 | 2010010A06Rik | Slain2 |
| Npas2 | Apod | Gcc1 | D030025E07Rik | Ripor3 |
| Ltbp2 | Arhgap29 | Ppp1r21 | G530011O06Rik | Ints6 |
| Mir3965 | 2810001G20Rik | Cap2 | Ulk4 | Ripor2 |
| Afap1 | Thap3 | Axin2 | 4930556M19Rik | Ifne |
| Zfp677 | Gm31108 | Zfp983 | 1700109K24Rik | Zfp365 |
| Kcnh5 | Psma1 | Enpp2 | Vsig8 | Ap3m1 |
| Card11 | 4930402H24Rik | Celf2 | Ano10 | Lama5 |
| Runx3 | Btbd6 | Tmem201 | Eps8l1 | Gm13032 |
| Trub1 | Hykk | Mical3 | Aff3 | Zeb1 |
| Prss16 | Spring1 | Trp53bp1 | Foxn3 | Adamts3 |
| Lefty1 | Bmp2k | Camk2a | Snn | Zkscan16 |
| Fam214b | Med13 | Slc16a1 | Tmem131 | Trak1 |
| 9030616G12Rik | Eomes | Tmem104 | Slc36a1os | Suds3 |
| Atp6v0a4 | Frs2 | Gm14164 | Gm16863 | Gm5148 |
| Gramd1c | Cnot6 | Tshz1 | Ostm1 | Slc30a9 |
| 4930486L24Rik | Ksr1 | Kcnj12 | E230016M11Rik | Prmt3 |
| Arid5b | Dtl | Ppm1h | 4921517D22Rik | Polr3e |
| Vti1a | Gpnmb | Prelid3b | Cd300a | Exog |
| D630024D03Rik | Hspb1 | S1pr3 | Shf | Timm8a2 |
| Exoc6 | Fam186b | Prkar1b | Dusp7 | Gpr179 |
| Zmym6 | Dip2b | Myrfl | Gm572 | Smim41 |
| Zfp839 | 8430436N08Rik | Acox2 | Ascc1 | Phf20 |
| Arid2 | Mir467h | Clstn3 | Slc5a1 | Fhit |
| Gm14569 | Wee2 | Il15ra | Mex3b | Spata31d1a |
| Slc6a9 | Tmcc3 | Xpnpep2 | Gm1604b | Rps6ka2 |
| Patl2 | Hoxb13 | Slc22a1 | D16Ertd472e | Myo5c |
| Ccn6 | Mtarc1 | Pla2g10 | Mcoln3 | Vps18 |
| Gnb1l | Arsi | Zfp658 | Adamts10 | Ppargc1b |
| B930018H19Rik | Prkag2 | 4933433G08Rik | Arsg | Grip2 |
| Siah1a | Lad1 | Ythdc1 | Irf2 | Drosha |
| Upk1a | Thsd1 | Trio | Nat9 | Lin52 |
| Rbpms | Gm5893 | Sh2d1a | Shc3 | Gnptab |
| Zfp236 | Klhl31 | Smg9 | Tlcd5 | Arhgap5 |
| Braf | Ccdc162 | Pipox | Ppp6r1 | A330076C08Rik |
| Clip2 | Wnt2b | 1700028D13Rik | Pcnx2 | Chrna9 |
| Sdr9c7 | Gm5535 | Cacna2d4 | Cytip | Wnt3 |
| Map2k2 | Ncf1 | Traf3ip3 | Neurl1b | 4921504E06Rik |
| 5430427M07Rik | Gm38404 | Mettl9 | Mir6897 | Thap12 |
| 1700066B19Rik | Lsm3 | Lrrc74a | Smim23 | Calr4 |
| Pde4d | Ptpn14 | Pds5b | Trpv3 | Dlk1 |
| BB019430 | Ilf3 | Car8 | Gm20098 | Actn3 |
| Gm29684 | Rhob | Lrp4 | Spry4 | Prune2 |
| Akr1b8 | Fam168b | Casp3 | Rfx3 | Focad |
| Mpp7 | Npy | Ggt7 | Gm10857 | Rab8b |
| Cyth1 | Tshz2 | 1700016G22Rik | Calcrl | Ptpru |
| Thap4 | Slc20a2 | Sall3 | Agk | Arf1 |
| Lcp2 | Ubash3b | Dnah6 | Ncor2 | Trim24 |
| Gm12610 | Pfdn1 | Akap8l | Herpud1 | Angpt4 |
| Cpa6 | Cenpf | Elfn2 | Spaca1 | Bank1 |
| Cited2 | Gadd45g | 4632415L05Rik | Dlgap1 | Hnrnpf |
| Pdgfc | Sil1 | Samd8 | Snrpf | Zfp330 |
| Nkx1-1 | C130080G10Rik | Gm33677 | Hs6st2 | Osbpl10 |
| Morn2 | Spo11 | Gpsm2 | Mgat4f | Htatip2 |
| Ppp6r3 | 0 | 0 | 0 | 0 |
| Tcf7 | Vipr1 | Cd52 | Kif1b | Acvrl1 |
| Gsn | Bach2 | Fntb | F2rl1 | Dop1a |
| Ttc28 | Rnf167 | Patj | Slc44a2 | Arsb |
| Ndrg3 | Wdr46 | Usp7 | Lmna | Rpl12 |
| Cd6 | Ranbp10 | Slc41a3 | Tecr | Il16 |
| Klhdc2 | Klhl3 | Rgs3 | Klf13 | Tbc1d1 |
| Ibtk | Sh3kbp1 | Il6st | Cct6a | Adh1 |
| Cpm | Rab3ip | Gramd3 | Arhgef11 | Utrn |
| Ublcp1 | Slc16a5 | Spsb1 | Ctnnbl1 | Ccnh |
| Slc28a2 | Esyt2 | Ecm1 | Foxo1 | Relt |
| Dnmt3a | Tcf20 | Izumo1r | Tmem131l | Tbc1d30 |
| Malat1 | Lipa | Hpcal1 | Rbm38 | Oxct1 |
| Lats2 | Map4k4 | Galnt6 | Dapl1 | Add3 |
| Chd2 | Txk | Rhoh | A630023P12Rik | Setx |
| Ggta1 | Me2 | Ccr7 | Sidt1 | Pdlim4 |
| Slc25a12 | Myl12a | Rev3l | Hsdl1 | Inpp4b |
| Ppm1g | Lrrfip1 | Adk | Ldhb | Slamf6 |
| Rpa1 | Arl4c | Chd7 | Rflnb | Gstp3 |
| Jak1 | Bcor | Tnrc6a | Smc6 | Sypl |
| Prkcq | Pdk1 | Ppp2r1b | Cers6 | Tsn |
| Abcb1a | Adpgk | Trappc12 | Fyb | Inpp5f |
| Fanci | Myo10 | Sp100 | S1pr1 | Usp28 |
| Ikbke | Ift80 | St3gal1 | Gm15708 | Scp2 |
| Actn1 | Sipa1l1 | Idh2 | Shld1 | Abhd2 |
| Smarca2 | Ldlrap1 | Nr4a3 | Eif3m | Pcca |
| Setd4 | Osbpl9 | Prkab1 | Fam111a | Naip2 |
| Afp | Rnf145 | Hbsl1 | Il1rl2 | Rcan3 |
| Dmtf1 | Nsg2 | Klf7 | Mctp2 | G3bp2 |
| Dnajc10 | Btg1 | Psma6 | Dennd2d | Itga7 |
| Ccdc126 | Mthfd1l | Rapgef6 | Tdrp | A430093F15Rik |
| Casp8 | Lgmn | Inpp5b | Tars | |

Table 2B provides a list of transcription factors whose binding motifs are specifically enriched in genes expressed in the npTh17 cell state and therefore are likely drivers of the npTh17 cell state.

TABLE 2B
Transcription Factors Expressed in
a Homeostatic Immune Cell State

| | | | | |
|---|---|---|---|---|
| HEB | FOXO1 | GABPA | FLI1 | ELK4 |
| EGR1 | TCF1 | ETS1 | | |
| Jun | Fosl2 | Junb | Fos | Jund |
| Tfe3 | Usf1 | Usf2 | Clock | Arntl |
| Arnt | Bhlhe40 | Tfeb | Mxi1 | Max |
| Myc | Nfyb | Nfya | Nfyc | Ddit3 |
| Atf4 | Cebpg | Zbtb7a | Mbd2 | Epas1 |
| Hif1a | Mecp2 | Xbp1 | Cux1 | Zbtb33 |
| E2f2 | E2f5 | Hinfp | Mtf1 | Nrf1 |
| Pax5 | Yy1 | Pou1f1 | Nfe2l1 | Tead2 |
| Arid5b | Sox5 | Foxp2 | Foxo1 | Foxk1 |
| Foxo3 | Nr1d1 | Nr1d2 | Rora | Rorc |
| Stat5a | Stat5b | Stat3 | Stat4 | Rel |
| Relb | Rela | Nfkb1 | Nfkb2 | Maff |
| Mafk | Maf | Mafg | Bach2 | Nfe2l2 |
| Bach1 | Batf | Foxj3 | Foxj2 | Foxo4 |
| Prdm1 | Irf7 | Irf9 | Lyl1 | Tcf12 |
| Tcf3 | Smad3 | Smad2 | Smad4 | Mef2c |
| Mef2a | Mef2d | Rfx3 | Rfx1 | Rfx2 |
| Rara | Rxra | Pparg | Nr1h3 | Nr2c2 |
| Ar | Nr3c1 | Pbx1 | Pknox1 | Esrra |
| Esrrg | Esr1 | Rarg | Stat6 | Nfatc1 |
| Nfatc2 | Nfatc3 | Ebf1 | Prdm16 | Atf2 |
| Crem | Atf1 | Creb1 | Nr2c1 | Rxrb |
| Mecom | Gata1 | Gata3 | Prdm5 | Tcf4 |
| Zeb1 | Hsf1 | Hsf2 | Ikzf1 | Ctcf |
| Zfx | Bcl6 | Nfia | Nfic | Smarca5 |
| Thap11 | Dbp | Nfil3 | Thra | Tbp |
| Pou2f1 | Pou2f2 | Zkscan1 | Gfi1 | Pbx2 |
| Rest | Srf | Nr4a1 | Prdm9 | Rbpj |
| Tbx21 | Srebf1 | Srebf2 | Taf1 | Tbx20 |
| Meis2 | Tgif1 | Maz | Klf3 | Klf6 |
| Sp1 | Zbtb17 | Sp4 | Sp2 | Sp3 |
| Egr1 | Egr2 | E2f6 | E2f1 | E2f4 |
| Spi1 | Spib | Ets1 | Elf1 | Elk1 |
| Gabpa | Elk4 | Fli1 | Ets2 | Elf2 |
| Etv6 | Cbfb | Runx3 | Runx1 | Runx2 |
| Lef1 | Tcf7 | Irf4 | Irf8 | Irf1 |
| Irf2 | Irf3 | Stat1 | Stat2 | |

All gene name symbols refer to the gene as commonly known in the art. The examples described herein that refer to the mouse gene names are to be understood to also encompasses human genes, as well as genes in any other organism (e.g., homologous, orthologous genes). Mouse gene symbols are generally italicized, with only the first letter in upper-case (e.g., Il17). Mouse protein symbols are generally not italicized, and all letters are in upper-case (e.g., IL-17). As used herein mouse gene symbols may be shown with only the first letter in upper-case and not italicized (e.g., Il17). Any reference to the gene symbol is a reference made to the entire gene or variants of the gene. Any reference to the gene symbol is also a reference made to the gene product (e.g., protein). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information.

### Exemplary Methods of Increasing/Decreasing Gene Expression

As previously described, the methods can include increasing and/or decreasing the expression and/or amount one or more target genes and/or gene products produced therefrom in a cell, particularly a Th17 cell. In some embodiments, decreasing the expression of a target gene comprises knocking-out a gene or knocking down a transcript transcribed therefrom so as to reduce or effectively eliminate its gene product. In some embodiments, decreasing the expression of a target gene comprises modifying a non-coding region controlling expression of the target gene so as to decrease the expression of the target gene. In some embodiments, increasing expression of a target gene can include introduction of one or more copies of the target gene, modification of the target gene such that it is a variant that is more highly expressed, and/or modification of a non-coding region controlling expression of the target gene such that expression is increased. In some embodiments, the epigenetic landscape of a target gene can be modified such that its expression is increased or decreased.

### Vector Based Gene Knock-Out

In one embodiment, the method for treating an inflammatory disease or disorder comprises delivering one or more recombinant expression vectors to a subject in need thereof, wherein the vectors are configured to knock-down expression of the one or more target genes of Tables 1A/1B. See e.g., Hendrie and Russell “Gene Targeting with Viral Vectors” Molecular Therapy 12(1):9-18 (2005). In general, a vector that is designed for gene targeting, including gene knock-out, comprises sequences that are homologous to regions of a target gene (referred to as the homology arms) so as to facilitate insertion of a vector sequence that is between the homology arms in the target gene via homologous recombination with the target gene between two homology arms. The homology arms are designed to hybridize with chromosomal DNA in the target gene. The types of modification that may be introduced include substitutions, deletions and/or insertions, that prevent expression of the target gene or modify the target gene such that a non-functional gene product is ultimately expressed (e.g., frame-shift mutations, introduction of pre-mature stop codon, introduction of alternative splice sites, introduction or removal of post-translation modification sites).

### Regulatory Elements

Recombinant expression vectors may include one or more regulatory elements, which may be selected on the basis of the host cells to be used for expression, that is operably linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). The term “operably linked” as used herein also refers to the functional relationship and position of a promoter sequence relative to a polynucleotide of interest (e.g., a promoter or enhancer is operably linked to a coding sequence if it affects the transcription of that sequence). Typically, an operably linked promoter is contiguous with the sequence of interest. However, enhancers need not be contiguous with the sequence of interest to control its expression. The term “promoter”, as used herein, refers to a nucleic acid fragment that functions to control the transcription of one or more polynucleotides, located upstream of the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. A “tissue-specific” promoter is only active in specific types of differentiated cells or tissues.

In another embodiment, the vector of the disclosure further comprises expression control sequences including, but not limited to, appropriate transcription sequences (i.e., initiation, termination, promoter, and enhancer), efficient RNA processing signals (e.g., splicing and polyadenylation (polyA) signals), sequences that stabilize cytoplasmic mRNA, sequences that enhance translation efficiency (i.e., Kozak consensus sequence), and sequences that enhance protein stability. A great number of expression control sequences, including promoters which are native, constitutive, inducible, or tissue-specific are known in the art and may be utilized according to the present disclosure.

In another embodiment, the vector of the disclosure further comprises a post-transcriptional regulatory region. In a preferred embodiment, the post-transcriptional regulatory region is the Woodchuck Hepatitis Virus post-transcriptional region (WPRE) or functional variants and fragments thereof and the PPT-CTS or functional variants and fragments thereof (see, e.g., Zufferey R, et al., J. Virol. 1999; 73:2886-2892; and Kappes J, et al., WO 2001/044481). In a particular embodiment, the post-transcriptional regulatory region is WPRE. The term “Woodchuck hepatitis virus posttranscriptional regulatory element” or “WPRE”, as used herein, refers to a DNA sequence that, when transcribed, creates a tertiary structure capable of enhancing the expression of a gene (see, e.g., Lee Y, et ah, Exp. Physiol. 2005; 90 (1): 33-37 and Donello J, et al, J. Virol. 1998; 72 (6): 5085-5092).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990).

Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as tissue cells (e.g., Th17 cells) or other particular cell types. Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Also encompassed by the term “regulatory element” are enhancer elements (e.g., adipose specific enhancers or Woodchuck Hepatitis Virus Posttranscriptional Regulatory Element (WPRE)). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein.

### Vector Selection

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g., adenoviruses, adeno-associated viruses as well as retroviruses and lentiviruses), as well as non-viral vectors such as plasmids.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes that they include. Such vectors are referred to herein as “expression vectors.” Vectors for and that result in expression in a eukaryotic cell can be referred to herein as “eukaryotic expression vectors.” In another example embodiment, the vector integrates the gene into the cell genome or is maintained episomally.

In one example embodiment, by means of an AAV viral vector. The terms “adeno-associated virus”, “AAV virion”, and “AAV particle”, as used interchangeably herein, refer to a virion composed of at least one AAV capsid protein (e.g., all capsid proteins of a particular AAV serotype) and an encapsidated polynucleotide AAV genome. If the particle comprises a heterologous polynucleotide flanked by AAV inverted terminal repeats (i.e., a polynucleotide that is not a wild-type AAV genome, e.g., a transgene is delivered to a mammalian cell), it is often referred to as an “AAV vector particle” or “AAV vector”. AAV refers to a virus belonging to the genus dependovirus parvoviridae. The AAV genome is approximately 4.7 kilobases long and consists of single-stranded deoxyribonucleic acid (ssDNA), which can be in either the positive or negative orientation. The genome comprises Inverted Terminal Repeats (ITRs), and two Open Reading Frames (ORFs), at both ends of the DNA strand: rep and cap. The Rep framework is formed by four overlapping genes encoding the Rep proteins required for the AAV life cycle. The cap framework contains overlapping nucleotide sequences of the capsid proteins: VP1, VP2, and VP3, which interact together to form an icosahedral symmetric capsid (see, e.g., Carter B, Adeno-assisted viruses and ado-assisted viruses vectors for genetic drive, Lassic D, et al, eds., “Gene Therapy: Therapeutic Mechanisms and Strategies” (Marcel Dekker, Inc., New York, NY, US, 2000); and Gao G, et al, J. Virol. 2004; 78 (12): 6381-6388). The term “adeno-associated virus ITR” or “AAV ITR” as used herein refers to inverted terminal repeats present at both ends of the DNA strand of the genome of an adeno-associated virus. The ITR sequences are required for efficient proliferation of the AAV genome. Another characteristic of these sequences is their ability to form hairpins. This property contributes to its own priming, which allows synthesis of the second DNA strand independent of the priming enzyme. It has also been shown that ITRs are essential for integration and rescue of wild-type AAV DNA into the host cell genome (i.e., chromosome 19 of humans) and for efficient encapsidation of AAV DNA that binds to the resulting fully assembled, DNase-resistant AAV particles.

The term “AAV vector” as used herein further refers to a vector comprising one or more polynucleotides of interest (or transgenes) flanked by AAV terminal repeats (ITRs). Such AAV vectors can be replicated and packaged as infectious viral particles when present in a host cell that has been transfected with a vector that can encode and express Rep and Cap gene products (i.e., AAV Rep and Cap proteins), and wherein the host cell has been transfected with a vector that encodes and expresses proteins from adenovirus open reading frame E4orf6. When an AAV vector is incorporated into a larger polynucleotide (e.g., a chromosome or another vector, such as a plasmid for cloning or transfection), then the AAV vector is typically referred to as a “protein-vector”. This protein-vector can be “rescued” by replication and encapsidation in the presence of AAV packaging functions and the necessary helper functions provided by E4orf6.

In one example embodiment, gene therapy uses an adeno-associated viral (AAV) vector comprising a recombinant viral genome wherein said recombinant viral genome comprises an expression cassette comprising an adipose tissue-specific transcriptional regulatory region operably linked to a polynucleotide encoding for a target gene in Tables 2A-2B or other agent to be delivered, such as a genetic modifying agent or component thereof, (AAV vectors can also be used for any compositions described herein, such as a programable nuclease). AAV according to the present disclosure can include any serotype of the 42 serotypes of AAV known. In another example embodiment, the AAV is as described previously for adipose tissue specific tropism (see, e.g., WO2014020149A1; and Bates R, Huang W, Cao L. Adipose Tissue: An Emerging Target for Adeno-associated Viral Vectors. Mol Ther Methods Clin Dev. 2020; 19:236-249). In particular, the AAV may include an adipocyte specific promoter.

In particular, the AAV of the present disclosure may belong to the serotype AAV1, AAV2, AAV3 (including types 3A and 3B), AAV4, AAV5, AAV6, AAV7, AAV8, AAV9, AAV10, AAV11 and any other AAV. In a preferred embodiment, the adeno-associated viral vector of the disclosure is of a serotype selected from the group consisting of the AAV6, AAV7, AAV8, and AAV9 serotypes. In more preferred embodiments, the adeno-associated viral vector of the disclosure is an AAV8 serotype. In more preferred embodiments, the adeno-associated viral vector of the disclosure is the engineered hybrid serotype Rec2 (see, e.g., Charbel Issa, et al., 2013, Assessment of tropism and effectiveness of new primate-derived hybrid recombinant AAV serotypes in the mouse and primate retina PLOS ONE, 8 (2013), p. e60361). In one example embodiment, Rec2 can be used for oral administration, as oral administration of Rec2 results in preferential transduction of BAT with absence of transduction in the gastrointestinal track.

The genome of the AAV according to the disclosure typically comprises the cis-acting 5′ and 3′ inverted terminal repeat sequences and an expression cassette (see, e.g., Tijsser P, Ed., “Handbook of Parvoviruses” (CRC Press, Boca Raton, FL, US, 1990, pp. 155-168)).

The polynucleotide of the disclosure can comprise ITRs derived from any one of the AAV serotypes. In a preferred embodiment, the ITRs are derived from the AAV2 serotype. The AAV of the disclosure comprises a capsid from any serotype. In particular embodiment, the capsid is derived from the AAV of the group of AAV1, AAV2, AAV4, AAV5, AAV6, AAV7, AAV8 and AAV9. In a preferred embodiment, the AAV of the disclosure comprises a capsid derived from the AAV8 or AAV9 serotypes.

In another particular embodiment, the AAV vector is a pseudotyped AAV vector (i.e., the vector comprises sequences or components originating from at least two distinct AAV serotypes). In a particular embodiment, the pseudotyped AAV vector comprises an AAV genome derived from one AAV serotype (e.g., AAV2), and a capsid derived at least in part from a distinct AAV serotype. In a preferred embodiment, the adeno-associated viral vector used in the method for transducing cells in vitro or in vivo has a serotype selected from the group consisting of AAV6, AAV7, AAV8, and AAV9, and the adeno-associated virus ITRs are AAV2 ITRs.

In one example embodiment, adeno-associated viral vectors of the AAV6, AAV7, AAV8, and AAV9 serotypes are capable of transducing Th17 cells efficiently. This feature makes possible the development of methods for the treatment of diseases which require or may benefit from the increased or decreased expression of a polynucleotide of interest (e.g., one or more target genes in Tables 1A-1B and 2A-2B) in. In particular, this finding facilitates decrease in the expression of polypeptide gene products of one or more genes identified in Tables 1A-1B or the increase in expression of polypeptides of interest, such as those polypeptide gene products of the genes identified in tables 2A-2B, to a subject in need thereof by administering the AAV vectors of the disclosure to the patient, thus generating Th17 cells having increased or decreased expression of one or more target genes or gene products thereof identified in Tables 1A-1B or 2A-2B, respectively.

In one embodiment the AAV vector contains one promoter with the addition of at least one target sequence of at least one miRNA. In some embodiments, the miRNA is endogenous to cells in which it is undesirable to express the polynucleotide cargo of the AAV (e.g., a non-Th17 cell). Without being bound by theory, the endogenous miRNA would bind to its target sequence in a transcript produced from the polynucleotide cargo of the AAV and result in degradation of that transcript in the non-target cells (e.g., the non-Th17 cells).

In one example embodiment, the vector is a “plasmid,” which refers to a circular double stranded DNA loop into which additional DNA segments can be inserted, such as by standard molecular cloning techniques.

In one example embodiment, the vector is an mRNA vector (see, e.g., Sahin, U, Kariko, K and Tureci, O (2014). mRNA-based therapeutics-developing a new class of drugs. Nat Rev Drug Discov 13: 759-780; Weissman D, Karikó K. mRNA: Fulfilling the Promise of Gene Therapy. Mol Ther. 2015; 23(9):1416-1417. doi: 10.1038/mt.2015.138; Kowalski P S, Rudra A, Miao L, Anderson D G. Delivering the Messenger: Advances in Technologies for Therapeutic mRNA Delivery. Mol Ther. 2019; 27(4):710-728. doi: 10.1016/j.ymthe.2019.02.012; Magadum A, Kaur K, Zangi L. mRNA-Based Protein Replacement Therapy for the Heart. Mol Ther. 2019; 27(4):785-793. doi:10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7 (5):319-334. doi: 10.4155/tde-2016-0006; and Khalil A S, Yu X, Umhoefer J M, et al. Single-dose mRNA therapy via biomaterial-mediated sequestration of overexpressed proteins. Sci Adv. 2020; 6(27):eaba2422). In an exemplary embodiment, mRNA encoding for COBLL1 is delivered using lipid nanoparticles (see, e.g., Reichmuth, et al., 2016) and administered directly to adipose tissue. In an exemplary embodiment, mRNA encoding for COBLL1 is delivered using biomaterial-mediated sequestration (see, e.g., Khalil, et al., 2020) and administered directly to adipose tissue. Sequences present in mRNA molecules, as described further herein, are applicable to mRNA vectors (e.g., Kozak consensus sequence, miRNA target sites and WPRE).

In one example embodiment, the non-viral vector for use in gene transfer and/or nanoparticle formulations is a lipid. In one example embodiment the non-viral lipid vector may comprise: 1,2-Dioleoyl-sn-glycero-3-phosphatidylcholine; 1,2-Dioleoyl-sn-glycero-3-phosphatidylethanolamine; Cholesterol; N-[1-(2,3-Dioleyloxy)propyl]N,N,N-trimethylammonium chloride; 1,2-Dioleoyloxy-3-trimethylammonium-propane; Dioctadecylamidoglycylspermine; N-(3-Aminopropyl)-N,N-dimethyl-2,3-bis(dodecyloxy)-1-propanaminium bromide; Cetyltrimethylammonium bromide; 6-Lauroxyhexyl ornithinate; 1-(2,3-Dioleoyloxypropyl)-2,4,6-trimethylpyridinium; 2,3-Dioleyloxy-N-[2 (sperminecarboxamido-ethyl]-N,N-dimethyl-1-propanaminium trifluoroacetate; 1,2-Dioleyl-3-trimethylammonium-propane; N-(2-Hydroxyethyl)-N,N-dimethyl-2,3-bis(tetradecyloxy)-1-propanaminium bromide; Dimyristooxypropyl dimethyl hydroxyethyl ammonium bromide; 3β-[N—(N′,N′-Dimethylaminoethane)-carbamoyl]cholesterol; Bis-guanidium-tren-cholesterol; 1,3-Diodeoxy-2-(6-carboxy-spermyl)-propylamide; Dimethyloctadecylammonium bromide; Dioctadecylamidoglicylspermidin; rac-[(2,3-Dioctadecyloxypropyl)(2-hydroxyethyl)]-dimethylammonium chloride; rac-[2(2,3-Dihexadecyloxypropyl-oxymethyloxy)ethyl]trimethylammonium bromide; Ethyldimyristoylphosphatidylcholine; 1,2-Distearyloxy-N,N-dimethyl-3-aminopropane; 1,2-Dimyristoyl-trimethylammonium propane; O,O′-Dimyristyl-N-lysyl aspartate; 1,2-Distearoyl-sn-glycero-3-ethylphosphocholine; N-Palmitoyl D-erythro-sphingosyl carbamoyl-spermine; N-t-Butyl-N0-tetradecyl-3-tetradecylaminopropionamidine; Octadecenolyoxy[ethyl-2-heptadecenyl-3 hydroxyethyl] imidazolinium chloride; N1-Cholesteryloxycarbonyl-3,7-diazanonane-1,9-diamine; 2-(3-[Bis(3-amino-propyl)-amino]propylamino)-N-ditetradecylcarbamoylme-ethyl-acetamide; 1,2-dilinoleyloxy-3-dimethylaminopropane; 2,2-dilinoleyl-4-dimethylaminoethyl-[1,3]-dioxolane; and dilinoleyl-methyl-4-dimethylaminobutyrate.

In one example embodiment, the non-viral vector for use in gene transfer and/or nanoparticle formulations is a polymer. In one example embodiment the non-viral polymer vector may comprise: Poly(ethylene)glycol; Polyethylenimine; Dithiobis(succinimidylpropionate); Dimethyl-3,3′-dithiobispropionimidate; Poly(ethylene imine) biscarbamate; Poly(L-lysine); Histidine modified PLL; Poly(N-vinylpyrrolidone); Poly(propylenimine); Poly(amidoamine); Poly(amido ethylenimine); Triethylenetetramine; Poly(β-aminoester); Poly(4-hydroxy-L-proline ester); Poly(allylamine); Poly(α-[4-aminobutyl]-L-glycolic acid); Poly(D,L-lactic-co-glycolic acid); Poly(N-ethyl-4-vinylpyridinium bromide); Poly(phosphazene)s; Poly(phosphoester)s; Poly(phosphoramidate)s; Poly(N-2-hydroxypropylmethacrylamide); Poly(2-(dimethylamino)ethyl methacrylate); Poly(2-aminoethyl propylene phosphate); Chitosan; Galactosylated chitosan; N-Dodacylated chitosan; Histone; Collagen; and Dextran-spermine.

### Targeted Tissue Vector Delivery and Expression

Vector expression, such as the expression of the vectors or polynucleotides thereof described herein, can be targeted to a particular cell or tissue type. In some embodiments, the vectors are configured to have expression of one or more vector polynucleotides or sequences regulated by cell or tissue specific regulatory elements, such as promoters, enhancers, inhibitors, and/or the like. This can be accomplished as described elsewhere herein by operatively linking a polynucleotide to be expressed to one or more tissue specific regulatory elements. Additionally, or alternatively, the vectors can be delivered using a targeted delivery vehicle, such as a virus or viral like particle or capsid that has a tissue specific tropism. In some embodiments, the delivery vehicle comprises a targeting moiety that specifically targets a molecule on the surface of a target cell thus facilitating targeted delivery of the vector and ultimately targeted expression.

### Targeted Delivery

As previously mentioned, the vector or other polynucleotide for gene knock out/down (or knock/in) can be delivered to the cell using a targeted delivery approach so as to limit delivery and thus expression of a polynucleotide to only cells in which it is delivered. In some embodiments, the delivery vehicle (or composition) used to deliver the vector or other polynucleotide can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety that can target a T cell, such as a CD4⁺ T cell or a Th17 cell. As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity where the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10 3 M). In some embodiments, the targeting moiety has increased binding with, association with, interaction with, or activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

In some embodiments, a targeting moiety effective to target a Th17 cells target CCR4, CCR6, CD3, CD4, IL-1RI, IL-6R alpha, IL-21R, IL-23R, and/or TGF-beta RII.

### Targeted Expression

In one example embodiment, the vectors are configured for specific expression in Th17 cells. In some embodiments, the vectors are configured for expression in one or more tissues where Th17 cells are generated, differentiated, or activated. In another example embodiment, the transcriptional regulatory region may comprise a promoter and, optionally, an enhancer region. In some embodiments, the promoter is specific for spleen, thymus, tonsils, mucosal-associated lymphoid tissues, or lymph other tissue. The enhancer need not be specific for spleen, thymus, tonsils, mucosal-associated lymphoid tissues, or lymph other tissue. Alternatively, the transcriptional regulatory region may comprise a spleen, thymus, tonsil, mucosal-associated lymphoid tissue, or other lymph tissue-specific promoter and a spleen or lymph tissue-specific enhancer. In one embodiment, the tissue-specific promoter is a spleen-thymus-, tonsil-, mucosal-associated lymphoid tissue-, or other lymph tissue-specific such as, for example, the promoters associated with the promoter region of Tef7, IL-17, RORγ, or RORγt or another pro. In certain embodiments, spleen- or lymph-specific promoters may include the T early α (TEA) promoter located on the 5′ of the TCR Ja segment (see, e.g., Eberl, G., &amp; Littman, D. R. (2003). The role of the nuclear hormone receptor RORγt in the development of lymph nodes and Peyer's patches. Immunological Reviews, 195(1), 81-90. doi.org/10.1034/j.1600-065x.2003.00074). In another example embodiment, the transcriptional regulatory region may comprise multiple promoters, and optionally, an enhancer region. In example embodiments, the multiple promoters are associated with the promoter regions of one or more of IL-17, RORγ or RORγt. In one embodiment, and without being bound by a theory, the enhancer region is associated with IL-17, RORγ or RORγt. In another example embodiment, one or more enhancers may be associated with the transcriptional regulatory region. In an example embodiment, and without being bound by a theory, the enhancers include enhancers of the region that associate with ETS family members and/or TCF1 in a cooperative manner. In another example embodiment, a spleen- or lymph-specific promoter is much less potent than that of a ubiquitous promoter. Thus, a ubiquitous promoter, such as hybrid cytomegalovirus enhancer/chicken β-actin (CBA or CAG) or cytomegalovirus (CMV) is used. In another example embodiment, a ubiquitous promoter is used in combination with any Th17 cell targeting strategy described herein or when the vector is administered locally to spleen, thymus, tonsil, mucosal-associated lymphoid tissue, or other lymph tissue. In another example embodiment, systemic delivery utilizes a spleen- or lymph-specific promoter with a higher dose, while local delivery utilizes a CBA or CMV promoter with a lower dosage.

In some embodiments, non-thymus, non-spleen, non-lymph, and/or non-Th17 cells are detargeted. In some embodiments, the vector contains at least one miRNA target sequence for an mRNA that is expressed in the cell to be detargeted. Without being bound by theory, in such cells, if RNA is transcribed from a vector sequence that includes the miRNA target sequence, such a sequence will be targeted and degraded by the miRNA that targets the miRNA target sequence and prevent translation of the RNA. In one embodiment, the vector contains at least one target sequence of at least one miRNA expressed in non-lymph or -spleen tissue. In another example embodiment, liver- and heart-specific abundant miRNAs are used to de-target or suppress transgene expression in liver and heart by embedding the miRNA target sequences in the vectors, in particular for AAV8 vectors. In one embodiment, the target sequence of at least one miRNA is located in the 3′ untranslated region (3′UTR) of cellular messenger RNA (mRNA). Exemplary target sequences of the at least one miRNA include, but are not limited to miR1 (miRbase database accession numbers MI0000651 and MI0000437), miR122 or miR122a (MI0000442), miR152 (MI0000462), miR199 (MI0000242), miR215 (MI0000291), miR192 (MI0000234), miR148a (MI0000253), miR194 (MI0000488), miR1 (MI0000651), miRT133 (MI0000450), miR206 (MI0000490), miR208 (MI0000251), miR124 (MI0000443), miR125 (MI0000469), miR216 (MI0000292), and miR130 (MI0000448). In preferred embodiments, the miRNA target sites are selected from miRNA122a and miRNA1. In another example embodiment, 1, 2, 3, or 4 repeat target sites for each miRNA can be used. Sequence references are publicly available and may be obtained from the miRbase (mirbase.org/). The term “microRNAs” or “miRNAs”, as used herein, are small (˜22-nt), evolutionarily conserved, regulatory RNAs involved in RNA-mediated gene silencing at the post-transcriptional level (see, e.g., Barrel DP. Cell 2004; 116:281-297). Through base pairing with complementary regions (most often in the 3′ untranslated region (3′UTR) of cellular messenger RNA (mRNA)), miRNAs can act to suppress mRNA translation or, upon high-sequence homology, cause the catalytic degradation of mRNA. Because of the highly differential tissue expression of many miRNAs, cellular miRNAs can be exploited to mediate tissue-specific targeting of gene therapy vectors. By engineering tandem copies of target elements perfectly complementary to tissue-specific miRNAs (miRT) within vectors, transgene expression in undesired tissues can be efficiently inhibited.

### Vector-Based Gene Knock-In

In one example embodiment, the method for treating an autoimmune or inflammatory disease or disorder comprises administering one or more recombinant vectors encoding one or more target genes of Tables 2A/2B. The vector may further comprise one or more regulatory elements to control expression of the one or more target genes or transcription factors. The vector may further comprise regulatory/control elements, e.g., promoters, enhancers, introns, polyadenylation signals, Kozak consensus sequences, or internal ribosome entry sites (IRES). The vector may further comprise cellular localization signals, such as a nuclear localization signal (NLS) or nuclear export signal (NES). The vector may further comprise a targeting moiety that directs the vector specifically to T cells or T cell progenitors. In another example embodiment, the vector may comprise a viral vector with a trophism specific for T cells and T cell progenitors. The sections regarding “Regulatory Elements,” “Vector Selection,” and “Targeted Delivery” discussed under “Vector-Based Gene Knock-out” equally apply to the recombinant vectors described in this section.

### RNAi and Antisense Oligonucleotides (ASO)

In one example embodiment, a method of treating an autoimmune or inflammatory disease and/or disorder comprising administering one or more RNAi agents directed to one or more genes from Tables 1A/B such that expression of the one or more genes from Table 1A/B is reduced. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%. Additionally, inhibitory nucleic acid molecules such as RNAi and ASOs can be used in vivo (see, e.g., Yan Y, Liu X Y, Lu A, Wang X Y, Jiang L X, Wang J C. Non-viral vectors for RNA delivery. J Control Release. 2022; 342:241-279).

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, about 19-30 base nucleotides, or about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Antisense therapy is a form of treatment that uses antisense oligonucleotides (ASOs) to target messenger RNA (mRNA). ASOs are capable of altering mRNA expression through a variety of mechanisms, including ribonuclease H mediated decay of the pre-mRNA, direct steric blockage, and exon content modulation through splicing site binding on pre-mRNA (see, e.g., Crooke S T, Liang X H, Baker B F, Crooke R M. Antisense technology: A review. J Biol Chem. 2021; 296:100416. doi: 10.1016/j.jbc.2021.100416). Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Commonly used antisense mechanisms to degrade target RNAs include RNase H1-dependent and RISC-dependent mechanisms. Preferred ASOs include Locked Nucleic Acid (LNA), Peptide Nucleic Acid (PNA), and morpholinos.

### Genetic Modification Systems

In one example embodiment, the method for treating an autoimmune or inflammatory disease and/or disorder comprises administering a genetic modification system to either decrease expression of one or more gene s from Tables 1A and/or 1B or increase expression of one or more genes from Tables 2A or 2B. The genetic modifying agent may comprise a programmable nuclease, such as, a CRISPR system, a zinc finger nuclease system, a TALEN, or a meganuclease, or an OMEGA system. In addition, a number of alternate gene modification systems have been developed by modifying Cas nuclease so that they are catalytically inactive (“dead Cas” or “dCas”) or cut only a single strand of DNA (“nickase”) and then coupling these modified Cas nuclease with a further functional domain such as base editors, reverse transcriptases, recombinases, transposases and retrotransposases. For sake of convenience these alternative systems (e.g., Base Editors, Prime Editors, CAST, Non-LTR Retrotransposon Systems, Epigenetic Editors) are described further below in the context of use with a modified Cas. However, it is further contemplated that the modified Cas could be substituted with another similarly modified programmable nuclease like a Zinc Finger nucleases, TALENs, Omega nucleases (e.g., Iscb, Isrb, TnpB, Fanzor), meganuclease. In example embodiments, the genetic modifying agent is administered using a vector, such as a viral vector or liposome. In example embodiments, the genetic modifying agent is targeted to tumor cells (see, e.g., Montaño-Samaniego M, Bravo-Estupiñan DM, Méndez-Guerrero O, Alarcón-Hernández E, Ibáñez-Hernández M. Strategies for Targeting Gene Therapy in Cancer Cells With Tumor-Specific Promoters. Front Oncol. 2020; 10:605380; and Jafari M, Kadkhodazadeh M, Shapourabadi M B, et al. Immunovirotherapy: The role of antibody based therapeutics combination with oncolytic viruses. Front Immunol. 2022; 13:1012806). In example embodiments, the genetic modifying agent is administered directly to a tumor. Programmable nucleases may use two different cell repair pathways to effectuate edits to one or more target sequences, non-homologous end joining (NHEJ) or homology-directed repair (HDR).

### Example NHEJ-Mediated Modifications

Programmable nuclease may be used to introduce insertions and deletions via NHEJ-mediated cell repair that control expression of one or more genes of Tables 1A, 1B, 2A, 2B, or combinations thereof. The modifications may be made in a non-coding region that controls expression of the one or more target genes, in a coding region encoding a gene expression product (e.g., a polypeptide) of the one or more target genes, or a combination thereof. More than one programmable nuclease type may be used, for example and in the case of CRISPR-Cas, to maximize targets sites adjacent to different PAMs.

NHEJ-Mediated Modifications that Decrease Expression by Targeting a Non-Coding Region

In one embodiment, the one or more programmable nucleases may be configured to introduce one or more insertions or deletion in a non-coding region controlling expression of one or more genes in Tables 1A or 1B such that expression of the one or more genes is reduced. In one embodiment, the insertions or deletions may disrupt the binding site in an enhancer of one or more proteins, such as a transcription factor or other regulatory proteins, needed to initiate transcription of one or more genes. In one embodiment, the one or more insertions or deletions may disrupt one or more promoters controlling expression of one or more genes in Tables 1A or 1B such that binding of transcription factors and/or RNA polymerase binding is blocked or reduced. In one embodiment, the one or more insertions or deletions may disrupt one or more insulator regions such that silencer regions or repressive chromatin structures controlling expression of the one or more genes in Tables 1A or 1B are no longer muted or blocked by the insulator region and can decrease gene expression.

NHEJ-Mediated Modification that Increase Expression by Targeting a Non-Coding Region

In one embodiment, the one or more programmable nucleases may be configured to introduce one or more insertions or deletions in a non-coding region controlling expression of one or more genes in Tables 2A or 2B such that expression of the one or more target genes is increased. In one embodiment, the one or more insertions or deletions modify one or more enhancer regions controlling expression of one or more genes in Tables 2A or 2B such that binding of transcription factors or other regulatory proteins is increased or strengthened and gene expression is increased. In one embodiment, the one or more insertions or deletions modify one or more promoter regions controlling expression of one or more genes in Tables 2A or 2B such that binding of transcription factors and/or RNA polymerase is increased or strengthened and gene expression is increased. In one embodiment, the one or more insertions or deletions disrupt one or more silencer regions controlling expression of one or more genes in Tables 2A or 2B, such that binding of transcriptional repressor is blocked or reduced and gene expression is increased.

NHEJ-Mediated Modification that Decrease Expression by Targeting a Coding Region

In one embodiment, the programmable nuclease is used to introduce one or more insertions or deletions to coding sequence of one or more genes in Tables 1A or 1B, such that one or more indels or insertions reduce expression or activity of one or more genes in Tables 1A or 1B. For example, the insertion or deletion may cause a frame shift in the coding sequence such that expression is reduced or such that the resulting gene product is non-functional or exhibits reduced activity relative to an unmodified gene. In one embodiment, the insertion(s) or deletion(s) may alter a splice site such that transcription or translation is reduced or such that a resulting gene product is non-functional or exhibits reduced activity relative to an unmodified gene. The insertion or deletion may introduce a premature stop codon such that expression is reduced. The insertion or deletion may alter a post-translational modification site such that the activity of the resulting gene product is reduced.

NHEJ-Mediated Modification that Increase Expression by Targeting a Coding Region

In one embodiment, the programmable nuclease is used to introduce one or more deletions or insertions in the coding sequence of one or more genes in Tables 2A or 2B such that expression of the one or more genes is increased. For example, the insertion or deletion may cause a frame shift in the coding sequence such that expression is increased or such that the resulting gene product exhibits increased activity relative to an unmodified gene. The insertion or deletion may alter a splice site such that transcription or translation is increased or such that a resulting gene product exhibits increased activity relative to an unmodified gene. The insertion or deletion may introduce a premature stop codon such that expression is increased. The insertion or deletion may alter a post-translational modification site such that the activity of the resulting gene product is increased.

### Example HDR-Mediated Modifications

In one example embodiment, a donor template is provided along with a programmable nuclease to facilitate homology direct repair (HDR) which results insertion of a donor sequence comprising one or more insertions, deletions, or substitutions relative to the target sequence it replaces. A donor template may comprise an insertion sequence flanked by two homology regions. The insertion sequence comprises an edited sequence to be inserted in place of the target sequence (e.g., a portion of genomic DNA to be edited). The homology regions comprise sequences that are homologous to the genomic DNA strands at the site of the CRISPR-Cas induced double-strand break (DSB). Cellular HDR mechanisms then facilitate insertion of the insertion sequence at the site of the DSB.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

The use of donor templates may be used to introduce insertions, deletions, or substitutions (modifications) that control expression of the one or more genes of Tables 1A, 1B, 2A, and 2B. The modifications may be made in a non-coding region that controls expression of the one or more target genes, in a coding region encoding a gene expression product (e.g., a polypeptide), or both. Example modifications are described in further detail below.

HDR-Based Modifications that Decrease Expression by Targeting Non-Coding Regions

In one example embodiment, the donor template is configured to introduce a deletion, insertion, or mutation in one or more enhancer regions such that binding of transcription factors or other regulatory proteins controlling expression of the one or more genes in Tables 1A or 1B is disrupted thereby reducing transcription initiation and gene expression. In one example embodiment, the donor template is configured to introduce a deletion, insertion, or mutation in one or more promoters controlling expression of one or more genes in Tables 1A or 1B to prevent or disrupt the binding of transcription factors and RNA polymerase such that transcription initiation and gene expression are blocked or reduced. In one example embodiment, the donor template is configured to introduce a silencer element into a non-coding region controlling expression of one or more genes in Tables 1A or 1B leading to the recruitment of transcriptional repressors that block or decrease gene expression. In one embodiment, the donor template is configured to modify or replace an existing silencer element controlling expression of one or more genes in Tables 1A or 1B such that the silencing function of the silencer element is increased relative to an unmodified silencer sequence. In another embodiment, the donor template is configured to disrupt or replace one or more insulator sequences controlling expression of one or more genes in Tables 1A or 1B such that nearby silencer element or repressive chromatin structures decrease gene expression.

HDR-Based Modifications that Increase Expression by Targeting Non-Coding Regions

In one embodiment, the programable nuclease and donor template may be configured to make one or more modifications (insertions, substitutions, deletions) in a non-coding region of one or more genes in Tables 2A or 2B that result in increased expression of the one or more genes in Tables 2A or 2B. In one embodiment, the one or more modifications modify one or more enhancer regions controlling expression of one or more genes in Tables 2A or 2B such that binding of transcription factors or other regulatory proteins is increased or strengthened and gene expression is increased. In another embodiment, the one or more modifications modify one or more promoters controlling expression of one or more genes in Tables 2A or 2B such that binding of transcription factors and/or RNA polymerase is increased or strengthened and gene expression is increased. In another embodiment, the one or modifications disrupt or remove one or more silencer elements that control expression of one or more genes in Tables 2A or 2B, such that binding of transcriptional repressors is prevented or weakened and gene expression is increased. In another embodiment, the one or more modifications introduce or strengthen insulator sequences controlling expression of the one or more genes in Tables 2A or 2B thereby reducing the influence of nearby silencer elements or repressive chromatin structures such that gene expression is increased.

HDR-Based Modifications that Decrease Expression by Targeting Coding Regions

In one embodiment, the programmable nuclease and donor template are configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of non-functional, truncated proteins or the triggering of nonsense-mediated mRNA decay (NMD) thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability, and localization. Post-translation modification may be both necessary to inhibit a protein's functions or to activate a protein's functions. Accordingly, modifications that introduce inhibitory PTMs or remove activating PTMs may be made to decrease protein function, stability, and/or degradation.

HDR-Based Modifications that Increase Expression by Targeting Coding Regions

In one embodiment, the programmable nuclease and donor template are configured such that one or more modifications (e.g., insertions, deletions, substitutions) are made in a coding region of the one or more genes of Tables 2A or 2B such that expression of the one or more genes is increased. In one embodiment, the one or more modifications comprise removing inhibitor sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modifications may comprise mutations or modifications within the coding region that improve catalytic activity, binding affinity, or other functional properties of the protein. This approach does not directly increase gene expression but can result in an overall increase in the functional output of the gene product.

### Example Programmable Nucleases

The following provides further details and nuclease specific considerations for example programmable nucleases that may be used to make the NHEJ-mediated and HDR-mediated modifications described above.

### CRISPR-Cas

In one example embodiment, the genetic modifying agent is a CRISPR-Cas system. CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence in one or more of the target genes. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to a desired target site at or near the one or more target genes. Additionally, CRISPR systems can be used in vivo (see, e.g., Chen H, Shi M, Gilam A, et al. Hemophilia A ameliorated in mice by CRISPR-based in vivo genome editing of human Factor VIII. Sci Rep. 2019; 9 (1): 16838; Hana S, Peterson M, Mclaughlin H, et al. Highly efficient neuronal gene knockout in vivo by CRISPR-Cas9 via neonatal intracerebroventricular injection of AAV in mice. Gene Ther. 2021; 28 (10-11): 646-658; and Rosenblum D, Gutkin A, Kedmi R, et al. CRISPR-Cas9 genome editing using targeted lipid nanoparticles for cancer therapy. Sci Adv. 2020; 6 (47): eabc9450).

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present disclosure described herein can be a Class 1 CRISPR-Cas system. In some embodiments, the CRISPR-Cas system that can be used to modify a polynucleotide of the present disclosure described herein can be a Class 2 CRISPR-Cas system.

### Class 1 CRISPR-Cas Systems

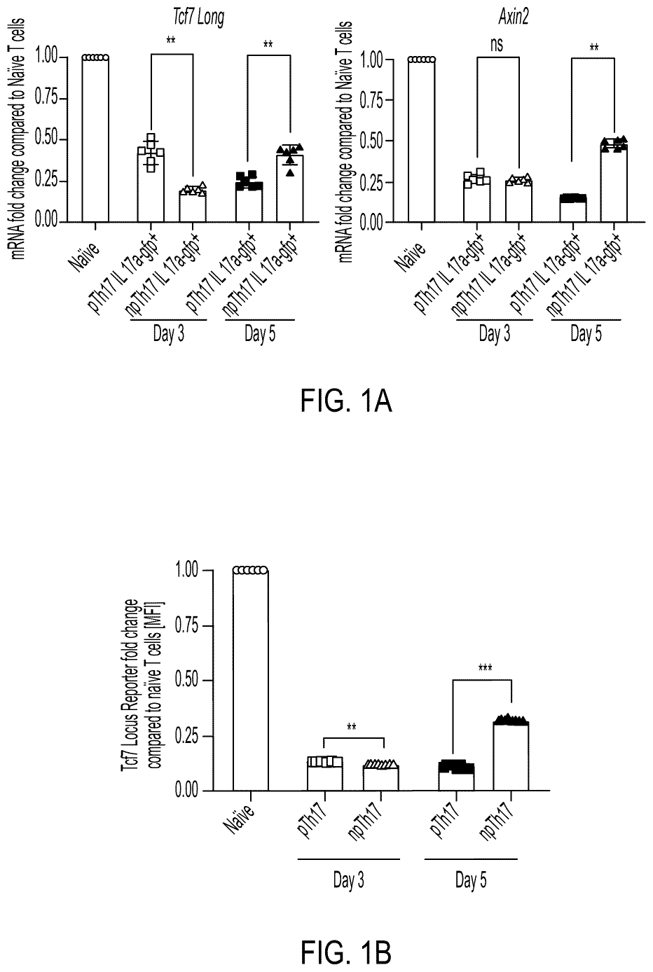

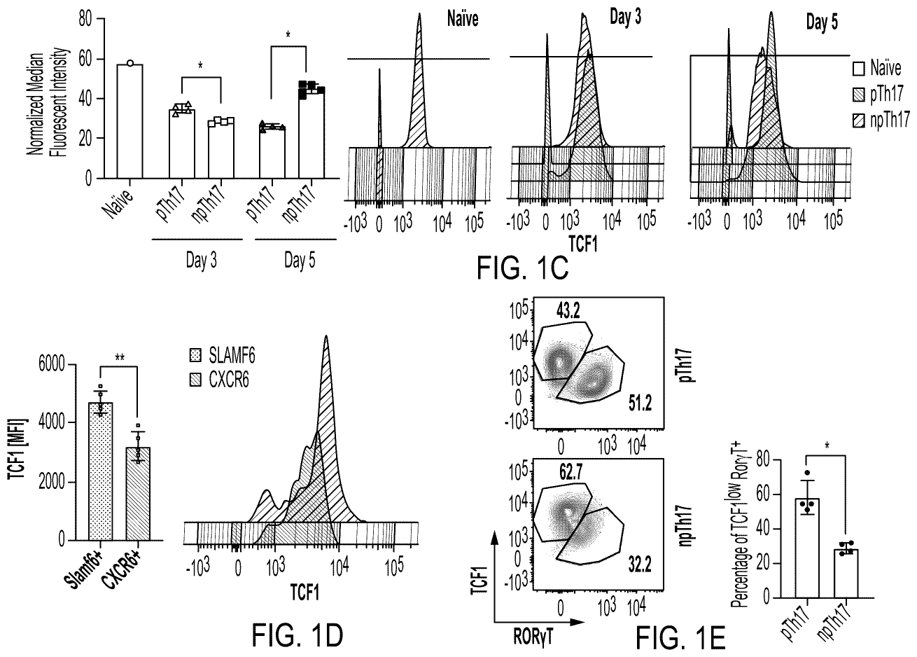

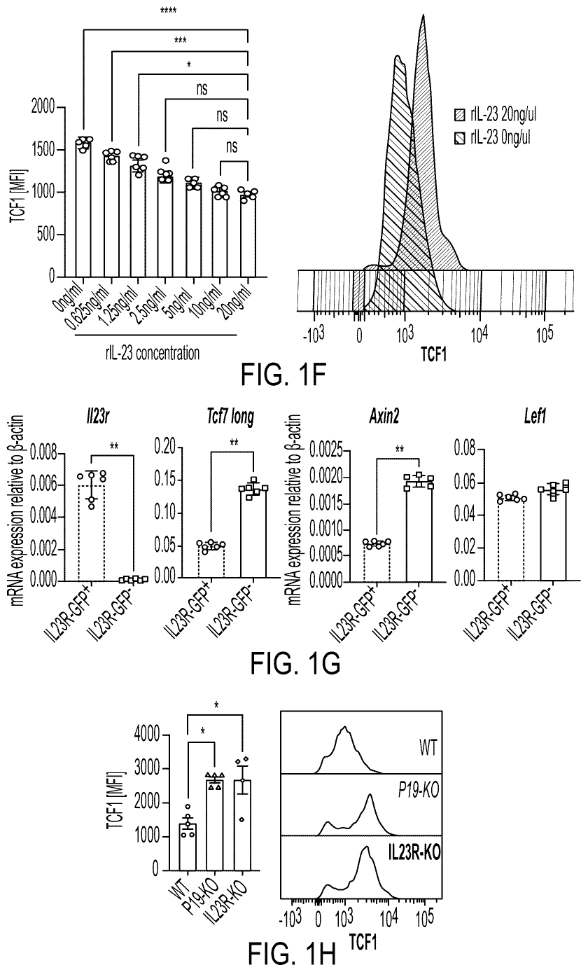

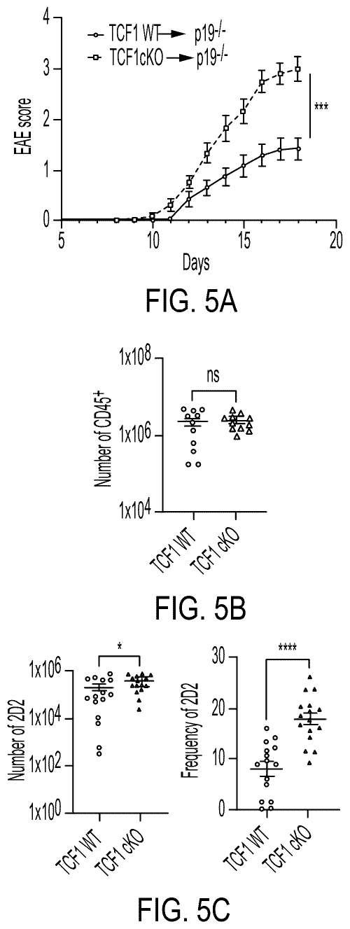

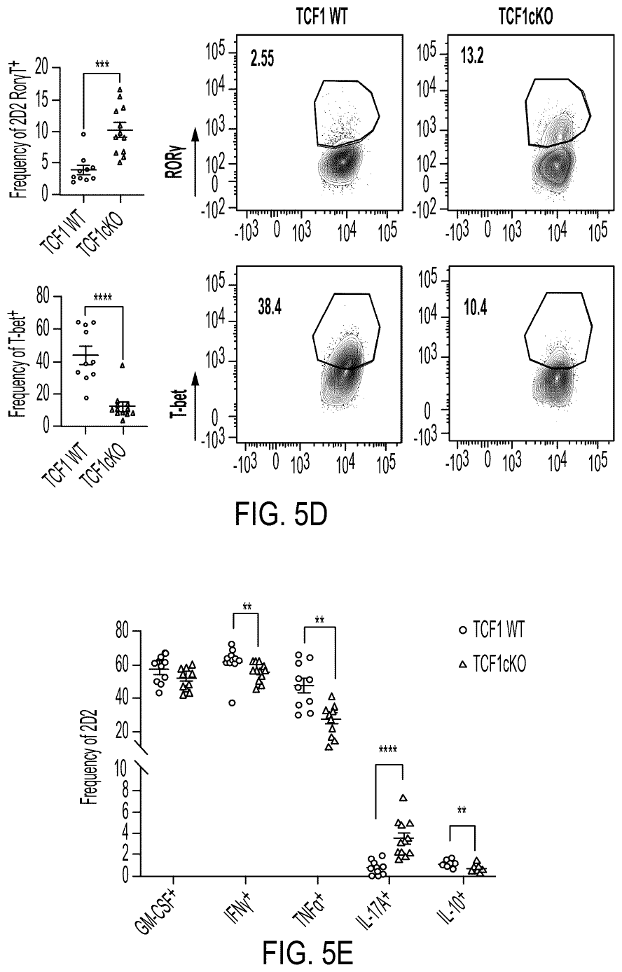

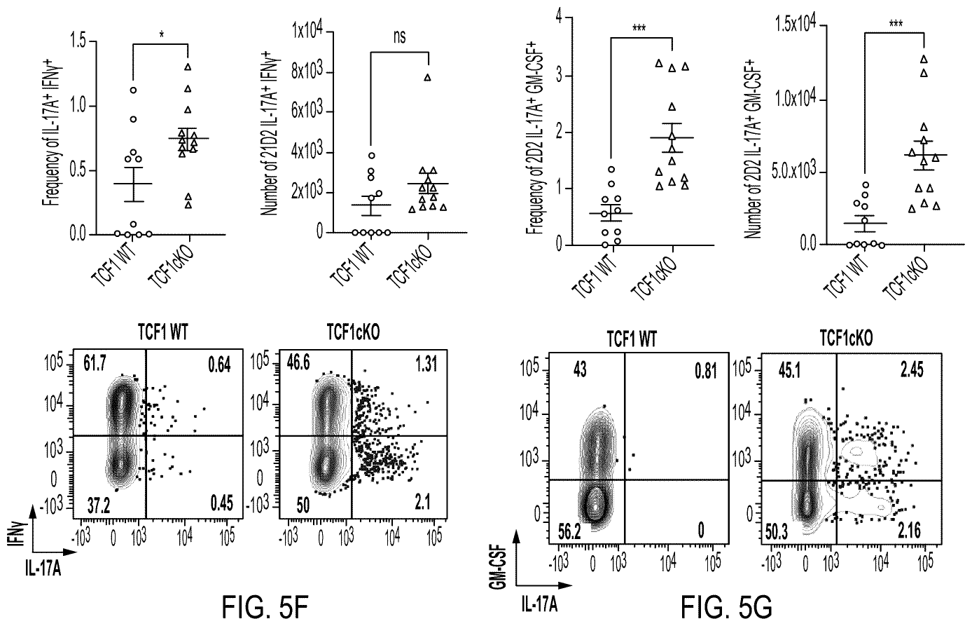

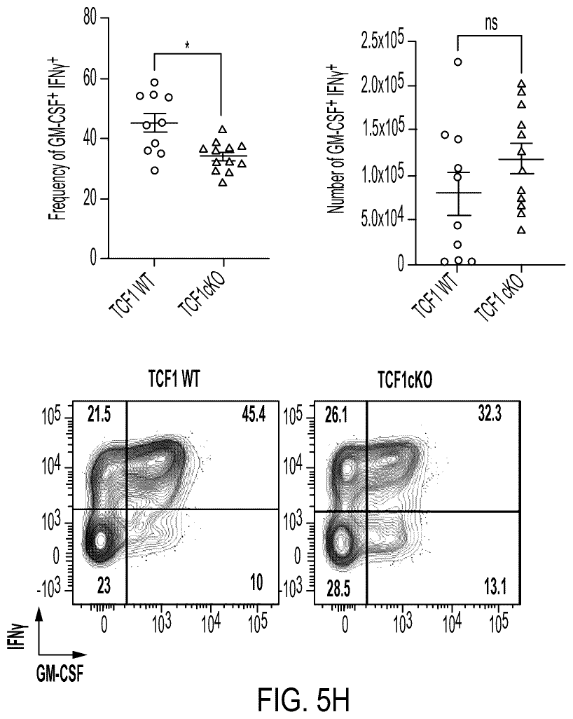

In some embodiments, the CRISPR-Cas system that can be used to modify a polynucleotide of the present disclosure described herein can be a Class 1 CRISPR-Cas system. Class 1 CRISPR-Cas systems are divided into types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and I-G). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al. 2018. The CRISPR Journal, v. 1, n5, FIG. 5.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas7 proteins. In some embodiments, the Cas6 protein is an RNAse, which can be responsible for pre-crRNA processing. When present in a Class 1 CRISPR-Cas system, Cas6 can be optionally physically associated with the effector complex.

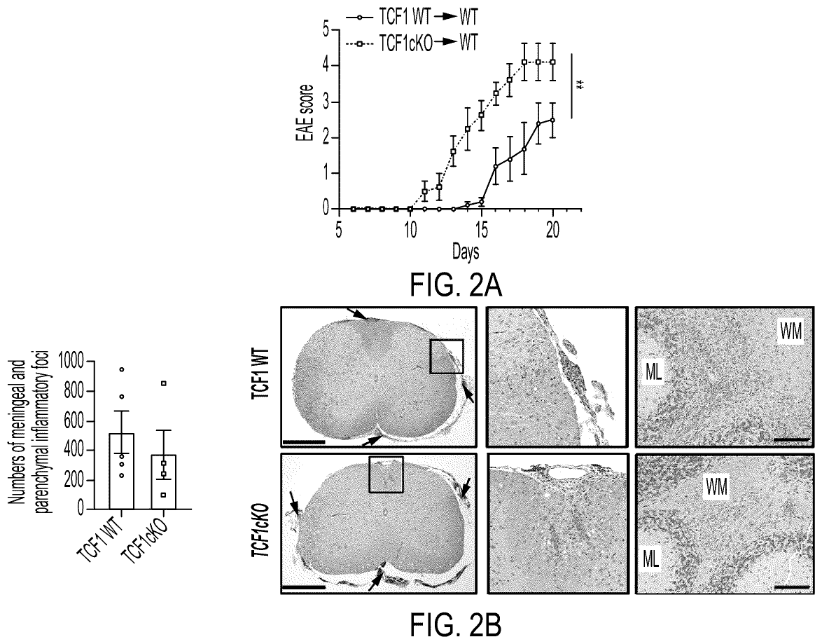

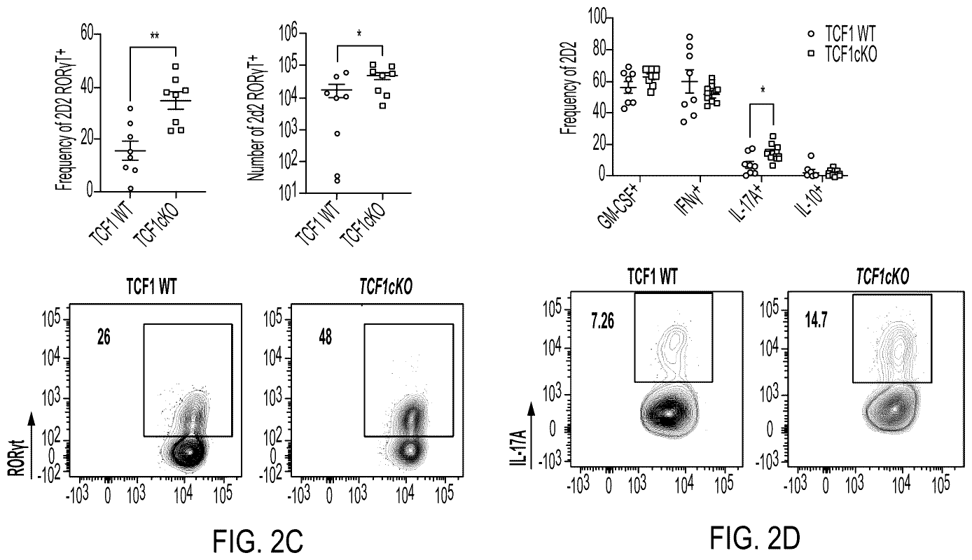

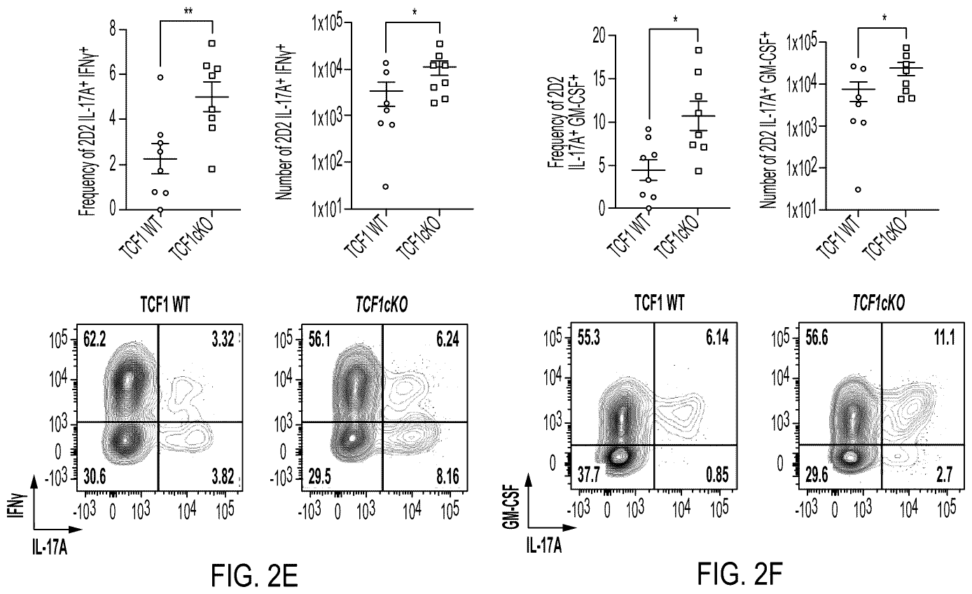

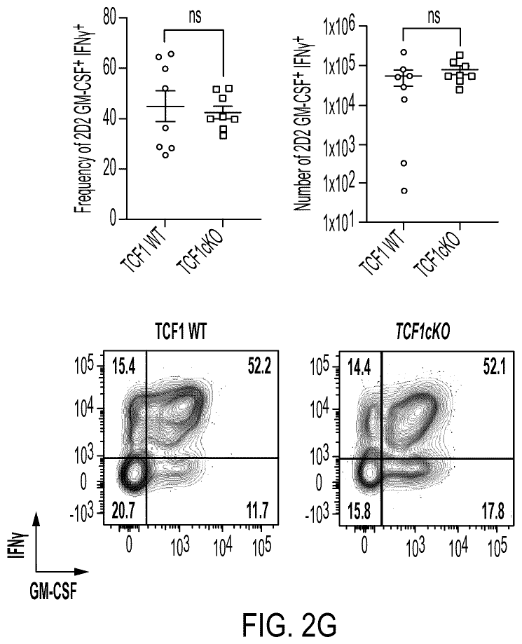

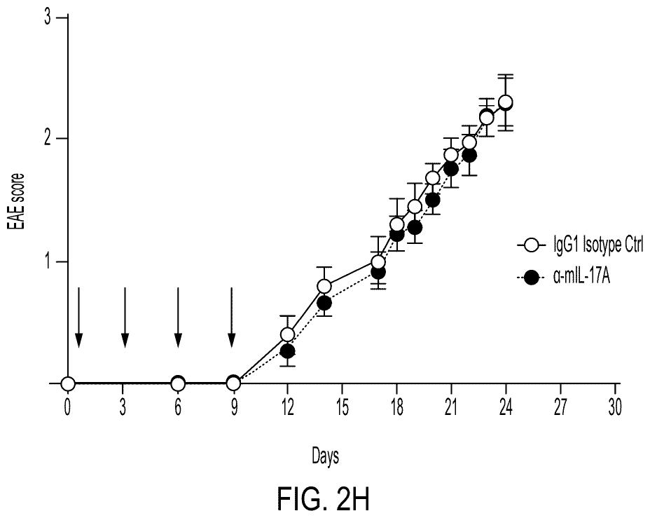

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374:20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374:20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2cl), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, optionally a single stem loop. In one example embodiment, the direct repeat sequence forms a stem loop, optionally a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the spacer sequence and tracr sequence, along the length of the shorter of the two sequences. Optimal alignment may be determined by any suitable alignment algorithm and may further account for secondary structures, such as self-complementarity within either the spacer sequence or tracr sequence. In some embodiments, the degree of complementarity between the tracr sequence and spacer sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 98.5% or 98% or 97.5% or 97% or 96.5% or 96% or 95.5% or 95% or 94.5% complementarity between the sequence and the guide.

In some embodiments according to the disclosure, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this disclosure. Various modifications may be used to increase the specificity of binding to the target sequence and/or increase the activity of the Cas protein and/or reduce off-target effects. Example guide sequence modifications are described in International Patent Application No. PCT/US2019/045582, specifically paragraphs [0178]-[0333], which is incorporated herein by reference.

### Target Sequences, PAMs, and PESs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4): 504-517. Table A (from Gleditzsch et al. 2019) below shows several Cas polypeptides and the PAM sequence they recognize.

TABLE A
Example PAM Sequences

| Cas Protein | PAM Sequence |
|---|---|
| SpCas9 | NGG (SEQ ID NO: 1)/NRG |
| | (SEQ ID NO: 2) |
| SaCas9 | NGRRT (SEQ ID NO: 3) or |
| | NGRRN (SEQ ID NO: 4) |
| NmeCas9 | NNNNGATT (SEQ ID NO: 5) |
| CjCas9 | NNNNRYAC (SEQ ID NO: 6) |
| StCas9 | NNAGAAW (SEQ ID NO: 7) |
| Cas12a (Cpf1) (including LbCpf1 | TTTV (SEQ ID NO: 8) |
| and AsCpf1) | |
| Cas12b (C2c1) | TTT (SEQ ID NO: 9), TTA (SEQ |
| | ID NO: 10), and TTC (SEQ ID |
| | NO: 11) |
| Cas12c (C2c3) | TA (SEQ ID NO: 12) |
| Cas12d (CasY) | TA (SEQ ID NO: 13) |
| Cas12e (CasX) | 5′-TTCN-3′ (SEQ ID NO: 14) |
| Cas1 | 5′-CTT-3′ (SEQ ID NO: 15) |
| Cas8e | 5′-ATG-3′ (SEQ ID NO: 16) |
| Type I-A | 5′-CCN-3′ (SEQ ID NO: 17) |
| Type I-B | TTC (SEQ ID NO: 18), ACT |
| | (SEQ ID NO: 19), TAA (SEQ ID |
| | NO: 20), TAT (SEQ ID NO: 21), |
| | TAG (SEQ ID NO: 22), and CAC |
| | (SEQ ID NO: 23) |
| Type I-C | NTTC (SEQ ID NO: 24) |
| Type I-E | 5′-AAG-3′ (SEQ ID NO: 25) |
| Type I-F | GG (SEQ ID NO: 26) |

In a preferred embodiment, the CRISPR effector protein may recognize a 3′ PAM. In one example embodiment, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. Doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3): 733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LshCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16 (4): 504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16 (4): 504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 27) or PKKKRKVEAS (SEQ ID NO: 28); the NLS from nucleoplasmin (e.g., the nucleoplasmin bipartite NLS with the sequence KRPAATKKAGQAKKKK (SEQ ID NO: 29)); the c-myc NLS having the amino acid sequence PAAKRVKLD (SEQ ID NO: 30) or RQRRNELKRSP (SEQ ID NO: 31); the hRNPAI M9 NLS having the sequence NQSSNFGPMKGGNFGGRSSGPYGGGGQYFAKPRNQGGY (SEQ ID NO: 32); the sequence RMRIZFKNKGKDTAELRRRRVEVSVELRKAKKDEQILKRRNV (SEQ ID NO: 33) of the IBB domain from importin-alpha; the sequences VSRKRPRP (SEQ ID NO: 34) and PPKKARED (SEQ ID NO: 35) of the myoma T protein; the sequence PQPKKKPL (SEQ ID NO: 36) of human p53; the sequence SALIKKKKKMAP (SEQ ID NO: 37) of mouse c-abl IV; the sequences DRLRR (SEQ ID NO: 38) and PKQKKRK (SEQ ID NO: 39) of the influenza virus NS1; the sequence RKLKKKIKKL (SEQ ID NO: 40) of the Hepatitis virus delta antigen; the sequence REKKKFLKRR (SEQ ID NO: 41) of the mouse Mx1 protein; the sequence KRKGDEVDGVDEVAKKKSKK (SEQ ID NO: 42) of the human poly(ADP-ribose) polymerase; and the sequence RKCLQAGMNLEARKTKK (SEQ ID NO: 43) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

In certain embodiments, the CRISPR-Cas protein and a functional domain protein (described further herein) are delivered to the cell or expressed within the cell as separate proteins. In these embodiments, each of the CRISPR-Cas and functional domain protein can be provided with one or more NLSs as described herein. In certain embodiments, the CRISPR-Cas and functional domain protein are delivered to the cell or expressed with the cell as a fusion protein. In these embodiments one or both of the CRISPR-Cas and functional domain protein is provided with one or more NLSs. Where the functional domain protein is fused to an adaptor protein (such as MS2) as described above, the one or more NLS can be provided on the adaptor protein, provided that this does not interfere with aptamer binding. In particular embodiments, the one or more NLS sequences may also function as linker sequences between the functional domain protein and the CRISPR-Cas protein.

In certain embodiments, guides of the disclosure comprise specific binding sites (e.g., aptamers) for adapter proteins, which may be linked to or fused to a functional domain protein or catalytic domain thereof. When such a guide forms a CRISPR complex (e.g., CRISPR-Cas protein binding to guide and target), the adapter proteins bind and the functional domain protein or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex), are modifications which are not intended. The one or more modified guide may be modified at the tetra loop, the stem loop 1, stem loop 2, or stem loop 3, as described herein, optionally at either the tetra loop or stem loop 2, and in some cases at both the tetra loop and stem loop 2.

In some embodiments, a component (e.g., the dead Cas protein, the functional domain protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively, or additionally, the NES or NLS may be at the N terminus of component. In some examples, the Cas protein and optionally said functional domain protein or catalytic domain thereof comprise one or more heterologous nuclear export signal(s) (NES(s)) or nuclear localization signal(s) (NLS(s)), optionally an HIV Rev NES or MAPK NES, optionally C-terminal.

### OMEGA Systems

In one example embodiment, the programmable nuclease to modify the one or more target genes is a transposon-encoded RNA-guided nuclease system, referred to herein as OMEGA (obligate mobile element-guided activity). See, e.g., Altae-Tran H, Kannan S, Demircioglu F E, et al. The widespread IS200/IS605 transposon family encodes diverse programmable RNA-guided endonucleases. Science. 2021; 374 (6563): 57-65. OMEGA systems include, but are not limited to IscB, IsrB, TnpB systems.

In some embodiments, the nucleic acid-guided nucleases herein may be an IscB protein (see, e.g., International patent application publication No. WO2022087494A1; and Altae-Tran H. et al. 2021). An IscB protein may comprise an X domain and a Y domain as described herein. In some examples, the IscB proteins may form a complex with one or more guide molecules. In some cases, the IscB proteins may form a complex with one or more hRNA molecules, which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. Doi: 10.1128/JB.00783-15, which is incorporated by reference herein in its entirety.

In some embodiments, the nucleic acid-guided nucleases herein may be an IsrB (Insertion sequence RuvC-like OrfB) protein (see, e.g., International patent application publication No. WO 2022/087494A1; and Altae-Tran H, et al. 2021). IsrB refers to a group of shorter, ˜350 aa IscB homologs that are also encoded in IS200/605 superfamily transposons. These proteins contain a PLMP domain and split RuvC, but lack the HNH domain.

In some embodiments, the nucleic acid-guided nucleases herein may be a TnpB protein (see, e.g., International patent application publication No. WO 2022/159892A1; and Altae-Tran H. et al. 2021). TnpB is a putative endonuclease distantly related to IscB and thought to be the ancestor of Cas12, the type V CRISPR effector. The TnpB system comprises a TnpB polypeptide and a nucleic acid component capable of forming a complex with the TnpB polypeptide and directing the complex to a target polynucleotide. The TnpB systems and TnpB/nucleic acid component complexes may also be referred to herein as OMEGA (Obligate Mobile Element Guided Activity) systems or complexes, or Ω systems or complexes for short. TnpB systems are a distinct type of Ω system, which further include IscB, IsrB, and IshB systems. The nucleic acid component of Ω systems is structurally distinct from other RNA-guided nucleases, such as CRISPR-Cas systems, and may also be referred to as a ωRNA. In certain example embodiments, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide.

Accordingly, it is contemplated within the scope of the present disclosure that OMEGA systems may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

Accordingly, it is contemplated within the scope of the present disclosure that Zn finger nucleases may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)—X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the disclosure can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the disclosure will bind. As used herein the monomers and at least one or more-half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the disclosure may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 44)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N.

An exemplary amino acid sequence of a C-terminal capping region is:

(SEQ ID NO: 45)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the disclosure.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, or % sequence identity. The software typically does this as part of the sequence comparison and generates a numerical result.

In some embodiments described herein, the TALE polypeptides of the disclosure include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the disclosure may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the disclosure may include any combination of the activities described herein.

Accordingly, it is contemplated within the scope of the present disclosure that TALE nucleases may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514; 8,133,697; 8,021,867; 8,119,361; 8,119,381; 8,124,369; and 8,129,134, which are specifically incorporated herein by reference.

Accordingly, it is contemplated within the scope of the present disclosure that meganucleases may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed below.

### Other Genetic Modification Systems

A number of alternative gene modification systems have been developed that utilize the target specificity of a programmable nuclease but that modify or replace that nuclease activity with another functional activity. For example, programmable nucleases may be modified such that they cleave only a single strand as opposed to both strands of a target polynucleotide. Such “nickases” may then be paired with other functional domains such as reverse transcriptases, recombinases and non-LTR retrotransposon polypeptides to make genetic modifications that do not rely on creating double strand breaks. Similarly, programmable nucleases may also be modified to eliminate the nuclease activity altogether. These catalytically inactive or “dead” nucleases may then be combined with other functional domains like nucleotide deaminases, transposases, non-LTR retrotransposon polypeptides, methylases, deactylases, and acetylases, among other domains. The following provides further examples of gene modification systems that may be used in the context of the present disclosure. For ease of reference the gene modifications systems that follow will be discussed in the context of using CRISPR-Cas as the programmable nuclease system, but it is contemplated within the scope of this disclosure that the nickase or dead Cas versions described below could be replaced by a comparable nickase or dead nuclease variant of other programmable nucleases/systems such as OMEGA systems, Zn finger nucleases, TALE nucleases, and meganucleases.

### DNA and RNA Base Editing

In one example embodiment, the method for treating an autoimmune or inflammatory disease and/or disorder comprises administering a DNA or RNA base editing system to either decrease expression of one or more genes from Tables 1A and/or 1B or increase expression of one or more genes from Tables 2A or 2B. In one example embodiment, a catalytically inactive Cas protein is connected or fused to a nucleotide deaminase. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems. Accordingly, in one example embodiment, the base editing system edits the target gene to reduce or eliminate its expression or to increase its expression.

In one example embodiment, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19 (12): 770-788, particularly at FIGS. 1b, 2a-2c, 3a-3f, and Table 1. In some embodiments, the base editing system includes a CBE and/or an ABE. In some embodiments, a polynucleotide of the present disclosure described elsewhere herein can be modified using a base editing system. Rees and Liu. 2018. Nat. Rev. Gent. 19 (12): 770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In one example embodiment, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358:1019-1027, International Patent Publication Nos. WO 2019/005884, WO 2019/005886, and WO 2019/071048, and International Patent Application Nos. PCT/US2018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Example DNA Base Editing Modifications to Increase or Decrease Expression of Target Genes

In one embodiment, a DNA base editing system may be configured to make one or more base edits in a non-coding region of one or more genes in Tables 1A or 1B that result in decreased expression of the one or more genes in Tables 1A or 1B or increase expression of one or more genes in Tables 2A or 2B.

DNA Base-Editing Modifications that Decrease Expression by Targeting Non-Coding Regions

In one embodiment, a DNA base editing system may be configured to make one or more base edits in a non-coding region of one or more genes in Tables 1A or 1B that result in decreased expression of the one or more genes in Tables 1A or 1B. In one embodiment, the one or more base edits introduce mutations in an enhancer region controlling expression of one or more genes in Tables 1A or 1B to prevent or disrupt the binding of transcription factors such that transcription initiation and gene expression are blocked or reduced. In one example embodiment, the one or more base edits introduce mutations in an promoter region controlling expression of one or more genes in Tables 1A or 1B to prevent or disrupt the binding of transcription factors and/or RNA polymerase such that transcription initiation and gene expression are blocked or reduced. In one example embodiment, the base editor is configured to make one or more base edits that introduce a new silence region or modify and strengthen an existing silence region controlling expression of one or more genes in Tables 1A or 1B leading to the recruitment of transcriptional repressors that block or decrease gene expression. In another embodiment, the base editor is configured to make one or more base edits that disrupt one or more insulator sequences controlling expression of one or more genes in Tables 1A or 1B such that nearby silencer elements or repressive chromatin structures are able to decrease gene expression. In one embodiment, a DNA base editing system may be configured to make one or more base edits in a non-coding region of one or more genes in Tables 2A or 2B that result in increased expression of the one or more genes.

DNA Base-Editing Modifications that Increase Expression by Targeting Non-Coding Regions

In one embodiment, a DNA base editing system may be configured to make one or more base edits in a non-coding region of one or more genes in Tables 2A or 2B that result in increased expression of the one or more genes in Tables 2A or 2B. In one embodiment, the base editing system is configured to introduce one or more base edits in one or more enhancer regions controlling expression of one or more genes in Tables 2A or 2B such that binding of transcription factors or other regulatory proteins is increased or strengthened and gene expression increased. In another embodiment, the base editing system is configured to introduce one or more base edits in one or more promoters controlling expression of one or more genes in Tables 2A or 2B such that binding of transcription factors and/or RNA polymerase is increased or strengthened and gene expression is increased. In another embodiment, the base editing system is configured to introduce one or more base edits that disrupt or remove one or more silencer elements that control expression of one or more genes in Tables 2A or 2B, such that binding of transcriptional repressors is prevented or weakened and gene expression is increased. In another embodiment, the base editing system is configured to introduce or strengthen insulator sequences controlling expression of the one or more genes in Tables 2A or 2B thereby reducing the influence of nearby silencer elements or repressive chromatin structures such that gene expression is increased.

DNA Base-Editing Modifications that Decrease Expression by Targeting Coding Regions

In one embodiment, a DNA base editing system may be configured to make one or more base edits in a coding region of one or more genes in Tables 1A or 1B that result in decreased expression of the one or more genes in Tables 1A or 1B. In one embodiment, the one or more base edits result in a frame-shift mutation leading to introduction of a premature stop codon and the production of non-functional truncated gene products, or the triggering of nonsense-mediated mRNA decay (NMD), thereby resulting in reduced expression or gene product activity. In another embodiment, the one or more base edits result in introduction of a premature stop codon within the coding region resulting in production of truncated non-functional proteins or the triggering of NMD. Thereby resulting in reduced gene expression or gene product activity. In another embodiment, the one or more base edits target specific functional domains within the coding region to create mutations that impair the function of the gene product. While this approach may not directly decrease gene expression, it can lead to the production of non-functional proteins, effectively resulting in a loss-of-function effect. In another embodiment, the one or more base edits may introduce mutations in the coding region at exon-intron boundaries or splice sites leading to aberrant splicing, production of non-function proteins or triggering NMD and thereby reducing gene expression or activity of a resulting gene product. In another embodiment, the one or more base edits may target regulatory elements within the coding regions that affect gene expression, such as internal ribosome entry sites (IRES). One or more modifications may be made at these regulatory elements to reduce gene expression. In another embodiment, the one or more base edits may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

DNA Base-Editing Modifications that Increase Expression by Targeting Coding Regions

In one embodiment, the base editing system is configured such that one or more base edits are made in a coding region of the one or more genes of Tables 2A or 2B such that expression of the one or more genes is increased. In one embodiment, the one or more base edits comprise removing or disrupting inhibitor sequences, such as IRESs or upstream open reading frames (uORFs), which can negatively affect expression. In one embodiment, the one or more base edits may comprise introducing specific mutations within the coding region that can potentially improve protein stability, folding, or resistance to degradation. While this does not directly increase gene expression, it can lead to higher protein levels and enhanced function. In one embodiment, the one or more base edits may comprise removal or disruption of a sequence encoding an inhibitory PTM site, removal or disruption of one or more ubiquitination sites, or introduction of PTM sites that stabilize or enhance protein function. In one embodiment, the one or more base edits may comprise mutations or modifications within the coding region that improve catalytic activity, binding affinity, or other functional properties of the protein. This approach does not directly increase gene expression but can result in an overall increase in the functional output of the gene product.

### Example RNA Base Editing Modifications to Increase or Decrease Expression of Target Genes

RNA base editors enable targeted RNA editing without modifying the underlying DNA sequence and may be useful where more temporal control of gene expression is desired.

In one embodiment, a RNA base editing system is used to introduce one or more base edits to one or more RNA molecules transcribed from one or more genes in Tables 1A or 1B. such that expression or activity of the gene product is reduced. In one embodiment, the one or more base edits introduce a frame-shift mutation leading to introduction of a premature stop code resulting in production of a truncated protein or triggering NMD, both which lead to decreased gene expression. In another embodiment, the one or more base edits introduce splice sites or splice regulatory elements that lead to aberrant splicing and production of non-functional proteins or mRNA that is degraded through NMD, thereby decreasing gene expression. In one embodiment, the one or more base edits target specific functional domains of the gene product encoded within the mRNA that impair the function of the gene product. While this approach may not directly decrease translation of the mRNA, it leads to the production of non-functional gene product or gene products with decreased function, effectively achieving a loss-of-function effect. In one embodiment, the one or more base edits modify regulatory elements within the mRNA. Some mRNAs have regulatory elements that can affect gene expression, such as upstream reading frames (uORFs) or IRESs and disrupting these elements may reduce gene expression. In one embodiment, the one or more base edits target translation initiation or elongation by introducing mutations in the mRNA's 5′ untranslated (5′UTR), 3′ untranslated region (3′UTR), or within the coding sequence, affecting translation initiation or elongation and resulting in decreased production of a gene product.

In one embodiment, a RNA base editing system is used to introduce one or more base edits to one or more RNA molecules transcribed from one or more genes in Tables 2A or 2B. such that expression or activity of the gene product is increased. In one embodiment, the one or more base edits are used to change suboptimal codons to more frequently used codes (while maintaining the same amino acid sequence) in the coding region of the mRNA, leading to improved translation efficiency and gene product production. In one embodiment, the one or more base edits remove inhibitor sequences in the mRNA. Some mRNAs contain regulatory elements, such as uORFs and IRESs, that inhibit gene expression. The one or more base edits may be used to disrupt or remove these inhibitor sequences thereby increasing gene product production. In one embodiment, the one or more base edits may be used to modify regulatory elements within the mRNA, such as mRNA stability elements, microRNA binding sites, or RNA binding protein sites and may enhance mRNA stability, translation efficiency, or prevent degradation, leading to increased gene expression. In one embodiment, the one or more base edits may introduce one or more mutations in the 5′ UTR or the 3′UTR that enhance translation initiation or elongation, resulting in increased gene product production. In one embodiment, the one or more base edits may be used to introduce specific point mutations or modifications within the coding region of the mRNA using RNA based editors that can potentially improve the catalytic activity, binding affinity, or other functional properties of the gene product. While this approach may not directly increase mRNA translation, it can result in an overall increase of the functional output of the gene product.

### ARCUS Base Editing

In one example embodiment, a target gene is modified with an ARCUS base editing system. Exemplary methods for using ARCUS can be found in U.S. Pat. No. 10,851,358, US Patent Application Publication No. 2020-0239544, and WIPO Publication No. 2020/206231 which are all incorporated herein by reference.

In certain embodiments, the ARCUS base editing system comprises a nuclease, derived from I-CreI endonuclease (hereinafter, an “ARC Nuclease”) with a recognition sequence for the one or more genes from Table 1A and/or Table 2A and/or transcription factors from Table 1B and/or Table 2B. In certain embodiments, the nuclease is a homing endonuclease or meganuclease as described in the section titled “Meganucleases”. In certain embodiments, the ARC Nuclease is an engineered meganuclease prepared to recognize a target gene or transcription factor, or region of a target gene or transcription factor. In certain embodiments, the ARC Nuclease comprises a single-component protein containing both a site-specific DNA recognition interface and endonuclease activity. The combination of both substrate-recognition and catalytic motifs into a single protein have been shown to allow for both viral and non-viral delivery modalities (see, e.g., Gorsuch et al. (2022). Targeting the hepatitis B cccdna with a sequence-specific arcus nuclease to eliminate hepatitis B virus in vivo. Molecular Therapy, 30 (9), 2909-2922. Doi.org/10.1016/j.ymthe.2022.05.013).

In one example embodiment, the ARC nuclease is configured to decrease the expression of the one or more genes or transcription factors from Tables 1A/1B or increase the expression of the one or more genes or transcription factors from Tables 2A/2B. In an example embodiment, the ARC nuclease scans a region of a target gene for the target site. For example, the ARCUS nuclease looks for a polynucleotide or region within one or more open reading frames of the one or more genes or transcription factors from Table 1A/1B. After binding to the target site, the DNA sequence is cut, created a sticky 4-base 3′ overhang wherein the cut target site is repaired via HDR or NHEJ. As discussed previously, NHEJ can result in insertions, deletions, substitutions, or otherwise a frameshift mutation that can interfere with gene expression. In one example embodiment, the interference with gene expression results in the decreased expression of the one or more genes or transcription factors from Tables 1A/B or increased expression of the one or more genes or transcription factors from Tables 2A/B. HDR or NHEJ methods for repaired joining, and optionally, specific templates that could be utilized, are described in the respective sections titled “HDR Template Based Editing” and “NHEJ-Based Editing”. In certain embodiments, an additional template may prevent off-site insertions or deletions.

### Prime Editors

In one example embodiment, the method for treating an autoimmune or inflammatory disease and/or disorder comprises administering a prime editing system to either decrease expression of one or more genes or transcription factors from Tables 1A and/or 1B or increase the expression of one or more genes or transcription factors from Tables 2A or 2B. Prime editing systems comprise a programable nuclease (e.g. Cas), most often a nickase, linked to a reverse transcriptase domain and a guide molecule (prime editing guide pegRNA), which comprises a target-specific spacer, a primer binding site, and RT template. See e.g., Anzalone et al. 2019. Nature. 576:149-157; and International Patent Application Publication No. WO 2022/150790A2. In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157, particularly at FIGS. 1b, 1c, related discussion, and Supplementary discussion.

Prime editing systems can also be used in tandem such that, the two pegRNAs template the synthesis of complementary DNA flaps on opposing strands of genomic DNA, which replace the endogenous DNA sequence between the PE-induced nick sites. See, e.g., Anzalone A V, Gao X D, Podracky C J, et al. Programmable deletion, replacement, integration and inversion of large DNA sequences with twin prime editing. Nat Biotechnol. 2022; 40 (5): 731-740. Thus, use of two pegRNAs allows for larger insertions or deletions because of the two overlapping 3′ flaps created by the two nicked sites. In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

### Recombinase-Mediated Modifications

Prime editing and twinPE systems can also be further combined with site-specific recombinases, such as integrases, to facilitate even larger insertions, substitutions and deletions. See e.g., WO 2021/138469; Anzalone A V, Gao X D, Podracky C J, et al. Programmable deletion, replacement, integration and inversion of large DNA sequences with twin prime editing. Nat Biotechnol. 2022; 40(5):731-740; Yarnall et al., Nat Biotechnol (2022). Doi.org/10.1038/s41587-022-01527-4, which is incorporated by reference as if expressed in its entirety herein. The prime editing system is used to insert a recombinase recognition site at the desired site of modification and an integrase facilitates the insertion of a donor sequence from a donor template. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regard to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “attR.” The attL and attR sites, using the terminology above, thus consist of BOP′ and POB′, respectively. In some representations herein, the “O” is omitted and attB and attP, for example, are designated as BB′ and PP′, respectively.

In example embodiments, the recombinase of the present disclosure is a serine integrase. In example embodiments, serine integrases specifically recombine when recognizing the two attachment sites specific for the integrase. In example embodiments, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or X is accessory proteins. Thus, in the absence of any accessory factors, integrase is unidirectional. In addition, DNA substrates identified by serine integrases (attP and attB) are relatively short (30-50 bp) and have a minimal length of approximately 34-40 base pairs (bp) (Groth A C et al., Proc. Natl. Acad. Sci. USA 97, 5995-6000 (2000)). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use in the present disclosure include all wildtype sequences as well as pseudo attB and attP sequences.

Recombination sites used in the present methods include those recognized by unidirectional, site-directed recombinases (e.g., integrases). Non-limiting examples of serine integrases and recombination sites applicable to the present disclosure include φC31 integrase, Bxb1, φBT1 integrase, A118, TP901-1, and R4 and the corresponding recombination sites for each (see, e.g., Groth, A. C. and Calos, M. P. (2004) J. Mol. Biol. 335, 667-678; Lei, et al., FEBS Lett. 2018 April; 592(8): 1389-1399; Singh, et al., Attachment Site Selection and Identity in Bxb1 Serine Integrase-Mediated Site-Specific Recombination, PloS Genet. 2013 May; 9 (5): e1003490; and Gupta, et al., Nucleic Acids Res. 2007 May; 35 (10): 3407-3419). Additional serine recombinases and recombination sites may be any of those disclosed in US 2018/0346934A1 and US 2010/0190178. In certain embodiments, a functional domain of the serine integrase is used.

In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

### Example Prime Editing Modifications for Decreasing or Increasing Expression of Target Genes

Prime Editing systems may be used to introduce insertions, deletions, or substitutions (modifications) that control expression of the one or more genes of Tables 1A, 1B, 2A, and 2B. The modifications may be made in a non-coding region that controls expression of the one or more target genes, in a coding region encoding a gene expression product (e.g., a polypeptide), or both. Example modifications are described in further detail below. As a threshold matter, Primer Editing systems are capable of making all 4 base edits (A, T, C, G) and thus can be used to make all of the same DNA base edits described above in the Base Editor section. The following examples focus on additional insertions, deletions and substitutions that may be made using prime editors beyond single base edits using standard prime editors (PE), twinPE, or PE/twinPE in combination with a recombinase, which for purposes of the following example modification sections will be referred to collectively as prime editors.

Prime Editing Modifications that Decrease Expression by Targeting Non-Coding Regions

In one example embodiment, the prime editing system is configured to introduce a deletion, insertion, or mutation in one or more non-coding controlling expression of one or more genes in Tables 1A and 1B such the expression of the one or more genes is reduced. In one embodiment, the one or more modifications remove, modify, or disrupt an enhancer such that binding of transcription factors or other regulatory proteins controlling expression is disrupted thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the prime editing system is configured to introduce a silencer element into the non-coding region leading to the recruitment of transcriptional repressors that block or decrease gene expression. In one embodiment, the prime editor is configured to modify or replace an existing silencer element such that the silencing function of the silencer element is increased relative to an unmodified silencer sequence. In another embodiment, the prime editor is configured to disrupt or replace one or more insulator sequences such that nearby silencer elements or repressive chromatin structures can decrease gene expression.

### Prime Editing Modifications That Increase Expression by Targeting Non-Coding Regions

In another embodiment, the prime editor is configured to introduce one or more enhancer regions controlling expression of one or more genes in Tables 2A or 2B such that binding of transcription factors or other regulatory proteins is increased or strengthened and gene expression is increased. In another embodiment, the primer editor is configured to introduce one or more modifications in one or more promoters, controlling expression of one or more genes in Tables 2A or 2B such that binding of transcription factors or RNA polymerase is increased or strengthened and gene expression is increased. In another embodiment, the prime editor is configured to introduce insertions/deletions/substitutions that disrupt or remove one or more silence elements thereby preventing binding of transcriptional repressors and increasing gene expression. In another embodiment, the prime editor is configured to introduce or strengthen insulator sequences, thereby reducing the influence of nearby silencer elements or repressive chromatin structures such that gene expression is increased.

Prime Editing Modifications that Decrease Expression by Targeting Coding Regions

In one embodiment, the prime editor is configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced gene expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability, and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

Prime Editing Modifications that Increase Expression by Targeting Coding Regions

In one embodiment, the programmable nuclease and donor template are configured such that one or more modifications (e.g., insertions, deletions, substitutions) are made in a coding region of the one or more genes of Tables 2A or 2B such that expression of the one or more genes is increased. In one embodiment, the one or more modifications comprise removing inhibitors sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modification may comprise mutations or modifications within the coding region that improve catalytic activity, binding affinity, or other functional properties of the protein. This approach does not directly increase gene expression but can result in an overall increase in the functional output of the gene product. In another embodiment, prime editing and/or twinPE are used in combination with a recombinase to insert an additional functional copy of the one or more genes from Table 2A or 2B.

### CRISPR Associated Transposase (CAST) Systems

In one example embodiment, the method for treating an autoimmune or inflammatory disease and/or disorder comprises administering a gene editing system to either decrease expression of one or more target genes or target transcription factors from Tables 1A and/or 1B or increase expression of one or more target genes or target transcription factors from Tables 2A and/or 2B, wherein the gene editing system configured to modify the one or more target genes or transcription factors is a CAST system.

CAST systems are able to insert DNA sequences at a target site in a DNA molecule without relying on host cell repair machinery. CAST systems can be Class 1 or Class 2 CAST systems. For example, a Class 1 system is described in Klompe et al. Nature, doi: 10.1038/s41586-019-1323, which is in incorporated herein by reference. An example Class 2 system is described in Strecker et al. Science. 10/1126/science. aax9181 (2019), and PCT/US2019/066835 which are incorporated herein by reference. Suitable hybrid systems have also been described such as those described in Tou et al. bioRxiv 2022.01.07.475005, doi.org/10.1101/2022.01.07.475005, which is incorporated herein by reference.

The CAST system may comprise a Cas linked to a transposase subunit to achieve RNA-guided DNA-transposition, optionally linked to a guide molecule. In some embodiments, the Cas may be catalytically inactive (e.g., Type I, IV, or Type V systems). Transposases suitable for the CAST system may be of any variety generally derived Tn7-like transposons (e.g. non-limiting examples, including TnsA, TnsB, TnsC, or TniQ). Guide molecules can guide the catalytically inactive Cas and a Tn7 or Tn7-like subunit to a target site to direct insertion of a donor at the target site.

CAST systems may require combinatorial transposases for efficient deposition. For example, TnsA is an endonuclease that cleaves the 5′-ends of the transposon and interacts with TnsB, TnsC, and DNA. TnsB is a recombinase capable of cleaving the 3′-end of the transposon. In some CAST systems with these components, the interaction between TnsA and TnsB achieves catalysis. In another instance, TnsC can direct TnsA and TnsB to the insertion site. In another instance, TniQ and DNA can be recognized by TnsC and enable the Cas complex to achieve insertion at the site.

In one example embodiment, the CAST system can be used to insert or replace a sequence into one or more target genes. In example embodiments, the insertion or replacement results in an inactive target gene or less active form of the target gene. In one example embodiment, a CAST system is used to replace all or a portion of an enhancer controlling the target gene expression. In an example embodiment, the enhancer controls the expression of one or more target genes or transcription factors selected from Tables 1A/B or 2A/B.

CAST systems may be used to introduce one or more modifications (insertions, deletions, substitutions) that modify expression of the one or more genes of Tables 1A, 1B, 2A, and 2B, or a combination thereof. The modifications may be made in a non-coding region that controls expression of the one or more target genes, in a coding region encoding a gene expression product (e.g., a polypeptide), or both. Example modifications are described in further detail below.

CAST Modifications that Decrease Expression by Targeting Non-Coding Regions

In one example embodiment, the CAST system is configured to introduce a deletion, insertion, or mutation in one or more non-coding regions controlling expression of one or more genes in Tables 1A and 1B such the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the one or more modifications comprise introduction of a silencer element into the non-coding region leading to the recruitment of transcriptional repressors that block or decrease gene expression. In one embodiment, the one or more modifications comprise modifying or replacing an existing silencer element such that the silencing function of the silencer element is increased relative to an unmodified silencer sequence. In another embodiment, the one or more modifications comprise disrupting or replacing one or more insulator sequences such that nearby silencer elements or repressive chromatin structures can decrease gene expression.

CAST Modifications that Increase Expression by Targeting Non-Coding Regions

In another embodiment, the CAST system is configured to introduce one or more modifications controlling expression of one or more genes in Tables 2A or 2B such that binding of transcription factors or other regulatory proteins is increased or strengthened and gene expression is increased. In one embodiment, the one or more modifications comprise one or more modifications in one or more promoters such that binding of transcription factors or RNA polymerase is increased or strengthened and gene expression is increased. In another embodiment, the one or more modifications comprise introduction of insertions/deletions/substitutions that disrupt or remove one or more silence elements thereby preventing binding of transcriptional repressors and increasing gene expression. In another embodiment, the one or more modifications comprise the introduction of insulator sequences, either new insulator sequences or modified versions of pre-existing insulator sequences, thereby reducing the influence of nearby silencer elements or repressive chromatin structures such that gene expression is increased.

CAST Modifications that Decrease Expression by Targeting Coding Regions

In one embodiment, the CAST systems are configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

CAST Modifications that Increase Expression by Targeting Coding Regions

In one embodiment, the CAST system are configured such that one or more modifications (e.g., insertions, deletions, substitutions) are made in a coding region of the one or more genes of Tables 2A or 2B such that expression of the one or more genes is increased. In one embodiment, the one or more modifications comprise removing inhibitor sequences, such as IRESs or upstream open reading frames (uORFs), that negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal, or disruption of one or more ubiquitination sites, or introduction of PTM sites that stabilize or enhance protein function. In one embodiment, the one or more modifications may comprise mutations or modifications within the coding region that improve catalytic activity, binding affinity, or other functional properties of the protein. This approach does not directly increase gene expression but can result in an overall increase in the functional output of the gene product. In another embodiment, the CAST system is used to insert an additional functional copy of the one or more genes from Table 2A or 2B.

### Non-LTR Retrotransposon Systems

In one example embodiment, the method for treating an autoimmune or inflammatory disease and/or disorder comprises administering a Non-LTR Retrotransposon system to either decrease expression of one or more target genes or target transcription factors from Tables 1A and/or 1B or increase expression of one or more target genes or transcription factors from Tables 2A and/or 2B, or a combination thereof.

The Non-LTR retrotransposon system may comprise one or more components of a retrotransposon, e.g., a non-LTR retrotransposon. Native or wild-type non-LTR retrotransposons encode the protein machinery necessary for their self-mobilization. The non-LTR retrotransposon element comprises a DNA element integrated into a host genome. The DNA element may encode one or two open reading frames (ORFs). For example, the R2 element of Bombyx mori encodes a single ORF containing reverse transcriptase (RT) activity and a restriction enzyme-like (REL) domain. L1 elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. In an example embodiment, the RNA-transposase complex nicks the genome and the 3′ end of the nicked DNA serves as a primer to allow the reverse transcription of the transposon RNA into cDNA. The transposase proteins may then integrate the cDNA into the genome.

Elements of these systems may be engineered to work within the context of the disclosure. For example, a non-LTR retrotransposon polypeptide may be fused to a programmable nuclease. The binding elements that allow a non-LTR retrotransposon polypeptide to bind to the native retrotransposon DNA element, may be engineered into a donor construct to facilitate entry of a donor polynucleotide sequence into a target polypeptide.

In certain embodiments, the protein component of the non-LTR retrotransposon may be connected to or otherwise engineered to form a complex with a programmable nuclease, e.g., a Cas polypeptide. The retrotransposon RNA may be engineered to encode a donor polynucleotide sequence. Thus, in certain example embodiments, the Cas polypeptide, via formation of a CRISPR-Cas complex with a guide sequence, directs the retrotransposon complex (i.e., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence. Example non-LTR retrotransposon systems are disclosed in WO 2021/102042, WO 2022/173830, which are incorporated herein by reference.

Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103 (47): 17602-7; Eickbush T H et al, Integration, Regulation, and Long-Term Stability of R2 Retrotransposons, Microbiol Spectr. 2015 April; 3 (2): MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16 (6): 793-805, which are incorporated by reference herein in their entireties.

Examples of the non-LTR retrotransposon polypeptides also include R2 from Clonorchis sinensis, or Zonotrichia albicollis. Example non-LTR retrotransposon polypeptides and binding components (5′ and 3′ UTRs) that may be used in the context of the disclosure are known in the art, as are codon optimized variants of the non-LTR retrotransposons for expression in eukaryotic cells.

A non-LTR retrotransposon may comprise multiple retrotransposon polypeptides or polynucleotides encoding same. In some embodiments, the retrotransposon polypeptides may form a complex. For example, a non-LTR retrotransposon is a dimer, e.g., comprising two retrotransposon polypeptides forming a dimer. The dimer subunits may be connected or form a tandem fusion. A Cas protein or polypeptide may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a Cas protein or polypeptide.

The retrotransposon polypeptides may be enzymes or variants thereof. In some examples, a retrotransposon polypeptide may be a reverse transcriptase, a nuclease, a nickase, a transposase, nucleic acid polymerase, ligase, or a combination thereof. In one example, a retrotransposon polypeptide is a reverse transcriptase. In another example, a retrotransposon polypeptide is a nuclease. In another example, a retrotransposon polypeptide is nickase. In a particular example, a non-LTR retrotransposon comprises a first retrotransposon polypeptide and a second retrotransposon polypeptide, wherein the second retrotransposon polypeptide comprises nuclease or nickase activity. In certain cases, a retrotransposon polypeptide may comprise an inactive enzyme. For example, a retrotransposon polypeptide may comprise a nuclease domain that is inactivated. Such inactivated domain may serve as a nucleic acid binding domain.

The retrotransposon polypeptides may comprise one or more modifications to, for example, enhance specificity or efficiency of donor polynucleotide recognition, target-primed template recognition (TPTR), and/or reduce or eliminate homing function. The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In some embodiments, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end, the 3′ end, or a location in between.

In some embodiments, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Donor Constructs

The systems may comprise one or more donor constructs comprising one or more donor polynucleotide sequences for insertion into a target polynucleotide. The donor construct comprises one or more binding elements. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for interacting to the retrotransposon polypeptide.

In certain example embodiments, the donor construct comprises a 5′ binding element and a 3′ binding element with a donor polynucleotide sequence located between the 5′ and 3′ prime binding element.

A donor polynucleotide may be any type of polynucleotides, including, but not limited to, a gene, a gene fragment, a non-coding polynucleotide, a regulatory polynucleotide, a synthetic polynucleotide, etc.

A target polynucleotide may comprise a protospacer adjacent motif (PAM) sequence. An example of the PAM sequence is AT.

The donor construct may further comprise one or more processing element. The processing element is an element that may be added to ensure accurate processing and incorporation of the donor polynucleotide sequence by the fusion proteins disclosed herein. Example processing elements include, but are not limited to, LRNA processing elements (e.g. GGCTCGTTGGGAGGTCCCGGGTTGAAATCCCGGACGAGCCCG (SEQ ID NO: 46)), human 28s processing elements (e.g. TAGCCAAATGCCTCGTCATCTAATTAGTGACGCGCATGAATGGATGAACGAGATTCC CACTGTCCCTACCTACTATCCAGCGAAACCACAGCCAAGGGAA (SEQ ID NO: 47)), and natural retrotransposon processing elements such as R2 processing elements from Bombyx mori

(e.g. tagccaaatgcctcgtcatctaattagtgacgcgcatgaatgga
ttaacgagattcccactgtccctatctactatctagcgaaaccacagcca
agggaacgggcttgggagaatcageggggaa (SEQ ID NO: 48)).

The donor construct may comprise one or more homology sequence. A homology sequence is a sequence that shares or complete or partial homology with a target sequence at the site the targeted site of insertion. The homology sequence may be located on the 5′ end, ‘3 end, or on both the 5’ and 3′ end of the donor construct. In certain example embodiments, the homology sequence is only located on the 5′ end of the donor construct. In certain example embodiments, the homology sequence is located only on the 3′ end of the donor construct. In certain example embodiments, the location of the homology sequence may depend on whether the site-specific nuclease is being directed to create a nick or cut 5′ or 3′ of the targeted insertion site, e.g. a 5′ homology sequence on the donor construct may be used when the site specific nuclease creates a nick or cut 5′ of the targeted insertion site and a 3′ homology sequence may be used when the site-specific nuclease is configured to create a nick or cut 3′ of the targeted insertion site. In certain example embodiments, the homology sequence is included on both the 5′ and 3′ ends of the donor construct regardless of whether the site-specific nuclease creates a nick or cut 5′ or 3′ of the targeted insertion site. In certain example embodiments the donor construct may comprise in a 5′ to 3′, a binding element, and the donor sequence. In certain example embodiments the donor construct may comprise in a 5′ to 3′ direction a homology sequence, a binding element, and the donor sequence. In certain example embodiments the donor construct may comprise in a 5′ to 3′ direction a homology sequence, a first binding element, the donor sequence, and second binding element. In certain example embodiments, the donor construct may comprise in a 5′ to 3′ direction a first homology sequence, a first binding element, the donor sequence, and a second homology sequence. In certain example embodiments the donor construct may comprise, in a 5′ to 3′ direction, a first homology sequence, a first binding element, the donor sequence, a second binding element, and a second homology sequence. In certain example embodiments, the donor construct may comprise, in a 5′ to 3′ direction, the donor sequence and a binding element. In certain example embodiments, the donor construct may comprise, in a 5′ to 3′ direction, the donor sequence, a binding element, and a homology sequence. A processing element may be further incorporated 3′ of the donor sequence in any of the above donor construct configurations.

The homology sequence may have at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 bases of homology to the target DNA. In certain example embodiments, the homology sequence may have between 8, 9, 10, 11, 12, 13, 14, 15, 16, 17, 18, 19, 20, 21, 22, 23, 24, or 25 base pairs of homology to the target sequence. In embodiments, with a homology sequence on both the 5′ and 3′ end of the donor construct, the size of the homology may be the same or different on each end. In some examples, the homology sequence comprises from 1 to 30, from 4 to 10, or from 10 to 25 nucleotides. For example, the homology sequence comprises from 4 to 10 nucleotides. For example, the homology sequence comprises from 10 to 25 nucleotides. For example, the homology sequence comprises 1 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

The donor polynucleotides may be inserted to the upstream or downstream of the PAM sequence of a target polynucleotide. For example, the donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

In a strand of a polynucleotide, anything towards the 5′ end of a reference point is “upstream” of that point, and anything towards the 3′ end of a reference point is “downstream” of that point. A location upstream of a PAM sequence refers to a location at the 5′ side of the PAM sequence on the PAM-containing strand of the target sequence. A location downstream of a PAM sequence refers to a location at the 3′ side of the PAM sequence on the PAM-containing strand of the target sequence.

The compositions and systems herein may be used to insert a donor polynucleotide with desired orientation. For example, appropriate homology sequence may be selected to control the orientation of insertion on the 5′ or 3′ strand of the target sequence.

The donor polynucleotide comprises a homology sequence of a region of the target sequence. The homology sequence may share at least 50%, at least 60%, at least 70%, at least 80%, at least 90%, at least 95%, or 100% sequence identity with the region of the target sequence. In an example, the homology sequence shares 100% sequence identity with the region of the target sequence.

In some embodiments, the donor polynucleotide may be inserted to the strand on the target sequence that contains the PAM (e.g., the PAM sequence of the site-specific nuclease such as Cas). In such cases, the donor polynucleotide may comprise a homology sequence of a region on the PAM containing strand of the target sequence. Such region may comprise the PAM sequence. The region may be at the 3′ side of the cleavage site of the site-specific nuclease. In some examples, the homology sequence may comprise from 4 to 10, or from 10 to 25 nucleotides in length. An example of such homology sequence may be of the “h1” region shown in FIG. 36.

In some embodiments, the donor polynucleotide may be inserted to the strand on the target sequence that binds to the guide, e.g., the strand that contains a guide-binding sequence. In such cases, the donor polynucleotide may comprise a homology sequence of a region that comprises at least a portion of the guide-binding sequence. In some cases, the region may comprise the entire guide-binding sequence. Such region may further comprise a sequence at the 3′ side of the guide-binding sequence. For example, the region may comprise from 5 to 15 nucleotides, e.g., 5, 6, 7, 8, 9, 10, 11, 12, 13, 14, 15 nucleotides from the 3′ side of the guide-binding sequence. In some cases, the region may be adjacent to the R-loop of the guide. For example, in the cases where the guide forms a RNA-DNA duplex with the guide-binding sequence, the region comprises a sequence at the 3′ side from the RNA-DNA duplex, e.g., from 5 to from 5 to 15 nucleotides, e.g., 5, 6, 7, 8, 9, 10, 11, 12, 13, 14, 15 nucleotides from the 3′ side from the RNA-DNA duplex. An example of such homology sequence may be of the “h2” region shown in FIG. 36.

In some examples, the homology sequence is of a region on the target sequence at 3′ side of a PAM-containing strand. In certain examples, the homology sequence is of a region on the target sequence 10 nucleotides from 3′ side of a RNA-DNA duplex formed by a guide molecule and a target sequence. For example, the guide molecule forms a RNA-DNA duplex with the target sequence, and the homology sequence is of a region on the target sequence 5 to 15 nucleotides from 3′ side of the RNA-DNA duplex. In some embodiments, the donor polynucleotide is inserted to a region on the target sequence that is 3′ side of a PAM-containing strand. In some cases, the donor polynucleotide is inserted to a region on the target sequence that is 3′ side of a sequence complementary to the guide molecule.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of the corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments, the donor may include, but not be limited to, genes or gene fragments, encoding proteins or RNA transcripts to be expressed, regulatory elements, repair templates, and the like. According to the disclosure, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 5 bases to 50 kb in length, e.g., from 50 to 40 kb, from 100 and 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, from 2800 bases to 3000 bases, from 2900 bases to 3100 bases, from 3000 bases to 3200 bases, from 3100 bases to 3300 bases, from 3200 bases to 3400 bases, from 3300 bases to 3500 bases, from 3400 bases to 3600 bases, from 3500 bases to 3700 bases, from 3600 bases to 3800 bases, from 3700 bases to 3900 bases, from 3800 bases to 4000 bases, from 3900 bases to 4100 bases, from 4000 bases to 4200 bases, from 4100 bases to 4300 bases, from 4200 bases to 4400 bases, from 4300 bases to 4500 bases, from 4400 bases to 4600 bases, from 4500 bases to 4700 bases, from 4600 bases to 4800 bases, from 4700 bases to 4900 bases, or from 4800 bases to 5000 bases in length.

### Example Non-LTR Retrotransposon-Mediated Modifications for Decreasing or Increasing Expression of Target Genes

Non-LTR retrotransposon systems may be used to introduce one or more modifications (insertions, deletions, substitutions) that modify expression of the one or more genes of Tables 1A, 1B, 2A, and 2B, or a combination thereof. The modifications may be made in a non-coding region that controls expression of the one or more target genes, in a coding region encoding a gene expression product (e.g., a polypeptide), or both. Example modifications are described in further detail below.

Non-LTR Modification that Decrease Expression by Targeting Non-Coding Regions

In one example embodiment, the non-LTR retrotransposon system is configured to introduce a deletion, insertion, or mutation in one or more non-coding regions controlling expression of one or more genes in Tables 1A and 1B such the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the one or more modifications comprise introduction of a silencer element into the non-coding region leading to the recruitment of transcriptional repressors that block or decrease gene expression. In one embodiment, the one or more modifications comprise modifying or replacing an existing silencer element such that the silencing function of the silencer element is increased relative to an unmodified silencer sequence. In another embodiment, the one or more modifications comprise disrupting or replacing one or more insulator sequences such that nearby silencer elements or repressive chromatin structures can decrease gene expression.

Non-LTR Modifications that Increase Expression by Targeting Non-Coding Regions

In another embodiment, the non-LTR retrotransposon system is configured to introduce one or more modifications controlling expression of one or more genes in Tables 2A or 2B such that binding of transcription factors or other regulatory proteins is increased or strengthened and gene expression is increased. In one embodiment, the one or more modifications comprise one or more modifications in one or more promoters such that binding of transcription factors or RNA polymerase is increased or strengthened and gene expression is increased. In another embodiment, the one or more modifications comprise introduction of insertions/deletions/substitution that disrupt or remove one or more silence elements thereby preventing binding of transcriptional repressors and increasing gene expression. In another embodiment, the one or more modifications comprise the introduction of insulator sequences, either new insulator sequences or modified versions of pre-existing insulator sequences, thereby reducing the influence of nearby silencer elements or repressive chromatin structures such that gene expression is increased.

Non-LTR Modifications that Decrease Expression by Targeting Coding Regions

In one embodiment, the non-LTR retrotransposon systems are configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability, and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

Non-LTR Modifications that Increase Expression by Targeting Coding Regions

In one embodiment, the non-LTR retrotransposon system is configured such that one or more modifications (e.g., insertions, deletions, substitutions) are made in a coding region of the one or more genes of Tables 2A or 2B such that expression of the one or more genes is increased. In one embodiment, the one or more modifications comprise removing inhibitors sequences, such as IRESs or upstream open reading frames (uORFs), that negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modifications may comprise mutations or modifications within the coding region that improve catalytic activity, binding affinity, or other functional properties of the protein. This approach does not directly increase gene expression but can result in an overall increase in the functional output of the gene product. In another embodiment, the non-LTR retrotransposon system is used to insert an additional functional copy of the one or more genes from Table 2A or 2B.

### Epigenetic Editing

In one example embodiment, the method for treating an autoimmune or inflammatory disease and/or disorder comprises administering an epigenetic modifier or modification system to either decrease expression of one or more genes from Tables 1A and/or 1B or increase expression of one or more genes from Tables 2A or 2B. or a combination thereof. In one example embodiment, the one or more agents is an epigenetic modifier polypeptide comprising a DNA binding domain linked to or otherwise capable of associating with an epigenetic modification domain such that binding of the DNA binding domain at target sequence on genomic DNA (e.g., chromatin) results in one or more epigenetic modifications by the epigenetic modification domain that increases or decreases expression of the one or more polypeptides disclosed herein. As used herein, “linked to or otherwise capable of associating with” refers to a fusion protein or a recruitment domain or an adaptor protein, such as an aptamer (e.g., MS2) or an epitope tag. The recruitment domain or an adaptor protein can be linked to an epigenetic modification domain or the DNA binding domain (e.g., an adaptor for an aptamer). The epigenetic modification domain can be linked to an antibody specific for an epitope tag fused to the DNA binding domain. An aptamer can be linked to a guide sequence.

In example embodiments, the DNA binding domain is a programmable DNA binding protein linked to or otherwise capable of associating with an epigenetic modification domain. Programmable DNA binding proteins for modifying the epigenome include, but are not limited to CRISPR systems, OMEGA systems, transcription activator-like effectors (TALEs), Zn finger proteins and meganucleases (see, e.g., Thakore P I, Black J B, Hilton I B, Gersbach C A. Editing the epigenome: technologies for programmable transcription and epigenetic modulation. Nat Methods. 2016; 13(2):127-137; and described further herein). In example embodiments, the DNA binding domain is a nuclease-deficient RNA-guided DNA endonuclease enzyme or a nuclease-deficient endonuclease enzyme. In example embodiments, a CRISPR system having an inactivated nuclease activity (e.g., dCas) is used as the DNA binding domain.

In example embodiments, the epigenetic modification domain is a functional domain and includes, but is not limited to a histone methyltransferase (HMT) domain, histone demethylase domain, histone acetyltransferase (HAT) domain, histone deacetylation (HDAC) domain, DNA methyltransferase domain, DNA demethylation domain, histone phosphorylation domain (e.g., serine and threonine, or tyrosine), histone ubiquitylation domain, histone sumoylation domain, histone ADP ribosylation domain, histone proline isomerization domain, histone biotinylation domain, histone citrullination domain (see, e.g., Epigenetics, Second Edition, 2015, Edited by C. David Allis; Marie-Laure Caparros; Thomas Jenuwein; Danny Reinberg; Associate Editor Monika Lachlan; Dawson M A, Kouzarides T. Cancer epigenetics: from mechanism to therapy. Cell. 2012; 150(1):12-27; Syding L A, Nickl P, Kasparek P, Sedlacek R. CRISPR/Cas9 Epigenome Editing Potential for Rare Imprinting Diseases: A Review. Cells. 2020; 9(4):993; and Zhang Y. Transcriptional regulation by histone ubiquitination and deubiquitination. Genes Dev. 2003; 17(22): 2733-2740). Example epigenetic modification domains can be obtained from, but are not limited to chromatin modifying enzymes, such as, DNA methyltransferases (e.g., DNMT1, DNMT3a and DNMT3b), TET1, TET2, thymine-DNA glycosylase (TDG), GCN5-related N-acetyltransferases family (GNAT), MYST family proteins (e.g., MOZ and MORF), and CBP/p300 family proteins (e.g., CBP, p300), Class I HDACs (e.g., HDAC 1-3 and HDAC8), Class II HDACs (e.g., HDAC 4-7 and HDAC 9-10), Class III HDACs (e.g., sirtuins), HDAC11, SET domain containing methyltransferases (e.g., SET7/9 (KMT7, NCBI Entrez Gene: 80854), KMT5A (SET8), MMSET, EZH2, and MLL family members), DOTIL, LSD1, Jumonji demethylases (e.g., KDM5A (JARIDIA), KDM5C (JARIDIC), and KDM6A (UTX)), kinases (e.g., Haspin, VRK1, PKCa, PKCB, PIMI, IKKa, Rsk2, PKB/Akt, Aurora B, MSK1/2, JNK1, MLTKa, PRK1, Chk1, DIk/ZIP, PKC8, MST1, AMPK, JAK2, Abl, BMK1, CaMK, S6K1, SIK1), Ubp8, ubiquitin C-terminal hydrolases (UCH), the ubiquitin-specific processing proteases (UBP), and poly(ADP-ribose) polymerase 1 (PARP-1). See, also, U.S. Pat. No. 11,001,829B2 for additional domains.

In example embodiments, histone acetylation is targeted to a target sequence using a CRISPR system (see, e.g., Hilton I B, et al. Epigenome editing by a CRISPR-Cas9-based acetyltransferase activates genes from promoters and enhancers. Nat Biotechnol. 2015). In example embodiments, histone deacetylation is targeted to a target sequence (see, e.g., Cong et al., 2012; and Konermann S, et al. Optical control of mammalian endogenous transcription and epigenetic states. Nature. 2013; 500:472-476). In example embodiments, histone methylation is targeted to a target sequence (see, e.g., Snowden A W, Gregory P D, Case C C, Pabo C O. Gene-specific targeting of H3K9 methylation is sufficient for initiating repression in vivo. Curr Biol. 2002; 12:2159-2166; and Cano-Rodriguez D, Gjaltema R A, Jilderda L J, et al. Writing of H3K4Me3 overcomes epigenetic silencing in a sustained but context-dependent manner. Nat Commun. 2016; 7:12284). In example embodiments, histone demethylation is targeted to a target sequence (see, e.g., Kearns N A, Pham H, Tabak B, et al. Functional annotation of native enhancers with a Cas9-histone demethylase fusion. Nat Methods. 2015; 12 (5): 401-403). In example embodiments, histone phosphorylation is targeted to a target sequence (see, e.g., Li J, Mahata B, Escobar M, et al. Programmable human histone phosphorylation and gene activation using a CRISPR/Cas9-based chromatin kinase. Nat Commun. 2021; 12 (1): 896). In example embodiments, DNA methylation is targeted to a target sequence (see, e.g., Rivenbark A G, et al. Epigenetic reprogramming of cancer cells via targeted DNA methylation. Epigenetics. 2012; 7:350-360; Siddique A N, et al. Targeted methylation and gene silencing of VEGF-A in human cells by using a designed Dnmt3a-Dnmt3L single-chain fusion protein with increased DNA methylation activity. J Mol Biol. 2013; 425:479-491; Bernstein D L, Le Lay J E, Ruano E G, Kaestner K H. TALE-mediated epigenetic suppression of CDKN2A increases replication in human fibroblasts. J Clin Invest. 2015; 125:1998-2006; Liu X S, Wu H, Ji X, et al. Editing DNA Methylation in the Mammalian Genome. Cell. 2016; 167 (1): 233-247.e17; Stepper P, Kungulovski G, Jurkowska R Z, et al. Efficient targeted DNA methylation with chimeric dCas9-Dnmt3a-Dnmt3L methyltransferase. Nucleic Acids Res. 2017; 45 (4): 1703-1713; and Pflueger C., Tan D., Swain T., Nguyen T., Pflueger J., Nefzger C., Polo J. M., Ford E., Lister R. A modular dCas9-SunTag DNMT3A epigenome editing system overcomes pervasive off-target activity of direct fusion dCas9-DNMT3A constructs. Genome Res. 2018; 28:1193-1206). In example embodiments, DNA demethylation is targeted to a target sequence using a CRISPR system (see, e.g., TET1, see Xu et al, Cell Discov. 2016 May 3; 2:16009; Choudhury et al, Oncotarget. 2016 Jul. 19; 7 (29): 46545-46556; and Kang J G, Park J S, Ko J H, Kim Y S. Regulation of gene expression by altered promoter methylation using a CRISPR/Cas9-mediated epigenetic editing system. Sci Rep. 2019; 9 (1): 11960). In example embodiments, DNA demethylation is targeted to a target sequence (see, e.g., TDG, see, Gregory D J, Zhang Y, Kobzik L, Fedulov A V. Specific transcriptional enhancement of inducible nitric oxide synthase by targeted promoter demethylation. Epigenetics. 2013; 8:1205-1212).

Example epigenetic modification domains can be obtained from, but are not limited to transcription activators, such as, VP64 (see, e.g., Ji Q, et al. Engineered zinc-finger transcription factors activate OCT4 (POU5F1), SOX2, KLF4, c-MYC (MYC) and miR302/367. Nucleic Acids Res. 2014; 42:6158-6167; Perez-Pinera P, et al. Synergistic and tunable human gene activation by combinations of synthetic transcription factors. Nat Methods. 2013; 10:239-242; Farzadfard F, Perli S D, Lu T K. Tunable and multifunctional eukaryotic transcription factors based on CRISPR/Cas. ACS Synth Biol. 2013; 2:604-613; Black J B, Adler A F, Wang H G, et al. Targeted Epigenetic Remodeling of Endogenous Loci by CRISPR/Cas9-Based Transcriptional Activators Directly Converts Fibroblasts to Neuronal Cells. Cell Stem Cell. 2016; 19 (3): 406-414; and Maeder M L, Linder S J, Cascio V M, Fu Y, Ho Q H, Joung J K. CRISPR RNA-guided activation of endogenous human genes. Nat Methods. 2013; 10 (10): 977-979), p65 (see, e.g., Liu P Q, et al. Regulation of an endogenous locus using a panel of designed zinc finger proteins targeted to accessible chromatin regions. Activation of vascular endothelial growth factor A. J Biol Chem. 2001; 276:11323-11334; and Konermann S, et al. Genome-scale transcriptional activation by an engineered CRISPR-Cas9 complex. Nature. 2015; 517:583-588), HSF1, and RTA (see, e.g., Chavez A, et al. Highly efficient Cas9-mediated transcriptional programming. Nat Methods. 2015; 12:326-328); KRAB (see, e.g., Beerli R R, Segal D J, Dreier B, Barbas C F., 3^{rd} Toward controlling gene expression at will: specific regulation of the erbB-2/HER-2 promoter by using polydactyl zinc finger proteins constructed from modular building blocks. Proc Natl Acad Sci USA. 1998; 95:14628-14633; Cong L, Zhou R, Kuo Y C, Cunniff M, Zhang F. Comprehensive interrogation of natural TALE DNA-binding modules and transcriptional repressor domains. Nat Commun. 2012; 3:968; Gilbert L A, et al. CRISPR-mediated modular RNA-guided regulation of transcription in eukaryotes. Cell. 2013; 154:442-451; and Yeo N C, Chavez A, Lance-Byrne A, et al. An enhanced CRISPR repressor for targeted mammalian gene regulation. Nat Methods. 2018; 15(8): 611-616).

In example embodiments, the epigenetic modification domain linked to a DNA binding domain recruits an epigenetic modification protein to a target sequence. In example embodiments, a transcriptional activator recruits an epigenetic modification protein to a target sequence. For example, VP64 can recruit DNA demethylation, increased H3K27ac and H3K4me. In example embodiments, a transcriptional repressor protein recruits an epigenetic modification protein to a target sequence. For example, KRAB can recruit increased H3K9me3 (see, e.g., Thakore PI, D'Ippolito A M, Song L, et al. Highly specific epigenome editing by CRISPR-Cas9 repressors for silencing of distal regulatory elements. Nat Methods. 2015; 12(12): 1143-1149). In an example embodiment, methyl-binding proteins linked to a DNA binding domain, such as MBD1, MBD2, MBD3, and MeCP2 recruits an epigenetic modification protein to a target sequence. In an example embodiment, Mi2/NuRD, Sin3A, or Co-REST recruit HDACs to a target sequence.

In example embodiments, the epigenetic modification domain can be a eukaryotic or prokaryotic (e.g., bacteria or Archaea) protein. In example embodiments, the eukaryotic protein can be a mammalian, insect, plant, or yeast protein and is not limited to human proteins (e.g., a yeast, insect, plant chromatin modifying protein, such as yeast HATs, HDACs, methyltransferases, etc.

In one aspect of the disclosure, is provided a fusion protein (epigenetic modification polypeptide) comprising from N-terminus to C-terminus, an epigenetic modification domain, an XTEN linker, and a nuclease-deficient RNA-guided DNA endonuclease enzyme or a nuclease-deficient endonuclease enzyme.

In aspects, the epigenetic modification polypeptide further comprises a transcriptional activator. In aspects, the transcriptional activator is VP64, p65, RTA, or a combination of two or more thereof. In another aspect, the epigenetic modification polypeptide further comprises one or more nuclear localization sequences. In embodiments, the epigenetic modification polypeptide comprises the nuclease-deficient RNA-guided DNA endonuclease enzyme. In embodiments, the fusion protein comprises the nuclease-deficient DNA endonuclease enzyme.

In some embodiments, the functional domains associated with the adaptor protein or the CRISPR enzyme is a transcriptional activation domain comprising VP64, p65, MyoD1, HSF1, RTA or SET7/9. Other references herein to activation (or activator) domains in respect of those associated with the adaptor protein(s) include any known transcriptional activation domain and specifically VP64, p65, MyoD1, HSF1, RTA or SET7/9 (see, e.g., U.S. Pat. No. 11,001,829B2).

In certain embodiments, the present disclosure provides a fusion protein comprising from N-terminus to C-terminus, an RNA-binding sequence, an XTEN linker, and a transcriptional activator. In aspects, the transcriptional activator is VP64, p65, RTA, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present disclosure provides a method of activating a target nucleic acid sequence in a cell, the method comprising: (i) delivering a first polynucleotide encoding a epigenetic modification polypeptide described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

### Transcription Repressors and Transcription Activators

In some embodiments, the method includes modulating gene expression of one or more target genes by modifying DNA binding sites and/or methylation sites for one or more DNA binding or interaction molecules or complexes. In some embodiments, the DNA binding or interaction molecules comprise, transcriptional activators, and/or transcriptional repressors. In some embodiments, the method comprises administering or otherwise introducing an engineered transcriptional activator or repressor to one or more T cells (e.g., Th17 cells) such that expression of a target gene in any one of Tables 1A/1B is decreased or repressed or the expression of a target gene in any one of Tables 2A/2B is increased or initiated.

### Exemplary Engineered Transcriptional Activators

In one example embodiment, a programmable nuclease is used to recruit an activator protein to a target gene (e.g., a gene identified in Table 2A and/or 2B) in order to enhance expression. In one example embodiment, the activator protein is recruited to the enhancer region of a TCI gene or another gene identified in Table 2A and/or 2B. In another example embodiment, the nuclease system is programmed to bind a sequence variant (i.e., a variant of a gene in Table 2A and/or 2B that has decreased expression relative to a wild-type) responsible for decreased expression of a gene identified in Table 2A and/or 2B. In another example embodiment, the programmable nuclease system is recruited to a binding site comprising a mutation that decreases or eliminates binding of a positive regulator of expression of a Tcf7 gene or another gene identified in Table 2A and/or 2B. In another example embodiment, the programmable nuclease system is recruited to an enhancer possessing the variant. For example, if a subject comprises a variant that prevents binding of a transcription factor to an enhancer controlling expression of a Tcf7 gene or another gene identified in Table 2A and/or 2B, a catalytically inactive Cas protein (“dCas”) fused to an activator can be used to recruit that activator protein to the mutated sequence. Accordingly, a guide sequence is designed to direct binding of the dCas-activator fusion such that the activator can interact with the target genomic region and induce expression of a TCI gene or another gene identified in Table 2A and/or 2B. In one example embodiment, the guide is designed to bind within 10, 20, 30, 40, 50, 60, 70, 80, 90, 100 or up to 500 base pairs of the variant nucleotide. In one example embodiment, a CRISPR guide sequence includes the specific variant nucleotide. The Cas protein used may be any of the Cas proteins described elsewhere herein. In one example protein, the Cas protein is a dCas9.

In one embodiment, the programmable nuclease system is a CRISPRa system (see, e.g., US20180057810A1; and Konermann et al. “Genome-scale transcriptional activation by an engineered CRISPR-Cas9 complex” Nature. 2014 Dec. 10. Doi: 10.1038/nature 14136). Numerous genetic variants associated with disease phenotypes are found to be in non-coding region of the genome, and frequently coincide with transcription factor (TF) binding sites and non-coding RNA genes. In one embodiment, a CRISPR system may be used to activate gene transcription, such as any of the genes identified in Tables 2A/2B. A nuclease-dead RNA-guided DNA binding domain, dCas9, tethered to transcriptional activator domains that promote gene activation (e.g., p65) may be used for “CRISPRa” that activates transcription. In one example embodiment, for use of dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, one or more activator domains are recruited. In one example embodiment, the activation domain is linked to the CRISPR enzyme. In another example embodiment, the guide sequence includes aptamer sequences that bind to adaptor proteins fused to an activation domain. In general, the positioning of the one or more activator domains on the inactivated CRISPR enzyme or CRISPR complex is one which allows for correct spatial orientation for the activator domain to affect the target with the attributed functional effect. For example, the transcription activator is placed in a spatial orientation which allows it to affect the transcription of the target. This may include positions other than the N-/C-terminus of the CRISPR enzyme.

Other programmable nucleases can be modified similarly to provide transcriptional activation. In another example embodiment, an OMEGA system is used to recruit an activation domain to a Tcf7 gene or another gene identified in Table 2A and/or 2B. In one example embodiment, the activation domain is linked to the OMEGA protein. In general, the positioning of the one or more activator domains on the OMEGA protein is one which allows for correct spatial orientation for the activator domain to affect the target with the attributed functional effect. Similar to CRISPRa, the recruitment of the activation domain can increase expression of the Tcf7 gene or another gene identified in Table 2A and/or 2B.

In another example embodiment, a zinc finger system is used to recruit an activation domain to a Tcf7 gene or another gene identified in Table 2A and/or 2B. In one example embodiment, the activation domain is linked to the zinc finger system. In general, the positioning of the one or more activator domains on the zinc finger system is one which allows for correct spatial orientation for the activator domain to affect the target with the attributed functional effect. Similar to CRISPRa, the recruitment of the activation domain can increase expression of the Tef7 gene or another gene identified in Table 2A and/or 2B.

In another example embodiment, a TALE system is used to recruit an activation domain to of a Tcf7 gene or another gene identified in Table 2A and/or 2B. In one example embodiment, the activation domain is linked to the TALE system. In general, the positioning of the one or more activator domains on the TALE system is one which allows for correct spatial orientation for the activator domain to affect the target with the attributed functional effect. For example, the transcription activator is placed in a spatial orientation which allows it to affect the transcription of the target. Similar to CRISPRa, the recruitment of the activation domain can increase expression of the Tcf7 gene or another gene identified in Table 2A and/or 2B.

In another example embodiment, a meganuclease system is used to recruit an activation domain to of a Tcf7 gene or another gene identified in Table 2A and/or 2B. In one example embodiment, the activation domain is linked to the meganuclease system. In general, the positioning of the one or more activator domains on the inactivated meganuclease system is one which allows for correct spatial orientation for the activator domain to affect the target with the attributed functional effect. For example, the transcription activator is placed in a spatial orientation which allows it to affect the transcription of the target. Similar to CRISPRa, the recruitment of the activation domain can increase expression of the Tcf7 gene or another gene identified in Table 2A and/or 2B.

### Exemplary Engineered Transcriptional Repressors

CRISPR interference (CRISPRi) is a CRISPR-Cas system variant that allows selective silencing or repression of gene expression by sterically repressing transcription by blocking transcription initiation or elongation (see e.g., Li et al., Cell. 152 (5): 1173-1183 (2013) of a target gene that is targeted by the dCas component of the system. A CRISPRi system comprises a dCas (e.g., dCas9) fused or otherwise linked to a repressor protein or domain (e.g., a KRAB (Krüppel-associated box) domain, mSin3A, NcoR (nuclear receptor co-repressor, Lsd1 (lysine-specific demethylase 1), MeCP2 (methyl-CpG-binding protein 2, HP1 (heterochromatin protein 1), and REST (RE1-silencing transcription factor)). In operation, the dCas portion is directed to a target gene whose expression is to be repressed, by a target gene specific guide RNA. The repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some exemplary embodiments, the CRISPRi system is configured to target one or more regions of a gene in Table 1A and/or 1B. In some exemplary embodiments, the CRISPRi system is configured to target one or more regions of a promoter or other regulatory region a gene in Table 1A and/or 1B.

In certain embodiments, the disclosure provides for a method of blocking transcription of one or more genes or transcription factors from Table 1A or Table 1B, comprising (i) a CRISPR guide targeting a genomic sequence encoding one or more of the genes or transcription factors from Tables 1A/1B or associated with RNA or DNA transcription of the one or more genes or transcription factors in Table 1A/1B and a modified Cas protein that is catalytically inactive, wherein the CRISPR guides optionally comprise a loop capable of binding a transcriptional activator domain or a transcription repressor domain. In certain embodiments, the modified Cas protein is optionally linked to a transcription activator domain or a transcription repressor domain.

In certain embodiments, the modified Cas protein is Cas9, Cpf1, C2cl, or C2c3. The modified Cas protein can be fused to a transcription activator domain or a transcription repressor domain. Alternatively, the CRISPR guides comprise a loop capable of binding a transcriptional activator domain or a transcription repressor domain.

Other programmable nucleases can be modified similarly to provide transcriptional repression. In some embodiments, an OMEGA is used to sterically inhibit transcription of one or more genes in tables 1A/1B. In one example embodiment, the OMEGA protein is fused or otherwise linked to a repressor protein or domain (e.g., a KRAB (Krüppel-associated box) domain, mSin3A, NcoR (nuclear receptor co-repressor, Lsd1 (lysine-specific demethylase 1), MeCP2 (methyl-CpG-binding protein 2, HP1 (heterochromatin protein 1), and REST (REI-silencing transcription factor)). In operation, the OMEGA protein directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some embodiments, a TALEN system is used to sterically inhibit transcription of one or more genes in tables 1A/1B. In one example embodiment, the TALEN is fused or otherwise linked to a repressor protein or domain (e.g., a KRAB (Krüppel-associated box) domain, mSin3A, NcoR (nuclear receptor co-repressor, Lsd1 (lysine-specific demethylase 1), MeCP2 (methyl-CpG-binding protein 2, HP1 (heterochromatin protein 1), and REST (REI-silencing transcription factor)). In operation, the TALEN directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some embodiments, a zinc finger system is used to sterically inhibit transcription of one or more genes in tables 1A/1B. In one example embodiment, the ZFN is fused or otherwise linked to a repressor protein or domain (e.g., a KRAB (Krüppel-associated box) domain, mSin3A, NcoR (nuclear receptor co-repressor, Lsd1 (lysine-specific demethylase 1), MeCP2 (methyl-CpG-binding protein 2, HP1 (heterochromatin protein 1), and REST (REI-silencing transcription factor)). In operation, the ZFN directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

In some embodiments, a Meganuclease system is used to sterically inhibit transcription of one or more genes in tables 1A/1B. In one example embodiment, the Meganuclease is fused or otherwise linked to a repressor protein or domain (e.g., a KRAB (Krüppel-associated box) domain, mSin3A, NcoR (nuclear receptor co-repressor, Lsd1 (lysine-specific demethylase 1), MeCP2 (methyl-CpG-binding protein 2, HP1 (heterochromatin protein 1), and REST (REI-silencing transcription factor)). In operation, the Meganuclease directs the system to a target gene whose expression is to be repressed and the repressor domain then represses transcription by blocking initiation and/or elongation. In some embodiments, repression of gene transcription is greater than 90%, 91%, 92%, 93%, 94%, 95%, 96%, 97%, 98%, 99%, up to and including 100%.

### CTCF Editing

In one example embodiment, the method for treating an autoimmune or inflammatory disease and/or disorder comprises administering a CTCF-based gene modification system. In one example embodiment, the CTCF-based gene modification system configured to modify the one or more target genes is a genetic modification system capable of editing one or more CTCF motifs in the cell genome. In an example embodiment, editing one or more CTCF motifs modifies one or more chromosome loops such that expression of one or more target genes and transcription factors from Tables 1A/1B is decreased and/or 2A/2B is increased.

As used herein, the terms “CTCF” refers to the architectural protein CCCTC-binding factor. As used herein, the term “CTCF binding motif” refers a consensus DNA sequence (typically, 5′-CCACNAGGTGGCAG-′3 (SEQ ID NO: 49)) to which CTCF and two other proteins of the chromosomal loop forming complex, SMC3 and RAD21 bind. A loop domain is defined between two convergent pairs of CTCF-binding motifs. A chromosome loop refers to the genomic sequences in close proximity to each other (in any degree) that lie on the same chromosome (configured in cis), and also includes the architectural machinery involved in maintaining them (e.g., proteins, non-coding RNAs, DNA regulatory elements, etc.). The CTCF or the chromosome loop is maintained by architectural or DNA machinery associated with the CTCF or the chromosome loop and can facilitate interactions between remote enhancers and their target gene promoters to modulate transcription. (See, e.g., Guo et al. (2018).

Accordingly, gene expression can be modified by modification of where chromosomal loops are formed. Chromosomal loops can be removed, e.g., by removing or disrupting one or both of the CTCF motifs, or introduced by creation of a convergent pair of CTCF motifs, either by adding one CTCF motif that is in convergent orientation to a pre-existing CTCF motif or by insertion of a convergent CTCF pair. Any programmable nuclease (CRISPR-Cas, OMEGA systems, Zn Finger Nucleases, TALENs, meganucleases, base editors, prime editors, CAST systems, non-LTR retrotransposons) may be configured to insert, disrupt, or remove CTCF motifs. Three-dimensional chromosome mapping techniques such as Hi-C may be used to determine where loop boundaries are found and accordingly which CTCF motifs should be targeted to remove a loop and silence gene expression or introduce a loop and promote increased gene expression. See, e.g., Rao et al. “A 3D Map of the Human Genome at Kilobase Resolution Reveals Principles of Chromatin Looping” Cell, 159 (7): 1665-1680 (2014).

In one embodiment, the system is configured to make a substitution, deletion or insertion of a CTCF motif or chromosome loop such that an enhancer and/or target gene promoter that regulates transcription of one or more target genes from Tables 1A or transcription factors from Table 1B does not initiate transcription of the target gene, thereby reducing the expression of the one or more target genes or transcription factors.

In certain embodiments, the substitution, deletion, or insertion of a CTCF motif or chromosome loop initiates transcription of one or more target genes or transcription factors such that one or more genes or transcription factors from Tables 2A/2B are increased.

### Example Inflammatory Diseases

Examples of inflammatory diseases or disorders that may treated using the methods described herein include, but are not limited to, asthma, allergy, allergic rhinitis, allergic airway inflammation, atopic dermatitis (AD), chronic obstructive pulmonary disease (COPD), inflammatory bowel disease (IBD), Irritable bowel syndrome (IBS), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR)

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis. All gene name symbols refer to the gene as commonly known in the art. The examples described herein that refer to the human gene names are to be understood to also encompasses genes in any other organism, for example mouse genes or any other gene used in a model of disease (e.g., homologous, orthologous genes). Any reference to the gene symbol is a reference made to the entire gene or variants of the gene. Any reference to the gene symbol is also a reference made to the gene product (e.g., protein). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). A signature as described herein may encompass any of the genes described herein. The example DNA, RNA, and protein sequences can be used for the design of gene editing agents, antisense oligonucleotides (ASO), antibodies, small molecules, and RNAi.

In one embodiment, the inflammatory disease or disorder may be an autoimmune disease. Examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopeniarpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anemia; polyarteritis nodosa; polymyositis; primary biliary cirrhosis; primary myxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

The MS may be any clinical variety or origin, and not limited to mammals. Non-limiting examples may include Experimental autoimmune encephalomyelitis (EAE), clinically isolated syndrome (CIS), Relapsing-remitting MS (RRMS), Secondary progressive MS (SPMS), or Primary progressive MS (PPMS).

### Administration of Pharmaceutical Compositions

A “pharmaceutical composition” refers to a composition that usually contains an excipient, such as a pharmaceutically acceptable carrier that is conventional in the art and that is suitable for administration to cells or to a subject.

The pharmaceutical composition according to the present disclosure can, in one alternative, include a prodrug. When a pharmaceutical composition according to the present disclosure includes a prodrug, prodrugs and active metabolites of a compound may be identified using routine techniques known in the art. (See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992)).

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally, or topically. In some embodiments, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the disclosure is intended to be used as an infusion. The skilled person will understand that compositions which are to be administered orally or topically will usually not comprise cells, although it may be envisioned for oral compositions to also comprise cells, for example when gastro-intestinal tract indications are treated. Each of the cells or active components (e.g., immunomodulants) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatin, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregelatinized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

In some embodiments, the pharmaceutical compositions may comprise a therapeutically effective amount of the specified immune cells and/or other active components (e.g., immunomodulants). The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15^{th} ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2): 1-60 (2000), Charman W N “Lipids, lipophilic drugs, and oral drug delivery-some emerging concepts.” J Pharm Sci. 89(8): 967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the disclosure are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the disclosure may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. Effective doses may be extrapolated from dose-response curves derived from in vitro or animal model test systems. Ultimately the attending physician will decide on the appropriate duration of therapy using compositions of the present disclosure. Dosage will also vary according to the age, weight and response of the individual patient.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

### Modified Immune Cells and Methods of Making

In another aspect, embodiments disclosed herein are directed to immune cells genetically modified ex vivo to increase or decrease expression of key immune cell regulators, genes, and transcription factors that maintain the immune cell in a homeostatic cell state and/or prevent or impede the cell's ability to transition to a pathogenic cell state, and used in adoptive cell-based therapies. In another aspect, the genetically modified immune cells are modified ex vivo with one or more modifications that help maintain the Th17 cells in a homeostatic or non-pathogenic cell state and/or decrease the likelihood that the Th17 cells will shift to an inflammatory or pathogenic cell state, particularly when deployed as a cell-based therapeutic. The ex vivo modifications may be made to an already differentiated Th17 cell or to a iPSC cell, a hematopoietic stem cell, a T cell progenitor, or a naïve CD4 cell, that can be differentiated into a Th17 cell either ex vivo or in vivo when delivered as a cell-based therapeutic. The engineered immune cell may comprise one or more modifications that decrease expression of one or more genes selected from Table 1A and/or 1B, increase expression of one or more genes selected from Table 2A and/or 2B, or a combination thereof.

### Preparation of Modified Cells for Use in Adoptive Cell Therapy

Methods to prepare cells for use in adoptive cell therapy (ACT) are known in the art. The isolation of T cells, or progenitor cells capable of being differentiated into a T cell, may be prepared as from the peripheral blood (see, e.g., Landwehr-Kenzel et al., (2018) Kidney Int. 93, 1452-1464), umbilical cord (see, e.g., Brunstein, C. G. et al. (2016). Blood 127, 1044-1051), thymus (see, e.g., Romano et al. (2021) Eur. J. Immunol. 51, 2086-2092), or via leukapheresis (see e.g., Peters et al. (2008) PloS One 3, e3161), or from any other source known in the art. In certain embodiments, cells may be prepared from a non-human source. Cell sorting or isolation other may be achieved by a closed system such as magnetically activated cell sorting (MACS) (Id.), fluorescently activated cell sorting (FACS) technologies (see, e.g., Au et al. (2019) Cytotherapy 21, e8), or any other method known in the art. In certain embodiments, cells may be prepared using an open system. In certain embodiments, sorted cells may yield a high purity of isolated cells. In certain embodiments, the isolation strategy could employ a positive selection, negative selection, or using a combination of positive and negative selection techniques for enrichment. The isolated cells may be autologous or allogenic.

The isolated cell may be a hematopoietic stem cell, an iPSC, or naïve CD4+ cell that is then differentiated into a Th17 cell. Th17 cell differentiation can be induced, for example, in vitro by the cytokines transforming growth factor b1 (TGF-β1) and interleukin-6 (IL-6). Ke et al. Journal of Investigative Dermatology Auto-Immunity, 136 (5), Supplement 1, S7, May 2016. Other studies have shown that IL-1β and IL-23 cytokines can promote and stabilize previously differentiated Th17 cells. Ke et al. Additionally, the transcription factor retinoic acid receptor-related orphan receptor-gamma-t (RORγt) plays a crucial role in the differentiation and function of Th17 cells. Huang et al. Medical Science Monitor, DOI: 10.12659/MSM.898824 (2017).

In certain embodiments, the prepared cells may be cryopreserved in order to effectuate preservation and eventual transfer to the subject. Prepared cell preservation methods may include any method known in the art. State of the art methods include those such as found in Amini et al. (2023). Clinical adoptive regulatory T cell therapy: State of the art, challenges, and prospective. Frontiers in Cell and Developmental Biology, 10. Doi.org/10.3389/fcell.2022.1081644.

In certain embodiments, the prepared cells prepared for transfer to the subject may also be subject to release criteria to prevent the risk of microbiological contaminations or accumulation of aberrations. In certain embodiments, the release criteria may include identity, purity or functionality controls. In certain embodiments, the release criteria may also include other safety parameters such as sterility, endotoxin content, absence of mycoplasma and removal of any expansion beads used. In further embodiments a genetic or otherwise biological property of the cell may be checked and controlled as release criteria.

In certain embodiments, other strategies known in the art may be used to optimize immune cell functionality before transfer to the subject. Strategies such as expanding donor-specific Tregs out of a lymphocyte reaction (MLR) (see, e.g., Guinan et al. (2016). Am. J. Transpl. 16, 2187-2195) or co-culturing with allogenic cells can be employed to obtain alloantigen-specific cells (see, e.g., Yamazaki et al. (2006). Proc. Natl. Acad. Sci. U.S.A 103, 2758-2763; Levitsky et al. (2011). Transplantation 91, 199-206) have been successfully performed in clinical applications. In example embodiments described elsewhere herein, a genetic modifying system (e.g., a CRISPR system) is used to generate antigen-specific immune cells or enhance the expression of the genes to which would otherwise be susceptible to interfering therapy (e.g. immunosuppressive pharmaceuticals).

In certain embodiments, the prepared immune cell may be genetically modified to express a T cell receptor (TCR) or the TCR expressed by the immune cell may be genetically modified. Various strategies may for example be employed to genetically modify T cells by altering the specificity of the TCR for example by introducing new TCR a and B chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells or natural killer cells (NK), specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). In certain embodiments, tumor infiltrating lymphocytes (TILS) may be prepared for ACT using various methods and strategies known in the art. See, e.g., Jin, J., Sabatino, M., Somerville, R., Wilson, J. R., Dudley, M. E., Stroncek, D. F., &amp; Rosenberg, S. A. (2012). Simplified method of the growth of human tumor infiltrating lymphocytes in gas-permeable flasks to numbers needed for patient treatment. Journal of Immunotherapy, 35(3), 283-292. doi.org/10.1097/cji.0b013e31824e801f

In general, CARs and CAARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand. In some embodiments, such as in the case of CAARs, the binding domain is an autoantigen or antigen targeting an autoantibody.

The antigen-binding domain of a CAR or CAAR is generally separated from the transmembrane domain by a hinge or spacer. The spacer is also not particularly limited, and it is designed to provide the CAR or CAAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR or CAAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, optionally between 2 and 10 amino acids in length may form the linkage between the transmembrane domain and the cytoplasmic signaling domain of the CAR. A glycine-serine doublet provides a particularly suitable linker.

### EXAMPLE MODIFICATIONS

A number of different strategies may be used to arrive at the modified immune cells of the present disclosure. The following sections provide example modifications that may be used to decrease expression of one or more genes from Tables 1A/1B, increase expression of one or more genes from Tables 2A/2B, or a combination thereof. In one embodiment, the modifications are made prior to differentiation and/or expansion of the immune cells. In one embodiment, the modifications made after differentiation and/or expansion of the immune cells.

### Exemplary Modifications to Decrease Gene Expression

In one embodiment, the cell is engineered to decrease expression of one or more genes in Tables 1A or 1B. The cell may be engineered to decrease expression of the one or more target genes by modifying non-coding regions controlling expression of the one or more genes, or coding regions of the one or more genes such that expression is decreased or the activity or function of a resulting gene product is decreased. The modifications may be made at a genomic level or may comprise the delivery of stable expression constructs the express genetic modification systems that decrease gene expression.

### Gene Knock-Out

In one embodiment, one or more non-coding regions controlling expression of the one or more genes may be modified using vector-based homologous recombination-mediated modification of the non-coding region controlling expression of one of the one or more genes from Tables 1A and 1B, or to a coding region of the one or more genes from Tables 1A and 1B. Example vector-based methods for modifying one or more non-coding regions are discussed above under the section entitled “Vector-Based Gene Knock-out.”

### Genetic Modification of Non-Coding Regions Controlling Expression of Target Genes

In one example embodiment, the cell is engineered to comprise decreased expression of one or more genes in Tables 1A or 1B by modifying one or more non-coding region that control the expression of the one or more genes such that gene expression is decreased. Genetic modifications to the one or more non-coding regions may be made using vector based gene therapy modifications, NHEJ-mediated or HDR-mediated modification using programmable nucleases, base editing, prime editing (with or without use of a recombinase), CAST-based editing, and Non-LTR retrotransposon systems.

NHEJ-Mediated Modifications that Decrease Expression by Targeting a Non-Coding Region

In one embodiment, the one or more programmable nucleases may be configured to introduce one or more insertions or deletions repair into one or more non-coding regions controlling expression of the one or more genes in Tables 1A or 1B via NHEJ-mediated cell repair such that expression of the one or more target genes is reduced. In one embodiment, the insertions or deletions may disrupt the binding site in an enhancer of one or more proteins, such as a transcription factor or other regulatory proteins, needed to initiation transcription of the one or more target genes. In one embodiment, the one or more insertions or deletions may disrupt one or more promoters controlling expression of the one or more genes such that binding of transcription factors and/or RNA polymerase binding is blocked or reduced. In one embodiment, the one or more insertions or deletions may disrupt one or more insulator regions such that silencer regions or repressive chromatin structures controlling expression of the one or more target genes are no longer muted or blocked by the insulator region and can decrease gene expression.

NHEJ-Mediated Modification that Decrease Expression by Targeting a Coding Region

In one embodiment, the programmable nuclease is used to introduce one or more insertions or deletions to coding sequence of one or more genes in Tables 1A or 1B, such that one or more indels or insertions reduce expression or activity of one or more genes in Tables 1A or 1B. For example, the insertion or deletion may cause a frame shift in the coding sequence such that expression is reduced or such that the resulting gene product is non-functional or exhibits reduced activity relative to an unmodified gene. In one embodiment, the insertion(s) or deletion(s) may alter a splice site such that transcription or translation is reduced or such a that resulting gene product is non-functional or exhibits reduced activity relative to an unmodified gene. The insertion or deletion may introduce a premature stop codon such that expression is reduced. The insertion or deletion may alter a post-translational modification site such that the activity of the resulting gene product is reduced.

HDR-Based Modifications that Decrease Expression by Targeting Non-Coding Regions

In one example embodiment, the one or more programmable nucleases str configured to introduce a deletion, insertion, or mutation in one or more enhancer regions such that binding of transcription factors or other regulatory proteins controlling expression of the one or more genes in Tables 1A or 1B is disrupted thereby reducing transcription initiation and gene expression. In one example embodiment, the programmable nuclease is configured to introduce a deletion, insertion, or mutation in one or more promoters controlling expression of one or more genes in Tables 1A or 1B to prevent or disrupt the binding of transcription factors and RNA polymerase such that transcription initiation and gene expression are blocked or reduced. In one example embodiment, the donor template is configured to introduce a silencer element into a non-coding region controlling expression of one or more genes in Tables 1A or 1B leading to the recruitment of transcriptional repressors that block or decrease gene expression. In one embodiment, the donor template is configured to modify or replace an existing silencer element controlling expression of one or more genes in Tables 1A or 1B such that the silencing function of the silencer element is increased relative to an unmodified silencer sequence. In another embodiment, the donor template is configured to disrupt or replace one or more insulator sequences controlling expression of one or more genes in Tables 1A or 1B such that nearby silencer element or repressive chromatin structures decrease gene expression.

HDR-Based Modifications that Decrease Expression by Targeting Coding Regions

In one embodiment, the programmable nuclease and donor template are configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of non-functional, truncated proteins or the triggering of nonsense-mediated mRNA decay (NMD) thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

DNA Base-Editing Modifications that Decrease Expression by Targeting Non-Coding Regions

In one embodiment, a DNA base editing system may be configured to make one or more base edits in a non-coding region of one or more genes in Tables 1A or 1B that result in decreased expression of the one or more genes in Tables 1A or 1B. In one embodiment, the one or more base edits introduce mutations in an enhancer region controlling expression of one or more genes in Tables 1A or 1B to prevent or disrupt the binding of transcription factors such that transcription initiation and gene expression are blocked or reduced. In one example embodiment, the one or more base edits introduce mutations in a promoter region controlling expression of one or more genes in Tables 1A or 1B to prevent or disrupt the binding of transcription factors and/or RNA polymerase such that transcription initiation and gene expression are blocked or reduced. In one example embodiment, the base editor configured to one or more base edits that introduce a new silence region or modify and strengthen an existing silence region controlling expression of one or more genes in Tables 1A or 1B leading to the recruitment of transcriptional repressors that block or decrease gene expression. In another embodiment, the base editor is configured to make one or more base edits that disrupt one or more insulator sequences controlling expression of one or more genes in Tables 1A or 1B such that nearby silencer elements or repressive chromatin structures are able to decrease gene expression.

DNA Base-Editing Modifications that Decrease Expression by Targeting Coding Regions

In one embodiment, a DNA base editing system may be configured to make one or more base edits in a coding region of one or more genes in Tables 1A or 1B that result in decreased expression of the one or more genes in Tables 1A or 1B. In one embodiment, the one or more base edits result in a frame-shift mutation leading to introduction of a premature stop codon and the production of non-functional truncated gene products, or the triggering of nonsense-mediated mRNA decay (NMD), thereby resulting in reduced expression or gene product activity. In another embodiment, the one or more base edits result in introduction of a premature stop codon within the coding region resulting in production of truncated non-functional proteins or the triggering of NMD. Thereby resulting in reduced gene expression or gene product activity. In another embodiment, the one or more base edits target specific functional domains within the coding region to create mutations that impair the function of the gene product. While this approach may not directly decrease gene expression, it can lead to the production of non-functional proteins, effectively resulting in a loss-of-function effect. In another embodiment, the one or more base edits may introduce mutations in the coding region at exon-intron boundaries or splice sites leading to aberrant splicing, production of non-function proteins or triggering NMD and thereby reducing gene expression or activity of a resulting gene product. In another embodiment, the one or more base edits may target regulatory elements within the coding regions that affect gene expression, such as internal ribosome entry sites (IRES). One or more modifications may be made at these regulatory elements to reduce gene expression. In another embodiment, the one or more base edits may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

Prime Editing Modifications that Decrease Expression by Targeting Non-Coding Regions

In one example embodiment, the prime editing system is configured to introduce a deletion, insertion, or mutation in one or more non-coding controlling expression of one or more genes in Tables 1A and 1B such the expression of the one or more genes is reduced. In one embodiment, the one or more modifications remove, modify, or disrupt an enhancer such that binding of transcription factors or other regulatory proteins controlling expression is disrupted thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the prime editing system is configured to introduce a silencer element into the non-coding region leading to the recruitment of transcriptional repressors that block or decrease gene expression. In one embodiment, the prime editor is configured to modify or replace an existing silencer element such that the silencing function of the silencer element is increased relative to an unmodified silencer sequence. In another embodiment, the prime editor is configured to disrupt or replace one or more insulator sequences such that nearby silencer elements or repressive chromatin structures can decrease gene expression. As described above, these modifications may be made using a prime editing system alone or a prime editing system in combination with a recombinase, with that later being useful for larger insertion, substitutions, and deletions.

Prime Editing Modifications that Decrease Expression by Targeting Coding Regions

In one embodiment, the prime editor are configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability, and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

CAST Modifications that Decrease Expression by Targeting Non-Coding Regions

In one example embodiment, the CAST system is configured to introduce a deletion, insertion, or mutation in one or more non-coding regions controlling expression of one or more genes in Tables 1A and 1B such the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the one or more modifications comprise introduction of a silencer element into the non-coding region leading to the recruitment of transcriptional repressors that block or decrease gene expression. In one embodiment, the one or more modifications comprise modifying or replacing an existing silencer element such that the silencing function of the silencer element is increased relative to an unmodified silencer sequence. In another embodiment, the one or more modifications comprise disrupting or replacing one or more insulator sequences such that nearby silencer elements or repressive chromatin structures can decrease gene expression.

CAST Modifications that Decrease Expression by Targeting Coding Regions

In one embodiment, the CAST systems are configured such that one or more modifications (e.g., insertions, deletions, substitutions) are made in a coding region of the one or more genes of Tables 1A and/or 1B such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

Non-LTR Modification that Decrease Expression by Targeting Non-Coding Regions

In one embodiment, the non-LTR retrotransposon system is configured to introduce a deletion, insertion, or mutation in one or more non-coding regions controlling expression of one or more genes in Tables 1A and 1B such the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the one or more modifications comprise introduction of a silencer element into the non-coding region leading to the recruitment of transcriptional repressors that block or decrease gene expression. In one embodiment, the one or more modifications comprise modifying or replacing an existing silencer element such that the silencing function of the silencer element is increased relative to an unmodified silencer sequence. In another embodiment, the one or more modifications comprise disrupting or replacing one or more insulator sequences such that nearby silencer elements or repressive chromatin structures can decrease gene expression.

Non-LTR Modifications that Decrease Expression by Targeting Coding Regions

In one embodiment, the non-LTR retrotransposon systems are configured such that one or more modifications (e.g., insertions, deletions, substitutions) are made in a coding region of the one or more genes of Tables 1A or 1B such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-function proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modification may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

### CTCF Editing

Gene expression can be modulated by modification of where chromosomal loops are formed. Chromosomal loops can be removed, e.g., by removing or disrupting one or both of a pair convergently oriented CTCF motifs, or introduced by creation of a convergent pair of CTCF motifs, either by adding one CTCF motif that is in convergent orientation to a pre-existing CTCF motif or by insertion of a convergent CTCF pair. Any programmable nuclease (CRISPR-Cas, OMEGA systems, Zn Finger Nucleases, TALENs, meganucleases, base editors, prime editors, CAST systems, non-LTR retrotransposons) may be configured to insert, disrupt, or remove CTCF motifs. Three-dimensional chromosome mapping techniques such as Hi-C may be used to determine where loop boundaries are found and accordingly which CTCF motifs should be targeted to remove a loop and silence gene expression or introduce a loop and promote increased gene expression. See e.g., Rao et al. “A 3D Map of the Human Genome at Kilobase Resolution Reveals Principles of Chromatin Looping” Cell, 159(7): 1665-1680 (2014).

In one embodiment, the system is configured to make a substitution, deletion or insertion of a CTCF motif or chromosome loop such that an enhancer and/or target gene promoter that regulates transcription of one or more target genes from Tables 1A or transcription factors from Table 1B does not initiate transcription of the target gene, thereby reducing the expression of the one or more target genes or transcription factors.

### Exemplary Modifications to Increase Gene Expression

The modified cells disclosed herein cell may be engineered to increase expression of one or more genes in Tables 2A or 2B. The cell may be engineered to increase expression of the one or more target genes by modifying non-coding regions controlling expression of the one or more genes, or coding regions of the one or more genes such that expression is increased, or the activity or function of a resulting gene product is increased. The modifications may be made at a genomic level or may comprise the delivery of stable expression constructs the express genetic modification systems that increase gene expression.

### Gene Knock-In

In one example embodiment, the cell is engineered to comprises one or more genes or transcription factors from Table 2A or Table 2B that have been knocked-in to increase expression of the one or more genes relative to a wild-type or unmodified cell. In one example embodiment, knock-in of the one or more genes may be achieved by delivery of vector capable of heterologous recombination with the one or more target genes. Example vectors suitable for knock-in of the one or more genes of Tables 2A/2B include those discussed above in the section titled “Vector Based Gene Knock-out.”

### Genetic Modification of Enhancer Regions to Increase Expression of Target Genes

In one example embodiment, the modified cells disclosed herein may be engineered to comprise increased expression of one or more genes in Tables 2A or 2B by modifying one or more non-coding region that control the expression of the one or more genes such that gene expression is decreased. Genetic modifications to the one or more non-coding regions may be made using vector based gene therapy modifications, NHEJ-mediated or HDR-mediated modification using programmable nucleases, base editing, prime editing (with or without use of a recombinase), CAST-based editing, and Non-LTR retrotransposon systems.

NHEJ-Mediated Modifications that Increase Expression by Targeting a Non-Coding Region

In one embodiment, the one or more programmable nucleases may be configured to introduce one or more insertions or deletions in a non-coding region controlling expression of one or more genes in Tables 2A or 2B such that expression of the one or more target genes is increased. In one embodiment, the one or more insertions or deletions modify one or more enhancer regions controlling expression of one or more genes in Tables 2A or 2B such that binding of transcription factors or other regulatory proteins is increased or strengthened and gene expression is increased. In one embodiment, the one or more insertions or deletions modify one or more promoter regions controlling expression of one or more genes in Tables 2A or 2B such that binding of transcription factors and/or RNA polymerase is increased or strengthened and gene expression is increased. In one embodiment, the one or more insertions or deletions disrupt one or more silencer regions controlling expression of one or more genes in Tables 2A or 2B, such that binding of transcriptional repressor is blocked or reduced and gene expression is increased.

NHEJ-Mediated Modifications that Increase Expression by Targeting a Coding Region

In one embodiment, the programmable nuclease is used to introduce one or more insertions or deletions to coding sequence of one or more genes in Tables 1A or 1B, such that one or more indels or insertions increase expression or activity of one or more genes in Tables 1A or 1B. For example, the insertion or deletion may cause a frame shift in the coding sequence such that expression is increased or such that the resulting gene product is functional or exhibits increased activity relative to an unmodified gene. In one embodiment, the insertion(s) or deletion(s) may alter a splice site such that transcription or translation is increased or such a that resulting gene product exhibits increased activity relative to an unmodified gene.

HDR-Based Modifications that Increase Expression by Targeting Non-Coding Regions

In one embodiment, the programable nuclease and donor template may be configured to make one or more modifications (insertions, substitutions, deletions) in a non-coding region of one or more genes in Tables 2A or 2B that result in increased expression of the one or more genes in Tables 2A or 2B. In one embodiment, the one or more modifications modify one or more enhancer regions controlling expression of one or more genes in Tables 2A or 2B such that binding of transcription factors or other regulatory proteins is increased or strengthened and gene expression increased. In another embodiment, the one or more modifications modify one or more promoters controlling expression of one or more genes in Tables 2A or 2B such that binding of transcription factors and/or RNA polymerase is increased or strengthened and gene expression is increased. In another embodiment, the one or modifications disrupt or remove one or more silencer elements that control expression of one or more genes in Tables 2A or 2B, such that binding of transcriptional repressors is prevented or weakened and gene expression is increased. In another embodiment, the one or more modifications introduce or strengthen insulator sequences controlling expression of the one or more genes in Tables 2A or 2B thereby reducing the influence of nearby silencer elements or repressive chromatin structures such that gene expression is increased.

HDR-Based Modifications that Increase Expression by Targeting Coding Regions

In one embodiment, the programmable nuclease and donor template are configured such that one or more modifications (e.g., insertions, deletions, substitutions) are made in a coding region of the one or more genes of Tables 2A or 2B such that expression of the one or more genes is increased. In one embodiment, the one or more modifications comprise removing inhibitors sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modification may comprise mutations or modifications within the coding region that improve catalytic activity, binding affinity, or other functional properties of the protein. This approach does not directly increase gene expression but can result in an overall increase in the functional output of the gene product.

DNA Base-Editing Modifications that Increase Expression by Targeting Non-Coding Regions

In one embodiment, a DNA base editing system may be configured to make one or more base edits in a non-coding region of one or more genes in Tables 2A or 2B that result in increased expression of the one or more genes in Tables 2A or 2B. In one embodiment, the base editing system is configured to introduce one or more base edits in one or more enhancer regions controlling expression of one or more genes in Tables 2A or 2B such that binding of transcription factors or other regulatory proteins is increased or strengthened and gene expression increased. In another embodiment, the base editing system is configured to introduce one or more base edits in one or more promoters controlling expression of one or more genes in Tables 2A or 2B such that binding of transcription factors and/or RNA polymerase is increased or strengthened and gene expression is increased. In another embodiment, the base editing system is configured to introduce one or more base edits that disrupt or remove one or more silencer elements that control expression of one or more genes in Tables 2A or 2B, such that binding of transcriptional repressors is prevented or weakened and gene expression is increased. In another embodiment, the base editing system is configured to introduce or strengthen insulator sequences controlling expression of the one or more genes in Tables 2A or 2B thereby reducing the influence of nearby silencer elements or repressive chromatin structures such that gene expression is increased.

Base-Editing Modifications that Increase Expression by Targeting Coding Regions

In one embodiment, the base editing system are configured such that one or more base edits are made in a coding region of the one or more genes of Tables 2A or 2B such that expression of the one or more genes is increased. In one embodiment, the one or more base edits comprise removing or disrupting inhibitor sequences, such as IRESs or upstream open reading frames (uORFs), which can negatively affect expression. In one embodiment, the one or more base edits may comprise introducing specific mutations within the coding region that can potentially improve protein stability, folding, or resistance to degradation. While this does not directly increase gene expression, it can lead to higher protein levels and enhanced function. In one embodiment, the one or more base edits may comprise removal or disruption of a sequence encoding an inhibitory PTM site, removal or disruption of one or more ubiquitination sites, or introduction of PTM sites that stabilize or enhance protein function. In one embodiment, the one or more base edits may comprise mutations or modifications within the coding region that improve catalytic activity, binding affinity, or other functional properties of the protein. This approach does not directly increase gene expression but can result in an overall increase in the functional output of the gene product.

Prime Editing Modifications that Increase Expression by Targeting Non-Coding Regions

In another embodiment, the prime editor is configured to introduce one or more enhancer regions controlling expression of one or more genes in Tables 2A or 2B such that binding of transcription factors or other regulatory proteins is increased or strengthened and gene expression is increased. In another embodiment, the primer editor is configured to introduce one or more modifications in one or more promoters controlling expression of one or more genes in Tables 2A or 2B such that binding of transcription factors or RNA polymerase is increased or strengthened and gene expression is increased. In another embodiment, the prime editor is configured to introduce insertions/deletions/substitution that disrupt or remove one or more silence elements thereby preventing binding of transcriptional repressors and increasing gene expression. In another embodiment, the prime editor is configured to introduce or strengthen insulator sequences, thereby reducing the influence of nearby silencer elements or repressive chromatin structures such that gene expression is increased.

Prime Editing Modifications that Increase Expression by Targeting Coding Regions

In one embodiment, the programmable nuclease and donor template are configured such that one or more modifications (e.g., insertions, deletions, substitutions) are made in a coding region of the one or more genes of Tables 2A or 2B such that expression of the one or more genes is increased. In one embodiment, the one or more modifications comprise removing inhibitors sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modification may comprise mutations or modifications within the coding region that improve catalytic activity, binding affinity, or other functional properties of the protein. This approach does not directly increase gene expression but can result in an overall increase in the functional output of the gene product. In another embodiment, prime editing and/or twinPE are used in combination with a recombinase to insert an additional functional copy of the one or more genes from Table 2A or 2B.

### CTCF Editing to Increase Expression

Gene expression may be modified by modification of where chromosomal loops are formed. Chromosomal loops can be removed, e.g., by removing or disrupting one or both of the CTCF motifs, or introduced by creation of a convergent pair of CTCF motifs, either by adding one CTCF motif that is in convergent orientation to a pre-existing CTCF motif or by insertion of a convergent CTCF pair. Any programmable nuclease (CRISPR-Cas, OMEGA systems, Zn Finger Nucleases, TALENs, meganucleases, base editors, prime editors, CAST systems, non-LTR retrotransposons) may be configured to insert, disrupt, or remove CTCF motifs. Three-dimensional chromosome mapping techniques such as Hi-C may be used to determine where loop boundaries are found and accordingly which CTCF motifs should be targeted to remove a loop and silence gene expression or introduce a loop and promote increased gene expression. See e.g., Rao et al. “A 3D Map of the Human Genome at Kilobase Resolution Reveals Principles of Chromatin Looping” Cell, 159 (7): 1665-1680 (2014).

In certain embodiments, the substitution, deletion, or insertion of a CTCF motif or chromosome loop initiates transcription of one or more target genes or transcription factors such that one or more genes or transcription factors from Tables 2A/2B are increased.

### Method Of Treating Inflammatory Disorders Using Adoptive Cell Therapy

In one embodiment, a method of treating an inflammatory disorder comprises administering the engineered cells described herein, or a pharmaceutical composition thereof, to a subject suffering from an inflammatory disorder. The engineered immune cells may be formulated for administration to subjects using suitable methods known in the art. See e.g., Rosenberg et al. Science, 348 (6230), 62-68 (2015); June et al. Science, 359(6382), 1361-1365 (2018); Maude et al. NEJB, 371(16), 1507-1517 (2014); Muranski et al. Blood, 112(2), 362-373. The engineered immune cells may be delivered intravenously or regionally. Regional delivery may include hepatic artery infusion for delivery to the liver, intrapleural or intraperitoneal infusion for delivery to the thoracic or abdominal cavities, intrathecal or intraventricular infusion for delivery to the central nervous system. The engineered cells may also be delivered via lymph node injection. The engineered cells may be formulated in implantable hydrogels for more site-specific delivery. See e.g., Sheih, A., Vo, T. N., & Shah, R. N. (2011). Hydrogels for local and systemic delivery of immunomodulatory cells. Macromolecular Bioscience, 11 (12), 1652-1658; and Smith, T. T., Moffett, H. F., Stephan, S. B., Opel, C. F., Dumigan, A. G., Jiang, X., . . . & Stephan, M. T. (2017). Biopolymers codelivering engineered T cells and STING agonists can eliminate heterogeneous tumors. The Journal of Clinical Investigation, 127 (6), 2176-2191.

Example inflammatory disease that may be treated using the engineered immune cells disclosed herein include those inflammatory diseases and disorders described above in the section entitled “Example Inflammatory Diseases.”

### Methods of Treating Diseases Utilizing a Th17 Transcriptional Regulatory Network

Described herein are methods for treating or preventing an inflammatory or immune disorder comprising a. detecting one or more genes selected from Table 1A and/or Table 2A in a subject thereby creating a gene signature; and b. assigning a differential accessibility (DA) or differential expression (DE) score to the gene signature by comparing the detected one or more genes selected from Table 1A and/or Table 2A to a naïve cell; and c. clustering the one or more genes in the gene signature that are operatively connected to transcription factors selected from Table 1B and/or Table 2B; and d. determining one or more chromatic accessibility scores and/or transcription factor motif scores and/or a enrichment score associated with the one or more genes linked to the transcription factors, thereby determining target regulatory elements; and e. administering a therapeutic agent described elsewhere herein if a chromatic accessibility score and/or transcription factor motif score is detected.

In certain embodiments, a transcriptional regulatory network is developed by integrating of RNA-seq and ATAC-seq data from immune cells. Target genes may be identified from differential accessibility (DA) and differential expression (DE) data by comparing a homeostatic cell state and a pathogenic cell state. In certain embodiments, peaks are assigned to genes as described in the examples. In certain embodiments, multiple peaks may be assigned to the genes. In certain embodiments, if multiple peaks are assigned to the same gene, only the one with lowest p-value may be kept. In certain example embodiments, genes that are associated with DA peaks but are not expressed or whose DE false discovery rate (FDR) is not available due to low expression or presence of outliers (Love et al., 2014) may not be labeled as DE. In certain embodiments, targets may be further classified where genes associated with peaks that are more accessible (e.g., when FDR<0.05 and/or fold change >1.2) and whose expression is upregulated (e.g. when FDR<0.05) in the same genotype may be defined as active targets, and genes associated with DA peaks but that were not labeled as DE are defined as poised targets. In certain embodiments, active target genes are genes whose locus is more open in the pathogenic state relative to cells in the homeostatic state and whose RNA is more highly expressed in the pathogenic state, or alternatively may represent that the genes are active in the pathogenic cell state and repressed in homeostatic cell state. In certain embodiments, genes associated with more open peaks but that have lower expression in the same genotype may be excluded if there is a small number of genes in this category and a lack of clear biological interpretation.

In certain embodiments, candidate transcription factors for comparison may be imported from a database (e.g. HOCOMOCO v11 database (mouse core collection) (Kulakovskiy et al., 2018)) and filtered by expression (e.g. TFs with an average expression >=1 TPM may be kept as potential TFs for analysis). In certain embodiments, a motif sequence scanner or program may be used to identify a link between two targets or a transcription factor and its gene target. In example embodiments, links between TFs and targets may be identified using a FIMO (Grant et al., 2011) to obtain the relative coordinates of the motif hits in the input sequences. In certain embodiments, identifying a link between a TF and a gene target builds a network showing TFs connected to at least one target. In example embodiments, adjacency matrices may be built for each target category such that each TF's motif binding is identified as present or not in each target. In certain embodiments, targets within each category may then be clustered into putative gene modules based on the presence of shared TF binding motifs. In certain embodiments, a hierarchical clustering (e.g. Euclidean distance and Wald agglomeration methods) (Murtagh and Legendre, 2014)) may be applied to the adjacency matrices and clusters previously identified using an adaptive branch pruning algorithm (e.g. cutreeDynamic function from the dynamicTreeCut program) (Langfelder et al., 2008)).

In certain embodiments, targets may be summarized into modules and selected TFs that were most significantly enriched in the homeostatic cell state or in the pathogenic cell state or were significantly enriched in one genotype and are known to be important in Th17 cells (e.g., examples included EGR1, FOXO1, STAT3). In certain embodiments, directed edges from TFs to targets may be weighted by connectivity and defined as the proportion of genes in a target module whose associated DA peaks contained at least one motif binding site for the TF pointing to it. In certain embodiments, the network may be further pruned to only include edges with absolutely or relatively high connectivity. In an example embodiment, a high threshold may be defined (e.g., was defined as connectivity >=0.3) and relatively high threshold may be defined as a significantly higher connectivity from the TF to the target module of interest than the average connectivity from the same TF to the rest of targets in the same target category. In certain embodiments, a chi-square test may be applied to test the significance in the differences (e.g., module-size-weighted average connectivity in the rest of modules is equivalent to the proportion of targets not belonging to the module of interest containing at least one TF motif binding site in associated DA peaks). In certain embodiments, the degree of target module nodes is computed after edge pruning and is defined as the number of TFs pointing to a node.

In certain embodiments, an agent or a population of modified cells as discussed elsewhere herein is administered to treat an autoimmune or inflammatory disease after determining gene and regulatory targets in a regulatory target network that can affect the state of an immune cell. In example embodiments, the one or more targets in a regulatory network are the one or more genes or transcription factors from Tables 1/2. In an example embodiment, an immune cell is a T cell. In an example embodiment, a T cell is a Th17 cell.

Methods for Detecting Target Genes and/or Gene Products

The disclosure provides biomarkers for the identification, diagnosis, prognosis and manipulation of disease phenotypes (e.g., immune state), for use in a variety of diagnostic and/or therapeutic indications. Biomarkers in the context of the present disclosure encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein (e.g., TCF7/TCF1 and genes and/or gene products thereof in Tables 1A-1B and 2A-2B).

The genes and/or products thereof in Tables 1A/1B and/or 2A/2B and/or their amount of activity and/or expression can be detected and/or quantified and compared to a suitable control so as to identify, diagnose, prognose, and/or treat or select ana appropriate treatment for a subject having or being suspected of having a disease, such as an autoimmune or inflammatory disease described herein. Accordingly, in certain embodiments, the disclosure provides uses of the biomarkers for predicting risk for a certain phenotype. In certain embodiments, the disclosure provides uses of the biomarkers for selecting a treatment. In certain embodiments, a subject having a disease can be classified based on severity of the disease. Detection and or other quantification of one or more biomarkers (e.g., any one or more of those in Tables 1A/1B and/or 2A/2B) can be used in combination with a suitable therapy or treatment for a disease or disorder in a subject in need thereof, such as an autoimmune or inflammatory disease or disorders. Exemplary autoimmune and inflammatory diseases are described in greater detail elsewhere herein.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR-), Youden index, or similar.

Detection of a biomarker may be by any means known in the art. Methods of detection include, but are not limited to enzymatic assays, flow cytometry, mass cytometry, fluorescence activated cell sorting (FACS), fluorescence microscopy, affinity separation, magnetic cell separation, microfluidic separation, RNA-seq (e.g., bulk or single cell), quantitative PCR, MERFISH (multiplex (in situ) RNA FISH), immunological assay methods by specific binding between a separable, detectable and/or quantifiable immunological binding agent (antibody) and the marker, mass spectrometry analysis methods, chromatography methods and combinations thereof. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), Western blot, etc. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilized metal affinity chromatography, and the like.

### Detection of Biomarkers

In one embodiment, the signature genes (see e.g., Tables 1A/1B and/or Tables 2A/2B), biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, WGS (described herein), WES (described herein), RNA-seq, single cell RNA-seq (described herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. Detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3): 317-25). In certain embodiments, cancer is diagnosed, prognosed, or monitored. For example, a tissue sample may be obtained and analyzed for specific cell markers (IHC) or specific transcripts (e.g., RNA-FISH). In one embodiment, tumor cells are stained for cell subtype specific signature genes. In one embodiment, the cells are fixed. In another embodiment, the cells are formalin fixed and paraffin embedded. Not being bound by a theory, the presence of the tumor subtypes indicate outcome and personalized treatments.

The present disclosure also may comprise a kit with a detection reagent that binds to one or more biomarkers or can be used to detect one or more biomarkers.

### Sequencing

In certain embodiments, sequencing is used to identify expression of genes or transcriptomes in single cells. In certain embodiments, sequencing comprises high-throughput (formerly “next-generation”) technologies to generate sequencing reads. Methods for constructing sequencing libraries are known in the art (see, e.g., Head et al., Library construction for next-generation sequencing: Overviews and challenges. Biotechniques. 2014; 56 (2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437: 376-80); Schneider and Dekker (Nat Biotechnol. 2012 Apr. 10; 30 (4): 326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, IRNA molecules, in a sample, for example, a single cell or a population of cells. In some embodiments, a transcriptome refers to 25%, 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In certain embodiments, the disclosure involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the present disclosure involves single cell RNA sequencing (scRNA-seq). In certain embodiments, the disclosure involves plate based single cell RNA sequencing (see, e.g., Picelli, S. et al., 2014, “Full-length RNA-seq from single cells using Smart-seq2” Nature protocols 9, 171-181, doi: 10.1038/nprot.2014.006).

In certain embodiments, the disclosure involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1): 44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14 (3): 302-308, 2017; Cao, et al., Comprehensive single-cell transcriptional profiling of a multicellular organism. Science, 357 (6352): 661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the disclosure involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14 (10): 955-958; International Patent Application No. PCT/US2016/059239, published as WO2017164936 on Sep. 28, 2017; International Patent Application No.PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, dimension reduction is used to cluster single cells based on differentially expressed genes. In certain embodiments, the dimension reduction technique may be, but is not limited to, Uniform Manifold Approximation and Projection (UMAP) or t-SNE (see, e.g., Becht et al., Evaluation of UMAP as an alternative to t-SNE for single-cell data, bioRxiv 298430; doi.org/10.1101/298430; and Becht et al., 2019, Dimensionality reduction for visualizing single-cell data using UMAP, Nature Biotechnology volume 37, pages 38-44).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS) n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (1125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

In certain embodiments, a subject can be categorized based on signature genes or gene programs expressed by a tissue sample obtained from the subject. In certain embodiments, the tissue sample is analyzed by bulk sequencing. In certain embodiments, subtypes can be determined by determining the percentage of specific cell subtypes expressing the identified interacting genetic variants in the sample that contribute to the phenotype. In certain embodiments, gene expression associated with the cells are determined from bulk sequencing reads by deconvolution of the sample. For example, deconvoluting bulk gene expression data obtained from a tumor containing both malignant and non-malignant cells can include defining the relative frequency of a set of cell types in the tumor from the bulk gene expression data using cell type specific gene expression (e.g., cell types may be T cells, fibroblasts, macrophages, mast cells, B/plasma cells, endothelial cells, myocytes and dendritic cells); and defining a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in the tumor or the same tumor type, and wherein the residual of the linear relationship defines the malignant cell-specific (MCS) expression profile (see, e.g., WO 2018/191553; and Puram et al., Cell. 2017 Dec. 14; 171 (7): 1611-1624.e24).

### Screening for T Cell Modulating Agents

In certain embodiments, the disclosure provides for screening of agents, genetic modifying agents, or therapeutic agents capable of modulating the immune cell state (e.g., pathogenic Th17 cell state). In certain embodiments, agents capable of modulating the Th17 regulators on T cells are screened. In certain embodiments, the method comprises: a) applying a candidate agent to a cell population comprising Th17 T cells; b) detecting modulation of one or more phenotypic aspects of the cell population by the candidate agent, thereby identifying the agent. The phenotypic aspects of the cell population that is modulated may be a gene signature or biological program specific to a cell type or cell phenotype or phenotype specific to a population of cells (e.g., a pathogenic phenotype). In certain embodiments, steps can include administering candidate modulating agents to cells, detecting identified cell (sub) populations for changes in signatures, or identifying relative changes in cell (sub) populations which may comprise detecting relative abundance of particular gene signatures. The phenotype may be a change in secretion of cytokines associated with Th17 T cells. In certain embodiments, candidate agents are screened in in vivo models of autoimmunity or inflammation (e.g., mouse models). In certain embodiments, anti-inflammatory activity in a model is detected.

The term “agent” in this context herein, broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

In certain embodiments, the agent is capable of modulating the pathogenicity, proliferation, differentiation, maturation, migration, cytokine expression, cytotoxicity and/or viability of the isolated immune cell or immune cell population. In certain embodiments, the agent is capable of inducing or repressing the pathogenicity, proliferation, differentiation, maturation, migration, cytokine expression, cytotoxicity and/or viability of the isolated immune cell or immune cell population.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present disclosure provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present disclosure may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present disclosure in silico.

Further embodiments are illustrated in the following Examples which are given for illustrative purposes only and are not intended to limit the scope of the disclosure.

### EXAMPLES

### Example 1

CD4⁺ T helper 17 (Th17) cells encompass a spectrum of cell states including cells that maintain homeostatic tissue functions, such as mucosal barrier integrity, and pro-inflammatory cells that drive autoimmune tissue damage/inflammation. Identifying the regulators that determine Th17 cell states thus provides the means to control tissue inflammation and restore homeostasis without compromising the physiological functions of Th17 cells. The present disclosure identified TCF1 as the key regulator that determines Th17 cell state. IL-23, a cytokine critical for inducing pathogenic Th17 cells, decreased TCF1 expression. Consistent with this observation, conditional deletion of Tcf7/TCF1 in mature myelin-specific T cells conferred pathogenicity to homeostatic Th17 cells independent of IL-23. Conversely, sustained TCF1 expression impaired acquisition of pathogenicity. Integration of transcriptional and chromatin accessibility data showed that TCF1 maintained homeostatic state through a regulatory network involving ETS family transcription factors, EGR1, and FOXO1 and by binding to and interfering with RORγt activity. The present disclosure provides mechanistic insight into how the homeostatic and pathogenic Th17 cell states are determined and into the association of genetic variants in TCF7 with susceptibility to autoimmunity.

### INTRODUCTION

CD4 interleukin-17 (IL-17)-producing T cells (Th17) are critical for the maintenance of tissue homeostasis and for host defense against extracellular bacterial and fungal pathogens. Th17 cells are also key mediators of pathogenesis in several autoimmune diseases, including multiple sclerosis (MS), rheumatoid arthritis (RA), psoriasis, type-1 diabetes (TID), and inflammatory bowel disease (IBD). The diverse roles of Th17 cells can be explained by the existence of several cell states in between two extremes marked by homeostatic or non-pathogenic Th17 (npTh17) and inflammatory or pathogenic Th17 (pTh17) cells in both mice and humans. npTh17 or homeostatic cells are induced and maintained by the gut microbiota in vivo and by in vitro differentiation of naïve CD4⁺ T cells with TGFβ1+IL-6. npTh17 produce IL-17A, IL-22, and IL-10, are important for maintaining mucosal barrier function and have been shown to rely mainly on oxidative phosphorylation (OXPHOS) for their energetic demands. pTh17 are induced during inflammatory states in vivo and by IL-1β+IL-6+IL-23 in vitro. pTh17 have the potential to fuel destructive autoimmunity and rely on both aerobic glycolysis and OXPHOS.

In the experimental autoimmune encephalomyelitis (EAE) model of central nervous system (CNS) autoimmunity that serves as a model for the human disease MS, naïve myelin-oligodendrocyte glycoprotein (MOG)-specific T cell receptor (TCR) transgenic CD4 T cells (2D2) differentiated into npTh17 induce little to no signs of disease, whereas cells differentiated into pTh17 cause severe disease. Evidence suggests that npTh17 are precursors of pTh17 and that IL-23 is the switch factor that governs the conversion of npTh17 to pTh17 in vivo. Indeed, IL-23 is essential for driving Th17 pathogenicity as the absence of either the IL-23-specific p19 subunit or IL-23R results in the inability of T cells to drive autoimmune inflammation. Further, STAT3 and STAT4, two useful mediators of IL-23 signaling, have also been identified as putative MS susceptibility genes, underscoring the relevance of IL-23 signaling in MS etiopathogenesis. Despite the critical role of IL-23 in Th17 pathogenicity, the drivers that maintain homeostatic or induce pathogenic Th17 cell state remain unclear.

T cell factor 1 (TCF1), encoded by Tcf7, is a transcription factor (TF) that has roles at multiple steps in T cell biology. Recently, it has come to the forefront with the observation that it antagonizes the development of dysfunction in CD8 T cells and, instead, maintains a population of CD8 T cells that retain features of stemness and seed the effector T cell response upon checkpoint blockade therapy. However, Tcf7/TCF1 also has a role during T cell development and in regulating peripheral T cell responses, including regulatory T cells (Treg).

TCF1 exerts its functions by multiple mechanisms: (i) repressing or activating transcription via binding to genomic regions containing Wnt response elements (WRE), (ii) modifying chromatin via its histone de-acetylase (HDAC) domain, and (iii) physically binding and trans-repressing the activity of other TFs. Importantly, a large genome-wide association study (GWAS) of 14,498 MS patients and 24,091 healthy subjects associated single nucleotide polymorphisms (SNPs) within the TCF7 locus with increased MS risk. This association was strengthened by analysis of genomic data from 47,429 MS and 68,374 control subjects, further supporting the link between SNPs in TCF7 and MS risk. Similarly, in a genome-wide analysis of EAE, Tcf7 was part of a gene network that significantly associated with disease severity, weight loss, and was enriched for T cell-specific gene pathways. Lastly, genetic variation in TCF7 has also been associated with TID and RA, where Th17 cells also play a major role.

However, the role of TCF1 in Th17 biology remains unclear due to conflicting data. Studies utilizing Tcf7 germline knockout mice, which have severely impaired T cell development, showed that Tcf7-deficient Th17 cells produced higher levels of IL-17A and induced exacerbated EAE, indicating a negative regulatory role for TCF1 in Th17 pathogenicity. In contrast, a study in CD4⁺ T cells from mice harboring constitutively active β-catenin signaling (CD4Cre-Ctnnb1^{ex3}), which activates TCF1, showed that TCF1 bound to accessible chromatin regions marked by lysine acetylation of histone 3 (H3KAc) in the Rorc locus (expressing RAR-related orphan receptor gamma (RORY), also known as RORC, NR1F3, RORG) and was associated with a pro-inflammatory gene program in Th17 cells. Further, TCF1 has been shown to mark long-lived Th17 cells that can eradicate tumors and drive autoimmune-like toxicity. The present disclosure showed that Tcf7 marks stem-like non-pathogenic precursors that can convert into pathogenic Th17 cells in an IL-23-dependent manner. Altogether, these studies suggest a role for TCF1 in dictating Th17 cell responses, but it is not clear whether Tcf7 is required for Th17 cells in general or whether it is has a role in Th17 fate specification. Deciphering the precise role of Tcf7 requires its perturbation specifically in mature T cells to avoid confounders stemming from the abnormal T cell development in germline Tcf7 knock-out mice or from indirect study of TCF1 activity downstream of constitutively active beta-catenin, which is known to have TCF1-independent effects.

The present disclosure, by employing conditional modulation of TCF1 in mature T cells, identified TCF1 as the critical regulator of Th17 cell state. IL-23 signaling decreased TCF1, which maintained homeostatic state through a regulatory network involving cooperation with ETS family members, EGR1, and FOXO1 and by binding to and interfering with RORγt activity. The present disclosure showed how Th17 cell state is determined and provides a framework for understanding the association of genetic variants in the TCF7 locus with susceptibility to autoimmune diseases.

### Results

### Differential Regulation of TCF1 in Th17 Subtypes is Driven by IL-23

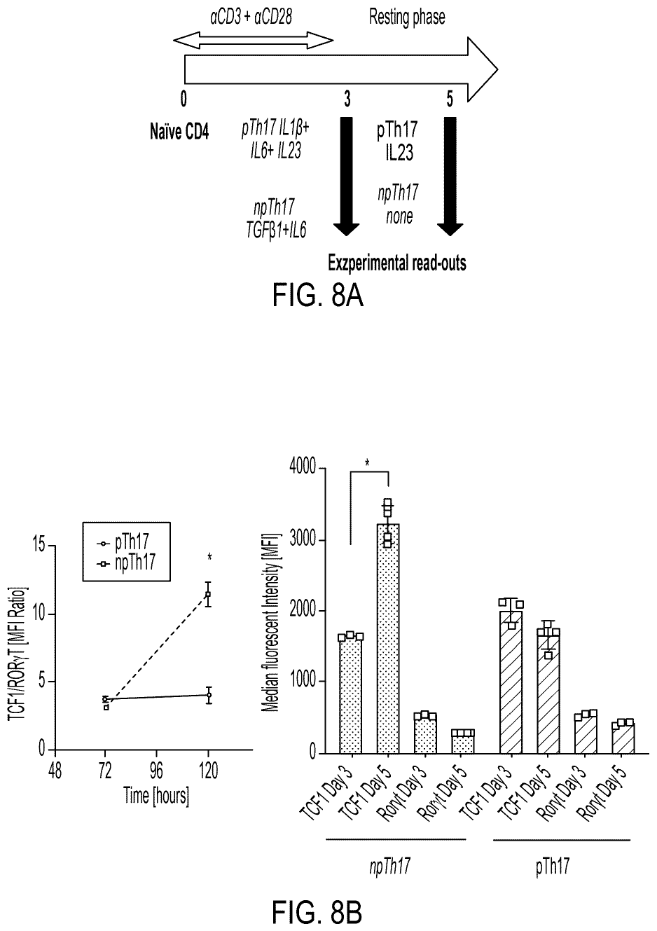

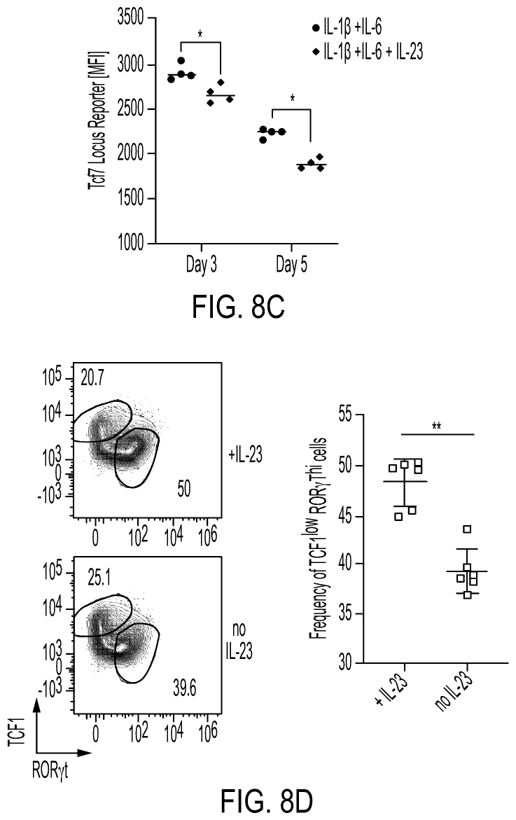

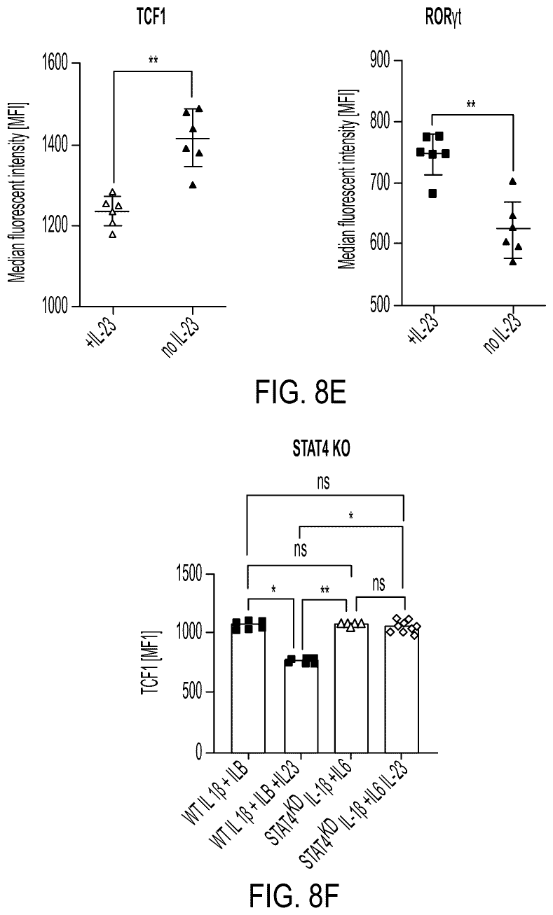

Naïve T cells express high levels of TCF-1, which is rapidly decreased upon TCR engagement. The present disclosure examined the dynamics of Tcf7 expression in IL17A-GFP pTh17 (IL-1β+IL-6+IL-23) and npTh17 (TGF-β1+IL-6) differentiated from Il17a-gfp reporter mice after primary activation with plate-bound anti-CD3+anti-CD28 and after a resting phase (FIG. 8A). Both pTh17 and npTh17 showed decreased mRNA expression of Tcf7 and its downstream target Axin2 compared to naïve T cells after the primary stimulation; however, only npTh17 re-expressed both Tcf7 and Axin2 during the resting phase (FIG. 1A). The present disclosure confirmed that the Tcf7 locus had increased activity in npTh17 relative to pTh17 using a Tcf7 reporter in which GFP expression is controlled by the Tcf7 locus (FIG. 1B). Examination of TCF1 protein confirmed that TCF1 re-expression occurred in npTh17 while expression in pTh17 remained low (FIG. 1C). Lastly, the present disclosure examined the expression of TCF1 in Th17 cells in vivo during EAE. SLAMF6 marks npTh17 and CXCR6 marks pTh17 in vivo. In line with the in vitro data, the present disclosure confirmed that TCF1 expression was significantly higher in TCRβ⁺CD4⁺RORγt⁺ Th17 cells that expressed SLAMF6 compared to those that expressed CXCR6 in mice with EAE (FIG. 1D).

It has been suggested that Tcf7 promotes RORγt (produced from an mRNA identical to that of RORY, except that the two 5′-most exons are replaced by an alternative exon, located downstream in the gene, which causes a different, shorter N-terminus) expression. The relationship of TCF1 and RORγt in npTh17 and pTh17 was examined. It was found that while pTh17 maintained stable TCF1 and RORγt expression throughout the differentiation and rest phase, npTh17 showed a sharp increase in TCF1 expression while Rorγt expression remained unchanged, resulting in an altered TCF1/RORγt ratio (FIG. 8B). Further, pTh17 but not npTh17 showed the emergence of a distinct TCF1^{lo}RORγt^{hi} population, indicating a potential reciprocal relationship between TCF1 and Rorγt (FIG. 1E). Given that pTh17 but not npTh17 receive IL-23, the disclosure next addressed whether IL-23 signaling was responsible for the decreased TCF1 expression in pTh17. Naïve CD4⁺ T cells were differentiated with IL-1B and IL-6 for 2 days, followed by the addition of increasing doses of IL-23. TCF1 protein expression decreased significantly in an IL-23 dose-dependent manner (FIG. 1F). The disclosure further differentiated CD4 T cells from the Tcf7 locus reporter strain with IL-1β+IL-6 with or without IL-23 for 3 days followed by 2 days of rest. Tcf7 locus reporter activity was significantly decreased by the addition of IL-23 and remained low throughout the rest period (FIG. 8C). These data indicated that IL-23 decreased TCF1 expression through transcriptional silencing. To further assess the impact of IL-23 stimulation on the observed reciprocal relationship between TCF1 and RORγt, pTh17 was differentiated for 3 days and then added fresh IL-23 in the culture media. IL-23 addition led to an increased proportion of TCF1^{lo}RORγt^{hi} cells (FIG. 8D). Conversely, cultures that did not receive fresh IL-23 had an increased proportion of TCF1hi RORγt^{lo} cells. These shifts in the balance of TCF1- and RORγt-expressing cells were stable as they persisted even after re-stimulation (FIG. 8E). In addition, pTh17 cells differentiated from IL-23r-gfp mice demonstrated lower levels of Tcf7 and Axin2 mRNA in IL-23R⁺ compared to IL-23R-cells (FIG. 1G). Importantly, the effect of IL-23 was specific to the Tcf7/TCF1 arm of the Wnt signaling pathway as Lef expression did not differ in the two populations. Lastly, the disclosure examined whether IL-23 signaling also regulated TCF1 expression in vivo. Wild type (WT), p19^{-/-}, and IL-23r-mice were immunized with MOG₃₅₋₅₅ peptide and found that TCF1 expression was significantly higher in TCRβ⁺CD4⁺RORγt⁺ Th17 cells harvested from p19^{-/-} and IL-23r^{-/-} mice compared to WT (FIG. 1H). Overall, the data indicated that IL-23 signaling dampened TCF1 expression and downstream activity, resulting in a shift in the balance of TCF1- and RORγt-expressing cells in Th17 subtypes.

### TCF1 Modulation Determines Th17 Pathogenicity

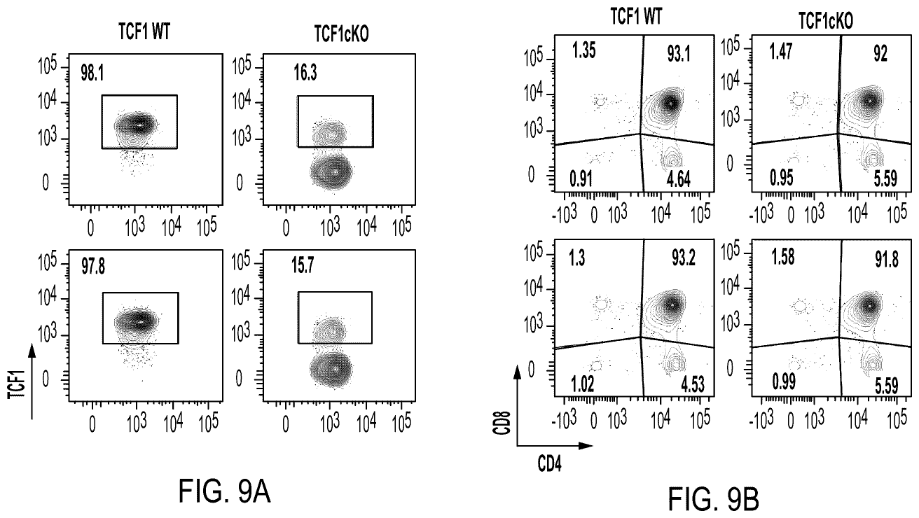

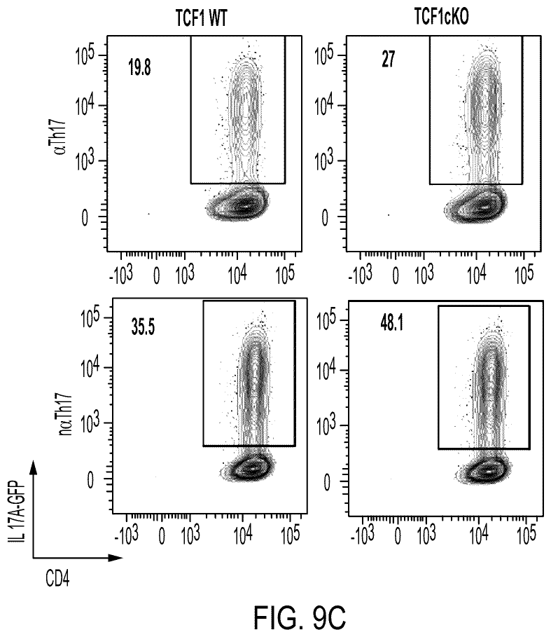

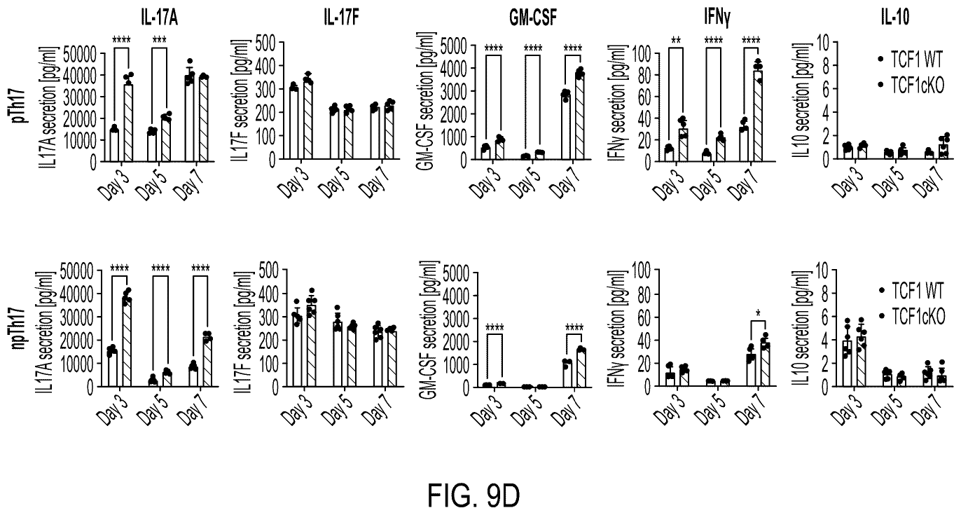

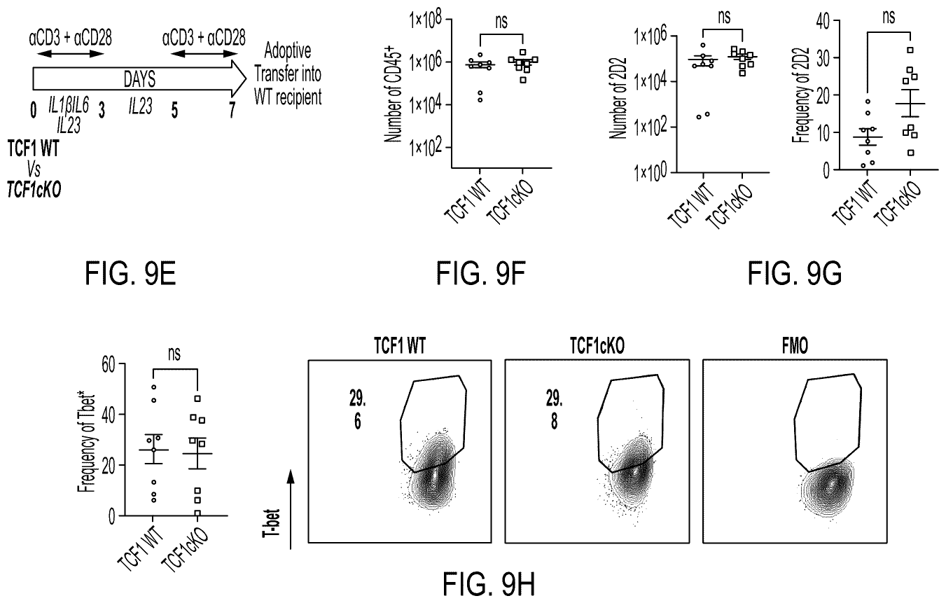

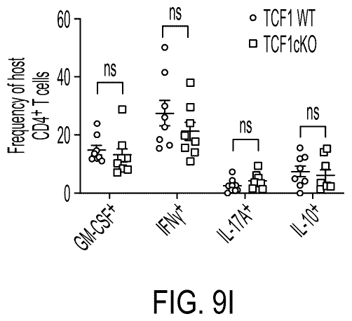

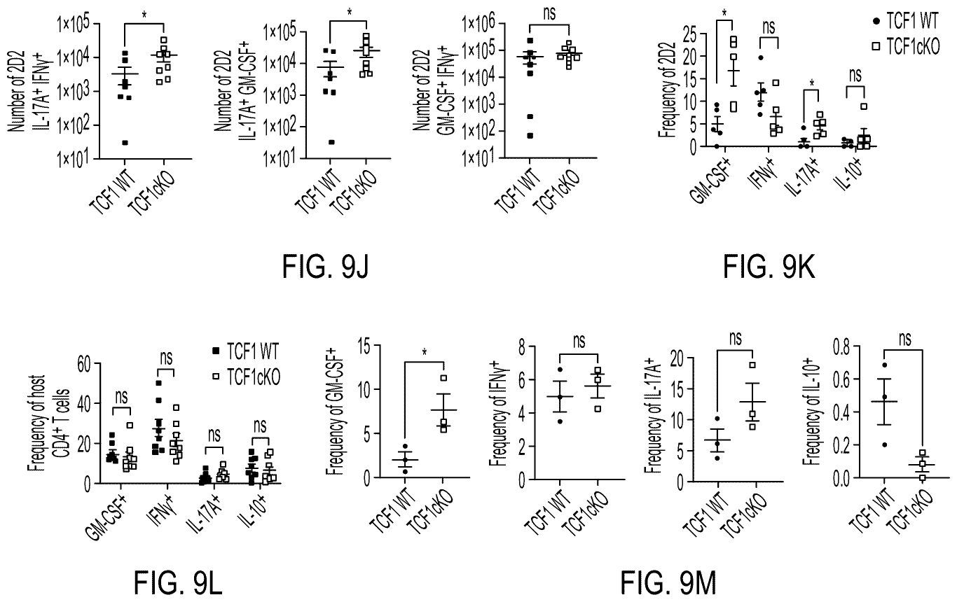

To investigate the role of TCF1 in Th17 differentiation and function, a mouse was generated that conditionally deletes Tcf7 after positive selection in the thymus (FIG. 9A) and reports IL-17A expression with GFP (dLck-cre Tef7^{flox/flox}il17agfp, hereafter TCF1cKO). By using this model, it was possible to bypass any defects stemming from loss of Tcf7 early during T cell development (FIG. 9B). This model was employed to investigate how loss of TCF1 impacts Th17 differentiation in vitro and in vivo during Th17-driven CNS autoimmunity. The present disclosure first examined the ability of naïve CD4⁺ T cells from TCF1 WT (Tef7^{flox/flox}il17agfp) and TCF1cKO mice to differentiate into pTh17 and npTh17 cells and found that both were able to differentiate into Th17 cells (FIG. 9C); however, TCF1cKO cells secreted higher levels of IL-17A, GM-CSF, and IFNγ, all of which have been implicated in driving pathogenic CNS tissue inflammation, compared to TCF1 WT cells (FIG. 9D). Other cytokines, such as IL-17F and IL-10, were unchanged indicating that TCF1 did not simply restrain general effector function, but rather it regulated a specific set of genes involved in pathogenic CNS tissue inflammation.

The present disclosure next examined how TCF1 impacted Th17 pathogenicity in vivo. The present disclosure differentiated naive CD4 T cells from myelin-oligodendrocyte-specific T cell receptor transgenic (2D2) TCF1 WT (2d2 Il17agfp Tef7flox flox) and TCF1cKO (2d2 dLck-cre⁺Tcf7^{flox/flox}Il17agfp⁺) mice into pTh17 and transferred them into WT recipient mice (FIG. 9E). 2D2 TCF1cKO cells induced more severe EAE, with an earlier onset, higher maximal disease score, and increased mortality (FIG. 2A; FIG. 8A-8E). Recipients of 2D2 TCF1 WT and TCF1cKO cells both displayed marked meningeal and parenchymal mononuclear cell infiltrates (FIG. 2B) and similar numbers of total CD45 cells infiltrating the CNS (FIG. 9F), suggesting that qualitative rather than quantitative differences were responsible for the observed increased in disease severity driven by 2D2 TCF1cKO pTh17 cells. Accordingly, the functional phenotype of 2D2 TCF1 WT and TCF1cKO cells harvested from the CNS at the peak of disease was examined. As expected, the total number and proportion of 2D2 cells infiltrating the CNS did not differ across groups (FIG. 9G); however, there was a higher proportion and number of 2D2 cells expressing RORγt among 2D2 TCF1cKO cells (FIG. 2C). Th17 cells that acquire T-bet expression and Th1-like features have been implicated in driving neuroinflammation. The present disclosure therefore examined T-bet expression in 2D2 TCF1 WT and TCF1cKO CNS-infiltrating cells but found no differences, suggesting that the increased pathogenicity displayed by TCF1cKO cells was not due to increased trans-differentiation into Th1-like cells (FIG. 9H). Indeed, the overall proportion of IFNγ-producing cells was not different in 2D2 TCF1 WT and TCF1cKO cells (FIG. 2D). GM-CSF-producing cells have also been implicated as having a key role in promoting pathogenic CNS tissue inflammation via the recruitment and maintenance of pro-inflammatory myeloid cells; however, no differences in the frequency of GM-CSF cells between 2D2 TCF1 WT and TCF1cKO cells were observed (FIG. 2D). The frequency of IL-10-producing cells that can suppress CNS tissue inflammation also did not differ. Interestingly, it was found that 2D2 TCF1cKO cells contained a higher proportion of IL-17A-producing cells (FIG. 2D), which translated into significantly more polyfunctional cells producing IL-17A⁺IFNγ⁺ and IL-17A GM-CSF, while the proportion of GM-CSF⁺IFNγ⁺ cells was unchanged (FIG. 2E-2G). Finally, host CNS-infiltrating CD4⁺ T cells in both groups exhibited similar cytokine production (FIG. 9J), indicating that the observed effects in TCF1cKO cells were cell-intrinsic. Altogether, the data indicated that loss of TCF1 resulted in heightened pathogenicity, which was associated with higher RORγt expression and frequency of polyfunctional IL-17A⁺ cells. Notably, polyfunctional IL17-producing T cells have been regarded as key drivers of CNS tissue inflammation.

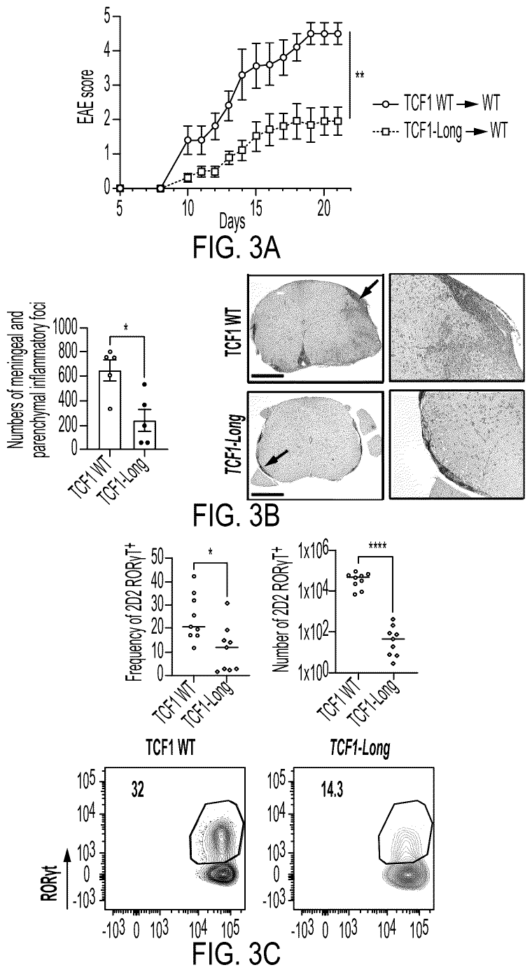

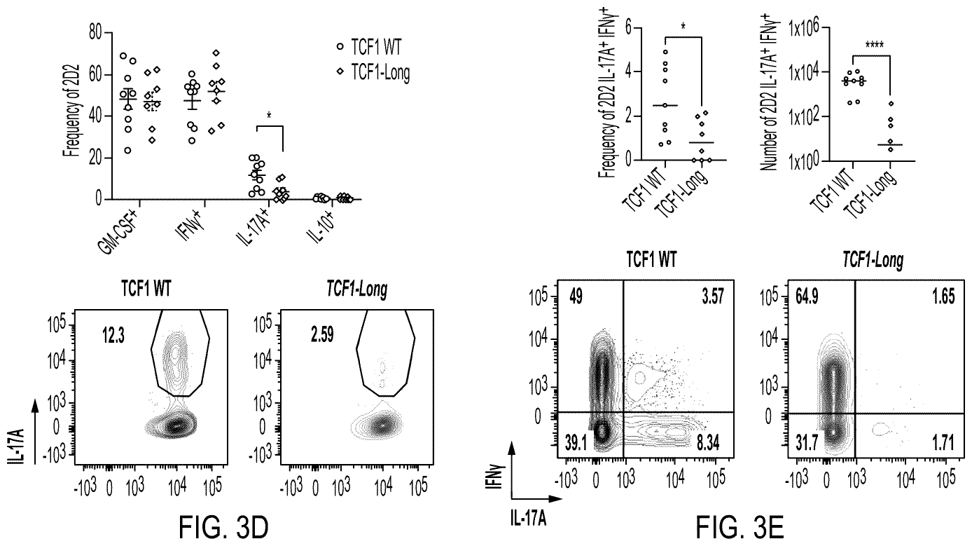

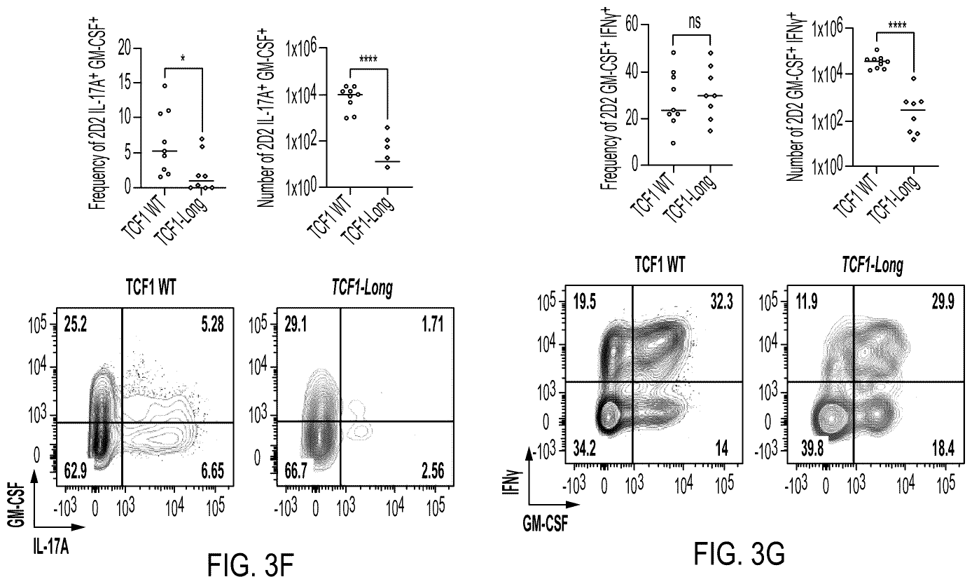

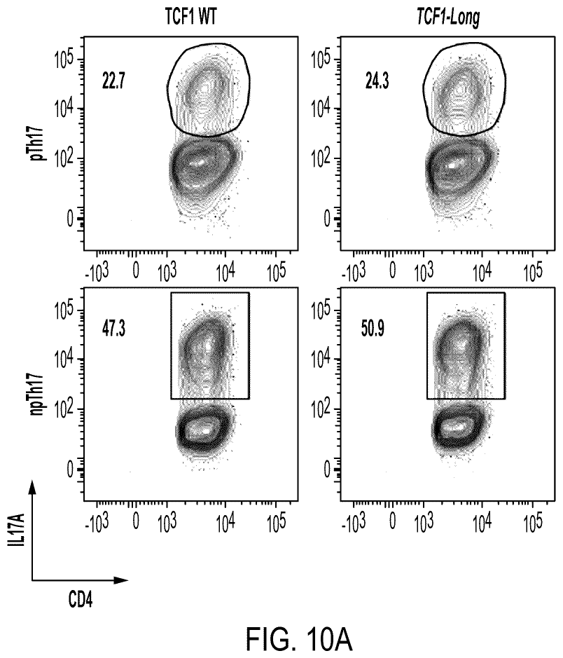

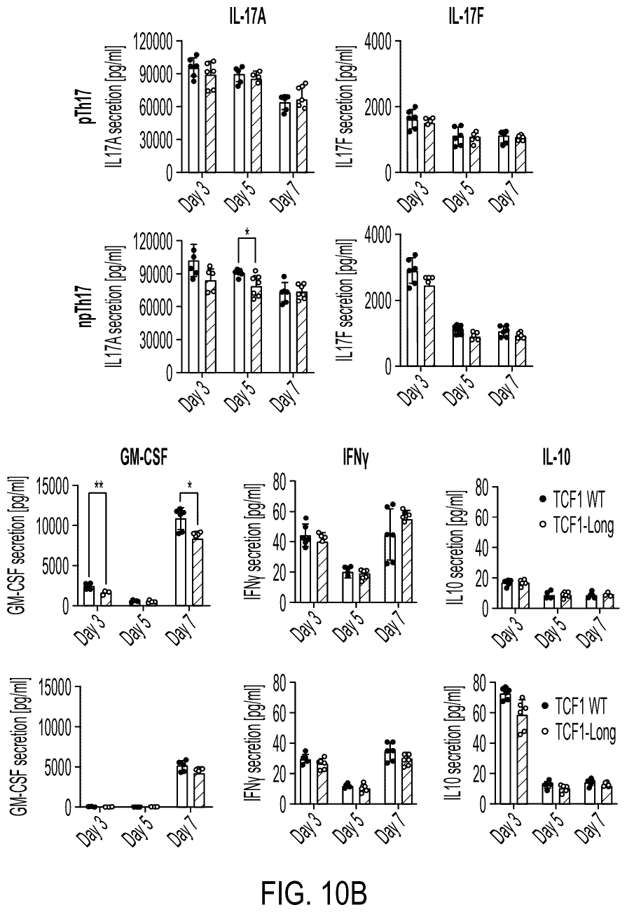

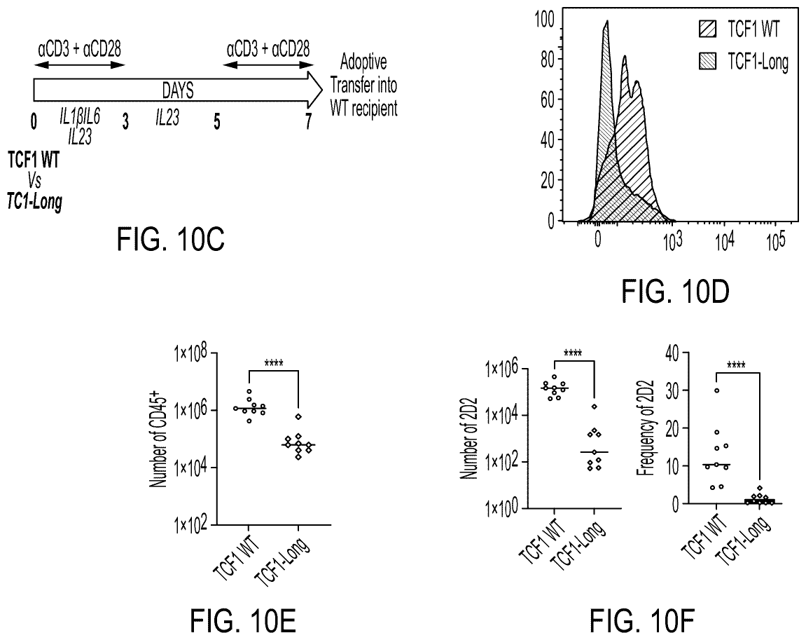

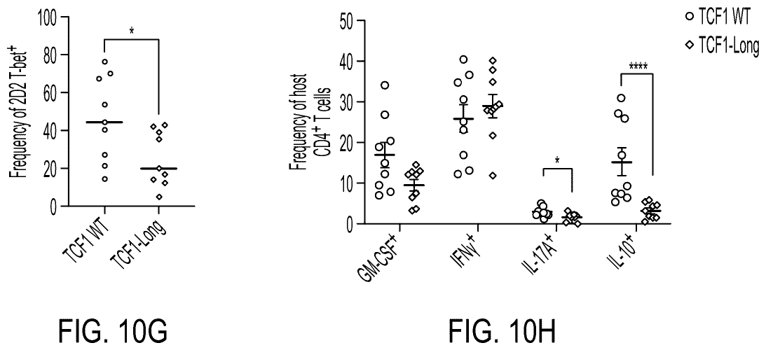

To further confirm these observations regarding the role of TCF1 in Th17 pathogenicity, the effect of TCF1 gain-of-function in Th17 cells was examined. For this, the disclosure generated a transgenic mouse that overexpressed the transcriptionally active long isoform of TCF1 in T cells (Cd2-Tcf7L, hereafter TCF1-Long). TCF1 transgenic expression did not impact the ability of naïve CD4⁺ T cells to differentiate into Th17 cells and did not affect cytokine production, apart from decreased secretion of GM-CSF (FIGS. 10A and 10B). To assess whether sustained TCF1 expression impacted Th17 pathogenicity in vivo, the techniques herein differentiated naive CD4⁺ T cells from 2D2 TCF1 WT and TCF1-Long mice into pTh17 and transferred them into WT recipient mice (FIG. 10C). Notably, 2D2 TCF1-Long cells maintained TCF1 expression at a physiologically high level with limited low-expressing cells when compared to 2D2 WT cells (FIG. 10D). It was then investigated how sustained, but not supra-physiological, TCF1 expression impacted the ability of Th17 cells to drive autoimmunity. 2D2 TCF1-Long cells drove a significantly milder disease, with similar onset and mortality but markedly lower disease score (FIG. 3A; FIG. 9A-91). The numbers of meningeal and parenchymal inflammatory foci were significantly lower in mice receiving 2D2 TCF1-Long cells, which was in line with the reduced number of total CD45 cells and 2D2 cells infiltrating the CNS (FIG. 3B; FIG. 10E-10F). 2D2 TCF1-Long cells further had a lower proportion and overall number of RORγt cells, as well as a lower proportion of T-bet cells (FIG. 3C; FIG. 10G). CNS-infiltrating 2D2 TCF1-Long cells did not exhibit any differences in GM-CSF- or IFNγ-producing cells compared to TCF1 WT cells; however, they had significantly decreased IL-17A-producing cells (FIG. 3D). The number and proportion of polyfunctional IL-17A⁺IFNγ⁺ and IL-17A⁺GM-CSF⁺ among 2D2 TCF1-Long cells was significantly reduced, however, the proportions of GM-CSF⁺IFNγ⁺ cells were similar in both groups (FIG. 3E-3G). Endogenous CD4⁺ T cells showed similar levels of GM-CSF⁺ and IFNγ⁺ but lower frequencies of IL-17A⁺ and IL-10⁺ cells in the CNS of TCF1-Long 2D2 cell recipient mice, findings compatible with the overall lower CNS tissue inflammation in recipients of 2D2 TCF1-Long pTh17 cells (FIG. 10H). Collectively, the TCF1 loss- and gain-of-function data indicated that TCF1 restrained the pathogenicity of Th17 cells and predominantly affected the proportion of RORγt-expressing and polyfunctional IL-17A-producing cells.

TCF1 Deficiency Confers Pathogenicity to npTh17 Cells

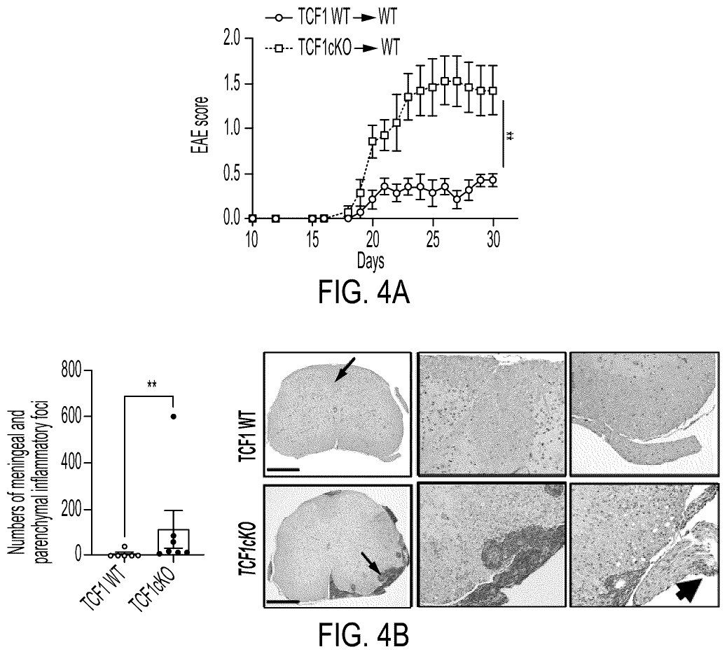

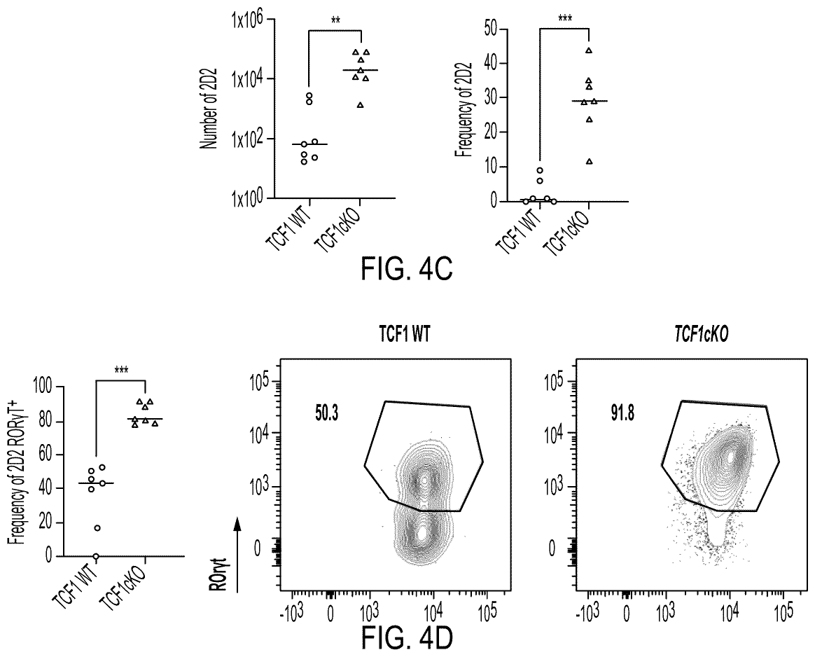

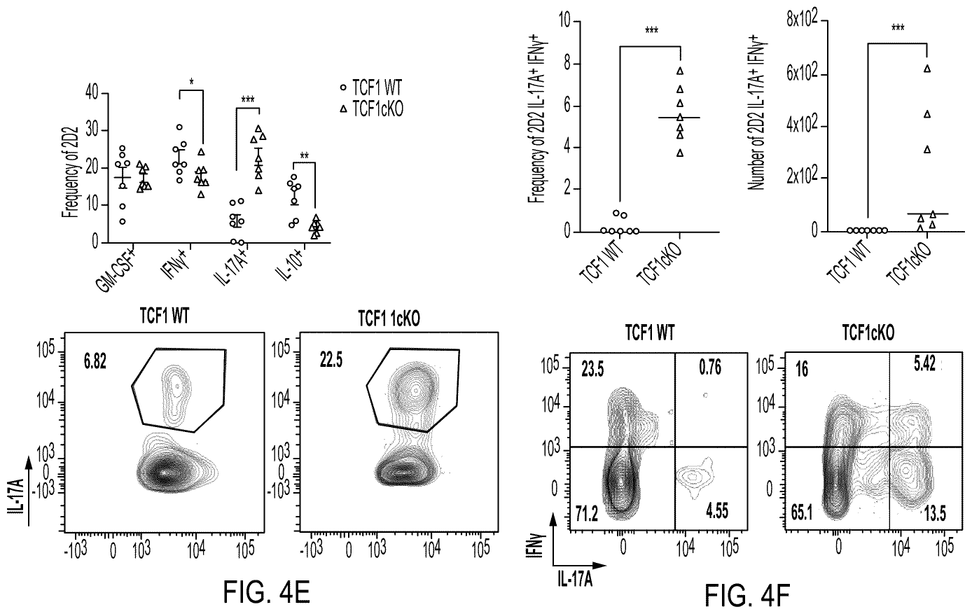

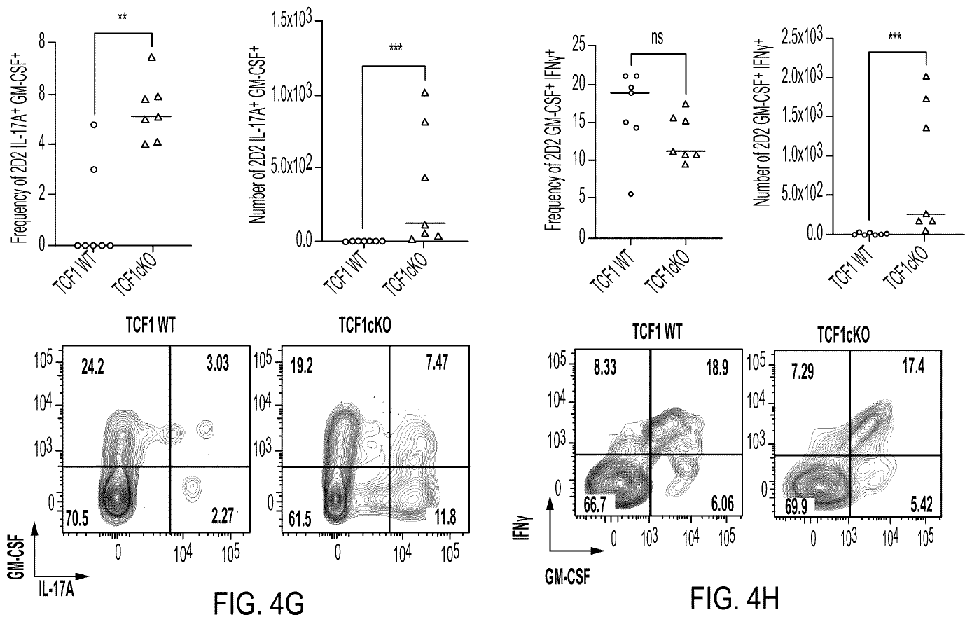

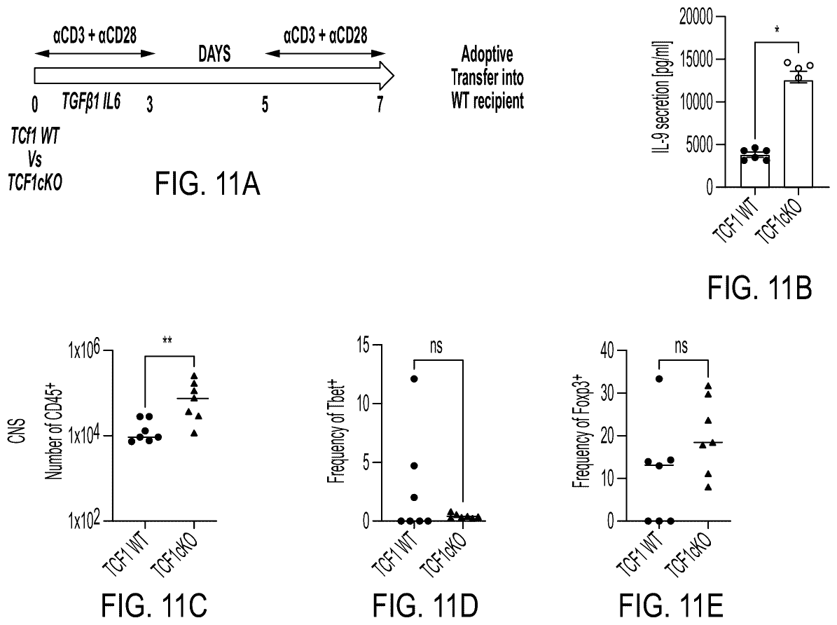

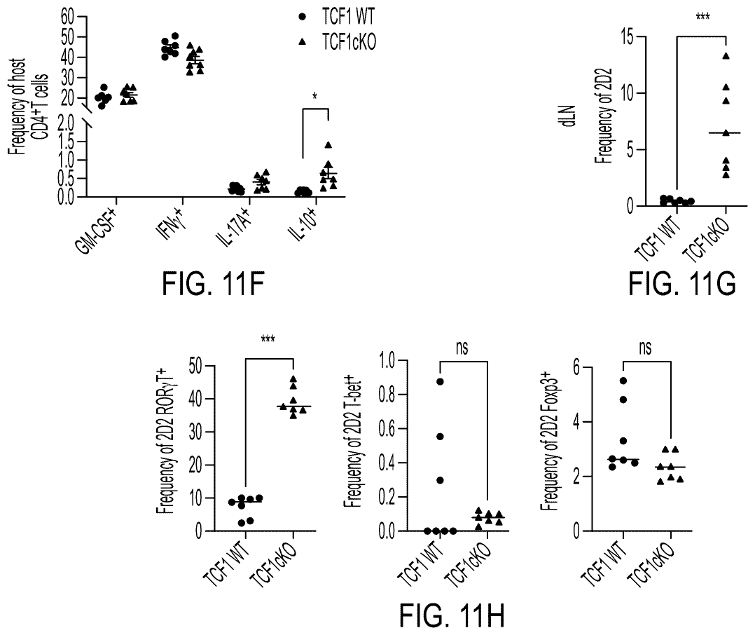

In vitro-differentiated npTh17 cells do not express IL-23R and induce little to no disease upon adoptive transfer. The data (FIG. 1A-1H) suggested that IL-23 may drive pathogenicity in part by down-regulating TCF1 expression. Hence, it was hypothesized herein that the loss of TCF1 could confer pathogenicity to npTh17, which do not express IL-23R. To test this, 2D2 TCF1 WT and TCF1cKO were differentiated into npTh17 and adoptively transferred them into WT recipient mice (FIG. 11A). Indeed, TCF1 deficiency rendered npTh17 2D2 cells able to induce disease, with earlier onset and higher mean maximal disease score (FIG. 4A; FIG. 3A-3G). Histopathological analysis confirmed that recipients of 2D2 TCF1 WT npTh17 displayed limited CNS inflammation, whereas recipients of 2D2 TCF1cKO npTh17 cells had marked leptomeningeal and parenchymal infiltrates, and some atypical inflammation in the spinal nerve root (FIG. 4B, black arrow). Of note, spinal nerve root inflammation is a feature of EAE pathology induced by T helper 9 (Th9) cells. Indeed, TCF1cKO npTh17 cells secreted significantly higher levels of IL9 compared to TCF1 WT npTh17 cells (FIG. 11B). Recipients of 2D2 TCF1cKO npTh17 cells had higher numbers of CD45 and 2D2 infiltrating the CNS (FIG. 4C and FIG. 11C). 2D2 TCF1cKO npTh17 cells were virtually all RORγt with no differences in T-bet expression, further supporting that heightened pathogenicity was not the result of Th 1-like trans-differentiation in the absence of TCF1 (FIG. 4D and FIG. 11D). As TGFB-induced Th17 cells can express Foxp3, its expression was evaluated in 2D2 TCF1 WT and TCF1cKO npTh17 cells but found no differences (FIG. 11E). Interestingly, 2D2 TCF1cKO npTh17cells had significantly decreased proportions of IFNγ- and IL10-producing cells but similar proportions of GM-CSF-producing cells compared to 2D2 TCF1 WT npTh17 cells (FIG. 4E). However, in accordance with their high RORγt expression, 2D2 TCF1cKO npTh17 cells had a significantly higher proportion of IL17A-producing cells (FIG. 4E). In line with the present observations with 2D2 TCF1cKO pTh17 cells (FIG. 2E-2G), 2D2 TCF1cKO npTh17 cells had a higher proportion and number of polyfunctional IL-17A⁺IFNγ⁺ and IL-17A⁺GM-CSF⁺, while the proportion of GM-CSF⁺IFNγ⁺ cells was similar to that observed in 2D2 TCF1 WT (FIG. 4F-4H). As observed in the pTh17 transfer experiments, the effects of TCF1 deficiency were cell-intrinsic as no differences in cytokine production in host CD4⁺ T cells was observed, except for a small increase in IL10-producing cells (FIG. 11F). Given the limited infiltration of 2D2 TCF1 WT npTh17 cells in the CNS of recipient mice, the draining lymph nodes (dLN) of recipient mice were analyzed. 2D2 TCF1cKO npTh17 cells were more abundant than 2D2 TCF1 WT npTh17 in the dLN (FIG. 11G). Mirroring the immunophenotype of CNS-infiltrating 2D2 cells, 2D2 TCF1cKO npTh17 cells in the dLN displayed higher levels of RORγt, but similar T-bet and Foxp3 expression compared to 2D2 TCF1 WT cells (FIG. 11H). Overall, these data indicated that TCF1 deficiency overcame the inability of npTh17 to cause CNS tissue inflammation, and this was associated with higher trafficking to the CNS, RORγt expression, and poly-functional IL-17-producing cells.

### TCF1 Deficiency Obviates the Need for IL-23 for Th17 Pathogenicity

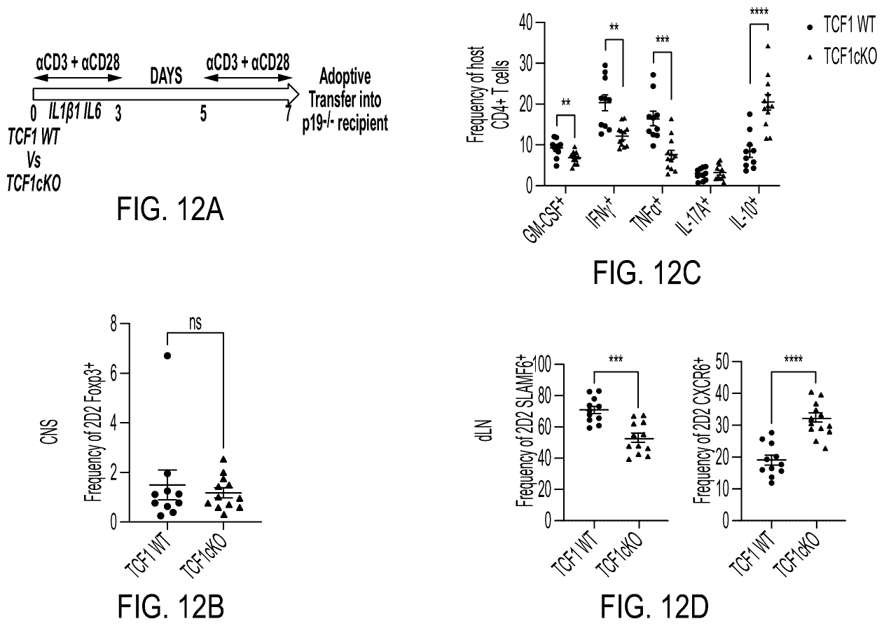

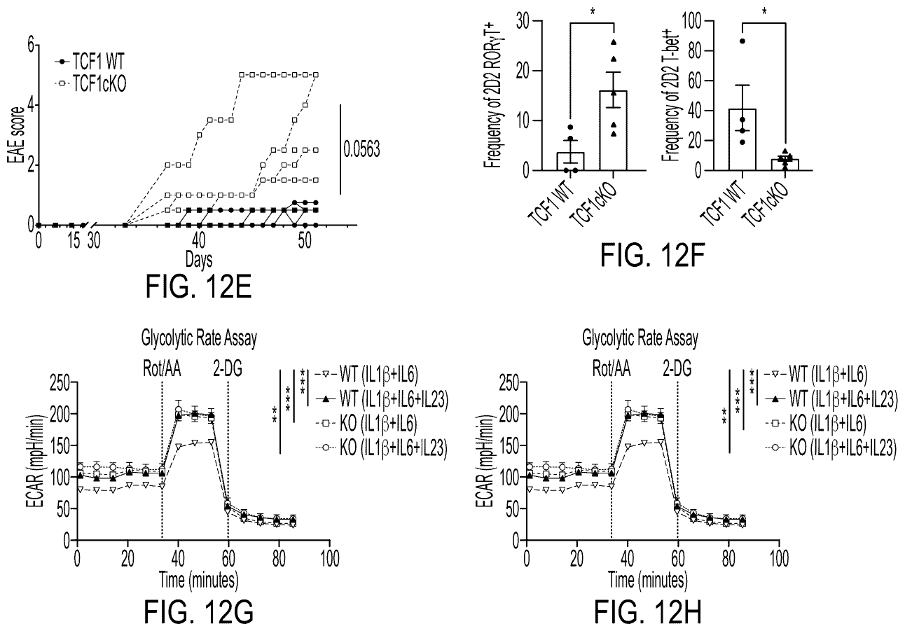

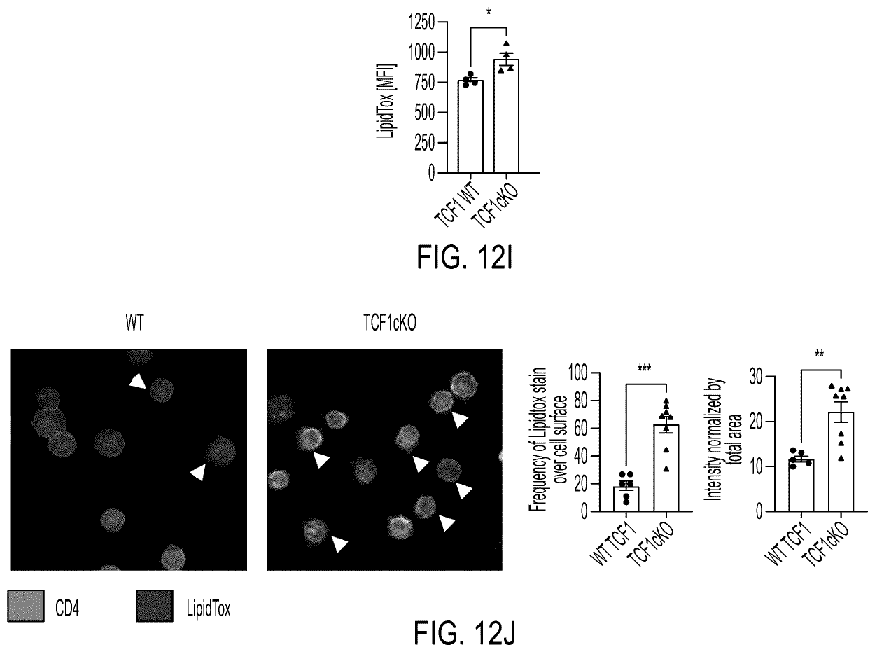

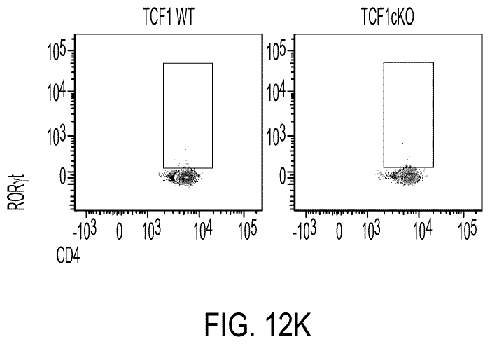

Adoptive transfer data showing that TCF1 deficiency conferred pathogenicity to npTh17 cells upon transfer into WT hosts suggested that TCF1 bypassed the requirement for IL-23 for Th17 pathogenicity (FIG. 4A). However, it was not possible to exclude the possibility that npTh17 cells might have responded to IL-23 produced in vivo in WT recipient mice. To test whether TCF1 deficient cells can induce disease in the complete absence of IL-23, naïve 2D2 TCF1 WT or TCF1cKO cells were differentiated with IL-1β+IL-6 and transferred them into p19 mice (FIG. 12A). Even in the complete absence of IL-23, 2D2 TCF1cKO cells induced more severe EAE, with earlier onset and higher mean maximal disease score (FIG. 5A; FIG. 11A-11H). The present disclosure found no difference in the total number of CD45⁺ cells infiltrating the CNS; however, the number and proportion of 2D2 TCF1cKO cells in the CNS was significantly higher (FIGS. 5B and 5C). Without IL-23, 2D2 TCF1 WT cells mainly differentiated into Th1-like cells expressing T-bet and very little RORγt (FIG. 5D). Conversely, 2D2 TCF1cKO cells differentiated into high RORγt- and low T-bet-expressing cells. No difference was observed in Foxp3 expression (FIG. 12B). In accordance with their T-bet and RORγt expression patterns, 2D2 TCF1 WT cells had higher proportions of IFNγ- and TNFα-producing cells, while 2D2 TCF1cKO cells had higher proportions of IL-17A-producing cells (FIG. 5E). No difference in GM-CSF-producing cells was detected. IL-10-producing cell frequencies were overall quite low but were significantly lower in 2D2 TCF1cKO compared to 2D2 TCF1 WT cells. In line with above disclosed observations, polyfunctional IL-17A³⁰ IFNγ⁺- and IL-17A⁺GM-CSF⁺-producing cells were higher in the 2D2 TCF1cKO group (FIG. 5F-5G). Different from previous observations, the proportion but not the absolute number of GM-CSF⁺IFNγ⁺-producing cells was decreased in 2D2 TCF1cKO compared to 2D2 TCF1 WT (FIG. 5H). Further, differential cytokine production by CNS-infiltrating host CD4⁺ T cells was observed in p19^{-/-} mice that received 2D2 TCF1cKO cells. Host cells produced less GM-CSF, IFNγ, and TNFα, same IL-17A, but significantly higher levels of IL10, the latter suggesting an ongoing effort by host CD4 T cells to dampen overt inflammation (FIG. 12C). These differences in the host CD4⁺ T cell response may have been due to the absence of IL-23. It was suggested that IL-23 is the switch factor that mediates the conversion of npTh17 (SLAMF6⁺) to pTh17 (CXCR6⁺) cells in vivo. The ability of 2D2 TCF1 WT and TCF1cKO cells to convert into pTh17 in the absence of IL-23 was therefore examined. It was found that 2D2 TCF1cKO cells exhibited increased conversion into pathogenic CXCR6⁺ with a concomitant decrease in non-pathogenic SLAMF6⁺ cells compared to 2D2 TCF1 WT cells, further supporting an increased shift towards pTh17 cells in the absence of TCF1 (FIG. 12D). As all of the present experiments employed adoptive transfer of 2D2 cells activated and differentiated in vitro, the disclosure next examined how TCF1 affects the natural differentiation of 2D2 cells in vivo. Naïve 2D2 TCF1 WT or TCF1cKO cells were transferred into RAG1^{-/-} mice. 2D2 TCF1cKO-recipient mice displayed earlier disease onset (p=0.033, FIGS. 12A-12K) and a trend toward heightened disease score (p=0.0563) compared to recipients of 2D2 TCF1 WT cells (FIG. 12E). Despite the variability in disease score, 2D2 TCF1cKO cells consistently expressed high RORγt and low T-bet compared to 2D2 TCF1 WT cells (FIG. 12F), again showing that in the absence of TCF1, cells adopted a pathogenic Th17 phenotype without acquiring Th1-like features.

Th17 pathogenicity is driven by IL-23 and has been associated with increased glycolytic activity and lipid metabolism, which in turn, supply crucial sterol ligands to boost RORγt transcriptional activity. In contrast, homeostatic npTh17 have stem-like properties and have low mTORC1 signaling and glycolysis. Accordingly, the disclosure examined the glycolytic rate of WT and TCF1cKO cells differentiated with IL-1β+IL-6 with or without IL-23. It was found that WT cells showed increased glycolysis (as determined by extracellular acidification rate; ECAR) in the presence of IL-23, consistent with the association of glycolysis with Th17 pathogenicity (FIG. 12G). Notably, TCF1cKO cells had a high ECAR irrespective of the presence of IL-23. To gain further insight into the association between TCF1, IL-23, and glycolysis, cells were stimulated with PMA+Ionomycin prior to the assessment of glycolysis. WT cells differentiated without IL-23 progressively lost their glycolytic activity after PMA+Ionomycin stimulation (FIG. 12H). However, TCF1cKO cells differentiated without IL-23 maintained a high glycolytic rate albeit somewhat lower than cells differentiated with IL-23. The disclosure further assessed the intra-cellular lipid content of 2D2 TCF1 WT and 2D2 TCF1cKO cells isolated from the CNS of p19^{-/-} mice. 2D2 TCF1cKO displayed higher neutral lipid content (FIGS. 12I and 12J). Collectively, these data further supported that IL-23-mediated down-regulation of TCF1 allowed for the full engagement of the bioenergetic program needed to sustain RORγt transcriptional activity and Th17 pathogenicity.

TCF1cKO npTh17 Cells Resemble IL-23-Induced pTh17 Cells

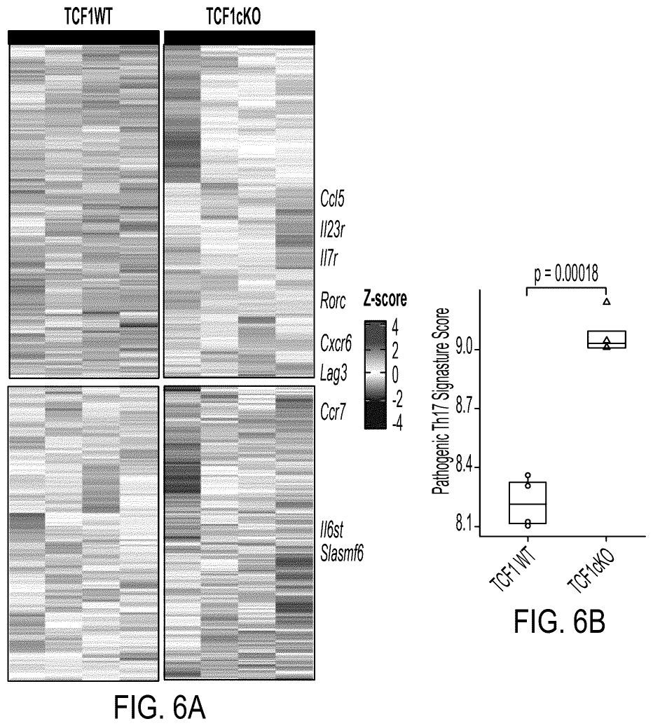

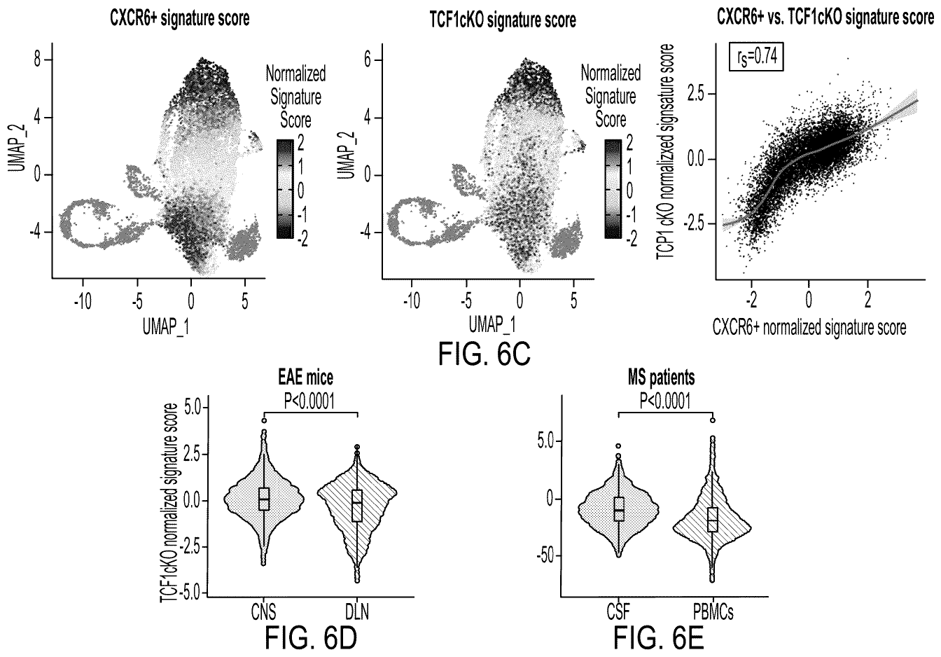

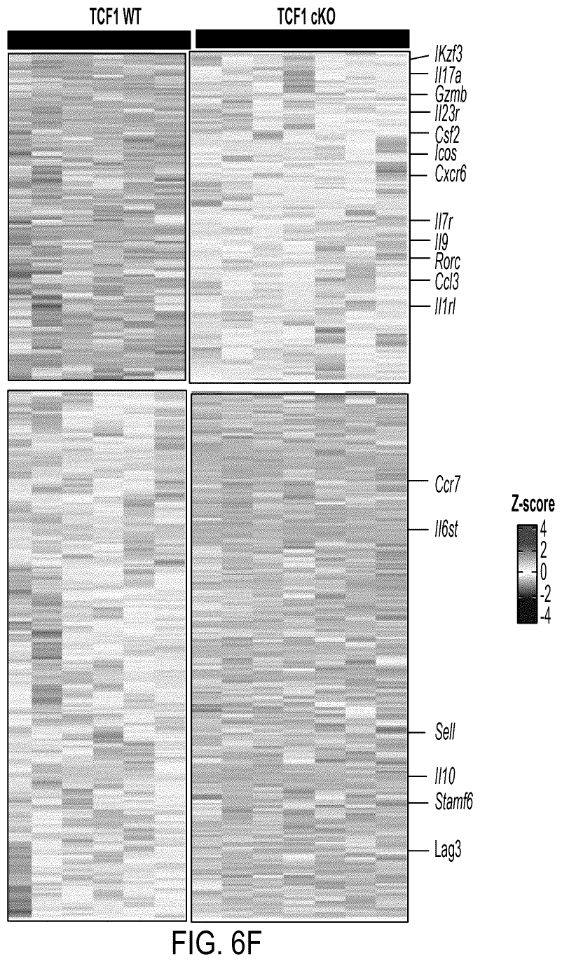

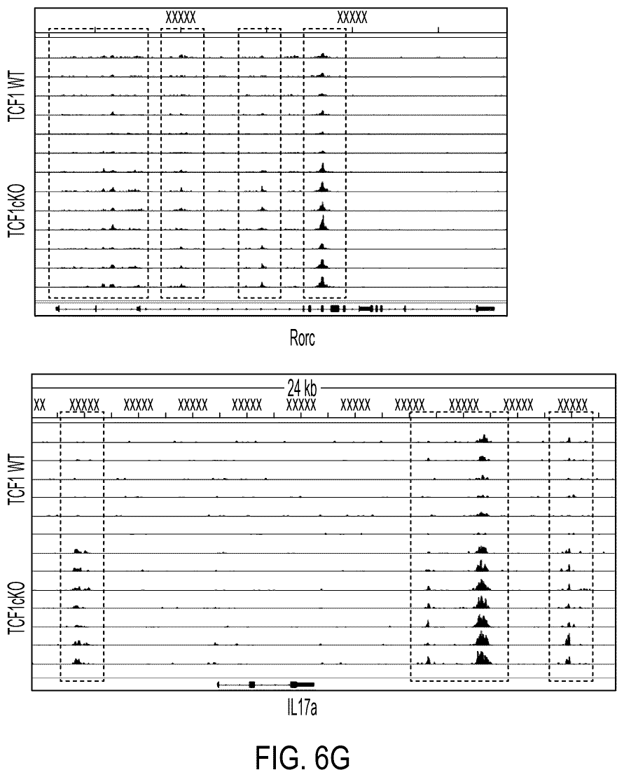

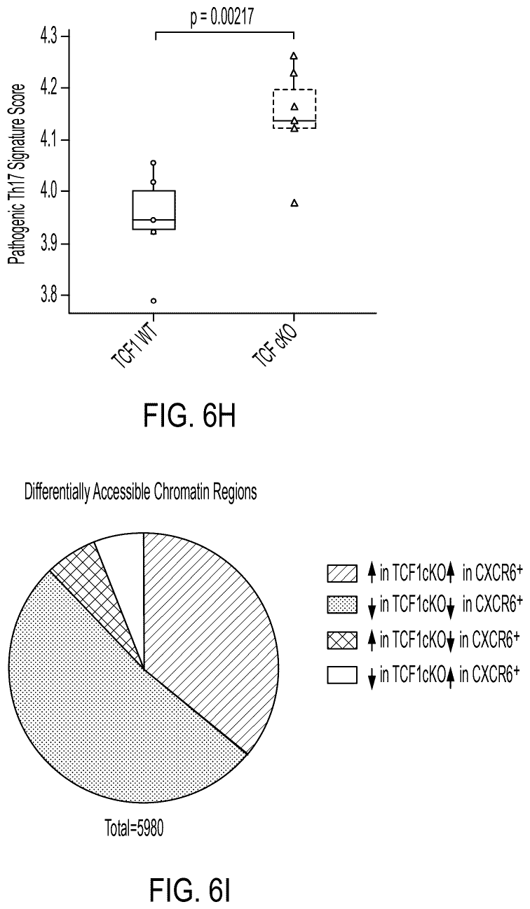

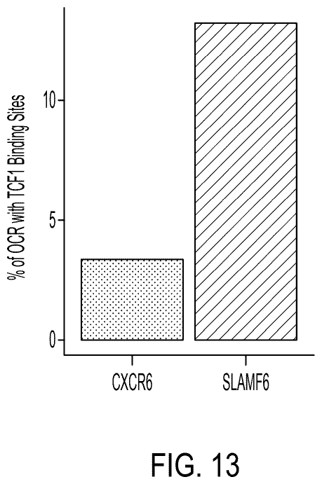

To determine how TCF1 achieves its effects, 2D2 TCF1 WT and TCF1cKO npTh17 cells were transferred into WT mice and then isolated 2D2 T cells from the dLN for analysis by RNA-Seq. The disclosure found 756 differentially expressed (DE) genes between 2D2 TCF1 WT and 2D2 TCF1cKO (400 up-regulated and 356 down-regulated in 2D2 TCF1cKO) (FIG. 6A; FIG. 13). In accordance with RORγt protein expression data (FIG. 11H), 2D2 TCF1cKO cells showed higher Rorc expression (FIG. 6A). To test whether TCF1 restrained the induction of the pathogenic Th17 program, the presence of the pathogenic Th17 signature was scored in 2D2 TCF1 WT and TCF1cKO cells and found that it was significantly higher in TCF1cKO cells (FIG. 6B, methods). It was next tested whether 2D2 TCF1cKO cells resembled pTh17 that differentiate in vivo during active EAE. For this the expression of the 2D2 TCF1cKO npTh17 gene signature was compared to the expression of the CXCR6 Th17 signature in single-cell RNA-seq (scRNA-seq) data from mice with active EAE. A high concordance was found in cells that scored highly for the presence of TCF1cKO and CXCR6 signatures (FIG. 6C, methods). Indeed, the 2D2 TCF1cKO gene signature was highly correlated with the pTh17 CXCR6 gene signature (Spearman correlation coefficient=0.74, p<0.0001). In line with these observations, Cxcr6 was more highly expressed in 2D2 TCF1cKO while Slamf6 was more highly expressed in 2D2 WT npTh17 cells (FIG. 6A). Further confirming that 2D2 TCF1cKO cells shared features with pathogenic cells, the 2D2 TCF1cKO versus TCF1 WT npTh17 gene signature scored significantly higher in CNS-infiltrating Th17 cells compared to Th17 from the dLN of mice with EAE (FIG. 6D). Together these data indicated that 2D2 TCF1cKO npTh17 cells resembled polyclonal T cells that differentiate into pTh17 naturally in vivo upon immunization with MOG. Lastly, it was tested whether 2D2 TCF1cKO npTh17 resembled human pro-inflammatory Th17 cells. For this the disclosure projected 2D2 TCF1cKO signature versus TCF1 WT npTh17 signature onto the scRNA-Seq data from CD4 T cells isolated from the cerebrospinal fluid (CSF) and peripheral blood mononuclear cells (PBMCs) of MS patients. The 2D2 TCF1cKO vs TCF1 WT npTh17 signature scored significantly higher in CD4 T cells from the CSF compared to PBMC (FIG. 6E), indicating that cells poised to enter the CNS of MS patients have reduced TCF1 activity. These data indicated that TCF1 may also play a critical role in restraining the pathogenic potential of human CD4 T cells, thus providing a basis for the association of TCF7 polymorphisms with MS risk.

TCF1 is known to have chromatin remodeling functions. Accordingly, ATAC-Seq was performed to determine whether epigenetic changes contributed to the increased pathogenicity of TCF1cKO cells. 9079 differentially accessible (DA) regions between 2D2 TCF1 WT and TCF1cKO npTh17 cells were found (3633 increased, 5446 decreased in 2D2 TCF1cKO) (FIG. 6F, FIG. 13). The Rorc and Il17a loci showed several regions of increased chromatin accessibility in TCF1cKO cells (FIG. 6G), further corroborating the disclosure's data showing that TCF1 deficiency poises Th17 cells to express more RORγt and produce high levels of IL-17A (FIG. 4D-4E). In line with the above RNA-seq data, the chromatin regions of genes associated with Th17 pathogenicity were significantly more accessible in 2D2 TCF1cKO cells (FIG. 6H). Next, the chromatin accessibility profiles of 2D2 TCF1cKO were compared to CXCR6 Th17 cells. It was found that 2D2 TCF1cKO and CXCR6 cells had 5250 shared DA chromatin regions compared to 2D2 TCF1 WT and SLAMF6 cells (2146 increased; 3104 decreased) with only 730 discordant regions (FIG. 6I). To ascertain whether TCF1 played a direct role in regulating the genes with DA loci, the presence of TCF1 binding motifs in the open chromatin regions (OCRs) was examined, and it was found that TCF1 binding motifs were highly enriched in the OCRs of SLAMF6 compared to CXCR6 cells (13.22% versus 3.37%) (FIG. 13). Collectively, these data indicated that TCF1 performed a major role both transcriptionally and epigenetically in determining the homeostatic versus pathogenic state of Th17 cells.

### a TCF1-Driven Transcriptional Network Determines Th17 Cell State

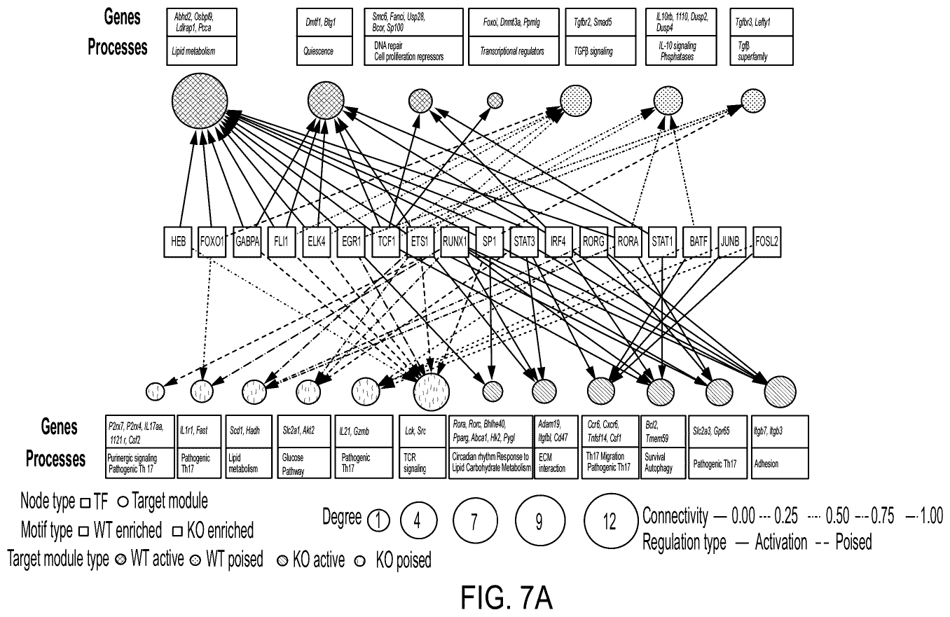

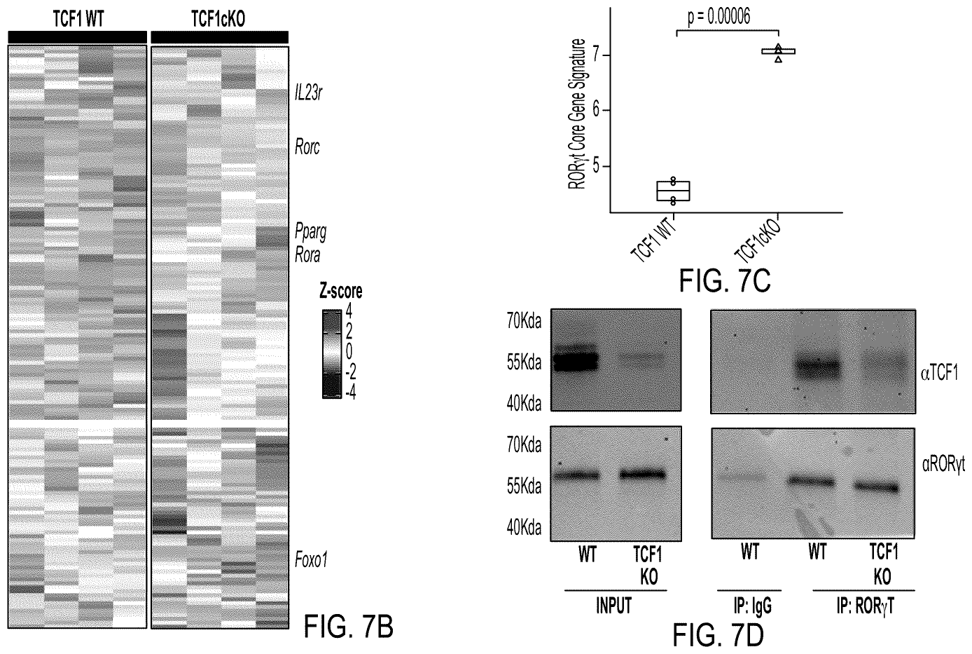

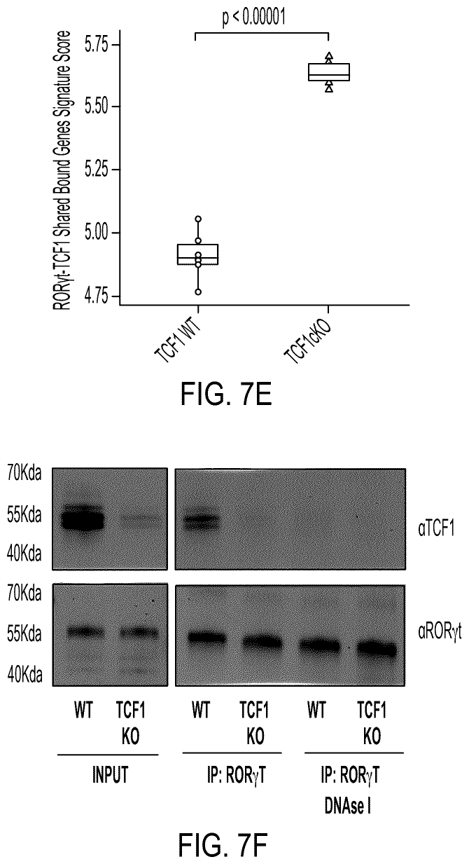

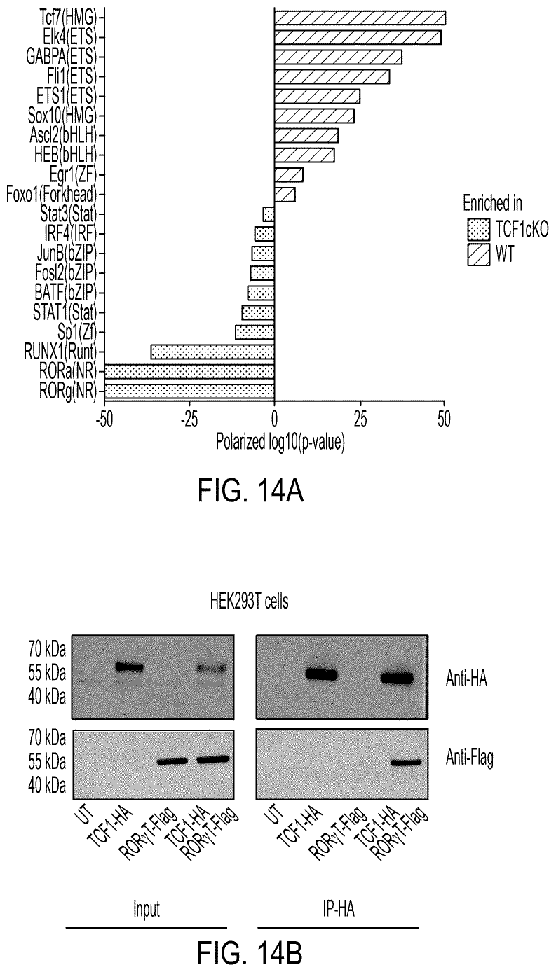

To better understand how TCF1 determined Th17 cell state, ATAC-seq and RNA-seq data comparing 2D2 TCF1 WT and TCF1cKO npTh17 cells was integrated to build a TF: target regulatory network (Methods). The disclosure identified genes that had loci that were DA (FDR<0.05, fold change >1.2) and that were DE (FDR<0.05) between 2D2 TCF1 WT and TCF1cKO cells (active targets) and genes that had loci that were DA but whose mRNA showed no significant differences between 2D2 TCF1 WT and TCF1cKO cells (poised targets). The network was built by linking TFs that were expressed in the data to target genes if the TF binding motifs were found in the OCRs associated with that target gene, resulting in a total of 187 TFs connected to at least one target and 4,545 targets (153 TCF1 WT active, 2,657 TCF1 WT poised, 179 TCF1cKO active, 1,556 TCF1cKO poised) connected to at least one TF (FIG. 14). Target genes were clustered based on the presence of shared TF binding motifs, which allowed for the identification of putative target gene modules. To build a representative network, a TF motif enrichment analysis (HOMER, FIG. 14A-14B, Methods) was run in the DA OCRs between 2D2 WT and TCF1cKO (FDR<0.05) and selected the most significant TFs as well as TFs that were significant and had been shown previously to be important in Th17 cells (FIG. 14A). The network displays TFs whose binding motifs are specifically enriched in WT (boxes at left containing HEB through ETS1, 8 TFs) or TCF1cKO (boxes at right containing RUNX1 through FOSL2, 10 TFs), and the edges depict connections to either active (solid line) or poised (dashed line) gene target modules, for which representative genes and biological processes are displayed (FIG. 7A). The node size represents the number of TFs regulating a target gene module and the edge width represents the proportion of genes in a given module that contain a binding motif for the connected TF.

In accordance with experimental data presented herein, TCF1/Tcf7 (HMG) motifs were the most significantly enriched in 2D2 TCF1 WT along with several member of the ETS TF family (ELK4, GABPA, FLI1, ET8), EGR TF family and FOXO1, all of which have been shown to oppose a pro-inflammatory Th17 program (FIG. 14A). 2D2 TCF1 WT-specific active target modules were enriched for genes involved in processes such as quiescence (Dmtf1, Btg1), DNA repair (Smc6, Fanci, Usp28) and repressors of cell proliferation (Bcor, Sp100), while poised gene modules were enriched for genes belonging to the TGFβ and IL-10 signaling pathways; all features consistent with the engagement of a stem-like and anti-inflammatory phenotype by 2D2 TCF1 WT npTh17. 2D2 TCF1cKO npTh17 showed significant enrichment of RORγt and RORα motifs, followed by SP1, RUNX, and basic leucine zipper domain (bZIP) TF family members (BATF, FOSL2, JUNB). RORγt and RORa are established drivers of Th17 pathogenicity. RORγt and RORa were linked to active targets that included Gpr65 and Slc2a3 (Glut3), both of which have been associated with pathogenic Th17. AP-1 (bZIP) family members, such as JUNB and FOSL2, have been shown to be essential for IL-23-driven pathogenicity and important for limiting Th17 plasticity towards other Th lineages. The AP-1 (bZIP) family members were linked with active genes involved in pathogenic Th17 phenotype and migration (Tnfsf14, Csf1, Ccr6, Cxcr6). Poised gene modules in TCF1cKO were enriched for genes involved in TCR signaling (Lck, Src), sensing of extracellular ATP (P2rx7, P2rx4), along with pro-inflammatory cytokines and effector genes (Il1r1, Gzmb).

As RORγt binding motifs were the most significantly enriched TF motifs in TCF1cKO cells (FIG. 14A) and RORγt was highly expressed in TCF1cKO Th17 cells (FIG. 2C and FIG. 4D), it was next determined which gene expression changes may be directly driven by the increase in RORγt. The DE genes were overlapped (FC>1.5) between 2D2 TCF1 WT and TCF1cKOnpTh17 cells with RORγt-bound genes. Genes that were increased in TCF1cKO cells, bound by RORγt, and important for Th17 cells included Rorc, Rora, and IL23r (FIG. 13). RORγt can also bind and inhibit the expression of target genes that oppose the Th17 program, such as Foxo1, which was decreased in 2D2 TCF1cKO npTh17 cells (FIG. 7A-7B). Indeed, it was found that 2D2 TCF1cKO cells scored higher for a RORγt-driven gene signature compared to 2D2 TCF1 WT cells (FIG. 7C). Together these data indicated increased RORγt activity in the absence of TCF1.

TCF1 has been shown to bind and physically trans-repress other transcription factors such as FOXP3. Given the data showing increased expression of RORγt and RORγt-driven genes in the absence of TCF1, it was next examined whether TCF1 can bind to RORγt and, thus, affect its activity. First, hemagglutinin (HA)-tagged TCF1 protein and Flag-tagged RORγt protein was expressed either alone or in combination in HEK-293T cells followed by immunoprecipitation using HA immunoaffinity beads. It was found that RORγt was immunoprecipitated with anti-HA antibody only in co-transfected cells, indicating that it was interacting with TCF1 (FIG. 14B). To test whether this interaction also occurred in primary Th17 cells, protein lysates were harvested from either WT or TCF1cKO npTh17 cells and performed immunoprecipitation with either IgG control or anti-RORγt antibodies. TCF1 was immunoprecipitated by RORγt but not by the IgG control. A faint TCF1 band was detected in lysates from TCF1cKO Th17 cells, reflecting the 15-20% residual TCF1 expression in TCF1cKO cells (FIGS. 7D and 9A). It has also been suggested that RORγt-bound genes may also be bound by TCF1. Thus, the chromatin accessibility of the previously reported RORγt-TCF1 shared genes (Bcl11b, Bcl2, Il17a, Il17f, Il2ra, Il7r, Pparg, Rara, Rora, Rorc) was queried in 2D2 TCF1 WT versus TCF1cKO npTh17 cells and found that TCF1 deficiency led to a significant increase in chromatin accessibility of these genes (FIG. 7E). It was therefore addressed whether the interaction between RORγt and TCF1 was DNA-mediated. Protein lysates from WT and TCF1cKO Th17 cells were treated with DNAse I prior to performing immunoprecipitation with anti-RORγt antibody. It was found that treatment with DNAse abolished the immunoprecipitation of TCF1 with anti-RORγt antibody, suggesting that the RORγt-TCF1 interaction was mediated through DNA binding (FIG. 7F). Together these data indicated that TCF1 bound RORγt in shared DNA regions, thereby restraining RORγt-driven gene programs.

### DISCUSSION

Despite a critical role for IL-23, the genetic and environmental factors that govern the decision between the homeostatic non-pathogenic and pathogenic Th17 cell states are poorly understood. Here, TCF1 was identified as the major regulator of Th17 cell state. TCF1 supported a transcriptional and epigenetic network driving stem-like and regulatory features and repressed inflammatory features by binding to and restraining RORγt activity. Importantly, the absence of TCF1 precluded the requirement for IL-23, underscoring that suppression of TCF1 is the major determinant of Th17 pathogenicity.

TCF1 deficiency significantly increased the pathogenic potential of myelin-specific pTh17 cells and conferred pathogenicity to npTh17 cells. In both cases, heightened pathogenicity was associated with high RORγt and IL-17A expression, and the emergence of polyfunctional IL-17A cells producing GM-CSF and IFNγ. While previous studies proposed that trans-differentiation into Th1-like cells was necessary to sustain neuroinflammation, TCF1cKO cells did not express higher levels of T-bet or Th1-related cytokines ex vivo. Even in the complete absence of IL-23 signaling, TCF1cKO cells drove severe CNS autoimmunity and exhibited high RORγt and IL-17A expression. Indeed, in this setting T-bet, IFNγ, and TNFα expression was significantly reduced, further highlighting that pathogenicity was not the result of heightened trans-differentiation into Th1-like cells. These findings are in line with human studies showing that frequency of Th17, but not Th1 cells, in the CSF correlated with disease activity in both relapse-remitting MS (RRMS) patients and in early MS episodes. Moreover, a recent study following discordant monozygotic twin pairs for MS showed that IL-17A was the most significantly increased cytokine in T helper cells upon re-activation ex vivo in twins with MS, further supporting the association between Th17 responses and MS etiopathology. GM-CSF (encoded by (sf2) has also been shown to play a critical role in neuroinflammation. Although, TCF1cKO cells did not produce more GM-CSF, they exhibited increased proportions of polyfunctional IL-17A GM-CSF cells. It was further observed that the Csf2 locus was more open in TCF1cKO cells, indicating that Th17 cells lacking TCF1 are poised to readily produce more GM-CSF and induce heightened immunopathology.

The data herein showed that TCF1 physically interacts with RORγt in a DNA-dependent manner and that pro-inflammatory genes are increased in TCF1cKO Th17 cells and is in line with the recent demonstration that TCF1 and FOXP3 co-bind in accessible genomic regions enriched for pro-inflammatory Th17 genes in Treg cells and that TCF1 deficiency in Treg results in increased expression of these genes. Thus, TCF1 co-binding with lineage determining TFs, such as FOXP3 and RORγt, and the associated TCF1-inhibition of a pro-inflammatory Th17 program may represent a general mechanism operative in different CD4⁺ T cell subsets. Such a mechanism would explain how the differential expression of TCF1 in npTh17 and pTh17 determines their pathogenic potential despite both expressing RORγt. Further, the heightened glycolysis and intracellular lipid content that was observed in TCF1cKO cells can provide increased lipid ligands that can feed RORγt transcriptional activity and boost pathogenicity.

TCF1 expression has been associated with stemness in T cells. This role of TCF1 has been heavily studied in CD8⁺ T cells, where it maintains a long-lived pool of cells that are crucial for anti-tumor immunity and control of viral infections. Similarly, it was found that TCF1 marks an intestinal stem-like Th17 population that provides a ready reservoir for differentiation into pathogenic effectors. The findings presented herein support a role for TCF1 in maintaining such a population. Further, the TF regulatory network identified herein suggests a co-operative role for TCF1 and ETS TF family members in sustaining the homeostatic and stem-like phenotype of Th17 cells. Indeed, a recent study in CD8 T cells that investigated direct and indirect genome-wide binding of TCF1 found that genomic regions indirectly bound by TCF1 were highly enriched for ETS family binding motifs. Importantly, despite sharing a highly conserved ETS domain and consensus motif, distinct ETS family members can bind specific enhancers which may dictate context-dependent responses during T cell activation. Thus, specific ETS family members may co-operate with TCF1 at specific genomic enhancers to safeguard the npTh17 cell state by restraining the engagement of a Th17 pro-inflammatory effector phenotype. Further investigation into this TCF1/ETS co-operativity in preserving the non-pathogenic/homeostatic Th17 cell state is warranted to design therapeutic means aimed at targeting pro-inflammatory Th17 features while sparing homeostatic functions.

It is important to note that the difference in TCF1 expression between non-pathogenic homeostatic and pathogenic Th17 cells both in vitro and in vivo is not all or none. Accordingly, the data herein indicates that the level of TCF1 expression dictates the Th17 cell fate decision. This has important clinical implications. Myelin-reactive T cells are readily found in the peripheral blood of healthy subjects within the memory CCR6′ pool and, therefore, exhibit features of previously activated T cells. Alterations in the TCF1 expression level may license these quiescent myelin-reactive Th17 memory cells to engage a pro-inflammatory program and kickstart autoimmune pathology in the presence of permissive genetic and environmental factors. Indeed, SNPs in the coding and non-coding regions of the TCF7 locus have been associated with increased risk of MS, RA, and TID. It is therefore tempting to speculate that these SNPs may affect the binding of regulators of TCF7 transcription, TCF7 mRNA stability, or TCF1 function, thereby resulting in decreased TCF1 expression and/or function and increased predisposition to autoimmunity.

Lastly, the data herein indicate that a major mechanism by which IL-23 drives Th17 pathogenicity is by suppressing TCF1. In this regard, TCF1 and Blimp1 (encoded by Prdm1) are known to counter-regulate each other in Th1 and Tfh cells and IL-23 has been reported to promote Th17 pathogenicity by inducing the expression of Blimp1. However, heightened Prdm1 expression was not observed in the RNA-Seq data of TCF1cKO cells and the Prdm1 locus was more closed in TCF1cKO cells, suggesting that Blimp1 was not responsible for the heightened Th17 pathogenicity observed, at least in the context of the current disclosure. It has also been proposed that IL-23 regulates the migratory properties of Th17 cells; however, the underlying mechanisms were unknown. The regulatory network data identified herein indicate that loss of TCF1 leads to the activation of AP-1, SP1, and STAT3, TFs that control the expression of multiple chemokine receptors, integrins, cell adhesion receptors, and metalloproteinases, thus providing a mechanism by which IL-23 drives accumulation of cells in tissue.

In summary, the present disclosure identified TCF1 as the key regulator of Th17 cell state, maintaining homeostatic state through a network including known, as well as important, TFs while concomitantly repressing pathogenic state by binding to and interfering with RORγt pro-inflammatory activity. The present findings provide a framework for understanding how TCF7 locus polymorphisms associate with susceptibility to MS and other Th17-driven autoimmune diseases in humans. Further, the identified regulatory network will serve as a roadmap for future investigation into regulators and targets to modify Th17 cell state therapeutically.

### Methods

### Experimental Model and Subject Details

6-8-week-old male or female C57BL/6J (WT), C57BL/6-Il17 atm 1Bcgen/J (Il17a^{gfp}), C57BL/6-Tg (Tcra2D2, Tcrb2D2) 1Kuch/J (2d2), B6.Cg-Tg (Lck-icre)3779Nik/J (dLck-Cre), B6.12914-Rag1tm1Mom/J (RAGI KO) were purchased from the Jackson laboratories. Embryonic stem cells with a targeted Tcf7 gene (Tef7flox) were obtained from Eucomm and generated as previously described. For the generation of CD2-Tcf7L-Tg mice, the full-length cDNA of Tef7L isoform was cloned into the human CD2 expression cassette and the construct microinjected directly into C57BL/6 oocytes. IL-23r-gfp, and p19KO mice were provided by Dr. Vijay K. Kuchroo. Tcf7-gfp reporter mice were provided by Dr. Hai-Hui Xue. Mice were housed under specific-pathogen-free conditions (SPF) at the Mass General Brigham mouse facility in Boston. All experiments that involved the use of mice were performed in accordance with the guidelines outlined by the Institutional Animal Care and Use Committee (IACUC) in Boston.

### Th17 In Vitro Experiments

Single-cell suspension were prepared from spleen and lymph nodes, and CD4 T cells isolated using the anti-CD4 microbeads (Miltenyi, catalogue #130-117-043). Subsequently, naïve CD4 T cells (CD62L⁺ CD44⁻ CD25⁻) were enriched through fluorescence activated cell sorting (FACS) performed on a BD FACSAria Ilu. Naïve CD4 T cells were activated for 3 days with 1 ug/ml of plate-bound anti-CD3 (BioXcell, BP0001) and anti-CD28 (BioXcell, BE0015), plus the addition of 20 ng/ml of IL-1B (R&D Systems), 25 ng/ml of IL-6 (R&D Systems), 20 ng/ml of IL-23 (R&D Systems), 20 ug/ml of anti-IFNγ (BioXcell, BE0055) and 20 μg/ml of anti-IL4 (BioXcell, BE0045) for pathogenic Th17; 2 ng/ml of TGFβ1, 25 ng/ml of IL-6, 20 μg/ml of anti-IFN and 20 μg/ml of anti-IL4 for non-pathogenic Th17. Cells were then rested in un-coated plates in absence of anti-CD3/anti-CD28 stimulation for 2 days. For some experiments, cells were re-stimulated with 1 ug/ml of anti-CD3/anti-CD28 for 1-2 days.

### Flow Cytometry and Intracellular Staining

Single-cell suspensions were firstly stained to discriminate live cells with UV Zombie (Biolegend) in PBS, and then in FACS buffer (2% bovine serum albumin in PBS) with antibodies for surface antigens. The following surface antibodies directly conjugated with different fluorochrome labels were used: TCR Vα3.2 (clone RR3-16), TCRVB11 (clone KT11), CD4 (RM4-5), CD45 (clone 30-F11), SLAMF6 (clone 330-AJ), CXCR6 (clone SA051D1), all purchased from Biolegend. For nuclear protein staining, cells were fixed with the eBioscience Foxp3/Transcription Factor Staining Buffer Set (Catalogue #00-5523-00) following manufacturer's instruction. The following antibodies for nuclear proteins were used: TCF1 (clone C63D9) from Cell Signaling Technology, RORγt (clone Q31-378) from BD biosciences, T-bet (clone 4B10) from Biolegend and Foxp3 (clone FJK-16s) from ThermoFisher Scientific. For cytokine staining, cells were re-stimulated with 50 ng/ml of Phorbol myristate acetate (PMA) and 1 μg/ml of ionomycin from Sigma, plus 1:1000 dilution of Golgiplug (brefeldin A) and Golgistop (monensin) from BD Biosciences, for 4 hours. After surface staining, cells were fixed with 4% PFA for 20 minutes at 4 degrees, and then stained in Saponin buffer for 20 minutes at 4 degrees with the following antibodies from Biolegend: GM-CSF (clone MP1-22E9), IFNγ (XMG1.2), IL-17A (clone TC11-18H10.1), IL-10 (clone JE12-16E3), IL-17F (clone 9D3.1C8) and TNFα (clone MP6-XT22). Fully stained samples were read either at a BD LSRFortessa or BD FACSymphony. The data were analyzed with FlowJo software (TreeStar) and GraphPad Prism software (GraphPad).

### Gene Expression Assay

In vitro polarized cells were harvested and lysed in RLT buffer plus 1% 2-Mercaptoethanol and RNA extracted with the RNeasy Plus Micro Kit (Qiagen) following manufacturer's instructions. For gene expression assay, the following Taqman gene expression assays were used: Tcf7 (Mm00493445_m1), Axin2 (Mm00443610_m1), IL-23r (Mm00519943_ml), Lef1 (Mm00550265_m1), Actin (Mm02619580_g1). Target gene (FAM-MGB) and housekeeping gene (Actin VIC-MGB) were multiplexed in the same well. Target gene expression was calculated as delta CT compared to housekeeping.

### Passive Experimental Autoimmune Encephalomyelitis (EAE)

Naïve CD4 T cells were harvested from Tg(Tera2D2, Tcrb2D2)1Kuch/J (2d2) mice and differentiated in Th17 cells as outlined. At the end of the in vitro differentiation, 1-4×10 6 cells were adoptively transferred into WT, p19^{-/-} or RAG1^{-/-} recipient mice by tail vein injection. Mice were followed for symptoms development and daily scored for both typical and atypical symptoms as follows: Score 0, healthy; Score 1, limp tail; Score 2, limp tail and weakness of hind legs or obvious sign of head tilting and poor balance; Score 2.5, dragging feet or no movement in one leg only or strong head tilt that causes the mouse to sporadically fall over; Score 3, hind legs paralysis or walking only along the edges of the cage or spinning when picked up by the base of the tail; Score 3.5, paralysis of hind legs which lie only on one side of the body or hind quarter of the body completely flat; Score 4, complete hid leg paralysis and partial front leg paralysis which allow minimal movement around the cage; Score 5, spontaneous rolling in the cage, or severe paralysis that doesn't allow any movement.

### Induction of Active Experimental Autoimmune Encephalomyelitis (EAE)

For active induction of EAE, mice were immunized subcutaneously with the myelin oligodendrocyte glycoprotein (MOG) peptide MOG₃₅₋₅₅ (50 μg/mouse per flank, Genemed Synthesis), emulsified in complete Freund's adjuvant (CFA) and Mycobacterium tuberculosis H37Ra extract (Becton Dickinson). Pertussis toxin (100 ng/mouse, List Biological Laboratories) was administered intravenously into the tail vein on day 0 and day 2 post immunization.

Immune Cells Isolation from Central Nervous System

Mice were euthanized and perfused with ice-cold PBS. Brain and spinal cord were harvested and digested in serum-free DMEM (Gibco) plus 2.5 mg/ml of Collagenase D (Roche) and 20 ug/ml of DNAse I (Sigma #10104159001), on a rotator shake for 30 minutes at 37 degrees. Myelin removal and immune cells enrichment was performed via Percoll (Amersham Biosciences) gradient (37% and 70%). The middle ring of cells was collected and used for downstream applications.

### Central Nervous System Histopathology

Mice were euthanized 21-28 days post-adoptive transfer. Brain, spinal cord and eyes were fixed in neutral-buffered formalin and processed for paraffin embedment and routine H&E staining. Inflammatory foci were quantified in the parenchyma and meninges by an experienced pathologist who did not know either the score of the mice or the experimental group.

### Bead-Based Immunoassay

Cytokines concentration in the culture supernatant were measured using the LegendPlex mouse T Helper Cytokine Panel (Biolegend) following the manufacturer's instruction. Data were acquired with a BD LSRFortessa flow cytometer and analyzed using the online Legendplex data analysis software.

### Transfection

0.8×10⁶ HEK-293T cells were seeded in 6-well plates in complete IMDM (10% FCS, 2 mM Glutamine, Penicillin/Streptomycin). After 24 hours, cells were transfected using Polyjet transfection reagent following manufacturer's instruction (Signagen, #SL100688). In each well, a total of 1.5 ug plasmid DNA was transfected. The following plasmids were used: pcDNA3.1 Tcf7-HA (0.5 μg/well), pcDNA3.1 N-HA (0.5 μg/well), pcDNA3.1 Rorc-FLAG (1 μg/well), pcDNA3.1 Empty-FLAG (1 μg/well). All plasmids were purchased from Genscript.

### Protein Co-Immunoprecipitation

24 hours post-transfection, HEK-293T cells were harvested and processed following Pierce™ HA-Tag IP/Co-IP Kit (Thermo Scientific) manufacturer's instruction. In brief, 250 ug of protein lysates were incubated with 20 μL of anti-HA agarose slurry (=35 ug of antibody) on a rotator shake, overnight at 4 degrees Celsius. After several washing steps, bound proteins were eluted in Non-Reducing Sample Buffer and prepared for reducing SDS-PAGE.

Naïve CD4 T cells were differentiated into Th17 cells and processed following Pierce™ Crosslink Magnetic IP/Co-IP Kit manufacturer's instructions. Magnetic beads were cross-linked with 2 ug of RORγt Monoclonal Antibody (AFKJS-9, eBioscience), and incubated with 500 ug of protein lysate for one hour on a rotator shake at room temperature. In some experiments, protein lysates were incubated for 15 minutes at room temperature with 50 ug/ml of DNAse I (Sigma #10104159001).

### Western Blot

Protein lysates were prepared in sample buffer plus 2 mM DTT. Samples were loaded and ran onto precasted gels (Mini-PROTEAN TGX, Biorad). Thereafter, gels were transferred on nitrocellulose membranes (Biorad) using the Trans-Blot Turbo system apparatus (Biorad). Membranes were blocked in TBS-T supplemented with 5% skim milk for 1 hour at RT on a rocking platform. After blocking, membranes were incubated overnight with the following antibodies: HA-Tag (C29F4) Rabbit mAB (CST, #3724), DYKDDDDK Tag (D6W5B) Rabbit mAb (CST, #14793), TCF1/TCF7 (C63D9) Rabbit mAb (CST, #2203), RORγt Monoclonal Antibody (AFKJS-9) (eBioscience, #14-6988-82). Diluent and dilutions were determined following manufacturer's instructions. Following day, membranes were incubated with Goat anti-Rabbit IgG (H+L) Secondary Antibody, HRP (Thermo Fisher, #65-6120) or Anti-rat IgG, HRP-linked Antibody (CST, #7077), for 1 hour at room temperature on a rocking platform. Images were acquired with the iBrightCL 1000 system (Invitrogen).

### Preprocessing and Analysis of Bulk RNA-Seg Data

The preprocessing was performed on Google Cloud via Terra scientific computing (app.terra.bio/). The BCL files were converted to FASTQ format using bcl2fastq (v2.20.0.422) (support.illumina.com/downloads/bcl2fastq-conversion-software-v2-20.html). Raw reads were aligned to mouse genome mm10 using STAR (v2.7.5a), and Picard MarkDuplicates (broadinstitute.github.io/picard/) was used to identify the duplicate reads. RNA-SeQC 2 was used for assessing sequencing depth and mapping quality. Raw counts and TPMs were computed using RSEM. Outliers were determined by principal component analysis. Downstream analyses were conducted in R (v4.1.3). Lowly expressed genes with a total count lower than 10 were prefiltered, then DESeq2 (v1.32.0) was applied on the raw counts for the identification of differentially expressed (DE) genes. Genes were determined as differentially expressed if their Benjamini-Hochberg (BH) adjusted p-value <0.05. Heatmaps were generated using R package ComplexHeatmap (v2.8.0) on TPMs and were scaled by row. The bulk RNA-seq signature scores were computed as the average of DESeq2 normalized counts of the genes in the signature. T-tests were performed on the original values of the signature scores, while the boxplots display log 2-transformed values for visual clarity. Boxplots were generated using the ggplot2 package (v3.3.5).

### Preprocessing and Analysis of Bulk ATAC-Seg Data

The BCL files were demultiplexed using bcl2fastq (v2.20.0.422) (support.illumina.com/downloads/bcl2fastq-conversion-software-v2-20.html). Next, ENCODE ATAC-seq pipeline (v1.5.4) (github.com/ENCODE-DCC/atac-seq-pipeline) (github.com/ENCODE-DCC/atac-seq-pipeline) was used for read alignment, filtering, peak calling, and quality control. Briefly, reads trimmed by cutadapt were aligned to the mm 10 genome with Bowtie2, then filtered for duplicates using Picard MarkDuplicates. After removing the mitochondrial reads, peaks were called using MAC9 with p-value threshold 0.01, smoothing window size 73, and IDR threshold 0.05. The top 100,000 most significant peaks from each replicate were merged into a set of union peaks using bedtools merge (v2.26.0). Read counts per peak were computed using featureCounts function in the R package Rsubread (v2.6.4). Peaks wider than 250 bp were selected for downstream analysis. Differentially accessible (DA) peaks were identified with R package DESeq2 (v1.32.0), applied to the raw counts and assessed as those peaks with Benjamini-Hochberg adjusted p-value <0.05. DA peaks were then annotated with their nearest genes using ChIPseeker (v1.28.3) (tssRegion=c(−3000, 3000), overlap=“all”). Genomic segments (intron, exon, 3′UTR, distal, intergenic, promoter) were annotated using TxDb.Mmusculus.UCSC.mm10.knownGene (v3.10.0) and org.Mm.eg.db (v3.13.0) was used for mapping between gene and transcript annotations. If a gene was associated with multiple peaks, it was assigned to the peak that has the lowest adjusted p-value. Heatmaps were created on the DESeq2 normalized counts scaled by genes across samples. Boxplots were generated using ggplot2 (v3.3.5). The bulk ATAC-seq signature scores were defined as the average of DESeq2 normalized counts of the genes in the signature. T-tests p-values were calculated on the original values from the scores, while the boxplots display log 2-transformed values.

### Single-Cell RNA-Seq Data Analyses

Processed expression data and UMAP coordinates for Th17 cells in the spleen, CNS and draining lymph nodes during EAE were obtained from (Schnell et al., 2021, Cell 184, 6281-6298).

scRNA-seq data for PBMC and CSF immune cells from Multiple Sclerosis (MS) patients were downloaded from GEO (GSE138266). Only cells where >400 genes were detected and ≤5% of total UMIs were mitochondrial RNA were retained. PBMC and CSF samples were separated and integrated samples in each group using Seurat v3 SCTransfrom integration workflow followed by dimension reduction and clustering. SelectIntegrationFeatures function was however applied to the combined data to ensure the same set genes will be integrated, and the number of features was set to infinity to allow as many variable features as possible (yielded 7,173 genes). FindNeighbors was applied to the top 15 principle components and the resolution was set to 0.5 in FindClusters. Default parameters were used otherwise. Clusters were annotated by examining the expression of top cell type specific genes (differential expression results were provided in Schafflick et al., 2020, Nat Commun 11, 247). The CD4-like clusters were selected and the dimension reduction was repeated, clustering and annotation procedure on cells in these clusters using the same parameters, and again selected the CD4-like cells in the new clustering. Integration of PBMCs and CSF CD4-like cells were then applied to the “integrated” assay in respective Seurat objects, using all gene in the assays to integrate and the log normalization in FindIntegrationAnchors.

Per cell signature score was defined as the difference between the total expression of positive genes (up-regulated in the population of interest) and that of negative genes (down-regulated in the population of interest). Z-normalization (mean centered to 0 and variance scaled to 1) was then applied across cells to transform the scores into a reasonable range.

### Motif Enrichment Analysis

Motif enrichment analysis was performed using HOMER v4.11 using the command findMotifsGenome. In the TCF1 WT versus TCF1cKO analysis, differentially accessible peaks (FDR<0.05) in one genotype (regarded as regions of interest) were compared to the other (regarded as the background). Masked mm10 genome was used, and de novo motif enrichment was turned off. Default parameters were used otherwise.

### TF: Target Network Analysis

A transcription factor to target gene (TF: target) regulatory network in 2D2 TCF1 WT versus TCF1cKO npTh17 was built by integrating of RNA-seq and ATAC-seq data. First, target genes were identified from differential accessibility (DA) and differential expression (DE) analysis comparing WT and KO samples. Peaks were assigned to genes as described above; if multiple peaks were assigned to the same gene, only the one with lowest p-value was kept. Genes that were associated with DA peaks but not expressed or whose DE FDR was not available due to low expression or presence of outliers were labeled as not DE. Targets were further classified as follows: genes associated with peaks that were more accessible (FDR<0.05, fold change >1.2) and whose expression was upregulated (FDR<0.05) in the same genotype were defined as active targets (153 in WT, 180 in KO), whereas genes associated with DA peaks but that were not DE were defined as poised targets (2267 in WT, 1277 in KO). Genes associated with more open peaks but that had lower expression in the same genotype were analyzed but not included in the final network due to the small number of genes in this category (47 open but downregulated in WT, 20 in KO) and a lack of clear biological interpretation. Second, candidate TFs were imported from the HOCOMOCO v11 database (mouse core collection) and filtered by expression (only keeping those with average expression >=1TPM). Next, links between TFs and targets were identified using FIMO (the runFimo function in the memes package) with default parameters except parse_genomic_coord was set to FALSE to obtain the relative coordinates of the motif hits in the input sequences. This step built a network containing a total of 187 TFs connected to at least one target and 4,545 targets (153 WT active, 2,657 WT poised, 179 KO active, 1,556 KO poised) in total. Adjacency matrices were built for each target category such that columns represented all TFs, rows represented targets in that category, values denoted the presence (1) or absence (0) of each TF's motif binding cites in each target. Targets within each category were then clustered into putative gene modules based on the presence of shared TF binding motifs. Hierarchical clustering (Euclidean distance and Wald agglomeration method) was applied to the adjacency matrices and clusters were identified using the adaptive branch pruning algorithm (cutreeDynamic function from the dynamicTreeCut package minimum cluster size was set to 3, default parameters were used otherwise).

To build a representative network, targets were summarized into modules and selected TFs that were most significantly enriched in 2D2 TCF1 WT or TCF1cKO (top 20 ranked by p-value) or were significantly enriched in one genotype and were known to be important in Th17 cells (EGR1, p=1e-8; FOXO1, p=1e-5; STAT3, p=1e-3). Directed edges from TFs to targets were weighted by connectivity, which was defined as the proportion of genes in a target module whose associated DA peaks contained at least one motif binding site for the TF pointing to it. The network was further pruned to only include edges with absolutely or relatively high connectivity, where absolutely high was defined as connectivity >=0.3, and relatively high was determined as a significantly higher connectivity from the TF to the target module of interest than the average connectivity from the same TF to the rest of targets in the same target category (p<0.05). A chi-square test was applied to test the significance in the differences (module-size-weighted average connectivity in the rest of modules is equivalent to the proportion of targets not belonging to the module of interest containing at least one TF motif binding site in associated DA peaks). The degree of target module nodes was computed after edge pruning and was defined as the number of TFs pointing to a node.

### Quantification and Statistical Analyses

Significant differences between two experimental groups were analyzed using GraphPad Prism 9 using unpaired two-tailed Mann-Whitney test or in case of multiple groups Kruskal-Wallis one-way ANOVA followed by Sidak post-hoc test. EAE disease curves were analyzed using linear mixed effects models to test the trajectory of disease between various genotypes over time. All the details about the statistical tests used and the exact value of n for each panel can be found in the figure legends. Statistical analyses for computational methods are described in the corresponding methods section. Values of *p<0.05, **p<0.01, ***p<0.001 and ****p<0.0001 were considered statistically significant and displayed in the figures.

For each experiment, mice were randomly assigned to each group. EAE cohorts consisted of a number of mice/group, which allowed for detection of a statistically significant (p<0.05) difference in disease course with at least 90% power, and were repeated at least twice to ensure reproducibility.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method of treating an autoimmune disease in a subject, comprising contacting a Th17 cell or a plurality of Th17 cells with an exogenous T cell modulating agent in an amount sufficient to: decrease expression of at least one pro-inflammatory Th17 transcription factor, increase expression of at least one anti-inflammatory Th17 transcription factor, or a combination thereof, and administering to the subject the Th17 cell or the plurality of Th17 cells, thereby treating the autoimmune disease.

2. The method of claim 1, wherein the autoimmune disease is selected from the group consisting of: acute disseminated encephalomyelitis (ADEM), Addison's disease, ankylosing spondylitis, antiphospholipid antibody syndrome (APS), aplastic anemia, autoimmune gastritis, autoimmune hepatitis, autoimmune thrombocytopenia, Behçet's disease, coeliac disease, dermatomyositis, diabetes mellitus type I, Goodpasture's syndrome, Graves' disease, Guillain-Barré syndrome (GBS), Hashimoto's disease, idiopathic thrombocytopenia purpura, inflammatory bowel disease (IBD) including Crohn's disease and ulcerative colitis, mixed connective tissue disease, multiple sclerosis (MS), myasthenia gravis, opsoclonus myoclonus syndrome (OMS), optic neuritis, Ord's thyroiditis, pemphigus, pernicious anemia, polyarteritis nodosa, polymyositis, primary biliary cirrhosis, primary myxedema, psoriasis, rheumatic fever, rheumatoid arthritis, Reiter's syndrome, scleroderma, Sjögren's syndrome, systemic lupus erythematosus, Takayasu's arteritis, temporal arteritis, vitiligo, warm autoimmune hemolytic anemia, Wegener's granulomatosis, asthma, allergy, allergic rhinitis, allergic airway inflammation, atopic dermatitis (AD), chronic obstructive pulmonary disease (COPD), Irritable bowel syndrome (IBS), arthritis, psoriasis, eosinophilic esophagitis, eosinophilic pneumonia, eosinophilic psoriasis, hypereosinophilic syndrome, graft-versus-host disease, uveitis, cardiovascular disease, pain, lupus, vasculitis, chronic idiopathic urticaria, and Eosinophilic Granulomatosis with Polyangiitis (Churg-Strauss Syndrome).

3. The method of claim 1, wherein the at least one pro-inflammatory Th17 transcription factor or the at least one anti-inflammatory Th17 transcription factor is selected from the group consisting of: HEB, TCF1, Jun, Fosl2, Junb, Fos, Jund, Tfe3, Usf1, Usf2, Clock, Arntl, Arnt, Bhlhe40, Tfeb, Mxi1, Max, Myc, Nfyb, Nfya, Nfyc, Ddit3, Atf4, Cebpg, Zbtb7a, Mbd2, Epas1, Hif1a, Mecp2, Xbp1, Cux1, Zbtb33, E2f2, E2f5, Hinfp, Mtf1, Nrf1, Pax5, Yy1, Poulf1, Nfe2l1, Tead2, Arid5b, Sox5, Foxp2, Foxo1, Foxk1, Foxo3, Nrld1, Nr1d2, Rora, Rorc, Stat5a, Stat5b, Stat3, Stat4, Rel, Relb, Rela, Nfkb1, Nfkb2, Maff, Mafk, Maf, Mafg, Bach2, Nfe212, Bach1, Batf, Foxj3, Foxj2, Foxo4, Prdm1, Irf7, Irf9, Lyl1, Tcf12, Tcf3, Smad3, Smad2, Smad4, Mef2c, Mef2a, Mef2d, Rfx3, Rfx1, Rfx2, Rara, Rxra, Pparg, Nr1h3, Nr2c2, Ar, Nr3cl, Pbx1, Pknox1, Esrra, Esrrg, Esr1, Rarg, Stat6, Nfatc1, Nfatc2, Nfatc3, Ebf1, Prdm16, Atf2, Crem, Atf1, Creb1, Nr2c1, Rxrb, Mecom, Gata1, Gata3, Prdm5, Tcf4, Zeb1, Hsf1, Hsf2, Ikzf1, Ctcf, Zfx, Bcl6, Nfia, Nfic, Smarca5, Thap11, Dbp, Nfil3, Thra, Tbp, Pou2f1, Pou2f2, Zkscan1, Gfi1, Pbx2, Rest, Srf, Nr4a1, Prdm9, Rbpj, Tbx21, Srebf1, Srebf2, Taf1, Tbx20, Meis2, Tgif1, Maz, Klf3, Klf6, Sp1, Zbtb17, Sp4, Sp2, Sp3, Egr1, Egr2, E2f6, E2f1, E2f4, Spi1, Spib, Ets1, Elf1, Elk1, Gabpa, Elk4, Fli1, Ets2, Elf2, Etv6, Cbfb, Runx3, Runx1, Runx2, Lef1, Tcf7, Irf4, Irf8, Irf1, Irf2, Irf3, Stat1, and Stat2.

4. The method of claim 1, wherein the at least one pro-inflammatory Th17 transcription factor is selected from the group consisting of RUNX1, SP1, STAT3, IRF4, RORG, RORA, STAT1, BATF, JUNB, and FOSL2.

5. The method of claim 1, wherein the at least one pro-inflammatory Th17 transcription factor is selected from the group consisting of Lef1, Bach2, and Rora.

6. The method of claim 1, wherein the at least one anti-inflammatory Th17 transcription factor is selected from the group consisting of HEB, FOXO1, GABPA, FLI1, ELK4, EGR1, TCF1, and ETS1.

7. The method of claim 1, wherein the at least one anti-inflammatory Th17 transcription factor is TCF1.

8. The method of claim 4, wherein expression of P2rx7, P2rx4, Il17a, Il21r, Csf2, Il1r1, Fasl, Scd1, Hadh, Slc2a1, Akt2, 1121, Gzmb, Lck, Src, Rora, Rorc, Bhlhe40, Pparg, Abca1, Hk2, Pygl, Adam 19, Itgfb1, Cd47, Ccr6, Cxc4r6, Tnfsf14, Csf1, Bcl2, Tmem59, Slc2a3, Gpr65, Itgb7, Itgb3, or any combination thereof is decreased.

9. The method of claim 7, wherein expression of Abhd2, Osbp19, Ldlrap1, Pcca, Dmtf1, Btg1, Smc6, Fanci, Usp28, Bcor, Sp100, Foxo1, Dnmt3a, Ppm1g, Tgfbr2, Smad5, Il10rb, I110, Dusp2, Dusp4, Tgfbr3, Leftyl, or any combination thereof is increased.

10. The method of claim 1, wherein the exogenous T cell modulating agent increases TCF1 expression or inhibits TCF1 expression from decreasing.

11. The method of claim 1, wherein the exogenous T cell modulating agent that decreases expression of the at least one pro-inflammatory Th17 transcription factor is selected from the group consisting of:
a. one or more vectors for knocking out one or more genes from Table 1A and/or Table 1B;
b. a RNAi agent for decreasing expression of one or more genes from Table 1A and/or Table 1B;
c. a genetic modification system linked to or otherwise capable of complexing with a transcription repressor that blocks transcription of one or more genes from Table 1A and/or Table 1B;
d. a genetic modification system capable of making one or more epigenetic edits to a cell genome such that expression of one or more genes from Table 1A and/or Table 1B is silenced;
e. a genetic modification system configured to modify one or more enhancers controlling expression of one or more genes in Table 1A and/or Table 1B such that binding of transcription factors or other regulatory proteins needed to initiate transcription is reduced;
f. a genetic modification system configured to remove or modify one or more promoters controlling expression of one or more genes in Table 1A and/or Table 1B, or configured to replace one or more promoters with a weakened promoter, such that binding of transcription factors and/or RNA polymerase binding is blocked or weakened;
g. a genetic modification system configured to introduce a silencer element, or to modify an existing silencer element, in a non-coding region controlling expression of one or more genes from Table 1A and/or Table 1B, such that transcriptional repressors that block or decrease expression are recruited to the non-coding region;
h. a genetic modification system configured to disrupt or replace an existing insulator region controlling expression of one or more genes from Table 1A and/or Table 1B such that insulator function on silencer elements or repressive chromatin structures is reduced;
i. a genetic modification system configured to introduce a frame-shift mutation in a coding region of one or more genes from Table 1A and/or Table 1B such that a pre-mature stop codon is introduced;
j. a genetic modification system configured to introduce insertions, deletions, or substitutions in coding regions of one or more genes from Table 1A and/or Table 1B that encode one or more functional domains of the expressed gene product such that a non-functional gene or gene products with reduced function or activity are produced;
k. a genetic modification system configured to introduce one or more modification at one or more exon-intron boundaries or splice sites leading to aberrant splicing or production of non-functional proteins or triggering of non-sense mediated RNA decay;
l. a genetic modification system configured to introduce one or more modifications to a regulatory element within the coding regions of one or more genes from Table 1A and/or Table 1B such the expression is decreased;
m. a genetic modification system configured to modify sequences encoding one or more post-translational modification (PTM) sites such that one or more activating PTM is removed, or one or more inhibitory PTM that decreases protein function or stability or increases protein degradation is increased;
n. a genetic modification system configured to introduce or remove one or more chromatin loops controlling expression of one or more genes in Table 1A and/or Table 1B such that gene expression is reduced; and
o. combinations thereof.

12. The method of claim 11, wherein the genetic modification system linked to or otherwise capable of complexing with a transcription repressor that blocks transcription is catalytically inactive;
wherein the genetic modification system for (d)-(n) is a CRISPR-Cas system, a base editing system, a prime editing system, a CAST system, a non-LTR retrotransposon, or a Cas-directed recombinase; or
wherein the genetic modification system for (d) is catalytically inactive Cas linked to or otherwise associated with an epigenetic modifier.

13. The method of claim 1, wherein the exogenous T cell modulating agent which increases expression of at least one anti-inflammatory Th17 transcription factor is selected from the group consisting of:
a. one or more vectors for introducing one or more additional copies of one or more genes from Table 2A and/or Table 2B;
b. a genetic modification system configured to modify one or more enhancer regions controlling expression of one or more genes in Table 2A and/or Table 2B such that binding of transcription factors or other regulatory proteins is increased;
c. a genetic modification system configured to modify one or more promoters controlling expression of one or more genes of Table 2A and/or Table 2B such that expression is increased;
d. a genetic modification system configured to modify one or more silencer regions controlling expression of one or more genes in Table 2A and/or Table 2B, such that binding of transcriptional repressor is blocked or reduced and gene expression is increased;
e. a genetic modification system configured to modify or remove one or more regulatory elements in a coding sequence of one or more genes in Table 2A and/or Table 2B;
f. a genetic modification system configured to introduce or strengthen one or more insulator regions controlling expression of one or more genes in Table 2A and/or Table 2B;
g. a genetic modification system configured to introduce one or more modifications in a coding region that increases gene product stability, folding, or resistance to degradation or enhances the function or activity of the gene product;
h. a genetic modification system configured to remove one or more sequences encoding an inhibitory PTM, remove one or more ubiquitination sites, and/or add one or more activating PTM sites;
i. a genetic modification system configured to introduce or remove one or more chromatin loops controlling expression of one or more genes in Table 2A and/or Table 2B such that gene expression is reduced; and
j. combinations thereof.

14. The method of claim 13, wherein the genetic modification system for any one of (b)-(i) is a CRISPR-Cas system, a base editing system, a prime editing system, a CAST system, a non-LTR retrotransposon, or a Cas-directed recombinase.

15. An engineered immune cell modified to:
a. decrease expression of one or more genes selected from Table 1A and/or one or more transcription factors selected from Table 1B;
b. increase expression of one or more genes selected from Table 2A and/or one or more transcription factors selected from Table 2B; or
c. a combination thereof.

16. The engineered immune cell of claim 15, wherein the immune cell is an iPSC or a hematopoietic stem cell.

17. The engineered immune cell of claim 15, wherein the immune cell is a CD4+ T cell.

18. A pharmaceutical composition comprising the engineered immune cell of claim 15.

19. A method of treating an inflammatory disorder comprising administering to a subject in need thereof the engineered cell of claim 15.

20. A method of preparing an engineered immune cell for use in therapeutic compositions, comprising: administering one or more agents to the immune cell that can decrease expression of one or more genes selected from Table 1A and/or Table 1B, or increase expression of one or more genes selected from Table 2A and/or Table 2B, or a combination thereof.


SEQUENCE LISTING (49 sequences)

SEQ ID NO: 1-26 - skipped (26 entries)

SEQ ID NO: 27 - protein, 7 aa, synthetic construct
  REGION 1..7: note = nuclear localization sequence
PKKKRKV

SEQ ID NO: 28 - protein, 10 aa, synthetic construct
  REGION 1..10: note = nuclear localization sequence
PKKKRKVEAS

SEQ ID NO: 29 - protein, 16 aa, synthetic construct
  REGION 1..16: note = nuclear localization sequence
KRPAATKKAGQAKKKK

SEQ ID NO: 30 - protein, 9 aa, synthetic construct
  REGION 1..9: note = nuclear localization sequence
PAAKRVKLD

SEQ ID NO: 31 - protein, 11 aa, synthetic construct
  REGION 1..11: note = nuclear localization sequence
RQRRNELKRSP

SEQ ID NO: 32 - protein, 38 aa, synthetic construct
  REGION 1..38: note = nuclear localization sequence
NQSSNFGPMKGGNFGGRSSGPYGGGGQYFAKPRNQGGY

SEQ ID NO: 33 - protein, 42 aa, synthetic construct
  REGION 1..42: note = nuclear localization sequence
RMRIZFKNKGKDTAELRRRRVEVSVELRKAKKDEQILKRRNV

SEQ ID NO: 34 - protein, 8 aa, synthetic construct
  REGION 1..8: note = nuclear localization sequence
VSRKRPRP

SEQ ID NO: 35 - protein, 8 aa, synthetic construct
  REGION 1..8: note = nuclear localization sequence
PPKKARED

SEQ ID NO: 36 - protein, 8 aa, synthetic construct
  REGION 1..8: note = nuclear localization sequence
PQPKKKPL

SEQ ID NO: 37 - protein, 12 aa, synthetic construct
  REGION 1..12: note = nuclear localization sequence
SALIKKKKKMAP

SEQ ID NO: 38 - protein, 5 aa, synthetic construct
  REGION 1..5: note = nuclear localization sequence
DRLRR

SEQ ID NO: 39 - protein, 7 aa, synthetic construct
  REGION 1..7: note = nuclear localization sequence
PKQKKRK

SEQ ID NO: 40 - protein, 10 aa, synthetic construct
  REGION 1..10: note = nuclear localization sequence
RKLKKKIKKL

SEQ ID NO: 41 - protein, 10 aa, synthetic construct
  REGION 1..10: note = nuclear localization sequence
REKKKFLKRR

SEQ ID NO: 42 - protein, 20 aa, synthetic construct
  REGION 1..20: note = nuclear localization sequence
KRKGDEVDGVDEVAKKKSKK

SEQ ID NO: 43 - protein, 17 aa, synthetic construct
  REGION 1..17: note = nuclear localization sequence
RKCLQAGMNLEARKTKK

SEQ ID NO: 44 - protein, 288 aa, synthetic construct
  REGION 1..288: note = N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 45 - protein, 183 aa, synthetic construct
  REGION 1..183: note = C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 46 - DNA, 42 bp, synthetic construct
  misc_feature 1..42: note = LRNA processing element
  source 1..42: mol_type = other DNA
ggctcgttgggaggtcccgggttgaaatcccggacgagcccg

SEQ ID NO: 47 - DNA, 100 bp, synthetic construct
  misc_feature 1..100: note = human 28s processing element
  source 1..100: mol_type = other DNA
tagccaaatgcctcgtcatctaattagtgacgcgcatgaatggatgaacgagattcccactgtccctacctactatccagcgaaaccacagccaagggaa

SEQ ID NO: 48 - DNA, 125 bp, Bombyx mori
  source 1..125: mol_type = genomic DNA
tagccaaatgcctcgtcatctaattagtgacgcgcatgaatggattaacgagattcccactgtccctatctactatctagcgaaaccacagccaagggaacgggcttgggagaatcagcggggaa

SEQ ID NO: 49 - DNA, 14 bp, synthetic construct
  misc_feature 1..14: note = CTCF binding motif
  misc_difference 5: note = n is a, c, g, or t
  source 1..14: mol_type = other DNA
ccacnaggtggcag